SEQUENCE LISTING (2 sequences)

SEQ ID NO: 1 - protein, 169 aa, Homo sapiens
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 486 aa, Homo sapiens
EEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

(19) United States
(12) Patent Application Publication
(10) Pub. No.: US 2022/0324862 A1
(43) Pub. Date: Oct. 13, 2022

(54) PYRIDOPYRIMIDINONE COMPOUNDS

(71) Applicant: Acerand Therapeutics (USA) Limited, Carmel, IN (US)

(72) Inventors: Kuo-Long Yu, Zionsville, IN (US); Cen Gao, Carmel, IN (US); Sanjeev Kumar, Waverly, IA (US); Bin Liu, Fishers, IN (US)

(21) Appl. No.: 17/709,021

(22) Filed: Mar. 30, 2022

Related U.S. Application Data
(60) Provisional application No. 63/168,408, filed on Mar. 31, 2021.

(51) Int. Cl. C07D 471/04; C07D 519/00; A61P 35/00

(52) U.S. Cl. CPC C07D 471/04; C07D 519/00; A61P 35/00

(57) ABSTRACT

Disclosed are compounds of formula (I):

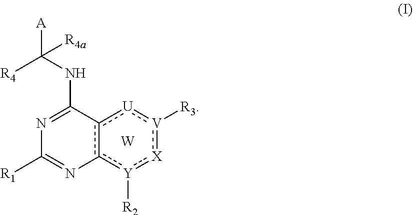

Variables R₁-R₄, R₄ₐ, A, U, V, X, and Y and ring W are defined herein. Also disclosed are a pharmaceutical composition containing such a compound and methods of using the compound for treating cancer and inhibiting SOS1.

### CROSS REFERENCE TO RELATED APPLICATION

The present application claims the benefit of priority to U.S. Provisional Application Ser. No. 63/168,408, filed on Mar. 31, 2021, the disclosure of which is hereby incorporated by reference in its entirety.

### BACKGROUND

The RAS family of GTPases, including KRAS (V-Ki-ras2 Kirsten rat sarcoma viral oncogene homolog), NRAS (Neuroblastoma RAS viral oncogene homolog), and HRAS (Harvey murine sarcoma virus oncogene), are recognized as major oncogenes, occurring in up to 20 to 30% of human cancers.

Due to very high affinity of RAS proteins for GDP/GTP, the exchange from RAS bound GDP to GTP is catalyzed by guanine nucleotide exchange factors (GEFs) such as Son of Sevenless 1 (SOS1) whereas the return to its inactive state (RAS-GDP bound form) is catalyzed by GTPase-activating proteins (GAPs). Once activated RAS in its GTP bound state interacts with a number of effectors to drive cell growth and differentiation.

In cancer cells, oncogenic activating mutations in RAS proteins, and/or the isoform SOS1, or inactivating mutations in GAPs result in constitutive activation of the RAS signaling pathway, which in turn leads to uncontrolled cancer cell proliferation and growth. Cancer patients with RAS mutations often have aggressive, metastatic disease with poor prognoses. Direct inhibition of RAS has proved extremely challenging. Alternative strategies to indirectly target the RAS signaling pathways have been explored, for example, inhibiting the enzymatic activity of GEFs such as SOS1 and or its interaction with RAS. These approaches should also lead to the attenuation of the RAS signaling pathway activity by preventing the formation of the active RAS-GTP bound form. Therefore, inhibition of SOS1 activity or its interaction with RAS is of therapeutic benefit. Indeed, certain benzylamino-substituted-pyridopyrimidione compounds and quinazoline compounds were developed as SOS1 inhibitors. See WO 2019/122129 and Hillig et al, Proc Natl Acad Sci USA, 2019, vol. 116(7), 2551-2560.

Nevertheless, there is still a large unmet need to develop SOS1 inhibitors for treating cancers and other diseases associated with or modulated by the SOS1 interaction with KRAS including cancers that harbor genetic alterations (mutations, fusions, translocations, amplification, and over-expression) in genes encoding ALK, AxL, BCR-ABL, c-Raf, c-Met, EGFR1-4, ErbB2, FGFR 1-4, Kras, NRas, HRas, NF1, NTRK, Ret, ROS, and other oncogenic signaling molecules.

### SUMMARY

The present invention is based on an unexpected discovery that certain pyridopyrimidinone compounds are effective SOS1 inhibitors, suitable for treating cancer and other diseases such as neurofibromatosis, Noonan syndrome (NS), cardiofaciocutaneous syndrome (CFC), and hereditary gingival fibromatosis type 1.

In one aspect, this invention relates to compounds of formula (I):

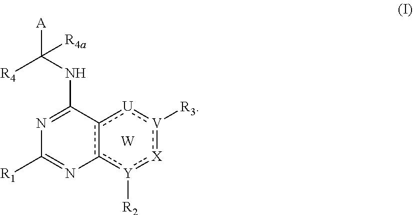

In this formula, R₁ is H, D, —CD₃, halogen, —CN, —CONHR₁ₐ, —NHR₁ₐ, —OR₁ₐ, C₁₋₆ alkyl, C₃₋₆ cycloalkyl, C₁₋₆ heterocycloalkyl, C₂₋₄ alkenyl, or C₂₋₄ alkynyl, in which R₁ₐ is H, C₁₋₆ alkyl, C₃₋₆ cycloalkyl, or C₁₋₆ heterocycloalkyl;

R₂ is deleted, H, D, —CD₃, halogen, —CN, C₁₋₆ alkyl, C₁₋₆ alkoxy, C₃₋₆ cycloalkyl, C₁₋₆ heterocycloalkyl, C₂₋₆ alkenyl, or C₂₋₆ alkynyl;

R₃ is L₁-R₅;

R₄ is C₂₋₄ alkenyl or C₂₋₄ alkynyl;

R₄ₐ is H or D;

L₁ is a bond, —C(O)—, —C(O)O—, —C(O)NH(CH₂)_{q}—, —S—, —S(O)₂—, —S(O)—, —C(O)NR₆—, —SO₂NR₆—, —NR₆—, —NR₆C(O), —NR₆S(O)₂—, —NHC(O)NR₆—, —NHC(O)—, —NH(CH₂)_{q}NHC(O)—, —NH(CH₂)_{q}—, —O—, —O—(CH₂)_{q}—, C₂₋₄ alkylene, C₂₋₄ alkenylene, or C₂₋₄ alkynylene, in which q is 0, 1, or 2;

R₅ is H, C₁₋₁₀ alkyl, C₃₋₁₀ cycloalkyl, C₁₋₁₀ heterocycloalkyl, C₂₋₁₀ alkenyl, C₂₋₁₀ alkynyl, aryl, or heteroaryl;

R₆ is H, C₁₋₁₀ alkyl, C₃₋₁₀ cycloalkyl, C₁₋₁₀ heterocycloalkyl, aryl, or heteroaryl;

A is aryl, heteroaryl, C₃₋₁₀ cycloalkyl, or C₁₋₁₀ heterocycloalkyl;

[symbol] is a single bond or a double bond, provided that the number of double bonds represented by [symbol] is 1, 2, or 3;

each of U and X, independently, is CH, C═O, or N;

each of V and Y, independently, is C or N; and

ring W is

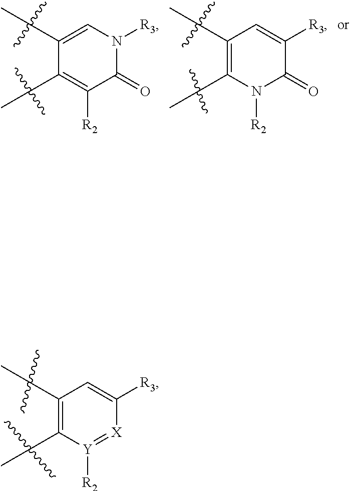

in which X is CH or N and Y is C or N, provided that when Y is N, R₂ is deleted.

A subset of the compounds of formula (I) includes the compounds of formula (IA):

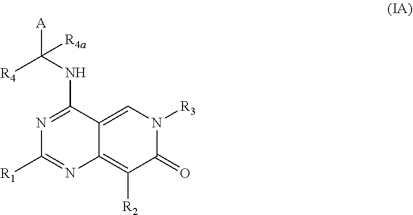

in which

R₁ is H, D, —CD₃, halogen, —CN, —NHR₁ₐ, —OR₁ₐ, C₁₋₃ alkyl, cyclopropyl, or C₂₋₃ alkenyl, R₁ₐ being H, C₁₋₃ alkyl, C₂₋₃ alkenyl, or cyclopropyl;

R₂ is H, D, —CD₃, halogen, —CN, —OR₁ₐ, C₁₋₄ alkyl, C₃₋₆ cycloalkyl, or C₁₋₆ heterocycloalkyl;

R₃ is L₁-R₅;

R₄ is C₂₋₄ alkenyl or C₂₋₄ alkynyl;

R₄ₐ is H or D;

L₁ is a bond or —NR₆—;

R₅ is C₁₋₁₀ alkyl, C₃₋₁₀ cycloalkyl, C₁₋₁₀ heterocycloalkyl, C₂₋₁₀ alkenyl, C₂₋₁₀ alkynyl, aryl, or heteroaryl;

R₆ is H, C₁₋₁₀ alkyl, C₃₋₁₀ cycloalkyl, C₁₋₁₀ heterocycloalkyl, aryl, or heteroaryl; and A is aryl or heteroaryl.

In certain preferred compounds, one or more of variables R₁-R₄ and A can be defined as follows:

R₁ is H, D, halogen, —CD₃, —CN, —NHR₁ₐ, —OR₁ₐ, C₁₋₃ alkyl, cyclopropyl, or C₂₋₃ alkenyl (e.g., H and methyl);

R₂ is H, D, halogen, —CD₃, —CN, —OR₁ₐ, C₁₋₄ alkyl, C₃₋₆ cycloalkyl, or C₁₋₆ heterocycloalkyl (e.g., H, halogen, and methyl).

R₃ is L₁-R₅, L₁ being a bond or —NR₆—, in which R₆ is H, C₁₋₃ alkyl, or C₃₋₆ cycloalkyl, and R₅ being C₁₋₁₀ alkyl, C₃₋₁₀ cycloalkyl, C₁₋₁₀ heterocycloalkyl, aryl, or heteroaryl;

R₄ is

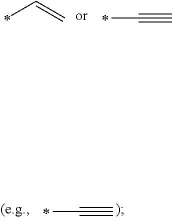

and

A is phenyl or phenyl fused with a C₄₋₆ cycloalkyl, C₁₋₆ heterocycloalkyl, or heteroaryl.

Optionally, each of phenyl and fused phenyl is substituted with one or more moieties selected from the group consisting of halogens, —OH, —NH₂, —CN, methyl, ethyl, C₁₋₃ alkoxy, C₁₋₃ haloalkoxy, fluoromethyl, difluoromethyl, trifluoromethyl, fluoroethyl, difluoroethyl, trifluoroethyl,

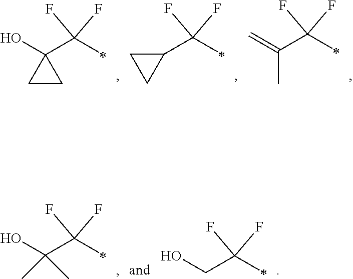

Specific examples of A include:

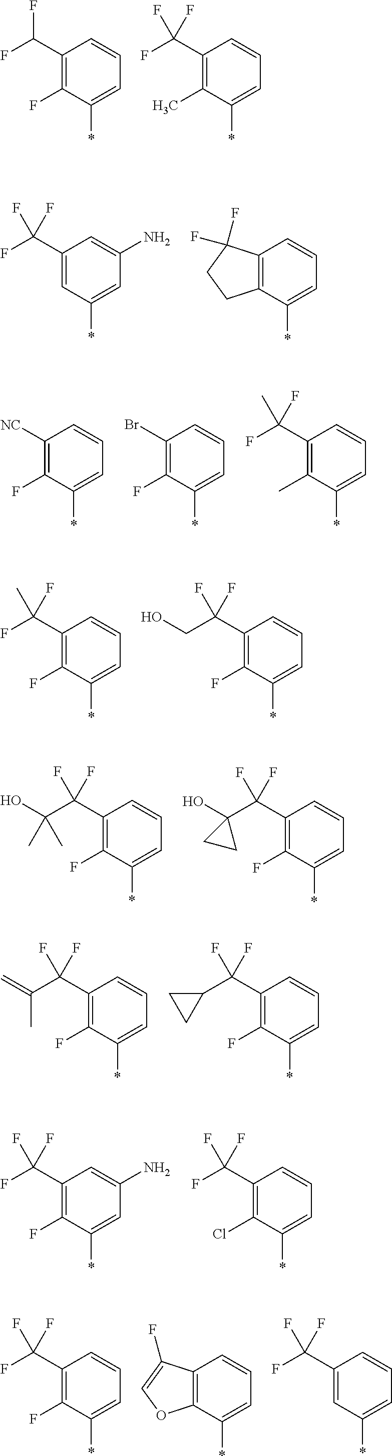

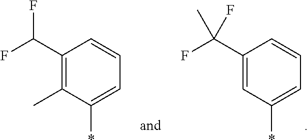 [SMILES: *c1cccc(C(F)F)c1C ; *c1cccc(C(C)(F)F)c1]

Preferably, A is:

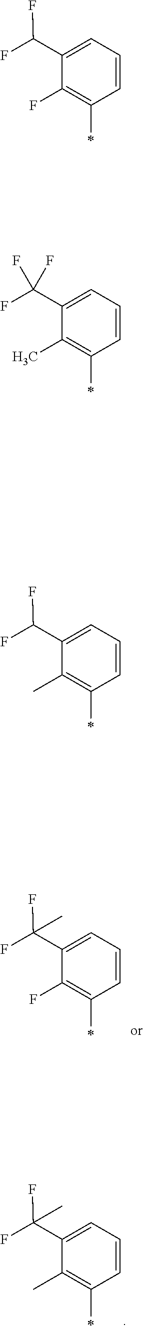

Turning to R₃, it can be:

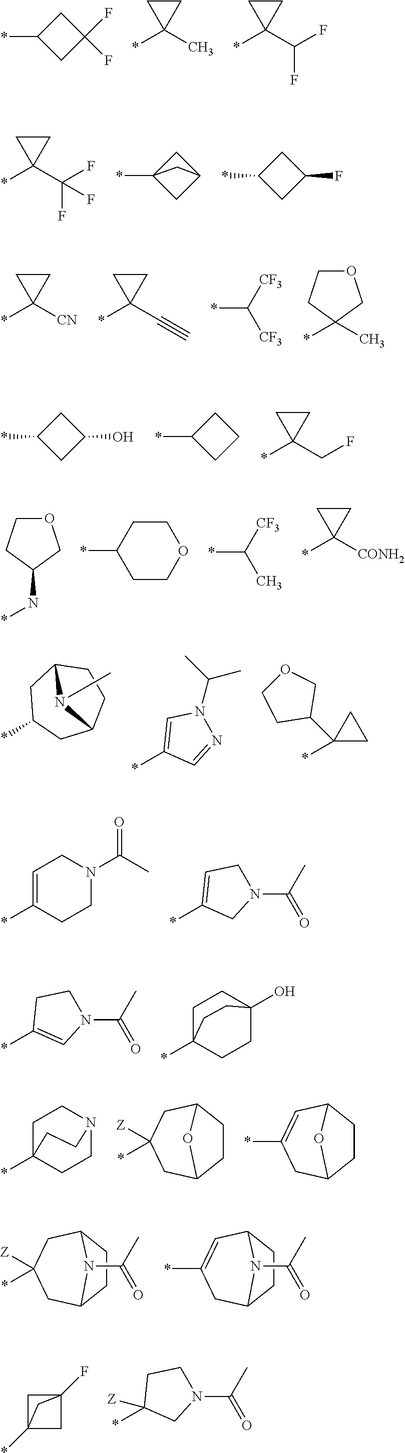

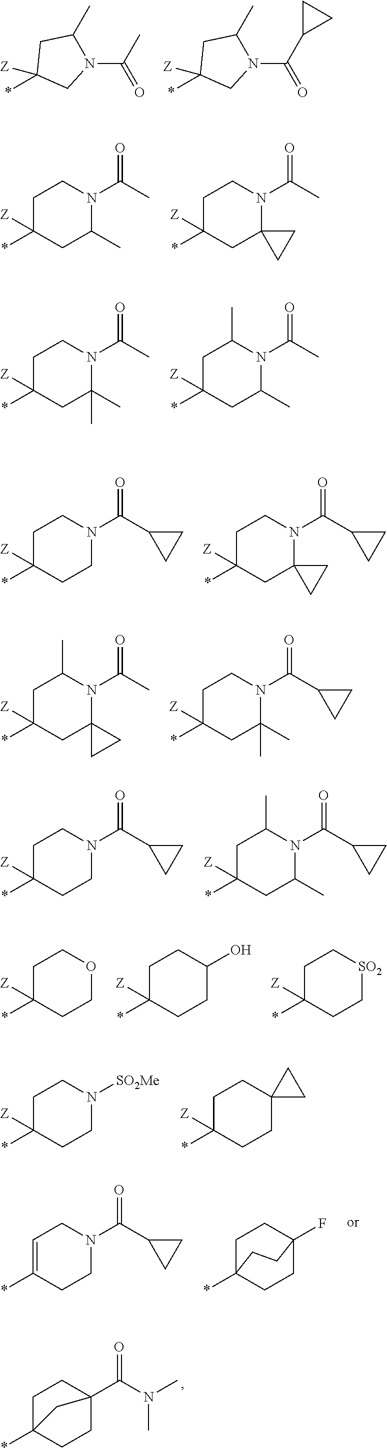

in which Z is H or methyl.

Preferably, R₃ is

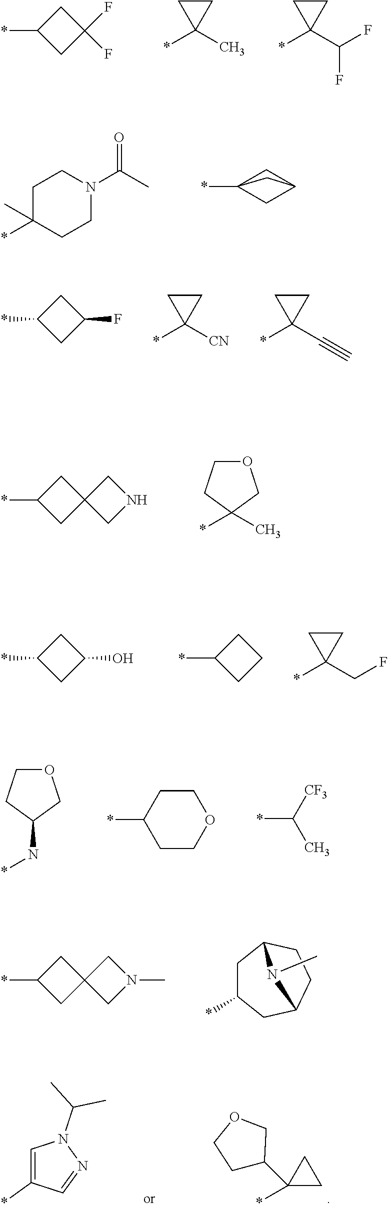

More preferably, R₃ is

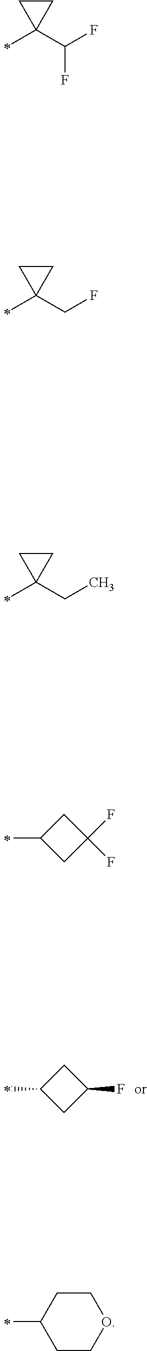

Another subset of the compounds of formula (I) includes the compounds of formula (IB):

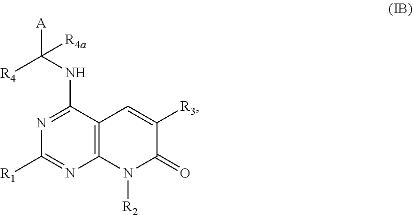

in which

R₁, R₄, R₄ₐ, and A are as defined above;

R₂ is H, CD₃, C₁₋₄ alkyl, C₃₋₆ cycloalkyl, or C₁₋₆ heterocycloalkyl;

R₃ is L₁-R₅;

L₁ is a bond, —C(O)—, —C(O)O—, —C(O)NH(CH₂)_{q}—, —S—, —S(O)₂—, —S(O)—, —C(O)NR₆—, —SO₂NR₆—, —NR₆—, —NR₆S(O)—, —NHC(O)NR₆—, —NHC(O)—, —NH(CH₂)_{q}NHC(O)—, —NH(CH₂)_{q}—, —O—, —O—(CH₂)_{q}—, C₁₋₄ alkylene, C₂₋₄ alkenylene, or C₂₋₄ alkynylene;

R₅ is C₁₋₁₀ alkyl, C₃₋₁₀ cycloalkyl, C₁₋₁₀ heterocycloalkyl, C₂₋₁₀ alkenyl, C₂₋₁₀ alkynyl, aryl, or heteroaryl; and

R₆ is H, C₁₋₁₀ alkyl, C₃₋₁₀ cycloalkyl, C₁₋₁₀ heterocycloalkyl, aryl, or heteroaryl.

In certain preferred compounds, one or more of variables R₁-R₄ and A can be defined as follows:

R₁ is H, D, CD₃, halogen, —CN, —NHR₁ₐ, —OR₁ₐ, C₁₋₃ alkyl, cyclopropyl, or C₂₋₃ alkenyl (e.g., H and methyl);

R₂ is H, CD₃, C₁₋₄ alkyl, C₃₋₆ cycloalkyl or C₁₋₆ heterocycloalkyl (e.g., H, methyl, or cyclopropyl);

R₃ is L₁-R₅, L₁ being a bond, —NR₆—, —O—, —NR₆C(O)—, or —C(O)NR₆—, in which R₆ is H, C₁₋₃ alkyl, or C₃₋₆ cycloalkyl, and R₅ being C₁₋₁₀ alkyl, C₃₋₁₀ cycloalkyl, C₁₋₁₀ heterocycloalkyl, aryl, or heteroaryl;

### R₄ is

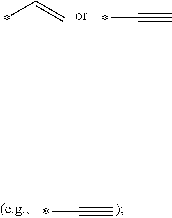

and

A is phenyl or phenyl fused with a C₄₋₆ cycloalkyl, C₁₋₆ heterocycloalkyl, or heteroaryl.

Optionally, each of phenyl and fused phenyl is substituted with one or more moieties selected from the group consisting of halogens, —OH, —NH₂, —CN, methyl, ethyl, C₁₋₃ alkoxy, C₁₋₃ haloalkoxy, fluoromethyl, difluoromethyl, trifluoromethyl, fluoroethyl, difluoroethyl, trifluoroethyl,

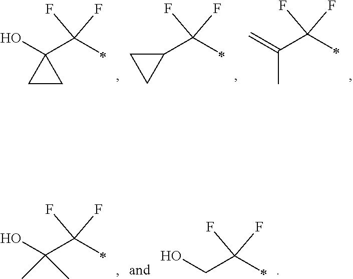

Exemplary A moieties are shown above.

Preferably, A is:

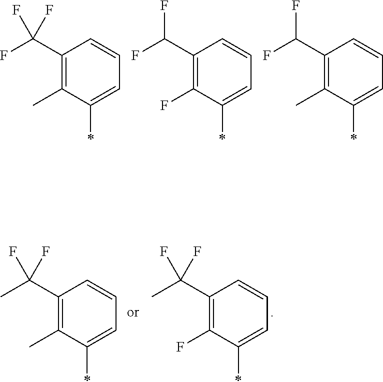

Similarly, R₃ can be:

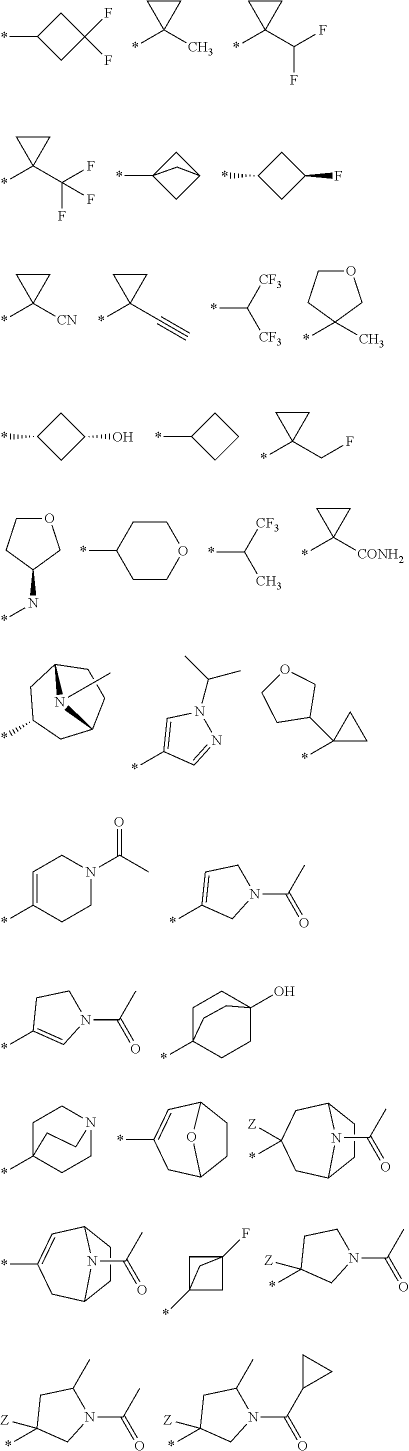

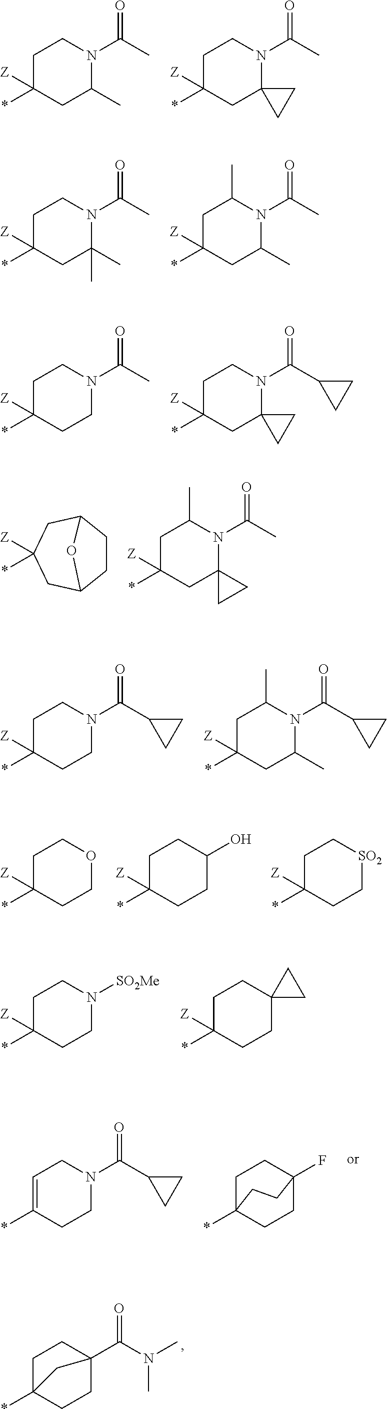

in which Z is H, methyl, methoxy, F, —OH, —CN, —NH₂, or —NH—COCH₃.

Preferably, R₃ is

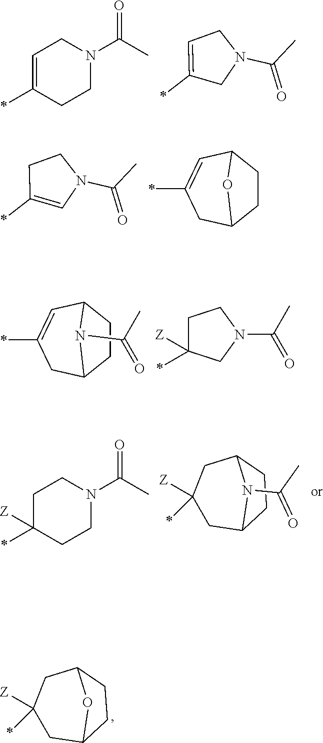

in which Z is H, methyl, methoxy, F, —OH, —CN, —NH₂, or —NH—COCH₃.

Another aspect of this invention relates to a pharmaceutical composition containing any of the compounds described above and a pharmaceutically acceptable carrier thereof.

Also within the scope of this invention is a method of treating cancer including the step of administering to a subject in need thereof an effective amount of any of the compounds described above.

Still within the scope of this invention is a method of inhibiting SOS1 by administering to a subject in need thereof an effective amount of any of the above-described compound.

Table 1 below shows 69 exemplary compounds of the present invention, i.e., Compounds 1-69, together with their structures and names.

TABLE 1

| Compound | | |
|---|---|---|
| No. | Structure | Name |
| 1 | 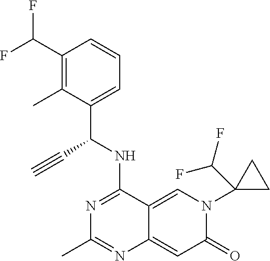 [SMILES: C#C[C@@H](Nc1nc(C)nc2cc(=O)n(C3(C(F)F)CC3)cc12)c1cccc(C(F)F)c1C] | (R)-4-((1-(3-(difluoromethyl)-2- methylphenyl)prop-2-yn-1-yl)amino)-6- (1-(difluoromethyl)cyclopropyl)-2- methylpyrido[4,3-d]pyrimidin-7(6H)- one |
| 2 | 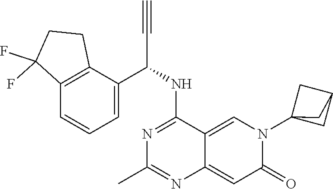 [SMILES: C#C[C@H](Nc1nc(C)nc2cc(=O)n(C34CC(C3)C4)cc12)c1cccc2c1CCC2(F)F] | (R)-6-(bicyclo[1,1,1]pentan-1-yl)-4-((1- (1,1-difluoro-2,3-dihydro-1H-inden-4- yl)prop-2-yn-1-yl)amino)-2- methylpyrido[4,3-d]pyrimidin-7(6H)- one |
| 3 | 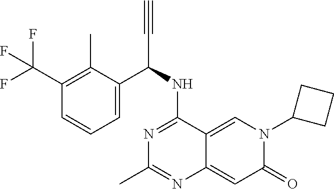 [SMILES: C#C[C@@H](Nc1nc(C)nc2cc(=O)n(C3CCC3)cc12)c1cccc(C(F)(F)F)c1C] | (R)-6-cyclobutyl-2-methyl-4-((1-(2- methyl-3-(trifluoromethyl)phenyl)prop- 2-yn-1-yl)amino)pyrido[4,3- d]pyrimidin-7(6H)-one |
| 4 | 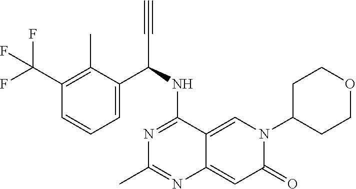 [SMILES: C#C[C@@H](Nc1nc(C)nc2cc(=O)n(C3CCOCC3)cc12)c1cccc(C(F)(F)F)c1C] | (R)-2-methyl-4-((1-(2-methyl-3- (trifluoromethyl)phenyl)prop-2-yn-1- yl)amino)-6-(tetrahydro-2H-pyran-4- yl)pyrido[4,3-d]pyrimidin-7(6H)-one |
| 5 | 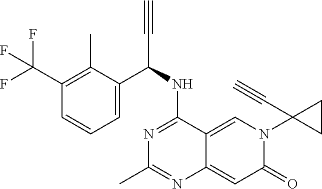 [SMILES: C#C[C@@H](Nc1nc(C)nc2cc(=O)n(C3(C#C)CC3)cc12)c1cccc(C(F)(F)F)c1C] | (R)-6-(1-ethynylcyclopropyl)-2-methyl- 4-((1-(2-methyl-3- (trifluoromethyl)phenyl)prop-2-yn-1- yl)amino)pyrido[4,3-d]pyrimidin-7(6H)- one |
| 6 | 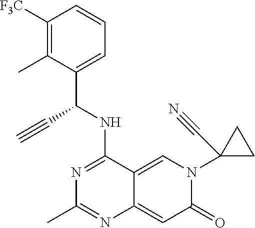 [SMILES: C#C[C@@H](Nc1nc(C)nc2cc(=O)n(C3(C#N)CC3)cc12)c1cccc(C(F)(F)F)c1C] | (R)-1-(2-methyl-4-((1-(2-methyl-3- (trifluoromethyl)phenyl)prop-2-yn-1- yl)amino)-7-oxopyrido[4,3-d]pyrimidin- 6(7H)-yl)cyclopropane-1-carbonitrile |
| 7 | 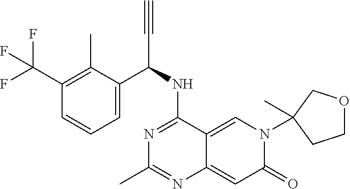 [SMILES: C#C[C@@H](Nc1nc(C)nc2cc(=O)n(C3(C)CCOC3)cc12)c1cccc(C(F)(F)F)c1C] | 2-methyl-4-(((R)-1-(2-methyl-3- (trifluoromethyl)phenyl)prop-2-yn-1- yl)amino)-6-(3-methyltetrahydrofuran- 3-yl)pyrido[4,3-d]pyrimidin-7(6H)-one (a mixture of diasteromers) |
| 8 | 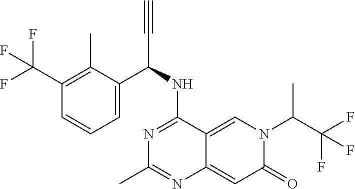 [SMILES: C#C[C@@H](Nc1nc(C)nc2cc(=O)n(C(C)C(F)(F)F)cc12)c1cccc(C(F)(F)F)c1C] | 2-methyl-4-(((R)-1-(2-methyl-3- (trifluoromethyl)phenyl)prop-2-yn-1- yl)amino)-6-(1,1,1-trifluoropropan-2- yl)pyrido[4,3-d]pyrimidin-7(6H)-one (a mixture of diastereomers) |
| 9 | 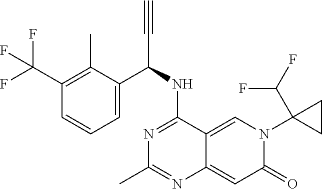 [SMILES: C#C[C@@H](Nc1nc(C)nc2cc(=O)n(C3(C(F)F)CC3)cc12)c1cccc(C(F)(F)F)c1C] | (R)-6-(1-(difluoromethyl)cyclopropyl)- 2-methyl-4-((1-(2-methyl-3- (trifluoromethyl)phenyl)prop-2-yn-1- yl)amino)pyrido[4,3-d]pyrimidin-7(6H)- one |
| 10 | 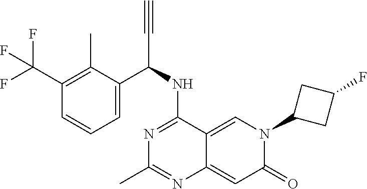 | 6-((1S,3S)-3-fluorocyclobutyl)-2- methyl-4-(((R)-1-(2-methyl-3- (trifluoromethyl)phenyl)prop-2-yn-1- yl)amino)pyrido[4,3-d]pyrimidin-7(6H)- one |
| 11 | 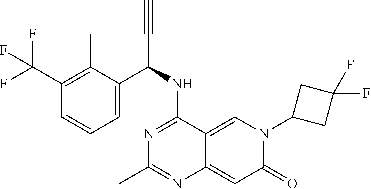 [SMILES: C#C[C@@H](Nc1nc(C)nc2cc(=O)n(C3CC(F)(F)C3)cc12)c1cccc(C(F)(F)F)c1C] | (R)-6-(3,3-difluorocyclobutyl)-2- methyl-4-((1-(2-methyl-3- (trifluoromethyl)phenyl)prop-2-yn-1- yl)amino)pyrido[4,3-d]pyrimidin-7(6H)- one |
| 12 | 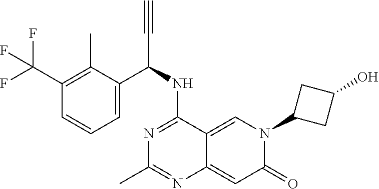 | 6-((1S,3S)-3-hydroxycyclobutyl)-2- methyl-4-(((R)-1-(2-methyl-3- (trifluoromethyl)phenyl)prop-2-yn-1- yl)amino)pyrido[4,3-d]pyrimidin-7(6H)- one |
| 13 | 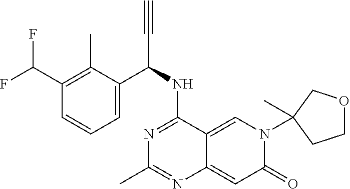 [SMILES: C#C[C@@H](Nc1nc(C)nc2cc(=O)n(C3(C)CCOC3)cc12)c1cccc(C(F)F)c1C] | 4-(((R)-1-(3-(difluoromethyl)-2- methylphenyl)prop-2-yn-1-yl)amino)-2- methyl-6-(3-methyltetrahydrofuran-3- yl)pyrido[4,3-d]pyrimidin-7(6H)-one (mixture of diastereomers) |
| 14 | 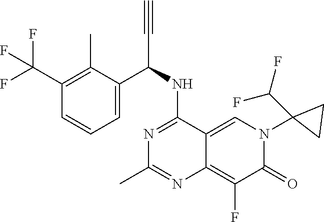 [SMILES: C#C[C@@H](Nc1nc(C)nc2c(F)c(=O)n(C3(C(F)F)CC3)cc12)c1cccc(C(F)(F)F)c1C] | (R)-6-(1-(difluoromethyl)cyclopropyl)- 8-fluoro-2-methyl-4-((1-(2-methyl-3- (trifluoromethyl)phenyl)prop-2-yn-1- yl)amino)pyrido[4,3-d]pyrimidin-7(6H)- one |
| 15 | 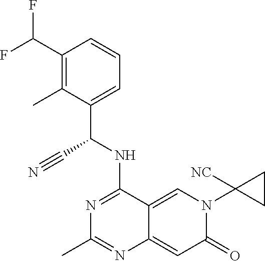 [SMILES: Cc1nc(N[C@H](C#N)c2cccc(C(F)F)c2C)c2cn(C3(C#N)CC3)c(=O)cc2n1] | (R)-1-(4-((1-(3-(difluoromethyl)-2- fluorophenyl)prop-2-yn-1-yl)amino)-2- methyl-7-oxopyrido[4,3-d]pyrimidin- 6(7H)-yl)cyclopropane-1-carbonitrile |
| 16 | 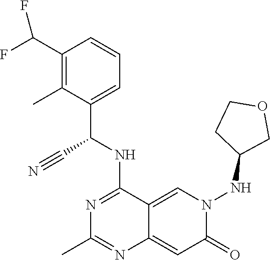 [SMILES: Cc1nc(N[C@H](C#N)c2cccc(C(F)F)c2C)c2cn(N[C@H]3CCOC3)c(=O)cc2n1] | 4-(((R)-1-(3-(difluoromethyl)-2- methylphenyl)prop-2-yn-1-yl)amino)-2- methyl-6-(((S)-tetrahydrofuran-3- yl)amino)pyrido[4,3-d]pyrimidin-7(6H)- one |
| 17 | 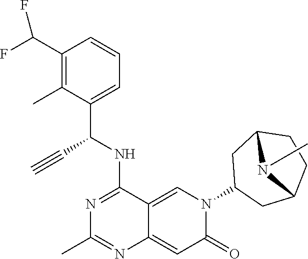 | 4-(((R)-1-(3-(difluoromethyl)-2- methylphenyl)prop-2-yn-1-yl)amino)-2- methyl-6-((1R,3r,5S)-8-methyl-8- azabicyclo[3.2.1]octan-3-yl)pyrido[4,3- d]pyrimidin-7(6H)-one |
| 18 | 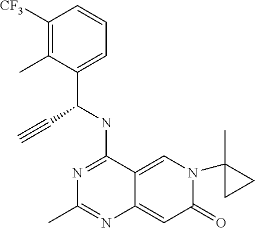 [SMILES: C#C[C@@H](Nc1nc(C)nc2cc(=O)n(C3(C)CC3)cc12)c1cccc(C(F)(F)F)c1C] | (R)-2-methyl-4-((1-(2-methyl-3- (trifluoromethyl)phenyl)prop-2-yn-1- yl)amino)-6-(1- methylcyclopropyl)pyrido[4,3- d]pyrimidin-7(6H)-one |
| 19 | 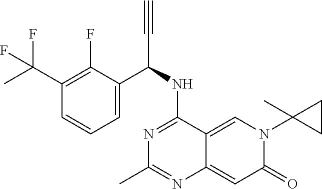 [SMILES: C#C[C@@H](Nc1nc(C)nc2cc(=O)n(C3(C)CC3)cc12)c1cccc(C(C)(F)F)c1F] | (R)-4-((1-(3-(1,1-difluoroethyl)-2- fluorophenyl)prop-2-yn-1-yl)amino)-2- methyl-6-(1- methylcyclopropyl)pyrido[4,3- d]pyrimidin-7(6H)-one |
| 20 | 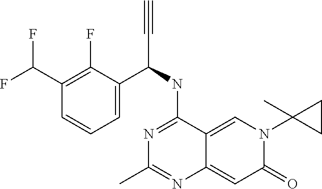 [SMILES: C#C[C@@H](Nc1nc(C)nc2cc(=O)n(C3(C)CC3)cc12)c1cccc(C(F)F)c1F] | (R)-4-((1-(3-(difluoromethyl)-2- fluorophenyl)prop-2-yn-1-yl)amino)-2- methyl-6-(1- methylcyclopropyl)pyrido[4,3- d]pyrimidin-7(6H)-one |
| 21 | 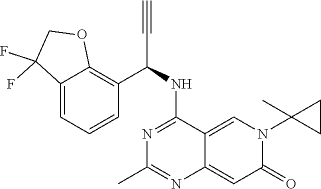 [SMILES: C#C[C@@H](Nc1nc(C)nc2cc(=O)n(C3(C)CC3)cc12)c1cccc2c1OCC2(F)F] | (R)-4-((1-(3,3-difluoro-2,3- dihydrobenzofuran-7-yl)prop-2-yn-1- yl)amino)-2-methyl-6-(1- methylcyclopropyl)pyrido[4,3- d]pyrimidin-7(6H)-one |
| 22 | 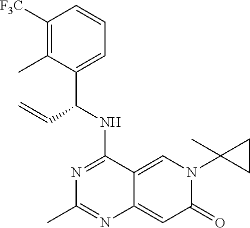 [SMILES: C=C[C@@H](Nc1nc(C)nc2cc(=O)n(C3(C)CC3)cc12)c1cccc(C(F)(F)F)c1C] | (R)-2-methyl-4-((1-(2-methyl-3- (trifluoromethyl)phenyl)allyl)amino)-6- (1-methylcyclopropyl)pyrido[4,3- d]pyrimidin-7(6H)-one |
| 23 | 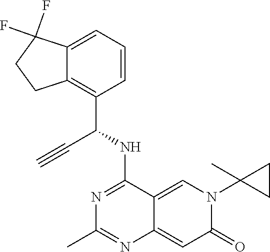 [SMILES: C#C[C@@H](Nc1nc(C)nc2cc(=O)n(C3(C)CC3)cc12)c1cccc2c1CCC2(F)F] | (R)-4-((1-(1,1-difluoro-2,3-dihydro-1H- inden-4-yl)prop-2-yn-1-yl)amino)-2- methyl-6-(1- methylcyclopropyl)pyrido[4,3- d]pyrimidin-7(6H)-one |
| 24 | 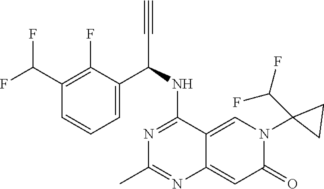 [SMILES: C#C[C@@H](Nc1nc(C)nc2cc(=O)n(C3(C(F)F)CC3)cc12)c1cccc(C(F)F)c1F] | (R)-4-((1-(3-(difluoromethyl)-2- fluorophenyl)prop-2-yn-1-yl)amino)-6- (1-(difluoromethyl)cyclopropyl)-2- methylpyrido[4,3-d]pyrimidin-7(6H)- one |
| 25 | 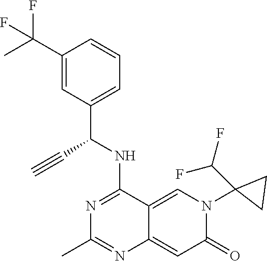 [SMILES: C#C[C@@H](Nc1nc(C)nc2cc(=O)n(C3(C(F)F)CC3)cc12)c1cccc(C(C)(F)F)c1] | (R)-4-((1-(3-(1,1-difluoroethyl)-2- fluorophenyl)prop-2-yn-1-yl)amino)-6- (1-(difluoromethyl)cyclopropyl)-2- methylpyrido[4,3-d]pyrimidin-7(6H)- one |
| 26 | 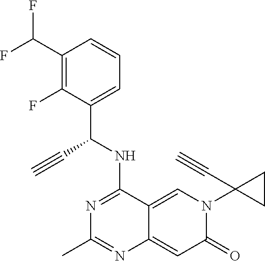 [SMILES: C#C[C@@H](Nc1nc(C)nc2cc(=O)n(C3(C#C)CC3)cc12)c1cccc(C(F)F)c1F] | (R)-4-((1-(3-(difluoromethyl)-2- fluorophenyl)prop-2-yn-1-yl)amino)-6- (1-ethynlcyclopropyl)-2-methylpyrido [4,3-d]pyrimidin-7(6H)-one |
| 27 | 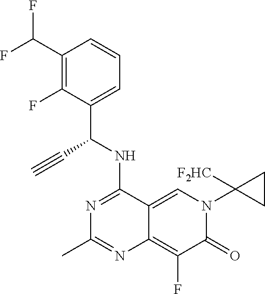 [SMILES: C#C[C@@H](Nc1nc(C)nc2c(F)c(=O)n(C3(C(F)F)CC3)cc12)c1cccc(C(F)F)c1F] | (R)-4-((1-(3-(difluoromethyl)-2- fluorophenyl)prop-2-yn-1-yl)amino)-6- (1-(difluoromethyl)cyclopropyl)-8- fluoro-2-methylpyrido[4,3-d]pyrimidin- 7(6H)-one |
| 28 | 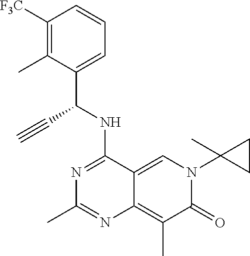 [SMILES: C#C[C@@H](Nc1nc(C)nc2c(C)c(=O)n(C3(C)CC3)cc12)c1cccc(C(F)(F)F)c1C] | (R)-2,8-dimethyl-4-((1-(2-methyl-3- (trifluoromethyl)phenyl)prop-2-yn-1- yl)amino)-6-(1- methylcyclopropyl)pyrido[4,3- d]pyrimidin-7(6H)-one |
| 29 | 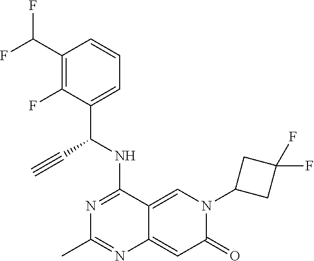 [SMILES: C#C[C@@H](Nc1nc(C)nc2cc(=O)n(C3CC(F)(F)C3)cc12)c1cccc(C(F)F)c1F] | (R)-6-(3,3-difluorocyclobutyl)-4-((1-(3- (difluoromethyl)-2-fluorophenyl)prop- 2-yn-1-yl)amino)-2-methylpyrido[4,3- d]pyrimidin-7(6H)-one |
| 30 | 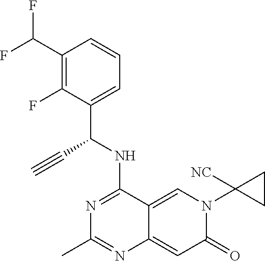 [SMILES: C#C[C@@H](Nc1nc(C)nc2cc(=O)n(C3(C#N)CC3)cc12)c1cccc(C(F)F)c1F] | (R)-1-(4-((1-(3-(difluoromethyl)-2- fluorophenyl)prop-2-yn-1-yl)amino)-2- methyl-7-oxopyrido[4,3-d]pyrimidin- 6(7H)-yl)cyclopropane-1-carbonitrile |
| 31 | 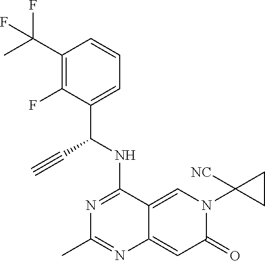 [SMILES: C#C[C@@H](Nc1nc(C)nc2cc(=O)n(C3(C#N)CC3)cc12)c1cccc(C(C)(F)F)c1F] | (R)-1-(4-((1-(3-(1,1-difluoroethyl)-2- fluorophenyl)prop-2-yn-1-yl)amino)-2- methyl-7-oxopyrido[4,3-d]pyrimidin- 6(7H)-yl)cyclopropane-1-carbonitrile |
| 32 | 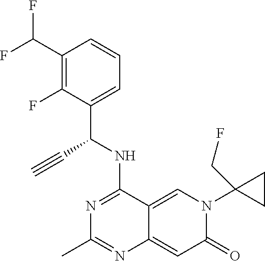 [SMILES: C#C[C@@H](Nc1nc(C)nc2cc(=O)n(C3(CF)CC3)cc12)c1cccc(C(F)F)c1F] | (R)-4-((1-(3-(difluoromethyl)-2- fluorophenyl)prop-2-yn-1-yl)amino)-6- (1-(fluoromethyl)cyclopropyl)-2- methylpyrido[4,3-d]pyrimidin-7(6H)- one |
| 33 | 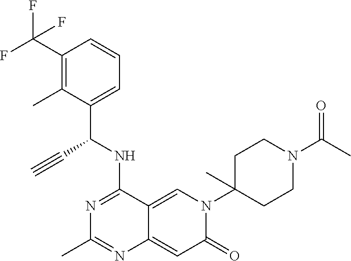 [SMILES: C#C[C@@H](Nc1nc(C)nc2cc(=O)n(C3(C)CCN(C(C)=O)CC3)cc12)c1cccc(C(F)(F)F)c1C] | (R)-6-(1-acetyl-4-methylpiperidin-4-yl)- 2-methyl-4-((1-(2-methyl-3- (trifluoromethyl)phenyl)prop-2-yn-1- yl)amino)pyrido[4,3-d]pyrimidin-7(6H)- one |
| 34 | 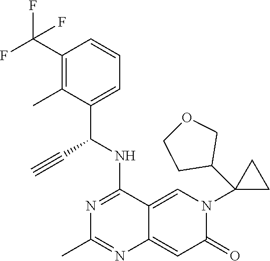 [SMILES: C#C[C@@H](Nc1nc(C)nc2cc(=O)n(C3(C4CCOC4)CC3)cc12)c1cccc(C(F)(F)F)c1C] | 2-methyl-4-(((R)-1-(2-methyl-3- (trifluoromethyl)phenyl)prop-2-yn-1- yl)amino)-6-(1-(tetrahydrofuran-3- yl)cyclopropyl)pyrido[4,3-d]pyrimidin- 7(6H)-one |
| 35 | 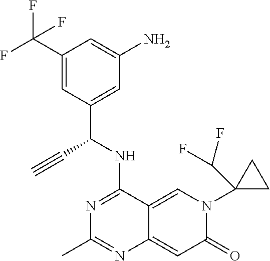 [SMILES: C#C[C@@H](Nc1nc(C)nc2cc(=O)n(C3(C(F)F)CC3)cc12)c1cc(N)cc(C(F)(F)F)c1] | (R)-4-((1-(3-amino-5- (trifluoromethyl)phenyl)prop-2-yn-1- yl)amino)-6-(1- (difluoromethyl)cyclopropyl)-2- methylpyrido[4,3-d]pyrimidin-7(6H)- one |
| 36 | 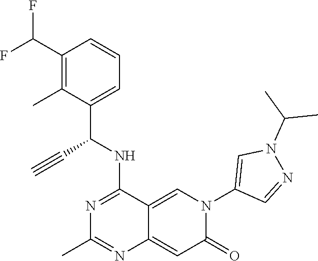 [SMILES: C#C[C@@H](Nc1nc(C)nc2cc(=O)n(-c3cnn(C(C)C)c3)cc12)c1cccc(C(F)F)c1C] | (R)-4-((1-(3-(difluoromethyl)-2- methylphenyl)prop-2-yn-1-yl)amino)-6- (1-isopropyl-1H-pyrazol-4-yl)-2- methylpyrido[4,3-d]pyrimidin-7(6H)- one |
| 37 | 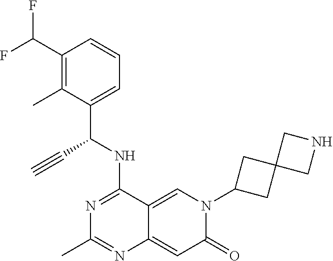 [SMILES: C#C[C@@H](Nc1nc(C)nc2cc(=O)n(C3CC4(CNC4)C3)cc12)c1cccc(C(F)F)c1C] | (R)-4-((1-(3-(difluoromethyl)-2- methylphenyl)prop-2-yn-1-yl)amino)-2- methyl-6-(2-azaspiro[3.3]heptan-6- yl)pyrido[4,3-d]pyrimidin-7(6H)-one |
| 38 | 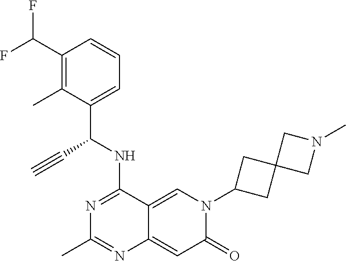 [SMILES: C#C[C@@H](Nc1nc(C)nc2cc(=O)n(C3CC4(C3)CN(C)C4)cc12)c1cccc(C(F)F)c1C] | (R)-4-((1-(3-(difluoromethyl)-2- methylphenyl)prop-2-yn-1-yl)amino)-2- methyl-6-(2-methyl-2- azaspiro[3.3]heptan-6-yl)pyrido[4,3- d]pyrimidin-7(6H)-one |
| 39 | 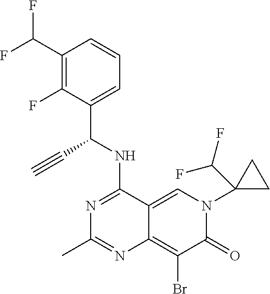 [SMILES: C#C[C@@H](Nc1nc(C)nc2c(Br)c(=O)n(C3(C(F)F)CC3)cc12)c1cccc(C(F)F)c1F] | (R)-8-bromo-4-((1-(3-(difluoromethyl)- 2-methylphenyl)prop-2-yn-1-yl)amino)- 6-(1-(difluoromethyl)cyclopropyl)-2- methylpyrido[4,3-d]pyrimidin-7(6H)- one |
| 40 | 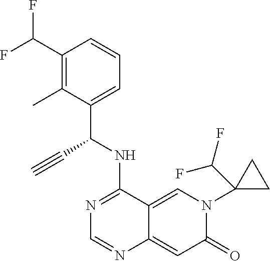 [SMILES: C#C[C@@H](Nc1ncnc2cc(=O)n(C3(C(F)F)CC3)cc12)c1cccc(C(F)F)c1C] | (R)-4-((1-(3-(difluoromethyl)-2- methylphenyl)prop-2-yn-1-yl)amino)-6- (1- (difluoromethyl)cyclopropyl)pyrido[4,3- d]pyrimidin-7(6H)-one |
| 41 | 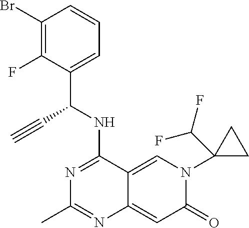 [SMILES: C#C[C@@H](Nc1nc(C)nc2cc(=O)n(C3(C(F)F)CC3)cc12)c1cccc(Br)c1F] | (R)-4-((1-(3-bromo-2- fluorophenyl)prop-2-yn-1-yl)amino)-6- (1-(difluoromethyl)cyclopropyl)-2- methylpyrido[4,3-d]pyrimidin-7(6H)- one |
| 42 | 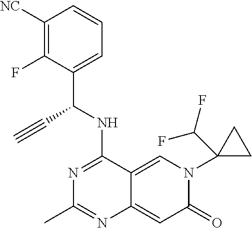 [SMILES: C#C[C@@H](Nc1nc(C)nc2cc(=O)n(C3(C(F)F)CC3)cc12)c1cccc(C#N)c1F] | (R)-3-(1-((6-(1- (difluoromethyl)cyclopropyl)-2-methyl- 7-oxo-6,7-dihydropyrido[4,3- d]pyrimidin-4-yl)amino)prop-2-yn-1- yl)-2-fluorobenzonitrile |
| 43 | 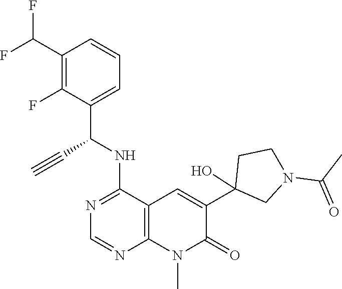 [SMILES: C#C[C@@H](Nc1ncnc2c1cc(C1(O)CCN(C(C)=O)C1)c(=O)n2C)c1cccc(C(F)F)c1F] | 6-(1-acetyl-3-hydroxypyrrolidin-3-yl)- 4-{[(1R)-1-[3-(difluoromethyl)-2- fluorophenyl]prop-2-yn-1-yl]amino}-8- methyl-7H,8H-pyrido[2,3-d]pyrimidin- 7-one |
| 44 | 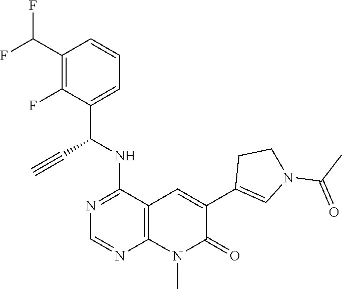 [SMILES: C#C[C@@H](Nc1ncnc2c1cc(C1=CN(C(C)=O)CC1)c(=O)n2C)c1cccc(C(F)F)c1F] | a mixture of 6-(1-acetyl-2,5-dihydro- 1H-pyrrol-3-yl)-4-{[(1R)-1-[3- (difluoromethyl)-2-fluorophenyl]prop- 2-yn-1-yl]amino}-8-methyl-7H,8H- pyrido[2,3-d]pyrimidin-7-one and 6-(1- acetyl-4,5-dihydro-1H-pyrrol-3-yl)-4- {[(1R)-1-[3-(difluoromethyl)-2- fluorophenyl]prop-2-yn-1-yl]amino}-8- methyl-7H,8H-pyrido[2,3-d]pyrimidin- 7-one |
| | 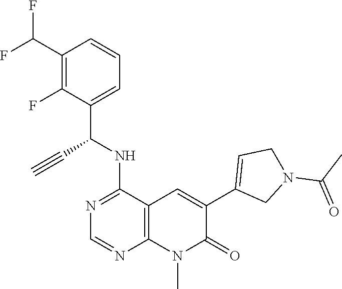 [SMILES: C#C[C@@H](Nc1ncnc2c1cc(C1=CCN(C(C)=O)C1)c(=O)n2C)c1cccc(C(F)F)c1F] | |
| 45 | 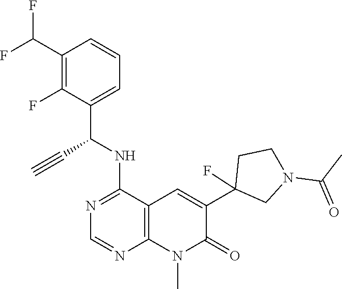 [SMILES: C#C[C@@H](Nc1ncnc2c1cc(C1(F)CCN(C(C)=O)C1)c(=O)n2C)c1cccc(C(F)F)c1F] | 6-(1-acetyl-3-fluoropyrrolidin-3-yl)-4- {[(1R)-1-[3-(difluoromethyl)-2- fluorophenyl]prop-2-yn-1-yl]amino}-8- methyl-7H,8H-pyrido[2,3-d]pyrimidin- 7-one |
| 46 | 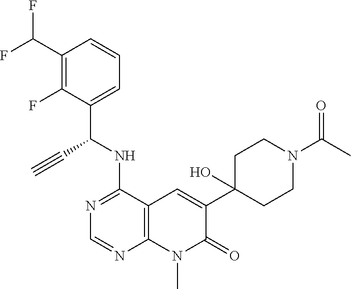 [SMILES: C#C[C@@H](Nc1ncnc2c1cc(C1(O)CCN(C(C)=O)CC1)c(=O)n2C)c1cccc(C(F)F)c1F] | 6-(1-acetyl-4-hydroxypiperidin-4-yl)-4- {[(1R)-1-[3-(difluoromethyl)-2- fluorophenyl]prop-2-yn-1-yl]amino}-8- methyl-7H,8H-pyrido[2,3-d]pyrimidin- 7-one |
| 47 | 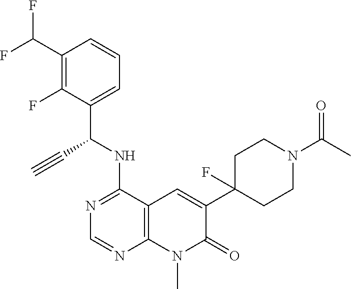 [SMILES: C#C[C@@H](Nc1ncnc2c1cc(C1(F)CCN(C(C)=O)CC1)c(=O)n2C)c1cccc(C(F)F)c1F] | 6-(1-acetyl-4-fluoropiperidin-4-yl)-4- {[(1R)-1-[3-(difluoromethyl)-2- fluorophenyl]prop-2-yn-1-yl]amino}-8- methyl-7H,8H-pyrido[2,3-d]pyrimidin- 7-one |
| 48 | 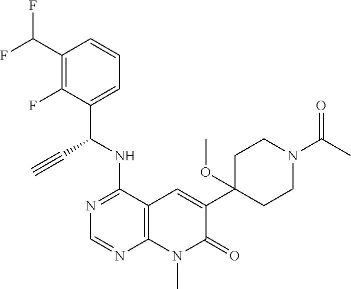 [SMILES: C#C[C@@H](Nc1ncnc2c1cc(C1(OC)CCN(C(C)=O)CC1)c(=O)n2C)c1cccc(C(F)F)c1F] | (R)-6-(1-acetyl-4-methoxypiperidin-4- yl)-4-((1-(3-(difluoromethyl)-2- fluorophenyl)prop-2-yn-1-yl)amino)-8- methylpyrido[2,3-d]pyrimidin-7(8H)- one |
| 49 | 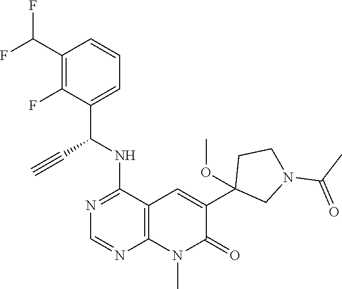 [SMILES: C#C[C@@H](Nc1ncnc2c1cc(C1(OC)CCN(C(C)=O)C1)c(=O)n2C)c1cccc(C(F)F)c1F] | 6-(1-acetyl-3-methoxypyrrolidin-3-yl)- 4-{[(1R)-1-[3-(difluoromethyl)-2- fluorophenyl]prop-2-yn-1-yl]amino}-8- methyl-7H,8H-pyrido[2,3-d]pyrimidin- 7-one |
| 50 | 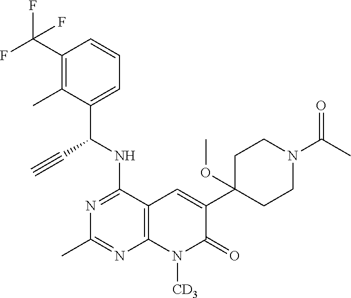 [SMILES: [2H]C([2H])([2H])n1c(=O)c(C2(OC)CCN(C(C)=O)CC2)cc2c(N[C@H](C#C)c3cccc(C(F)(F)F)c3C)nc(C)nc21] | 6-(1-acetyl-4-methoxypiperidin-4-yl)-8- (\hat{ }2H₃)methyl-2-methyl-4-{[(1R)-1-[2- methyl-3-(trifluoromethyl)phenyl]prop- 2-yn-1-yl]amino}-7H,8H-pyrido[2,3- d]pyrimidin-7-one |
| 51 | 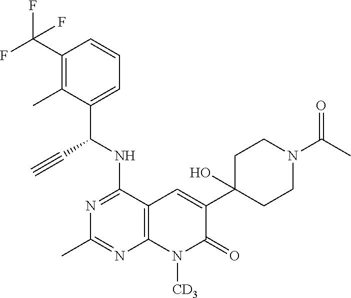 [SMILES: [2H]C([2H])([2H])n1c(=O)c(C2(O)CCN(C(C)=O)CC2)cc2c(N[C@H](C#C)c3cccc(C(F)(F)F)c3C)nc(C)nc21] | 6-(1-acetyl-4-hydroxypiperidin-4-yl)-8- (\hat{ }2H₃)methyl-2-methyl-4-{[(1R)-1-[2- methyl-3-(trifluoromethyl)phenyl]prop- 2-yn-1-yl]amino}-7H,8H-pyrido[2,3- d]pyrimidin-7-one |
| 52 | 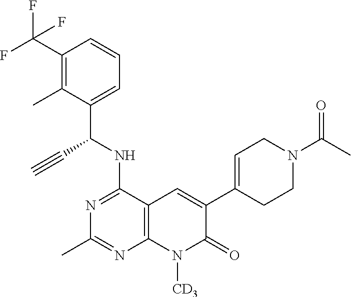 [SMILES: [2H]C([2H])([2H])n1c(=O)c(C2=CCN(C(C)=O)CC2)cc2c(N[C@H](C#C)c3cccc(C(F)(F)F)c3C)nc(C)nc21] | 6-(1-acetyl-1,2,3,6-tetrahydropyridin-4- yl)-8-(\hat{ }2H₃)methyl-2-methyl-4- {[(1R)-1-[2-methyl-3- (trifluoromethyl)phenyl]prop-2-yn-1- yl]amino}-7H,8H-pyrido[2,3- d]pyrimidin-7-one |
| 53 | 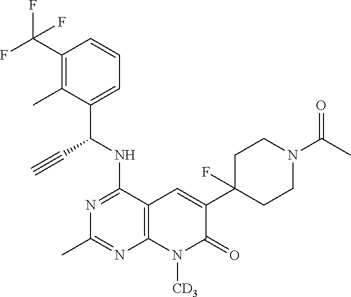 [SMILES: [2H]C([2H])([2H])n1c(=O)c(C2(F)CCN(C(C)=O)CC2)cc2c(N[C@H](C#C)c3cccc(C(F)(F)F)c3C)nc(C)nc21] | 6-(1-acetyl-4-fluoropiperidin-4-yl)-8- (\hat{ }2H₃)methyl-2-methyl-4-{[(1R)-1-[2- methyl-3-(trifluoromethyl)phenyl]prop- 2-yn-1-yl]amino}-7,8- dihydroquinazolin-7-one |
| 54 | 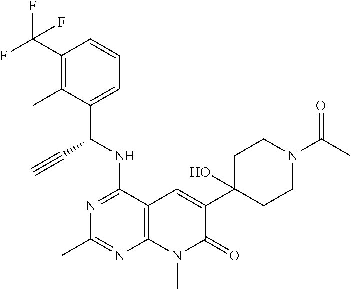 [SMILES: C#C[C@@H](Nc1nc(C)nc2c1cc(C1(O)CCN(C(C)=O)CC1)c(=O)n2C)c1cccc(C(F)(F)F)c1C] | 6-(1-acetyl-4-hydroxypiperidin-4-yl)- 2,8-dimethyl-4-{[(1R)-1-[2-methyl-3- (trifluoromethyl)phenyl]prop-2-yn-1- yl]amino}-7H,8H-pyrido[2,3- d]pyrimidin-7-one |
| 55 | 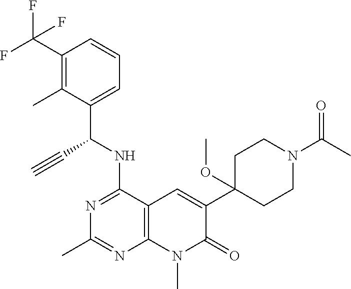 [SMILES: C#C[C@@H](Nc1nc(C)nc2c1cc(C1(OC)CCN(C(C)=O)CC1)c(=O)n2C)c1cccc(C(F)(F)F)c1C] | 6-(1-acetyl-4-methoxypiperidin-4-yl)- 2,8-dimethyl-4-{[(1R)-1-[2-methyl-3- (trifluoromethyl)phenyl]prop-2-yn-1- yl]amino}-7H,8H-pyrido[2,3- d]pyrimidin-7-one |
| 56 | 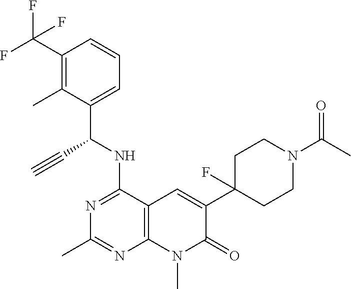 [SMILES: C#C[C@@H](Nc1nc(C)nc2c1cc(C1(F)CCN(C(C)=O)CC1)c(=O)n2C)c1cccc(C(F)(F)F)c1C] | 6-(1-acetyl-4-fluoropiperidin-4-yl)-2,8- dimethyl-4-{[(1R)-1-[2-methyl-3- (trifluoromethyl)phenyl]prop-2-yn-1- yl]amino}-7H,8H-pyrido[2,3- d]pyrimidin-7-one |
| 57 | 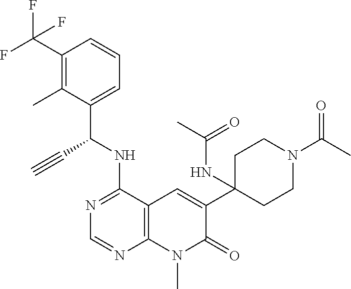 [SMILES: C#C[C@@H](Nc1ncnc2c1cc(C1(NC(C)=O)CCN(C(C)=O)CC1)c(=O)n2C)c1cccc(C(F)(F)F)c1C] | (R)-N-(1-acetyl-4-(8-methy-4-((1-(2- methyl-3-(trifluoromethyl)phenyl)prop- 2-yn-1-yl)amino)-7-oxo-7,8- dihydropyrido[2,3-d]pyrimidin-6- yl)piperidin-4-yl)acetamide |
| 58 | 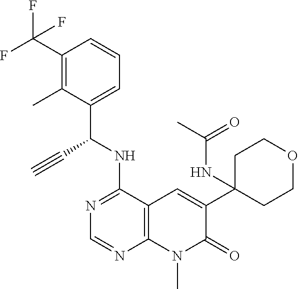 [SMILES: C#C[C@@H](Nc1ncnc2c1cc(C1(NC(C)=O)CCOCC1)c(=O)n2C)c1cccc(C(F)(F)F)c1C] | (R)-N-(4-(8-methy-4-((1-(2-methyl-3- (trifluoromethyl)phenyl)prop-2-yn-1- yl}amino)-7-oxo-7,8- dihydropyrido[2,3-d]pyrimidin-6- yl)tetrahydro-2H-pyran-4-yl)acetamide |
| 59 | 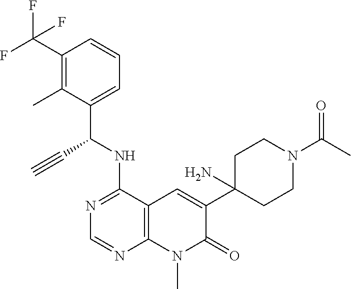 [SMILES: C#C[C@@H](Nc1ncnc2c1cc(C1(N)CCN(C(C)=O)CC1)c(=O)n2C)c1cccc(C(F)(F)F)c1C] | 6-(1-acetyl-4-aminopiperidin-4-yl)-2,8- dimethyl-4-{[(1R)-1-[2-methyl-3- (trifluoromethyl)phenyl]prop-2-yn-1- yl]amino}-7H,8H-pyrido[2,3- d]pyrimidin-7-one |
| 60 | 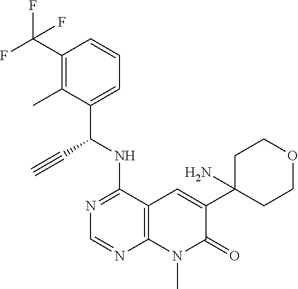 [SMILES: C#C[C@@H](Nc1ncnc2c1cc(C1(N)CCOCC1)c(=O)n2C)c1cccc(C(F)(F)F)c1C] | (R)-6-(4-aminotetrahydro-2H-pyran-4- yl)-8-methy-4-((1-(2-methyl-3- (trifluoromethyl)phenyl)prop-2-yn-1- yl)amino)-pyrido[2,3-d]pyrimidin-6- yl)pyrimidin-7(8H)-one |
| 61 | 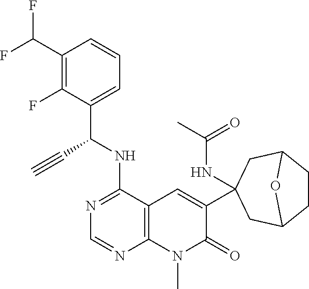 [SMILES: C#C[C@@H](Nc1ncnc2c1cc(C1(NC(C)=O)CC3CCC(C1)O3)c(=O)n2C)c1cccc(C(F)F)c1F] | N-(3(4-(((R)-1-3-(difluoromethyl)-2- fluorophenyl)prop-2-yn-1-yl)amino-8- methyl-7-oxo-7,8-dihydorpyrido[2,3- d]pyrimidin-6-yl)-8- oxabicyclo[3.2.1]octan-3-yl)acetamide |
| 62 | 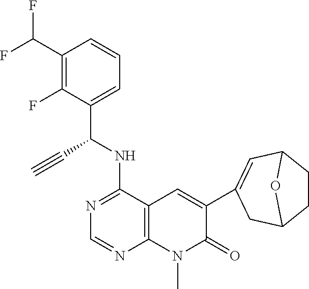 [SMILES: C#C[C@@H](Nc1ncnc2c1cc(C1=CC3CCC(C1)O3)c(=O)n2C)c1cccc(C(F)F)c1F] | 6-(8-oxabicyclo[3.2.1]oct-2-en-3-yl)-4- (((R)-1-(3-(difluoromethyl)-2- fluorophenyl)prop-2-yn-1-yl)amino-8- methylpyrido[2,3-d]pyrimidin-7)8H)- one |
| 63 | 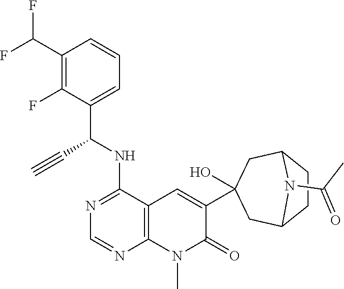 [SMILES: C#C[C@@H](Nc1ncnc2c1cc(C1(O)CC3CCC(C1)N3C(C)=O)c(=O)n2C)c1cccc(C(F)F)c1F] | 6-(8-acetyl-3-hydroxy-8- azabicyclo[3.2.1]octan-3-yl)-4-(((R)-1- (3-(difluoromethyl)-2- fluorophenyl)prop-2-yn-1-yl)amino-8- methylpyrido[2,3-d]pyrimidin-7)8H)- one |
| 64 | 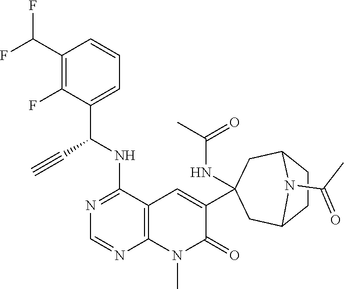 [SMILES: C#C[C@@H](Nc1ncnc2c1cc(C1(NC(C)=O)CC3CCC(C1)N3C(C)=O)c(=O)n2C)c1cccc(C(F)F)c1F] | 6-(8-acetyl-3-4-(((R)-1-(3- (difluoromethyl)-2-fluorophenyl)prop- 2-yn-1-yl)amino-8-methyl-7-oxo-7,8- dihydropyrido[2,3-d]pyrimidin-6-yl)-8- azabicyclo[3.2.1]octan-3-yl-acetamide |
| 65 | 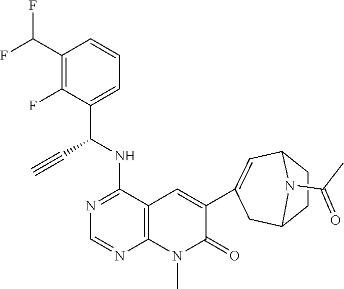 [SMILES: C#C[C@@H](Nc1ncnc2c1cc(C1=CC3CCC(C1)N3C(C)=O)c(=O)n2C)c1cccc(C(F)F)c1F] | 6-(8-acetyl-8-azabicyclo[3.2.1]oct-2-en- 3-yl)-4-(((R)-1-(3-(difluoromethyl)-2- fluorophenyl)prop-2-yn-1-yl)amino-8- methyl-pyrido[2,3-d]pyrimidin-7(8H)- one |
| 66 | 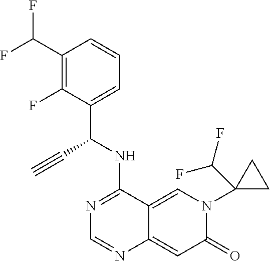 [SMILES: C#C[C@@H](Nc1ncnc2cc(=O)n(C3(C(F)F)CC3)cc12)c1cccc(C(F)F)c1F] | (R)-4-((1-(3-(difluoromethyl)-2- fluorophenyl)prop-2-yn-1-yl)amino)-6- (1- (difluoromethyl)cyclopropyl)pyrido[4,3- d]pyrimidin-(6H)-one |
| 67 | 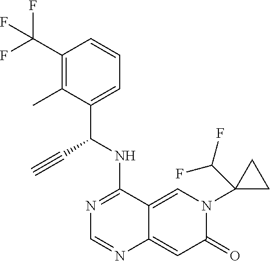 [SMILES: C#C[C@@H](Nc1ncnc2cc(=O)n(C3(C(F)F)CC3)cc12)c1cccc(C(F)(F)F)c1C] | (R)-6-(1-(difluoromethyl)cyclopropyl)- 4-((1-(2-methyl-3- (trifluoromethyl)phenyl)prop-2-yn-1- yl)amino)pyrido[4,3-d]pyrimidin-7(6H)- one |
| 68 | 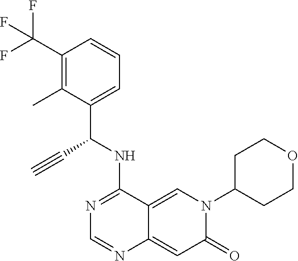 [SMILES: C#C[C@@H](Nc1ncnc2cc(=O)n(C3CCOCC3)cc12)c1cccc(C(F)(F)F)c1C] | (R)-4-((1-(2-methyl-3- (trifluoromethyl)phenyl)prop-2-yn-1- yl)amino)-6-(tetrahydro-2H-pyran-4- yl)pyrido[4,3-d]pyrimidin-7(6H)-one |
| 69 | 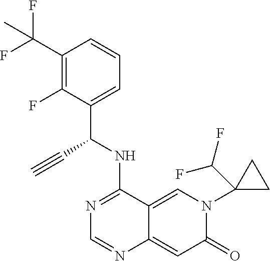 [SMILES: C#C[C@@H](Nc1ncnc2cc(=O)n(C3(C(F)F)CC3)cc12)c1cccc(C(C)(F)F)c1F] | (R)-4-((1-(3-(1,1-difluoroethyl)-2- fluorophenyl)prop-2-yn-1-yl)amino)-6- (1- (difluoromethyl)cyclopropyl)pyrido[4,3- d]pyrimidin-7(6H)-one |

Preferred compounds include Compounds 1, 4, 9, 24, and 25. Another set of preferred compounds are Compounds 40 and 66-69.

The term “halogen” herein refers to a fluoro, chloro, bromo, or iodo radical. A particular halogen is a fluoro radical.

The term “alkyl” refers to a straight or branched hydrocarbon group, containing 1-20 carbon atoms (e.g., C₁₋₁₀, C₁₋₆, C₁₋₄, and C₁₋₃) and a monovalent radical center derived by the removal of a hydrogen atom from a carbon atom of a parent alkane. Exemplary alkyl groups are methyl, ethyl, n-propyl, isopropyl, n-butyl, iso-butyl, tert-butyl, n-pentyl, and n-hexyl. The term “alkylene” refers to a straight or branched hydrocarbon group, containing 1-20 carbon atoms (e.g., C₁₋₁₀, C₁₋₆, C₁₋₄, and C₁₋₃) and two monovalent radical centers derived by the removal of two hydrogen atoms from the same or two different carbon atoms of a parent alkane.

The term “haloalkyl” refers to alkyl substituted with one or more halogens (fluoro, chloro, bromo, or iodo). Examples include fluoromethyl, difluoromethyl, trifluoromethyl, fluoroethyl (e.g., 1-fluoroetyl and 2-fluoroethyl), difluoroethyl (e.g., 1,1-, 1,2-, and 2,2-difluoroethyl), and trifluoroethyl (e.g., 2,2,2-trifluoroethyl).

The term “alkoxy” refers to an —O-alkyl group. Examples are methoxy, ethoxy, propoxy, and isopropoxy. Alkoxy also includes haloalkoxy, namely, alkoxy substituted with one or more halogens, e.g., —O—CH₂Cl and —O—CHClCH₂Cl.

The term “cycloalkyl” refers to a nonaromatic, saturated or unsaturated monocyclic, bicyclic, tricyclic, or tetracyclic hydrocarbon group containing 3 to 12 carbons (e.g., C₃₋₁₀ and C₃₋₆). Cycloalkyl also includes fused, bridged, and spiro ring systems. Examples are cyclopropyl, cyclobutyl, cyclopentyl, cyclopentenyl, cyclohexyl, cyclohexenyl, cycloheptyl, cyclooctyl, bicyclo[1.1.1]pentyl, bicyclo[2.1.1]hexyl, bicyclo[2.1.1]heptyl, bicyclo[2.2.2]octanyl, and decahydronaphthalene.

The term “heterocycloalkyl” refers to a nonaromatic, saturated or unsaturated, 3-8 membered monocyclic, 8-12 membered bicyclic, or 11-14 membered tricyclic ring system having one or more heteroatoms (e.g., O, N, P, and S). The term also includes fused, bridged, and spiro ring systems. Examples include aziridinyl, azetidinyl, pyrrolidinyl, dihydrofuranyl, tetrahydrofuranyl, tetrahydropyranyl, tetrahydrothiophenyl, tetrahydro-2-H-thiopyran-1,1-dioxidyl, piperazinyl, piperidinyl, morpholinyl, imidazolidinyl, azepanyl, dihydrothiadiazolyl, dioxanyl, 2-azaspiro[3.3]heptanyl, quinuclidinyl, and 8-azabicyclo[3.2.1]octanyl.

The term “alkenyl” refers to a straight or branched, monovalent, unsaturated aliphatic chain having 2 to 20 carbon atoms (e.g., C₂₋₄, C₂₋₆, and C₂₋₁₀) and one or more carbon-carbon double bonds. Examples are ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, and 2-pentenyl. The term “alkenylene” refers to a straight or branched, bivalent, unsaturated aliphatic chain having 2 to 20 carbon atoms (e.g., C₂₋₄, C₂₋₆, and C₂₋₁₀) and one or more carbon-carbon double bonds.

The term “alkynyl” refers to a straight or branched aliphatic chain having 2 to 20 carbon atoms (e.g., C₂₋₄, C₂₋₆, and C₂₋₁₀) and one or more carbon-carbon triple bonds. Examples are ethynyl, 2-propynyl, 2-butynyl, 3-methylbutnyl, and 1-pentynyl. The term “alkynylene” refers to a straight or branched, bivalent, unsaturated aliphatic chain having 2 to 20 carbon atoms (e.g., C₂₋₄, C₂₋₆, and C₂₋₁₀) and one or more carbon-carbon triple bonds.

The term “aryl” refers a 6-carbon monocyclic, 10-carbon bicyclic, 14-carbon tricyclic aromatic ring system wherein each ring can have one or more (e.g., 1 to 10, 1 to 5, and 1 to 3) substituents. Examples include phenyl, biphenyl, 1- or 2-naphthyl, 1,2-dihydronaphthyl, 1,2,3,4-tetrahydronaphthyl, indenyl, and indanyl.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include pyridinyl, pyrimidinyl, quinolinyl, isoquinolinyl, indolyl, benzimidazolyl, benzoxazolyl, benzothiophenyl, benzofuranyl, pyrazolyl, triazolyl, oxazolyl, thiadiazolyl, tetrazolyl, oxazolyl, isoxazolyl, carbazolyl, furyl, imidazolyl, thienyl, quinolinyl, indolyl, thiazolyl, and benzothiazolyl.

Alkyl, alkoxyl, cycloalkyl, heterocycloalkyl, alkenyl, alkynyl, aryl, and heteroaryl mentioned herein include both substituted and unsubstituted moieties. Examples of a substituent include halogen (e.g., F, Cl, and Br), amino, hydroxy, alkyl and haloalkyl (e.g., methyl, fluoromethyl, difluoromethyl, trifluoromethyl, difluoroethyl, and 1,1-difluoro-2-hydroxylethan-1-yl), alkenyl and haloalkenyl (e.g., ethylenyl and 3,3-difluoro-2-methylpropen-3-yl), cycloalkyl (e.g., cyclopropyl and cyclobutyl), cycloheteroalkyl (e.g., tetrahydrofuranyl), —CN, —CONR₇R₈, —NR₇R₈, —NR₇COR₈, —NR₇SO₂R₈, —N₇COOR₈, —COR₇, —COOR₇, —SR₇, —SONR₇R₈, and —OR₉, wherein R₇, R₈, and R₉ are on each occurrence independently selected from the group consisting of hydrogens, C₁₋₆ alkyl, C₂₋₄ alkenyl, and C₂₋₄ alkynyl optionally substituted with 1-3 halogens. All substituents can be further substituted.

The term “compound”, when referring to a compound of this invention, also includes its salts, solvates, and prodrugs. The pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereometric mixture, a cis-isomer, or a trans-isomer. Compounds of such isomeric forms are within the scope of this invention. They can be present as a mixture or can be isolated using chiral synthesis or chiral separation technologies.

The depiction of an asterisk (*) in a chemical formula represents the point of attachment of the group to the corresponding parent formula.

The present invention also features use of one or more of the above-described compounds for treating cancer.

The term “treating” or “treatment” refers to administering one or more of the compounds to a subject with the purpose to confer a therapeutic effect, e.g., to slow, interrupt, arrest, control, or stop of the progression of an existing disorder and/or symptoms thereof, but does not necessarily indicate a total elimination of all symptoms. “An effective amount” refers to the amount of a compound that is required to confer the therapeutic effect. Effective doses will vary, as recognized by those skilled in the art, depending on the types of symptoms treated, route of administration, excipient usage, and the possibility of co-usage with other therapeutic treatment.

The cancer is caused by KRAS mutation, SOS1 oncogenic mutation, or oncogenic mutation/overexpression of receptor tyrosine kinases such as EGFR, FGFR, etc. and selected from the group consisting of pancreatic cancer, lung cancer, colorectal cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcoma. Preferably, the cancer is pancreatic, non-small cell lung cancer, cholangiocarcinoma, or colorectal cancer.

The term “subject” refers to an animal such as a mammal including a human. A human is a preferred subject.

A compound of this invention may be administered alone or in the form of a pharmaceutical composition with pharmaceutically acceptable carriers, diluents or excipients. Such pharmaceutical compositions and processes for making the same are known in the art (See, e.g., Remington: The Science and Practice of Pharmacy, A. Adejare, Editor, 23rd Edition, Academic Press, 2020).

This invention further features treating diseases by inhibiting SOS1 activity, defined as a RASopathy. The disease is selected from the group consisting of Neurofibromatosis type 1 (NF1), Noonan Syndrome (NS), Noonan Syndrome with Multiple Lentigines (NSML) (also referred to as LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome), and Hereditary gingival fibromatosis.

To practice the method of the present invention, a composition or a kit containing one or more of the above-described compounds can be administered alone or co-administered with at least one other pharmacologically active substance simultaneously, concurrently, sequentially, successively, alternately, or separately. Simultaneous administration, also referring to as concomitant administration, includes administration at substantially the same time. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

The elements of the combinations of this invention may be administered (whether dependently or independently) by methods customary to the skilled person, e.g., by oral, enteral, parenteral, nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

The term “parenteral” as used herein refers to subcutaneous, intracutaneous, intravenous, intraperitoneal, intramuscular, intraarticular, intraarterial, intrasynovial, intrasternal, intrathecal, intralesional, or intracranial injection, as well as any suitable infusion technique.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents.

A composition having one or more of the above-described compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples include colloidal silicon oxide, magnesium stearate, cellulose, sodium lauryl sulfate, and D&C Yellow #10.

The details of one or more embodiments of the invention are set forth in the description below. Other features, objects, and advantages of the invention will be apparent from the description and from the claims.

### DETAILED DESCRIPTION

The present invention is based on a surprising discovery that the compounds of formula (I) are effective in inhibiting SOS1 activity and treating cancer. In vivo studies have demonstrated their efficacy in treating cancer.

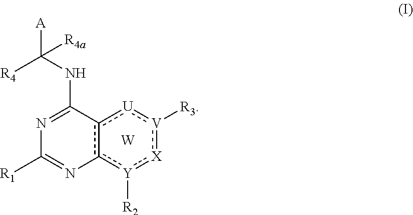

Variables R₁-R₄, R₄ₐ, A, U, V, X, Y, and ring W are defined above. Described in detail below are compounds of formula (I), as well as their syntheses and their use in treating cancer and/or inhibiting SOS1.

Subsets of the compounds of formula (I) include compounds of formulas (IA), (IB), (IA-1), and (IB-1) as shown below:

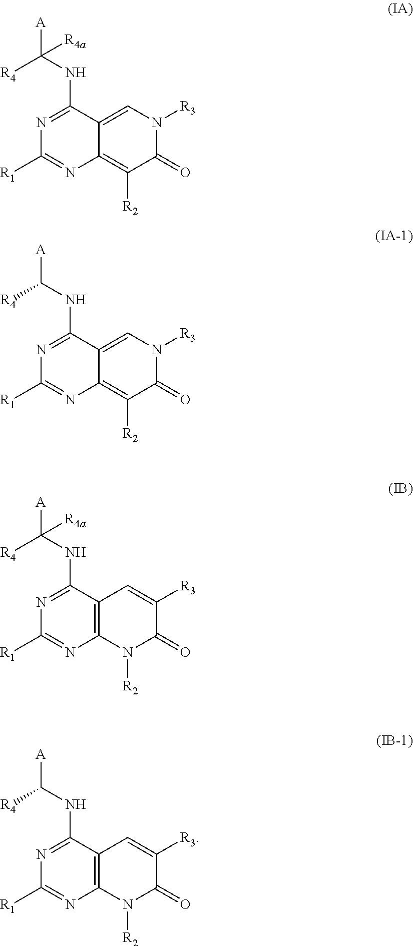

Each of the above formula contains a fused pyrimidine core structure. The compounds of formulas (IA) and (IB) include all enantiomers, diastereomers, and mixtures thereof in any ratio. Preferred stereoisomers are those of formula (IA-1) and (IB-1). In a stereoisomer mixture, a compound of formula (IA-1) or (IB-1) is preferably present at a level of at least 60% (e.g., at least 80%, at least 95%, at least 98%, and at least 99.5%).

Another subset of compounds of formula (I) are compounds of formula (IC) below.

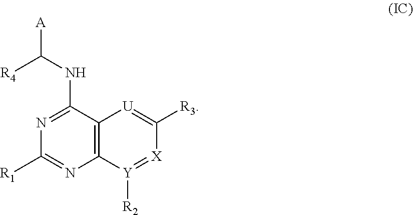

In this formula, each of U and X, independently, is CH or N and Y is C or N.

Examples include:

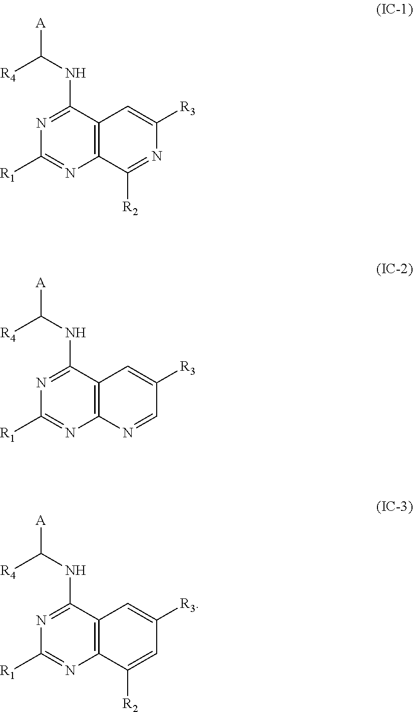

Compounds of formula (IC) can be prepared following known methods (e.g., those described in International Application Publication numbers WO2019/201848 and WO 2021/074227), using commercially available reagents.

Other core structures can also be used such as those described in International Application Publication Numbers WO 2020/180770, WO 2018/115380, WO2018/172250, WO 2019/122129, WO 2020/180768A, WO 2020/234103, WO 2020/254451, WO 2021/0127429, WO2021/092115, WO 2021/105960, WO 2021/130731, WO 2021/173524, WO 2021/203768, WO 2021/228028, WO 2021/249475, WO 2021/249519, WO2021/259972, WO 2022/017339, WO 2022/026465, WO 2022/028506, WO 2022/058344, and WO 2022/060583, and Chinese Application Publication Numbers CN 113200981, CN 113801114, and CN113912608.

More specifically, compounds of formula (ID) are also suitable.

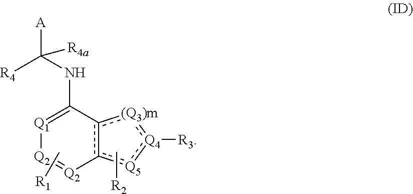

In this formula, R₁, R₂, R₃, R₄, R₄ₐ, and A are as defined above. Q₁ is CH or N; Q₄ is CH, C, or N; each Q₂ is independently C—R₁ or N, in which one Q₂ is N and the other Q₂ is C—R₁; Q₃ is C(R′)₂, NR″, CO, O, S, or SO₂, in which each R′ is independently H, F, Cl, Br, or 6-10 membered aryl, and each R″ is independently H, C₁₋₆ alkyl, or 6-10 membered aryl; Q₅ is CR₂, CO, OCR₂, SCR₂, CHCR₂, COCR₂, or NCR₂; at least one of Q₁, Q₂, Q₃, Q₄, and Q₅ is N, NR″, O, or SO₂; m is 0, 1, 2, or 3; and each [symbol] is a single bond or a double bond, provided that the number of the double bonds represented by [symbol] is 1, 2, or 3.

Examples of the core structure

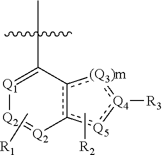

include:

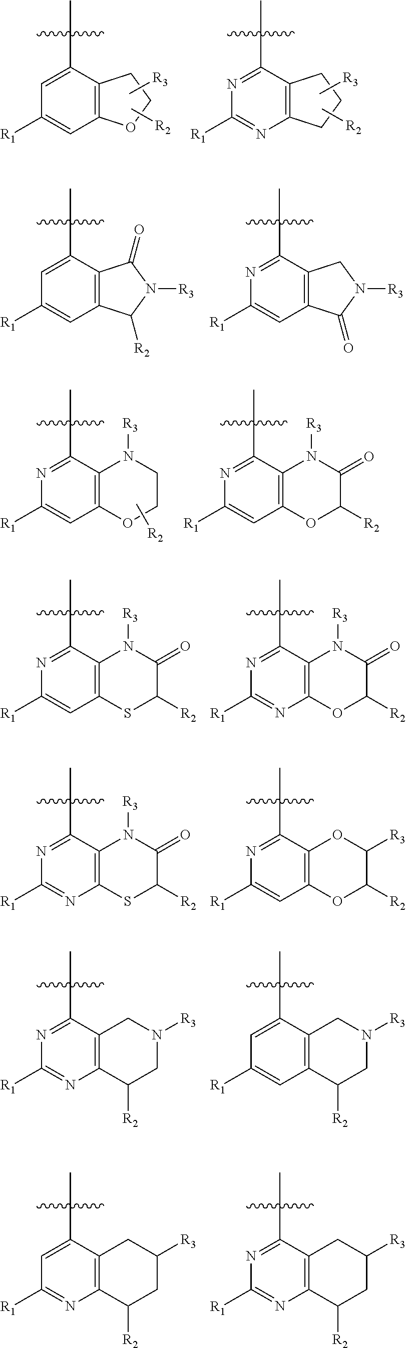

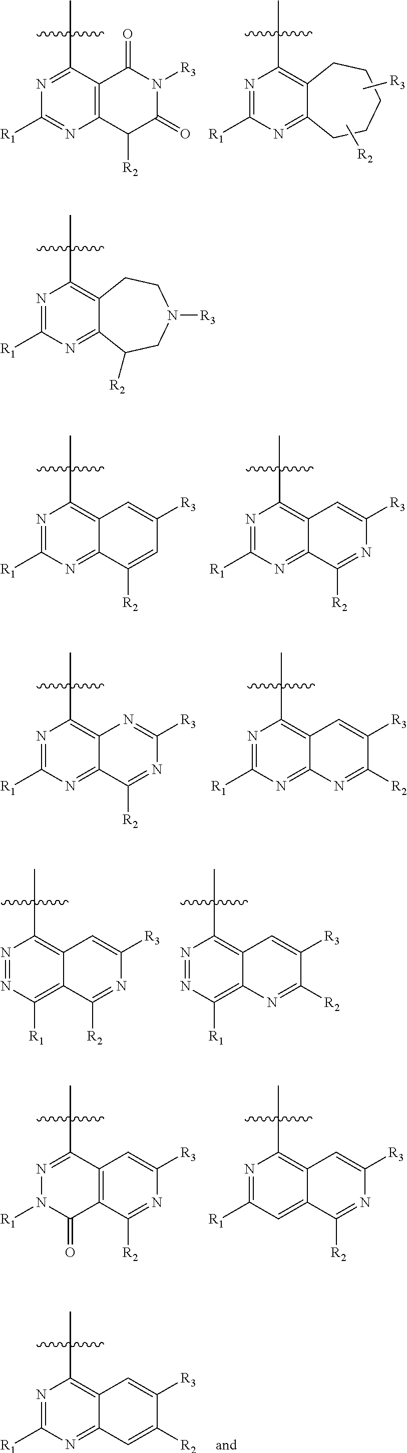

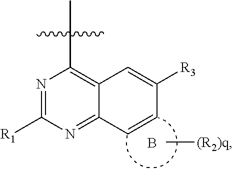

in which B is a 5- or 6-membered, saturated or unsaturated cycloalkyl or heterocycloalkyl, aryl, or heteroaryl ring, and q is 0, 1, 2, or 3.

The compounds of any of the above formulas can be prepared by synthetic methods well known in the art. See, e.g., R. Larock, Comprehensive Organic Transformations (3^{rd} Ed., John Wiley and Sons 2018); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2^{nd} ed., John Wiley and Sons 2009) and subsequent editions thereof.

The compounds thus prepared can be purified following conventional methods such as crystallization, distillation/vacuum distillation, flash chromatography over silica, and preparative liquid chromatography.

Efficacy of the compounds of this invention can be initially determined using in vitro homogeneous time-resolved fluorescence (HTRF) based protein-protein interaction assay, pERK potency assay, or 3D cell proliferation assay, all described below following the Examples. The selected compounds can be further tested to verify their efficacy, e.g., by administering it to an animal. Based on the results, an appropriate dosage range and administration route can be determined.

A compound of this invention is preferably formulated into a pharmaceutical composition containing a pharmaceutical carrier. The pharmaceutical composition is then given to a subject in need thereof to inhibit SOS1 thus treating cancer.

Without further elaboration, it is believed that one skilled in the art can, based on the above description, utilize the present invention to its fullest extent. The following examples are to be construed as merely illustrative and not limitative of the remainder of the disclosure in any way whatsoever.

All publications cited herein are hereby incorporated by reference in their entirety.

Set forth below are examples illustrating preparation and efficacy evaluation of compounds of this invention.

The abbreviations as used herein are provided with the corresponding definitions below.

| Abbreviation | Name | Abbreviation | Name |
|---|---|---|---|
| MS | mass spectrometry | NMR | nuclear magnetic resonance |
| TLC | thin layer chromatography | HPLC | High-Performance Liquid Chromatography |
| LCMS | Liquid Chromatography-Mass Spectrometry | DMF | dimethylformamide |
| DCE | 1,2-dichloroethane | PE | petroleum ether |
| DCM | dichloromethane | DMSO | dimethyl sulfoxide |
| IPA | Isopropyl alcohol | DEA | diethylamine |
| MTBE | methyl tert-butyl ether | THF | tetrahydrofuran |
| Pd(dppf)Cl₂ | [1,1′- bis(diphenylphosphino)- ferrocene] dichloropallad- ium (II) | HATU | l[bis(dimethylamino)methylene]- 1H-1,2,3-triazolo[4,5-b] pyridinium 3-oxide hexafluorophosphate |
| TFA | trifluoroacetic acid | TEA | Triethylamine |
| LiHMDS | lithium hexamethyldisilylamide | DIPEA | N,N-diisopropylethylamine |
| Dmap | 4-(dimethylamino)- pyridine | TsOH | p-toluenesulfonic acid |
| HCCP | phosphonitrilic chloride trimer | DMF-DMA | N, N-Dimethylformamide dimethyl acetal |
| TMSA | (trimethylsilyl)acetylene | LDA | lithium diisopropylamide |
| X-PHOS | 2- dicyclohexylphosphino- 2′,4′,6′- triisopropylbiphenyl | Pd₂(dba)₃ | tris(dibenzylideneacetone) dipalladium (0) |
| GSD | geometric standard deviation | | |

### General Synthetic Procedure

A compound of formula (I), e.g., formula (IA-1), may be synthesized using the procedure shown in Scheme 1 below. More specifically, a compound of formula II is reacted with ethylene glycol and an acid such as TsOH in a solvent such as toluene to provide a compound of formula III. The compound of formula III is reacted with a R₂ substituted malonic ester in the presence of base such as Cs₂CO₃ in a solvent such as DMSO to provide a compound of formula IV. The compound of formula IV is decarboxylated to provide a compound of formula V.

The compound of formula V is reacted with a compound of formula VI in the presence of a base such DIPEA to provide a compound of formula VII. The reaction is conveniently carried out in a solvent such as DMSO. The compound of formula VII is reacted under saponification conditions to provide a compound of formula IIX. The compound of formula IIX is reacted with a compound of formula IX in the presence of a coupling agent such as HATU and a base such as DIPEA to provide a compound of formula X. The compound of formula X is reacted with an acid such aqueous HCl in a solvent such MeCN to provide a compound of formula IA-1.

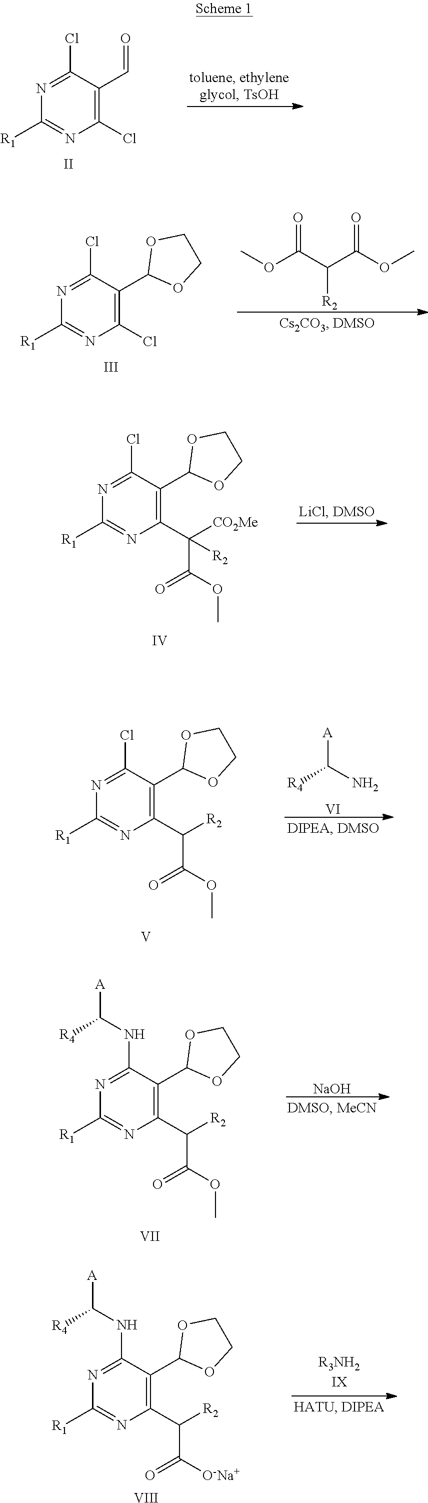

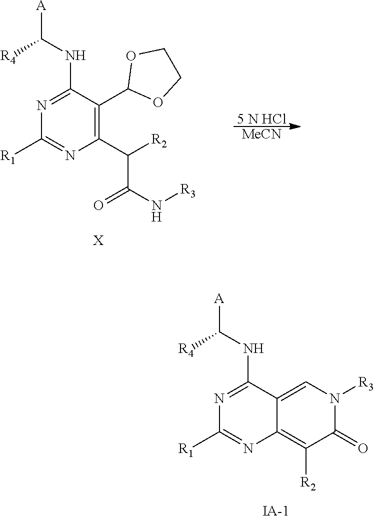

Alternatively, a compound of formula IA-1 having R₁ being methyl may be prepared by sequential reaction of a pyrimidinylpyridone of formula XVI with phosphonitrilic chloride trimer (HCCP) followed by coupling the chloro intermediate with a compound of formula VI as shown in Scheme 2 below.

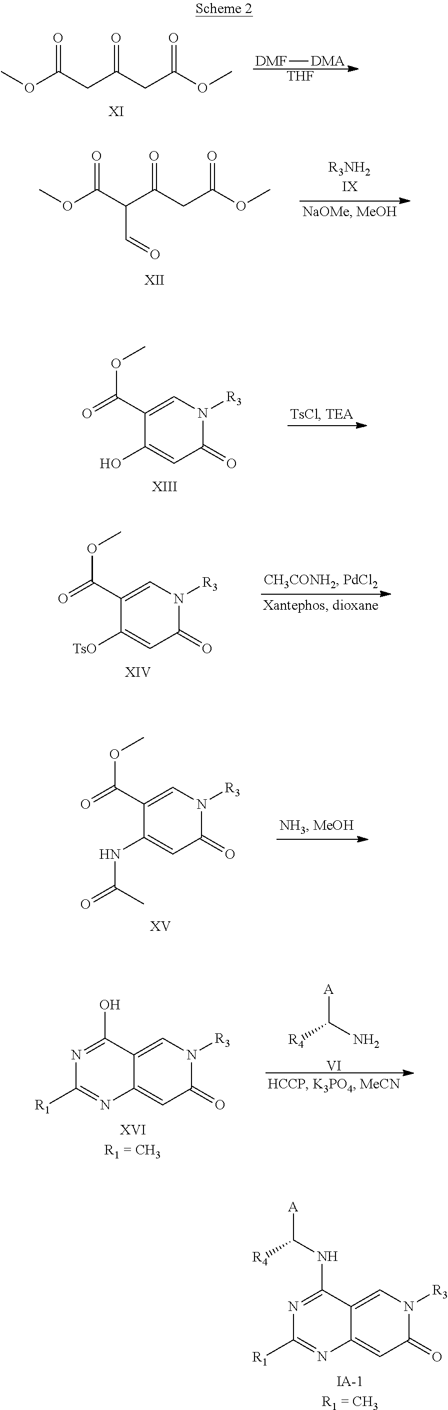

A compound of formula XVI, one the other hand, may be prepared by procedures known in the chemical arts including procedures described in WO2019/122129 also shown in Scheme 2. More specifically, a compound of formula XI is reacted with DMF-DMA in a solvent such as THF to provide a compound of formula XII. The compound of formula XII is condensed with a compound of formula IX under intramolecular cyclization conditions to provide a compound of formula XIII. The compound of formula XIII is reacted with TsCl in TEA to provide a compound of formula XIV. The compound of formula XIV is reacted with acetamide in the presence of a catalyst such as PdCl₂ to provide a compound of formula XV. The compound of formula XV is reacted with ammonia in a solvent such as methanol to provide a compound of formula XVI. The compound of formula XVI is reacted with a compound of formula VI in the presence of HCCP and K₃PO₄ in a solvent such as MeCN to provide a compound of formula IA-1 having R₁ being methyl.

A compound of formula XVI may also be prepared from a compound of formula Va as shown in Scheme 3 below. More specifically, a compound of formula Va is reacted with sodium methanethiolate in DMSO to provide a compound of formula XVII. The compound of formula XVII is reacted with a base such as NaOH in a solvent such as ethanol and water to provide a compound of formula XVIII. The compound of formula XVIII is reacted with a compound of formula IX in the presence of HATU and TEA to provide a compound of formula XIX. The reaction is conveniently carried out in a solvent such as acetonitrile and DMSO. The compound of formula XIX is reacted with an acid such as aqueous HCl to provide a compound of formula XVI having R₁ being methyl.

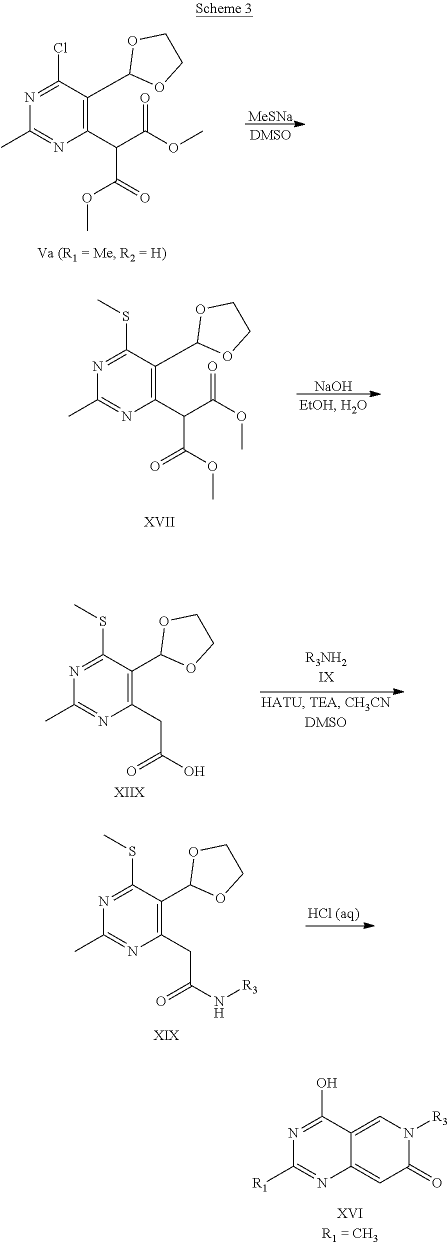

Additionally, a compound of formula XVI may be prepared from a compound of formula Vb as shown in Scheme 4. Specifically, a compound Vb is reacted with a base such as NaOH in a solvent such as EtOH to provide a compound of formula XX. The compound of formula XX is reacted with a compound of formula IX in the presence of HATU and TEA to provide a compound of formula XXI. The compound of formula XXI is reacted with an acid such as aqueous HCl to provide a compound of formula XVI. A compound of formula Vb may be prepared by procedures known in the chemical arts including those described in WO 2019/122129.

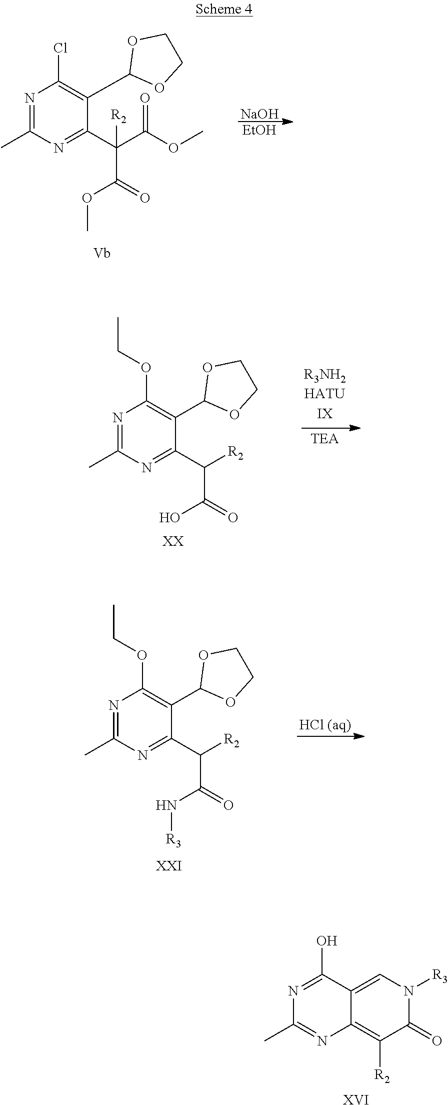

A compound of formula VI may be prepared from a compound of formula XXVI as shown in Scheme 5.

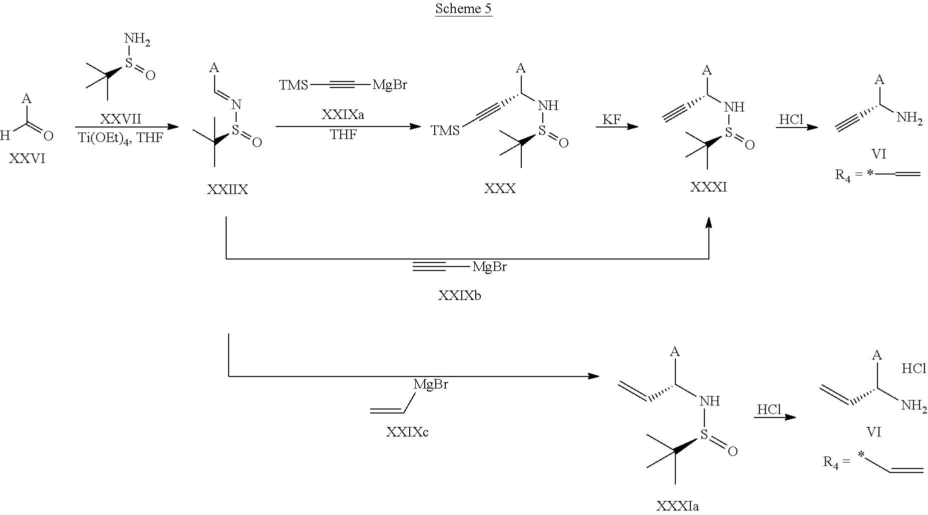

Specifically, a compound of formula XXVI is condensed with a chiral auxiliary such as (S)-2-methylpropane-2-sulfinamide (XXVII) in the presence of Lewis acid such as Ti(OEt)₄ to provide a compound of formula XXIIX. A stereospecific addition of ((trimethylsilyl)ethynyl)magnesium bromide (XXIXa), ethynyl magnesium bromide (XXIXb) or ethenyl magnesium bromide (XXIXc) to the compound of formula XXIIX provides a sulfinamide of formula XXX, XXXI and XXXIa, respectively. The resulting diastereomers of formula XXX, XXXI or XXXIa may be purified by normal or reverse phase chromatography to further enrich the chiral purity. The compound of formula XXX is reacted with potassium fluoride to provide a compound of formula XXXI. The compound of formula XXXI or XXXIa is reacted with an acid such aqueous HCl to provide a compound of formula VI having R₄ being acetylenyl or ethylenyl, respectively (Angew. Chem. Int. Ed. (2011), 50(14), 3236-3239; J. Org. Chem. (2010), 75(3), 941-944). A compound of formula XXVI is commercially available or may be prepared by procedures known in the chemical arts or prepared according to the procedures described in the sections below.

Some of Compounds 1-69 can be prepared using certain moieties, i.e., VIa-VIf, A-2, A-3, A-4, and A-5.

Preparation of these moieties and compounds 1-69 of this invention are described below.

Preparation of moieties VIa-VIh, A-2, A-3, A-4, and A-5

All chemicals and reagents were purchased from commercial suppliers (e.g., Sigma-Aldrich from St. Louis, Mo.), unless otherwise provided. Reactions were carried out under an atmosphere of dry nitrogen and monitored by thin-layer chromatography. The term “dried and concentrated” generally refers to drying of a solution in an organic solvent over either sodium sulfate or magnesium sulfate, followed by filtration and removal of the solvent from the filtrate (generally under reduced pressure and at a temperature suitable to the stability of the material being prepared). Purification was performed by column chromatography, preparative thin layer chromatography, or preparative high performance liquid chromatography (HPLC). The column chromatography was carried out with regular gravity or flash chromatography, or pre-packed silica gel cartridges using a medium pressure chromatography apparatus (e.g., Biotage Isolera One) eluting with the solvent or solvent mixture indicated. The preparative thin layer chromatography was carried out using 20 cm×20 cm×0.5 mm or 20 cm×20 cm×1 mm silica gel plates developed in a suitable solvent system. The HPLC was carried out using a reverse phase column (e.g., Waters Sunfire C18, Waters Xbridge C18) of a size appropriate to the quantity of material being separated, generally eluting with a gradient of increasing concentration of methanol or acetonitrile in water, also containing 0.05% or 0.1% formic acid (or trifluoroacetic acid) or 10 mM ammonium acetate, at a rate of elution suitable to the column size and separation to be achieved. Proton (¹H) nuclear magnetic resonance spectra were measured on a Varian Mercury-300 or Varian Mercury-400 spectrometer. Chemical shifts were recorded in parts per million (ppm) on the delta (6) scale relative to the resonance of the solvent peak. The following abbreviations were used to describe coupling: s=singlet; d=doublet; t=triplet; q=quartet; quin=quintet; br=broad; and m=multiplet. LCMS data were determined on an Agilent 6125B Single Quadrupole LC/MS system. Chemical names are generated using ChemDraw Professional version 19.1.

Synthesis of Moiety VIa: (R)-1-(2-methyl-3-(trifluoromethyl)phenyl)prop-2-yn-1-amine

Moiety VIa was prepared according to the scheme below:

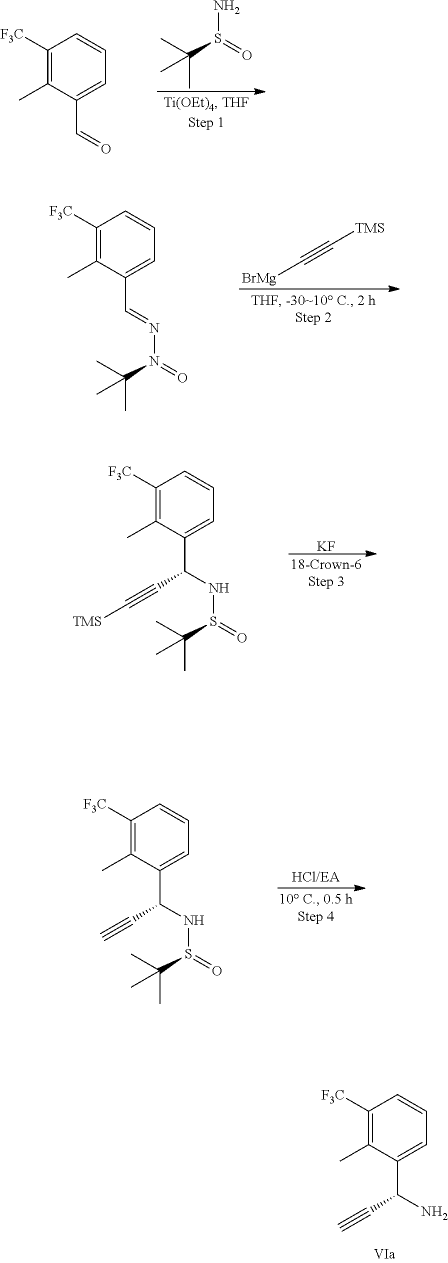

### Step 1. Synthesis of 2-methyl-N-[(1E)-[2-methyl-3-(trifluoromethyl)phenyl]-methylidene]propane-2-sulfinamide

To a solution of 2-methyl-3-(trifluoromethyl)benzaldehyde (23.0 g, 122 mmol) and (S)-(−)-2-methyl-2-propanesulfinamide (22.2 g, 183 mmol) in THF (300 mL) Ti(OEt)₄ (77 mL, 0.37 mol) was added at 15° C. under N₂. The reaction mixture was stirred at 80° C. for 12 hours. The mixture was diluted with EtOAc (1.0 L) and water (50 mL), then filtered. The organic layer was washed with brine (200 mL), dried over anhydrous Na₂SO₄, and filtered. The filtrate was concentrated under reduced pressure to give the title compound (32.9 g, 113 mmol, 92% yield) as a white solid, which was used in the next step without further purification.

¹H NMR (400 MHz, CDCl₃) δ 8.96 (s, 1H), 8.11 (d, J=7.8 Hz, 1H), 7.76 (d, J=7.8 Hz, 1H), 7.38 (t, J=7.8 Hz, 1H), 2.68 (d, J=1.0 Hz, 3H), 1.27 (s, 9H).

LC-MS m/e: 292 (MH⁺).

### Step 2. Synthesis of (S)-2-methyl-N-[(1S)-1-[2-methyl-3-(trifluoromethyl)phenyl]-3-(trimethylsilyl)prop-2-yn-1-yl]propane-2-sulfinamide

To a solution of (S)-2-methyl-N-[(1E)-[2-methyl-3-(trifluoromethyl) phenyl]methylidene] propane-2-sulfinamide (5.00 g, 17.2 mmol) in THF (100 mL) was added trimethylsilylethynyl magnesium bromide in THF (1 M, 51.5 mL, 51.5 mmol) at −30° C. under N₂. The reaction mixture was gradually warmed up to 10° C. and stirred for 1 hour, followed by addition of saturated NH₄Cl aqueous solution (10 mL). Brine (50 mL) was added and the resultant solution was extracted with EtOAc (100 mL×2). The combined extracts were dried over anhydrous Na₂SO₄ and filtered. The filtrate was concentrated under reduced pressure and purified by flash chromatography (gradient, PE/EtOAc=10/1 to 5/1) to give the title compound (6.3 g, 16.2 mmol, 94% yield) as a colorless oil.

¹H NMR (400 MHz, DMSO-d₆) δ 7.92 (d, J=7.7 Hz, 1H), 7.65 (d, J=7.7 Hz, 1H), 7.44 (t, J=7.8 Hz, 1H), 6.27 (d, J=7.6 Hz, 1H), 5.49 (d, J=7.6 Hz, 1H), 2.45 (s, 3H), 1.11 (s, 9H), 0.16 (s, 9H).

LC-MS m/e: (MH⁺).

### Step 3. Synthesis of (S)-2-methyl-N-[(1R)-1-[2-methyl-3-(trifluoromethyl)phenyl]prop-2-yn-1-yl]propane-2-sulfinamide

To a solution of (S)-2-methyl-N-[(1S)-1-[2-methyl-3-(trifluoromethyl)phenyl]-3-(trimethylsilyl)prop-2-yn-1-yl]propane-2-sulfinamide (6.69 g, 17.2 mmol) in THF (166 mL) and H₂O (3.4 mL) at 0° C., a solution of 18-crown-6 (5.0 g, 18.9 mmol) and potassium fluoride (0.443 mL, 18.9 mmol) in a mixture of THF (61.75 mL) and H₂O (1.25 mL) was added. The reaction solution was stirred at 0° C. for 2 hours. The reaction mixture was concentrated under reduced pressure. The resultant crude product was purified by flash chromatography (gradient, EtOAc/PE=1/10 to 1/2) to give the title compound (5.3 g, 16.7 mmol, 97% yield) as a colorless oil.

¹H NMR (400 MHz, DMSO-d₆) δ 7.94 (d, J=7.8 Hz, 1H), 7.66 (d, J=7.7 Hz, 1H), 7.45 (t, J=7.8 Hz, 1H), 6.22 (d, J=7.8 Hz, 1H), 5.45 (dd, J=7.7, 2.4 Hz, 1H), 3.60 (d, J=2.4 Hz, 1H), 2.45 (s, 3H), 1.08 (d, J=23.6 Hz, 9H).

LC-MS m/e: (MH⁺).

### Step 4. Synthesis of (R)-1-(2-methyl-3-(trifluoromethyl)phenyl)prop-2-yn-1-amine (VIa)

To a solution of (S)-2-methyl-N-[(1R)-1-[2-methyl-3-(trifluoromethyl)phenyl]prop-2-yn-1-yl]propane-2-sulfinamide (5.30 g, 16.7 mmol) in EtOAc (40 mL) in an ice bath, a solution of HCl in EtOAc (2.0 N, 20.9 mL, 41.8 mmol) was slowly added and stirred at 10° C. for 0.5 hours. The reaction mixture was concentrated under reduced pressure. The residue was diluted with MTBE (30 mL) and stirred at 25° C. for 0.5 hours and then filtered. The precipitate was washed with MTBE (20 mL) and dried in vacuum to provide the title compound (3.50 g, 16.4 mmol, 98.3% yield) as a white solid.

¹H NMR (400 MHz, DMSO-d₆) δ 9.12 (s, 3H), 7.99 (d, J=7.8 Hz, 1H), 7.78 (d, J=7.8 Hz, 1H), 7.56 (t, J=7.9 Hz, 1H), 5.65 (d, J=2.3 Hz, 1H), 3.96 (d, J=2.4 Hz, 1H), 2.50 (s, 3H).

LC-MS m/e: (MH⁺).

### Synthesis of moiety A-2: 3-(difluoromethyl)-2-methylbenzaldehyde

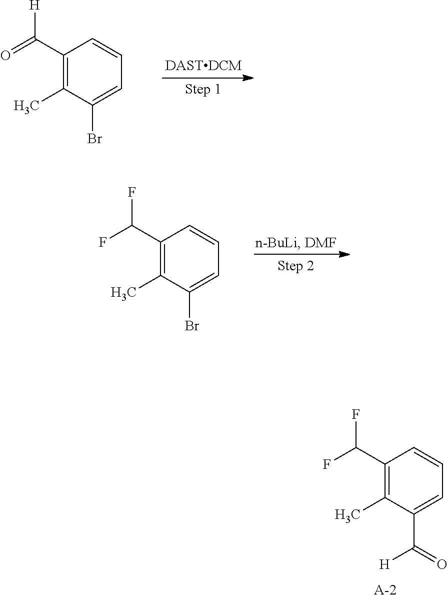

### Step 1. Synthesis of 1-bromo-3-(difluoromethyl)-2-methylbenzene

To a solution of 3-bromo-2-methylbenzaldehyde (4.00 g, 20.1 mmol) in DCM (15 mL) was added DAST (10.6 mL, 80.38 mmol) at 0° C. The resultant mixture was stirred at 30° C. for 4 hours. After the reaction is completed, the mixture was slowly poured into ice water (50 mL) and extracted with DCM (50 ml×2). The combine organic layers were washed with brine, dried over anhydrous Na₂SO₄, filtered, and concentrated. The residue was purified by column chromatography on silica gel (gradient, PE to 10% EtOAc/PE) to afford the title compound (3.5 g, 15.8 mmol, 79% yield) as a colorless oil.

¹H NMR (400 MHz, DMSO-d₆) δ 7.79 (d, J=8.0 Hz, 1H), 7.57 (d, J=7.7 Hz, 1H), 7.29 (t, J=7.9 Hz, 1H), 7.22 (t, J=54.6 Hz, 1H), 2.45 (s, 3H).

### Step 2. Synthesis of 3-(difluoromethyl)-2-methylbenzaldehyde (A-2)

To a solution of 1-bromo-3-(difluoromethyl)-2-methylbenzene (3.50 g, 15.8 mmol) in THF (15 mL), n-BuLi in hexane (2.5M, 7.6 mL, 19.00 mmol) was slowly added at −65° C. The resultant mixture was stirred at −65° C. under N₂ atmosphere for 1 hour. DMF (2.4 mL, 31.67 mmol) was slowly added at −65° C. and the mixture was stirred at the temperature for an additional hour. The resultant mixture was quenched by saturated NH₄Cl aqueous solution and extracted with EtOAc (50 mL×2). The combined organic layers were washed with brine, dried over anhydrous Na₂SO₄, and filtered. The filtrate was concentrated and purified by column chromatography on silica gel (gradient, PE to 10% EtOAc/PE) to afford the title compound (2.00 g, 11.8 mmol, 74% yield) as a colorless oil.

¹H NMR (400 MHz, DMSO-d₆) δ 10.35 (s, 1H), 7.97 (d, J=7.7 Hz, 1H), 7.83 (d, J=7.7 Hz, 1H), 7.55 (t, J=7.7 Hz, 1H), 7.31 (t, J=54.5 Hz, 1H), 2.70 (s, 3H).

### Synthesis of Moiety A-3: 1,1-difluoro-2,3-dihydro-1H-indene-4-carbaldehyde

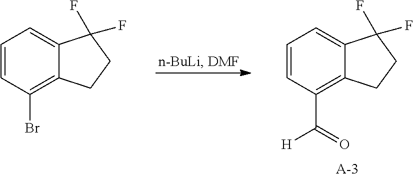

To a solution of 4-bromo-1,1-difluoro-2,3-dihydro-1H-indene (2.50 g, 10.7 mmol) (obtained according to the procedure described in WO 2019122129) in THF (15 mL) was slowly added n-BuLi in hexane (2.5M, 6.4 mL, 16.1 mmol) at −65° C. The resultant mixture was stirred at −65° C. under N₂ atmosphere for 1 hour. Then DMF (1.66 mL, 21.5 mmol) was slowly added at −65° C. and the mixture was stirred for an additional hour at −60° C. The resultant mixture was quenched with saturated NH₄Cl aqueous solution and extracted with EtOAc (50 mL×2). The combined organic layers were washed with brine, dried over anhydrous Na₂SO₄, and filtered. The filtrate was concentrated under reduced pressure and purified by column chromatography on silica gel (gradient, PE to 10% EtOAc/PE) to afford the title compound (1.20 g, 6.59 mmol, 61% yield) as a colorless oil.

¹H NMR (400 MHz, DMSO-d₆) δ 10.16 (s, 1H), 8.08 (dd, J=7.5, 0.7 Hz, 1H), 7.88 (dd, J=7.6, 0.9 Hz, 1H), 7.66 (t, J=7.6 Hz, 1H), 3.37 (ddd, J=10.5, 6.9, 3.5 Hz, 2H), 2.65 (ddd, J=21.6, 14.8, 6.9 Hz, 2H).

### Synthesis of Moiety A-4: 3,3-difluoro-2,3-dihydrobenzofuran-7-carbaldehyde

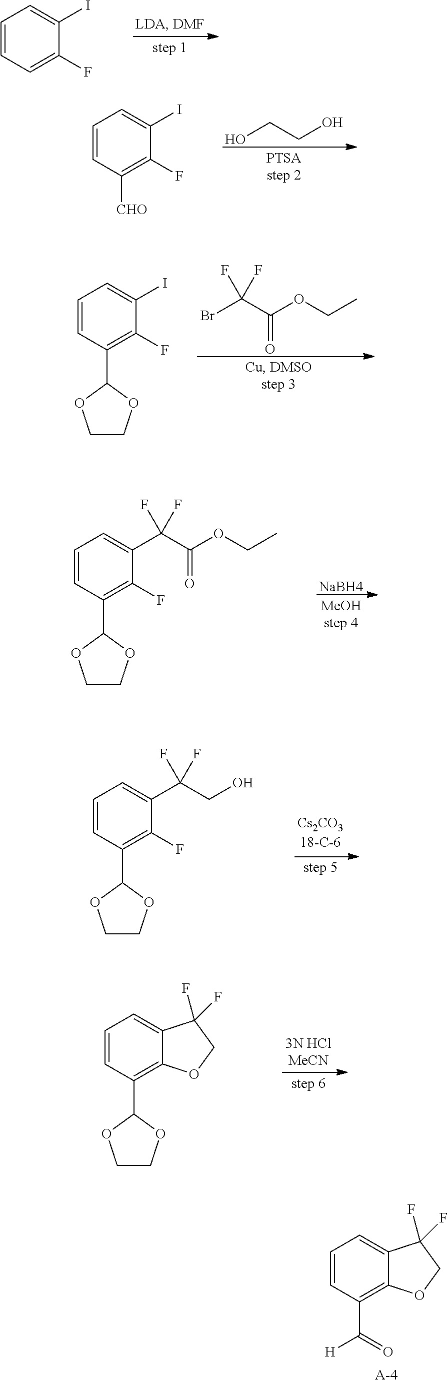

### Step 1. Synthesis of 2-fluoro-3-iodobenzaldehyde

To a solution of 1-fluoro-2-iodobenzene (21.00 g, 94.6 mmol) in THF (200 mL), LDA in THF (2M, 52.0 mL, 104 mmol) was slowly added at −70° C. and stirred at the temperature for 30 minutes. DMF (11.0 mL, 141.9 mmol) was slowly added and the solution was stirred at −70° C. for 1 hour. The resultant mixture was quenched with saturated NH₄Cl aqueous solution (100 mL) and extracted with EtOAc (100 mL×3). The combined organic layers were concentrated and purified by flash chromatography (gradient: PE to 10% EtOAc/PE) to give the title compound (16.0 g, 64.0 mmol, 68% yield) as a yellow oil.

¹H NMR (400 MHz, CDCl₃) δ 10.33 (1H), 8.01 (ddd, J=7.8, 6.0, 1.7 Hz, 1H), 7.84 (ddd, J=7.9, 6.4, 1.7 Hz, 1H), 7.06 (t, J=7.8 Hz, 1H).

### Step 2. Synthesis of 2-(2-fluoro-3-iodophenyl)-1,3-dioxolane

To a stirred solution of 2-fluoro-3-iodobenzaldehyde (16.0 g, 64.0 mmol) in toluene (150 mL) was added ethane-1,2-diol (3.6 mL, 64.0 mmol) and TsOH (1.0 mL, 6.4 mmol). The mixture was stirred at 110° C. for 16 hours, cooled down to room temperature, and quenched with saturated NaHCO₃ aqueous solution (100 mL), and extracted with EtOAc (50 mL×3). The combined organic layers were concentrated and purified by flash chromatography (gradient: PE to 10% EtOAc/PE) to give the title compound (12.0 g, 40.8 mmol, 64% yield) as a yellow oil.

¹H NMR (400 MHz, CDCl₃) δ 7.75 (ddd, J=7.7, 5.9, 1.7 Hz, 1H), 7.51 (d, J=7.7 Hz), 6.93 (t, J=7.8 Hz, 1H), 6.06 (s, 1H), 4.14 (dt, J=12.5, 8.2 Hz, 2H), 4.06 (dt, J=9.1, 8.2 Hz, 2H).

### Step 3. Synthesis of ethyl 2-(3-(1,3-dioxolan-2-yl)-2-fluorophenyl)-2,2-difluoroacetate

To a stirred solution of 2-(2-fluoro-3-iodophenyl)-1,3-dioxolane (5.00 g, 17.0 mmol) in DMSO (100 mL), ethyl 2-bromo-2,2-difluoroacetate (5.50 mL, 42.6 mmol) and copper (2.72 g, 42.51 mmol) were added under N₂. The reaction mixture was stirred at 80° C. for 16 hours, cooled to room temperature, poured into water (300 mL), and then extracted with EtOAc (100 mL×2). The combined organic layers were dried over Na₂SO₄ and filtered. The filtrate was concentrated and purified by flash chromatography (gradient: PE to 20% EtOAc/PE) to give the title compound (4.50 g, 15.5 mmol, 91% yield) as a yellow oil.

¹H NMR (400 MHz, CDCl₃) δ 7.74-7.62 (m, 2H), 7.30-7.26 (m, 1H), 6.08 (s, 1H), 4.35 (q, J=7.1 Hz, 2H), 4.18-4.10 (m, 2H), 4.11-4.02 (m, 2H), 1.32 (t, J=7.1 Hz, 3H).

### Step 4. Synthesis of 2-(3-(1,3-dioxolan-2-yl)-2-fluorophenyl)-2,2-difluoroethan-1-ol

To a stirred solution of ethyl 2-[3-(1,3-dioxolan-2-yl)-2-fluorophenyl]-2,2-difluoroacetate (4.50 g, 15.5 mmol) in MeOH (100 mL) was added NaBH₄ (0.59 g, 15.5 mmol) at 0° C. The resultant mixture was stirred for additional 2 hours, quenched with H₂O (100 mL), and extracted with DCM (50 mL×3). The combined organic layers were concentrated to give the title compound (3.50 g, 14.1 mmol, 91% yield) as a white solid, which was used in the next step without further purification.

LC-MS m/e: 249 (MH⁺).

### Step 5. Synthesis of 7-(1,3-dioxolan-2-yl)-3,3-difluoro-2,3-dihydrobenzofuran

To a stirred solution of 2-[3-(1,3-dioxolan-2-yl)-2-fluorophenyl]-2,2-difluoroethan-1-ol (4.00 g, 16.1 mmol) in THF (50 mL), Cs₂CO₃ (15.7 g, 48.4 mmol) and 18-crown-6 (0.43 g, 1.6 mmol) were added. The resultant mixture was stirred at 80° C. for 16 hours, cooled down to room temperature, quenched with H₂O (100 mL), and extracted with EtOAc (50 mL×3). The combined organic layers were concentrated and purified by flash chromatography (gradient: PE to EtOAc:PE=4:6) to give the title compound (1.20 g, 5.26 mmol, 33% yield) as a yellow oil.

LC-MS m/e: 229 (MH⁺).

### Step 6. Synthesis of 3,3-difluoro-2,3-dihydrobenzofuran-7-carbaldehyde (A-4)

To a stirred solution of 7-(1,3-dioxolan-2-yl)-3,3-difluoro-2,3-dihydro-1-benzofuran (1.20 g, 5.26 mmol) in CH₃CN (10 mL), aqueous HCl solution (2.65 N, 2.0 mL, 5.3 mmol) was added. The resultant mixture was stirred at 50° C. for 3 hours, cooled to room temperature, and extracted with EtOAc (30 mL×3). The combined organic layers were concentrated and purified by flash chromatography (gradient: PE to PE:EtOAc=6:4) to give the title compound (0.80 g, 4.3 mmol, 83% yield) as a white solid.

¹H NMR (400 MHz, CDCl₃) δ 10.25 (s, 1H), 7.95 (d, J=7.7 Hz, 1H), 7.78 (dd, J=7.5, 1.4 Hz, 1H), 7.20 (t, J=7.6 Hz, 1H), 4.78 (t, J=15.7 Hz, 2H).

### Synthesis of Moiety A-5: 3-(1,1-difluoroethyl)-2-fluorobenzaldehyde

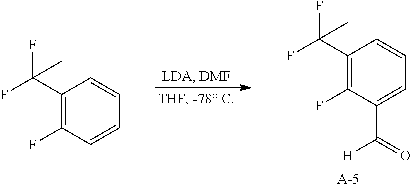

To a solution of 1-(1,1-difluoroethyl)-2-fluorobenzene (16.0 g, 100.0 mmol) in THF (400.0 mL), LDA in THF (2.5 M, 52.0 mL, 130 mmol) was slowly added at −78° C. The resultant mixture was stirred at the temperature for 2 hours. DMF (8.76 g, 120 mmol) was then slowly added and the solution was stirred at −78° C. for additional 2 hours. The reaction mixture was then quenched with cold saturated NH₄Cl aqueous solution (50 mL) and extracted with EtOAc (600 mL×2). The combined organic layers were washed with brine, dried over anhydrous Na₂SO₄, and filtered. The filtrate was concentrated and purified by silica gel chromatography (gradient: PE to PE:EtOAc=20:1) to give the title compound as a green oil (5.90 g, 31.4 mmol, 31% yield).

¹H NMR (400 MHz, CDCl₃) δ 10.40 (s, 1H), 8.11-7.89 (m, 1H), 7.81 (td, J=7.7, 1.7 Hz, 1H), 7.33 (t, J=7.7 Hz, 1H), 2.04 (td, J=18.6, 1.0 Hz, 3H).

### Synthesis of Moiety VIb: (R)-1-(3-(difluoromethyl)-2-fluorophenyl)prop-2-yn-1-amine hydrochloride

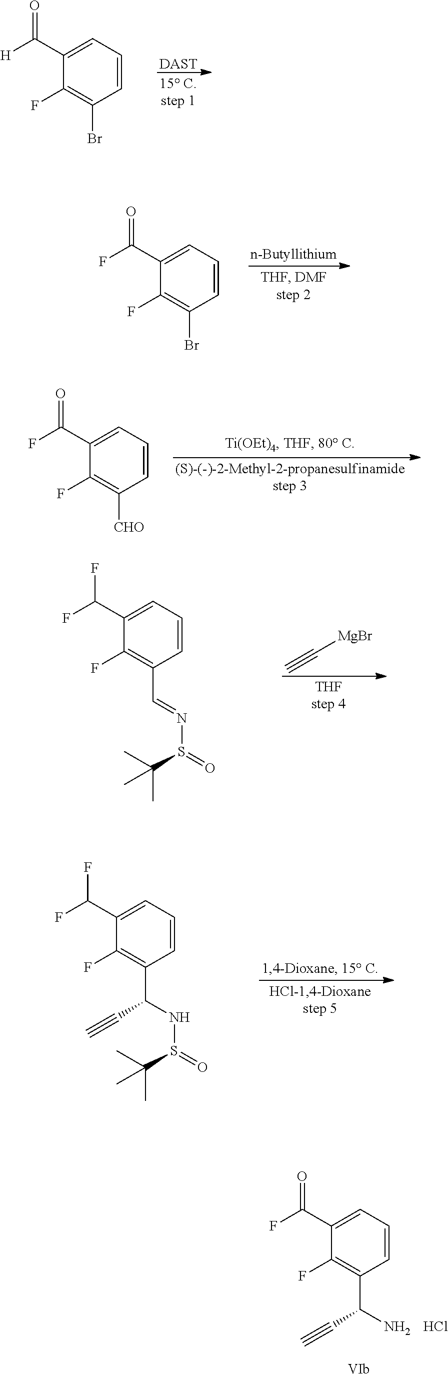

### Step 1. Synthesis of 1-bromo-3-(difluoromethyl)-2-fluorobenzene

To a solution of (3-bromo-2-fluorophenyl)(hydrogenio)formaldehyde (2.00 g, 9.85 mmol) in DCM (45 mL) was added DAST (2.6 mL, 19.68 mmol) dropwise at −10° C. The reaction mixture was stirred in ice bath and gradually warmed to RT over 16 hours. The mixture was then poured into saturated aqueous NaHCO₃ solution (50 mL) and extracted with EtOAc (50 mL×3). The combined organic layers were dried over Na₂SO₄ and filtered. The filtrate was concentrated to afford the crude product (1.50 g, 6.67 mmol, 68% yield) as a colorless oil, which was used in the next step without further purification.

¹H NMR (400 MHz, DMSO-d₆) δ 7.93 (t, J=7.4 Hz, 1H), 7.68 (t, J=7.0 Hz, 1H), 7.51-7.33 (m, 1H), 7.27 (t, J=54.1 Hz, 1H).

### Step 2. Synthesis of 3-(difluoromethyl)-2-fluorobenzaldehyde

To a solution of 1-bromo-3-(difluoromethyl)-2-fluorobenzene (500 mg, 2.222 mmol) in THF (15 mL) n-butyllithium in hexane (2.5 M, 1.0 mL, 2.5 mmol) was added dropwise at −70° C. The mixture was stirred at the temperature for 1 hour. DMF (0.34 mL, 4.44 mmol) was then slowly added at −70° C. and the resultant solution was stirred for additional 2 hours. The solution was then quenched with saturated aqueous NH₄Cl (20 mL) and extracted with EtOAc (30 mL×3). The combined organic layers were dried over anhydrous Na₂SO₄ and filtered. The filtrate was condensed under reduced pressure to give a crude product, which was purified by flash chromatography (gradient: PE to PE:EtOAc=10:1) to give the title compound (200 mg, 1.15 mmol, 52% yield) as a brown oil.

¹H NMR (400 MHz, CDCl₃) δ 10.37 (1H), 8.01 (t, J=7.1 Hz, 1H), 7.87 (t, J=7.1 Hz, 1H), 7.40 (t, J=7.7 Hz, 1H), 6.95 (t, J=54.0 Hz, 1H).

### Step 3. Synthesis of (S, E)-N-(3-(difluoromethyl)-2-fluorobenzylidene)-2-methylpropane-2-sulfinamide

To a solution of 3-(difluoromethyl)-2-fluorobenzaldehyde (3.20 g, 18.38 mmol) in THF (10 mL), titanium ethoxide (11.0 mL, 55.13 mmol) and (S)-(−)-2-methyl-2-propanesulfinamide (3.0 mL, 27.57 mmol) were added. The resultant mixture was stirred at 80° C. for 16 hours, cooled to room temperature, quenched with ice water (100 mL) and ethyl acetate (100 mL), and filtered. The filtrate was extracted with EtOAc (30 mL×3). The combined organic layers were dried over anhydrous Na₂SO₄ and filtered. The resultant filtrate was concentrated under reduced pressure to give a crude product, which was purified by column chromatography (gradient: PE to PE:EtOAc=10:1) to give the title compound (3.20 g, 11.54 mmol, 63% yield) as a yellow solid.

¹H NMR (400 MHz, CDCl₃) δ 8.90 (s, 1H), 8.13 (t, J=7.2 Hz, 1H), 7.77 (t, J=6.6 Hz, 1H), 7.36 (t, J=7.8 Hz, 1H), 6.94 (t, J=54.0 Hz, 1H), 1.29 (s, 9H).

### Step 4. Synthesis of (S)—N—((R)-1-(3-(difluoromethyl)-2-fluorophenyl)prop-2-yn-1-yl)-2-methylpropane-2-sulfinamide

To a solution of (S)—N-[(1E)-[3-(difluoromethyl)-2-fluorophenyl]methylidene]-2-methylpropane-2-sulfinamide (500 mg, 1.803 mmol) in THF (10 mL), ethynylmagnesium bromide in THF (0.5 M, 36.0 mL, 18.0 mmol) was added dropwise at −60° C. The reaction mixture was gradually warmed to 15° C. and stirred for additional 3 hours. The mixture was then quenched with saturated aqueous NH₄Cl solution (20 mL) and extracted with EtOAc (50 mL×3). The combined organic layers were dried over anhydrous Na₂SO₄ and filtered. The filtrate was concentrated under reduced pressure and purified by column chromatography (gradient: PE to PE:EtOAc=2:1) to give the title compound (250 mg, 0.82 mmol, 46% yield) as a brown oil.

¹H NMR (400 MHz, DMSO-d₆) δ 7.87 (t, J=7.2 Hz, 1H), 7.62 (t, J=7.0 Hz, 1H), 7.40 (t, J=7.7 Hz, 1H), 7.23 (t, J=54.2 Hz, 1H), 6.32 (d, J=7.9 Hz, 1H), 5.46 (dd, J=7.9, 2.4 Hz, 1H), 3.65 (d, J=2.5 Hz, 1H), 1.09 (s, 9H).

### Step 5. Synthesis of (R)-1-(3-(difluoromethyl)-2-fluorophenyl)prop-2-yn-1-amine hydrochloride (VIb)

To a solution of (S)—N-[(1R)-1-[3-(difluoromethyl)-2-fluorophenyl]prop-2-yn-1-yl]-2-methylpropane-2-sulfinamide (500 mg, 1.65 mmol) in 1,4-dioxane (5 mL), a solution of HCl in dioxane (4 N, 5 mL, 20.0 mmol) was added dropwise at room temperature. The resultant mixture was stirred at room temperature for 2 hours. The solvent was then evaporated under reduced pressure. The residue was triturated in methyl tertbutyl ether and filtrated to afford the title compound (300 mg, 1.51 mmol, 91% yield) as a white solid.

¹H NMR (400 MHz, DMSO-d₆) δ 9.10 (s, 1H), 7.94 (t, J=7.3 Hz, 1H), 7.76 (t, J=6.9 Hz, 1H), 7.50 (t, J=7.8 Hz, 1H), 7.28 (t, J=54.0 Hz, 1H), 5.67 (d, J=2.4 Hz, 1H), 4.01 (d, J=2.4 Hz, 1H).

Each of moieties VIc-VIh shown in Table 2 was synthesized following a procedure similar to that used to prepare moiety VIb described above.

TABLE 2

| | | LC-MS | |
|---|---|---|---|
| Moi- | | m/e | |
| ety | Structure | (MH⁺) | ¹H NMR |
| VIc | 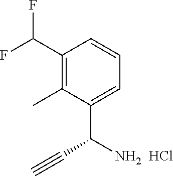 | 196.2 | ¹H NMR (400 MHz, DMSO-d₆) δ 9.12 (s, 3H), 7.87 (d, J = 7.8 Hz, 1H), 7.62 (d, J = 7.6 Hz, 1H), 7.49 (t, J = 7.8 Hz, 1H), 7.27 (t, J = 54.6 Hz, 1H), 5.59 (d, J = 2.2 Hz, 1H), 3.93 (d, J = 2.2 Hz, 1H), 2.46 (s, 3H) |
| VId | 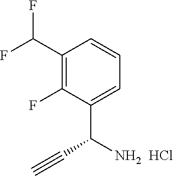 | 200.2 | ¹H NMR (400 MHz, DMSO-d₆) δ 9.10 (s, 3H), 7.94 (t, J = 7.3 Hz, 1H), 7.76 (t, J = 6.9 Hz, 1H), 7.50 (t, J = 7.8 Hz, 1H), 7.28 (t, J = 54.0 Hz, 1H), 5.67 (d, J = 2.4 Hz, 1H), 4.01 (d, J = 2.4 Hz, 1H). |
| VIe | 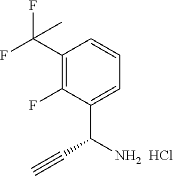 | 214.2 | ¹H NMR (400 MHz, DMSO-d₆) δ 9.24 (s, 3H), 7.93 (t, J = 7.0 Hz, 1H), 7.68 (t, J = 7.2 Hz, 1H), 7.45 (t, J = 7.8 Hz, 1H), 5.62 (d, J = 2.3 Hz, 1H), 3.99 (d, J = 2.1 Hz, 1H), 2.03 (t, J = 19.2 Hz, 3H). |
| VIf | 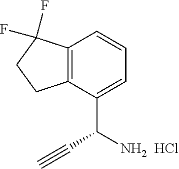 | 208.2 | ¹H NMR (400 MHz, DMSO-d₆) δ 9.13 (s, 3H), 7.84 (d, J = 7.5 Hz, 1H), 7.64 (d, J = 7.6 Hz, 1H), 7.55 (t, J = 7.6 Hz, 1H), 5.44 (d, J = 2.4 Hz, 1H), 3.95 (d, J = 2.4 Hz, 1H), 3.29-3.12 (m, 2H), 2.69-2.55 (m, 2H) |
| VIg | 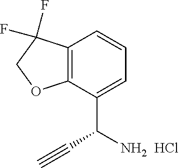 | 210.2 | ¹H NMR (400 MHz, DMSO-d₆) δ 9.15 (s, 3H), 7.87 (d, J = 7.6 Hz, 1H), 7.84-7.69 (m, 1H), 7.27 (t, J = 7.7 Hz, 1H), 5.44 (d, J = 2.2 Hz, 1H), 5.06-4.79 (m, 2H), 3.96 (d, J = 2.4 Hz, 1H) |
| VIh | 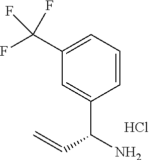 | 216.2 | ¹H NMR (400 MHz, DMSO-d₆) δ 8.89 (s, 3H), 7.87 (d, J = 7.9 Hz, 1H), 7.74 (d, J = 7.7 Hz, 1H), 7.54 (t, J = 7.9 Hz, 1H), 6.09 (ddd, J = 17.0, 10.5, 6.3 Hz, 1H), 5.45- 5.21 (m, 3H), 2.46 (s, 3H). |

### Examples 1-65: Syntheses of Compounds 1-65

Compounds 1-65 of this invention were prepared following the procedures provided below. Unless otherwise described above such as preparation of moieties compounds, all other reagents are commercially available from various suppliers.

### Synthesis of Compound 1: (R)-4-((1-(3-(difluoromethyl)-2-methylphenyl)prop-2-yn-1-yl)amino)-6-(1-(difluoromethyl)cyclopropyl)-2-methylpyrido[4,3-d]pyrimidin-7 (6H)-one

The scheme below depicts synthesis of Compound 1:

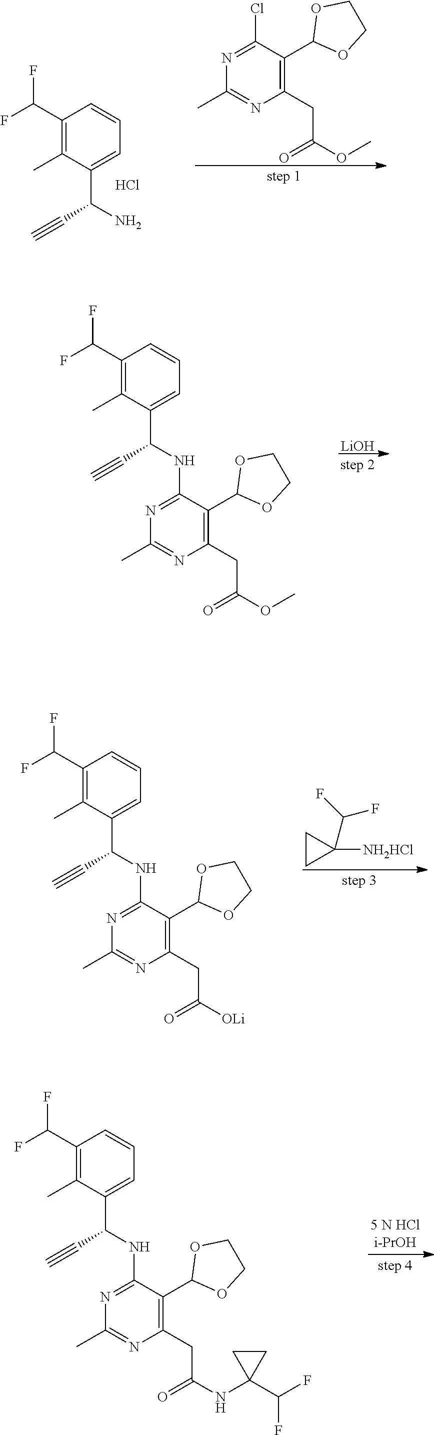

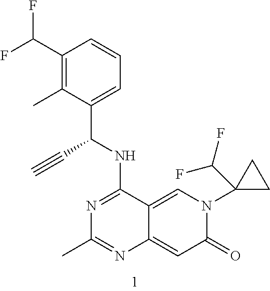 [SMILES: C#C[C@@H](Nc1nc(C)nc2cc(=O)n(C3(C(F)F)CC3)cc12)c1cccc(C(F)F)c1C]

### Step 1. Synthesis of methyl (R)-2-(6-((1-(3-(difluoromethyl)-2-methylphenyl)prop-2-yn-1-yl)amino)-5-(1,3-dioxolan-2-yl)-2-methylpyrimidin-4-yl)acetate

To a solution of (1R)-1-[3-(difluoromethyl)-2-methylphenyl]prop-2-yn-1-amine hydrochloride (509.7 mg, 2.20 mmol) in DMSO (10 mL) was added methyl 2-(6-chloro-5-(1,3-dioxolan-2-yl)-2-methylpyrimidin-4-yl)acetate (500 mg, 1.83 mmol) (obtained according to the procedure described in WO2019/122129) and DIPEA (0.91 mL, 5.50 mmol). The resultant mixture was stirred at 100° C. overnight. The mixture was then cooled down to room temperature, diluted with water (30 mL), and extracted with EtOAc (100 mL×2). The combined organic layers were washed with brine, dried over anhydrous Na₂SO₄, and filtered. The filtrate was concentrated under reduced pressure and purified by column chromatography on silica gel (gradient, EtOAc/PE=30% to 60%) to afford the title compound (400 mg, 0.93 mol, 51% yield) as a yellow oil.

LC-MS m/e: 432.4 (MH⁺).

### Step 2. Synthesis of lithium (R)-2-(6-((1-(3-(difluoromethyl)-2-methylphenyl)prop-2-yn-1-yl)amino)-5-(1,3-dioxolan-2-yl)-2-methylpyrimidin-4-yl)acetate

To a solution of methyl 2-(6-{[(1R)-1-[3-(difluoromethyl)-2-methylphenyl]prop-2-yn-1-yl]amino}-5-(1,3-dioxolan-2-yl)-2-methylpyrimidin-4-yl)acetate (400 mg, 0.927 mmol) in MeCN (5 mL), a solution of LiOH (42.8 mg, 1.02 mmol) in H₂O (1 mL) was added. The mixture was stirred at room temperature overnight and then concentrated under reduced pressure to give a residue. The residue was dried in vacuum to afford the title compound (390 mg, 0.92 mmol, 99% yield) as an off-white solid, which was used in next step without further purification.

LC-MS m/e: 418.2 (MH⁺).

### Step 3. Synthesis of (R)-2-(6-((1-(3-(difluoromethyl)-2-methylphenyl)prop-2-yn-1-yl)amino)-5-(1,3-dioxolan-2-yl)-2-methylpyrimidin-4-yl)-N-(1-difluoromethyl)cyclopropyl) acetamide

To a solution of lithium (R)-2-(6-((1-(3-(difluoromethyl)-2-methylphenyl)prop-2-yn-1-yl)amino)-5-(1,3-dioxolan-2-yl)-2-methylpyrimidin-4-yl)acetate (100 mg, 0.23 mmol) and 1-(difluoromethyl)cyclopropan-1-amine hydrochloride (37 mg, 0.26 mmol) (obtained according to the procedure described in WO2019/122129) in a mixture of DMSO (4 mL) and MeCN (2 mL), TEA (0.1 mL, 0.71 mmol) and HATU (98.7 mg, 0.26 mmol) were added. The resultant mixture was stirred at room temperature for 1 hour, diluted with EtOAc (50 mL), and washed with brine (30 mL). The organic layer was dried over anhydrous Na₂SO₄ and filtered. The filtrate was concentrated to afford the title compound (100 mg, 0.25 mmol, 84% yield) as a yellow oil.

LC-MS m/e: 507.3 (MH⁺).

### Step 4. Synthesis of (R)-4-((1-(3-(difluoromethyl)-2-methylphenyl)prop-2-yn-1-yl)amino)-6-(1-(difluoromethyl)cyclopropyl)-2-methylpyrido[4,3-d]pyrimidin-7 (6H)-one (Compound 1)

To a solution of (R)-2-(6-((1-(3-(difluoromethyl)-2-methylphenyl)prop-2-yn-1-yl)amino)-5-(1,3-dioxolan-2-yl)-2-methylpyrimidin-4-yl)-N-(1-(difluoromethyl)cyclopropyl)-acetamide (100 mg, 0.20 mmol) in isopropyl alcohol (10 mL), 5N HCl aqueous solution (1.0 mL, 5.00 mmol) was added. The resultant mixture was stirred at 50° C. for 5 hours. The mixture was then adjusted to pH˜7 with saturated NaHCO₃ aqueous solution and extracted with EtOAc (30 mL×2). The combined organic layers were washed with brine, dried over anhydrous Na₂SO₄, and filtered. The filtrate was concentrated to give a residue, which was purified by Pre-HPLC (SilaSep™ C18 silica flash cartridge, gradient, 5% to 55% MeCN in H₂O with 0.1% formic acid) to afford the title compound (14.3 mg, 0.03 mmol, 16% yield) as a light yellow solid.

¹H NMR (400 MHz, DMSO-d₆) δ 9.37 (s, 1H), 9.17 (s, 1H), 7.87 (d, J=7.7 Hz, 1H), 7.53 (d, J=7.5 Hz, 1H), 7.43 (t, J=7.7 Hz, 1H), 7.23 (t, J=54.8 Hz, 1H), 6.63 (s, 1H), 6.30 (t, J=57.0 Hz, 1H), 6.13 (s, 1H), 3.64 (d, J=2.3 Hz, 1H), 2.42 (s, 3H), 2.28 (s, 3H), 1.51-1.27 (m, 4H).

LC-MS m/e: 445.4 (MH⁺).

### Syntheses of Compounds 2-17

Compounds 2-17 shown in Table 3 below were prepared in a manner similar to that used to prepare Compound 1 set forth above.

TABLE 3

| | | LC-MS | |
|---|---|---|---|
| Compound | | m/e | |
| No. | Structure | (MH⁺) | ¹H NMR |
| 2 | 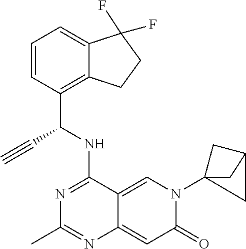 [SMILES: C#C[C@@H](Nc1nc(C)nc2cc(=O)n(C34CC(C3)C4)cc12)c1cccc2c1CCC2(F)F] | 433.4 | H NMR (400 MHz, CD₃OD) δ 8.88 (s, 1H), 7.91 (d, J = 7.4 Hz, 1H), 7.54-7.43 (m, 2H), 6.73 (d, J = 2.4 Hz, 1H), 6.30 (s, 1H), 3.15-3.07 (m, 3H), 2.70 (s, 1H), 2.64-2.54 (m, 2H), 2.49-2.39 (m, 9H). |
| 3 | 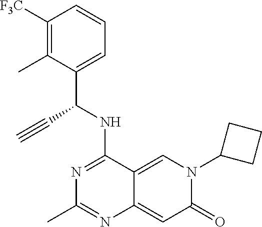 [SMILES: C#C[C@@H](Nc1nc(C)nc2cc(=O)n(C3CCC3)cc12)c1cccc(C(F)(F)F)c1C] | 427.1 | ¹H NMR (400 MHz, DMSO-d₆) δ 9.28 (s, 1H), 8.01 (d, J = 7.6 Hz, 1H), 7.73 (d, J = 7.7 Hz, 1H), 7.53 (t, J = 7.8 Hz, 1H), 6.81 (d, J = 2.5 Hz, 1H), 6.16 (s, 1H), 4.96-5.02 (m, 1H), 3.75 (d, J = 2.5 Hz, 1H), 2.48 (3H), 2.46 (s, 3H), 2.49-2.28 (m, 4H), 1.82 (td, J = 9.9, 4.8 Hz, 2H). |
| 4 | 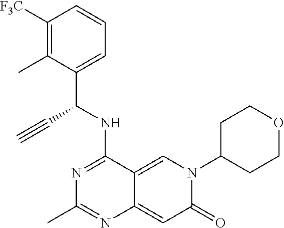 [SMILES: C#C[C@@H](Nc1nc(C)nc2cc(=O)n(C3CCOCC3)cc12)c1cccc(C(F)(F)F)c1C] | 457.2 | ¹H NMR (400 MHz, DMSO-d₆) δ 9.14 (s, 1H), 7.99 (d, J = 7.7 Hz, 1H), 7.70 (d, J = 7.7 Hz, 1H), 7.51 (t, J = 7.8 Hz, 1H), 6.64 (d, J = 2.1 Hz, 1H), 6.17 (s, 1H), 5.15 (ddd, J = 12.1, 8.3, 3.9 Hz, 1H), 4.09-4.00 (m, 2H), 3.69 (d, J = 2.5 Hz, 1H), 3.62-3.58 (m, 3H), 2.52 (s, 3H), 2.28 (s, 3H), 2.09-1.94 (m, 2H), 1.85-1.73 (m, 2H). |
| 5 | 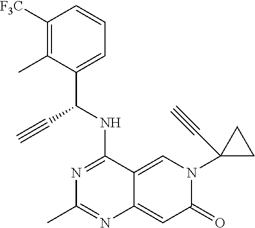 [SMILES: C#C[C@@H](Nc1nc(C)nc2cc(=O)n(C3(C#C)CC3)cc12)c1cccc(C(F)(F)F)c1C] | 437.1 | ¹H NMR (400 MHz, DMSO-d₆) δ 9.33 (s, 1H), 8.03 (d, J = 7.8 Hz, 1H), 7.72 (d, J = 7.7 Hz, 1H), 7.52 (t, J = 7.8 Hz, 1H), 6.67 (s, 1H), 6.13 (s, 1H), 3.74 (s, 1H), 3.32 (s, 3H), 2.33 (s, 3H), 1.55 (s, 4H), 1.53 (br s, 2H) |
| 6 | 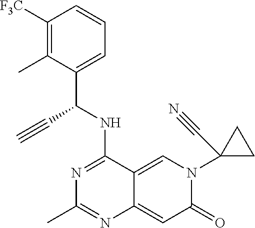 [SMILES: C#C[C@@H](Nc1nc(C)nc2cc(=O)n(C3(C#N)CC3)cc12)c1cccc(C(F)(F)F)c1C] | 438.2 | ¹H NMR (400 MHz, DMSO-d₆) δ 9.34 (d, J = 7.3 Hz, 1H), 9.19 (s, 1H), 8.02 (d, J = 8.1 Hz, 1H), 7.71 (d, J = 7.6 Hz, 1H), 7.51 (t, J = 7.9 Hz, 1H), 6.63 (dd, J = 7.2, 2.3 Hz, 1H), 6.18 (s, 1H), 3.70 (d, J = 2.4 Hz, 1H), 2.28 (s, 3H), 1.95 (s, 3H), 1.83-1.68 (m, 4H). |
| 7 | 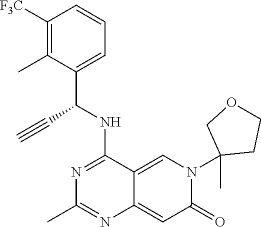 [SMILES: C#C[C@@H](Nc1nc(C)nc2cc(=O)n(C3(C)CCOC3)cc12)c1cccc(C(F)(F)F)c1C] | 457.2 | ¹H NMR (400 MHz, CD₃OD δ 8.94 (s, 1H), 8.06 (d, J = 7.9 Hz, 1H), 7.67 (d, J = 7.7 Hz, 1H), 7.43 (t, J = 7.9 Hz, 1H), 6.82 (t, J = 2.4 Hz, 1H), 6.35 (s, 1H), 4.47 (d, J = 9.4 Hz, 1H), 4.08-3.92 (m, 3H), 3.09 (d, J = 2.5 Hz, 1H), 2.64-2.47 (m, 2H), 2.59 (s, 3H), 2.46-2.35 (m, 3H), 1.69 (d, J = 3.7 Hz, 3H). (mixture of diastereomers) |
| 8 | 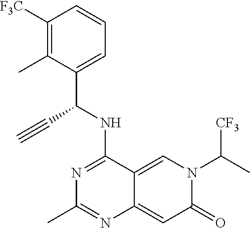 [SMILES: C#C[C@@H](Nc1nc(C)nc2cc(=O)n(C(C)C(F)(F)F)cc12)c1cccc(C(F)(F)F)c1C] | 469.1 | ¹H NMR (400 MHz, CD₃OD δ 9.14 (s, 1H), 8.05 (d, J = 7.8 Hz, 1H), 7.68 (d, J = 7.7 Hz, 1H), 7.43 (t, J = 7.8 Hz, 1H), 6.81 (d, J = 2.4 Hz, 1H), 6.43 (s, 1H), 6.22-6.08 (m, 1H), 3.11 (t, J = 2.6 Hz, 1H), 2.54 (s, 3H), 2.42 (dt, J = 5.5, 3.6 Hz, 3H), 1.76 (t, J = 7.8 Hz, 3H). (mixture of diastereomers) |
| 9 | 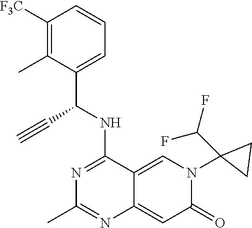 [SMILES: C#C[C@@H](Nc1nc(C)nc2cc(=O)n(C3(C(F)F)CC3)cc12)c1cccc(C(F)(F)F)c1C] | 463.2 | ¹H NMR (400 MHz, DMSO-d₆) δ 9.41 (d, J = 7.3 Hz, 1H), 9.18 (s, 1H), 8.00 (d, J = 7.7 Hz, 1H), 7.70 (d, J = 7.7 Hz, 1H), 7.51 (t, J = 7.9 Hz, 1H), 6.64 (dd, J = 7.3, 2.4 Hz, 1H), 6.31 (t, J = 57.0 Hz, 1H), 6.14 (s, 1H), 3.70 (d, J = 2.5 Hz, 1H), 2.50 (s, 3H), 2.28 (s, 3H), 1.30-1.50 (m, 4H). |
| 10 | 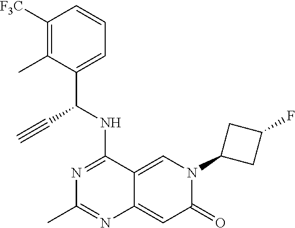 [SMILES: C#C[C@@H](Nc1nc(C)nc2cc(=O)n([C@H]3C[C@H](F)C3)cc12)c1cccc(C(F)(F)F)c1C] | 445 | ¹H NMR (400 MHz, DMSO-d₆) δ 9.48 (s, 1H), 9.06 (d, J = 18.6 Hz, 1H), 8.02 (d, J = 7.8 Hz, 1H), 7.70 (d, J = 7.7 Hz, 1H), 7.51 (t, J = 7.8 Hz, 1H), 6.70 (s, 1H), 6.13 (s, 1H), 5.35-5.23 (m, 1H), 5.17-4.56 (m, 1H), 3.71 (t, J = 2.7 Hz, 1H), 2.97-2.66 (m, 4H), 2.51 (s, 3H), 2.30 (s, 3H). |
| 11 | 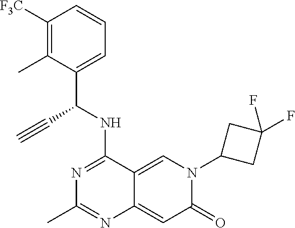 [SMILES: C#C[C@@H](Nc1nc(C)nc2cc(=O)n(C3CC(F)(F)C3)cc12)c1cccc(C(F)(F)F)c1C] | 463.4 | ¹H NMR (400 MHz, DMSO-d₆) δ 9.36 (br s, 1H), 9.00 (s, 1H), 8.02 (d, J = 7.7 Hz, 1H), 7.71 (d, J = 7.6 Hz, 1H), 7.53 (s, 2H), 6.70 (s, 1H), 6.15 (s, 1H), 4.82-4.86 (m, 1H), 3.71 (d, J = 2.4 Hz, 2H), 3.24-3.16 (m, 5H), 2.29 (s, 5H). |
| 12 | 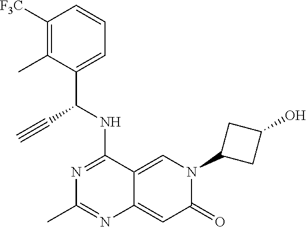 | 443.4 | ¹H NMR (400 MHz, DMSO-d₆) δ 9.41 (br s, 1H), 9.07 (s, 1H), 8.01 (d, J = 7.8 Hz, 1H), 7.70 (d, J = 7.5 Hz, 1H), 7.51 (t, J = 7.5 Hz, 1H), 6.69 (s, 1H), 6.11 (s, 1H), 5.44-5.23 (m, 2H), 4.36-4.42 (m, 1H), 3.70 (d, J = 2.4 Hz, 2H), 2.66-2.59 (m, 2H), 2.40-2.35 (m, 2H), 2.28 (s, 3H) |
| 13 | 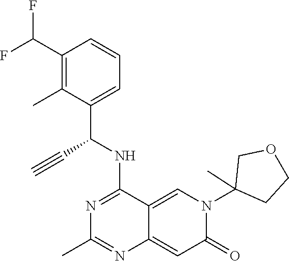 [SMILES: C#C[C@@H](Nc1nc(C)nc2cc(=O)n(C3(C)CCOC3)cc12)c1cccc(C(F)F)c1C] | 439.2 | ¹H NMR (400 MHz, DMSO-d₆) δ 7.85 (d, J = 7.4 Hz, 1H), 7.51 (d, J = 7.5 Hz, 1H), 7.41 (d, J = 7.5 Hz, 1H), 7.37 (t, J = 7.5 Hz, 1H), 7.22 (t, J = 54.8 Hz, 1H), 6.64-6.47 (m, 1H), 6.37 (d, J = 3.2 Hz, 1H), 4.82 (d, J = 5.3 Hz, 1H), 4.35 (d, J = 8.4 Hz, 1H), 4.16-4.00 (m, 3H), 3.68 (dd, J = 12.0, 3.5 Hz, 1H), 3.50 (d, J = 2.4 Hz, 1H), 2.42 (s, 3H), 2.21 (s, 3H), 2.00-1.87 (m, 1H), 1.56 (s, 3H), 1.43-1.47 (m, 1H). (mixture of diastereomers) |
| 14 | 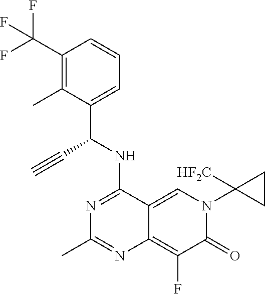 [SMILES: C#C[C@@H](Nc1nc(C)nc2c(F)c(=O)n(C3(C(F)F)CC3)cc12)c1cccc(C(F)(F)F)c1C] | 481.1 | ¹H NMR (400 MHz, DMSO-d₆) δ 9.54 (d, J = 7.3 Hz, 1H), 9.05 (d, J = 1.2 Hz, 1H), 8.00 (d, J = 7.8 Hz, 1H), 7.71 (d, J = 7.7 Hz, 1H), 7.51 (t, J = 7.8 Hz, 1H), 6.66 (dd, J = 7.3, 2.4 Hz, 1H), 6.31 (t, J = 56.4 Hz, 1H), 3.72 (d, J = 2.4 Hz, 1H), 2.33 (s, 3H), 1.41-1.49 (m, 4H). |
| 15 | 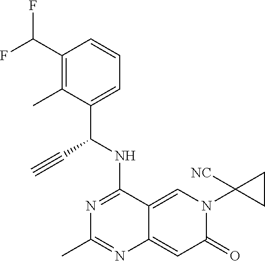 [SMILES: C#C[C@@H](Nc1nc(C)nc2cc(=O)n(C3(C#N)CC3)cc12)c1cccc(C(F)F)c1C] | 420.2 | ¹H NMR (400 MHz, CD₃OD δ 9.14 (s, 1H), 7.96 (d, J = 7.8 Hz, 1H), 7.53 (d, J = 7.6 Hz, 1H), 7.38 (t, J = 7.8 Hz, 1H), 6.96 (t, J = 55.1 Hz, 1H), 6.75 (d, J = 2.4 Hz, 1H), 6.40 (s, 1H), 3.05 (d, J = 2.3 Hz, 1H), 2.47 (s, 3H), 2.42 (s, 3H), 1.94 (m, 2H), 1.82-1.73 (m, 2H). |
| 16 | 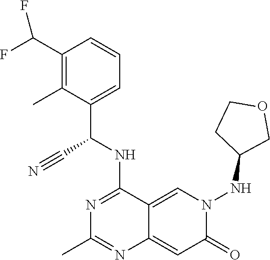 [SMILES: Cc1nc(N[C@H](C#N)c2cccc(C(F)F)c2C)c2cn(N[C@H]3CCOC3)c(=O)cc2n1] | 440.2 | ¹H NMR (400 MHz, CD₃OD δ 9.20 (s, 1H), 7.95 (d, J = 7.8 Hz, 1H), 7.53 (d, J = 7.8 Hz, 1H), 7.37 (t, J = 7.8 Hz, 1H), 6.96 (t, J = 55.1 Hz, 1H), 6.76 (d, J = 2.4 Hz, 1H), 6.48 (s, 1H), 4.08 (ddd, J = 9.0, 5.1, 2.6 Hz, 1H), 4.02 (dd, J = 15.5, 7.9 Hz, 1H), 3.82 (dt, J = 8.3, 4.1 Hz, 1H), 3.79-3.76 (m, 1H), 3.73 (dd, J = 9.8, 5.0 Hz, 1H), 3.03 (d, J = 2.5 Hz, 1H), 2.47 (s, 3H), 2.44 (s, 3H), 2.07-2.00 (m, 1H), 1.97-1.88 (m, 1H). |
| 17 | 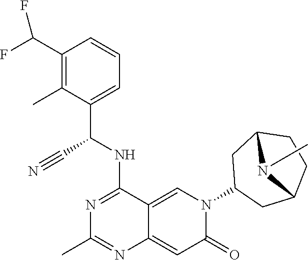 | 478.2 | ¹H NMR (400 MHz, DMSO-d₆) δ 9.18 (s, 1H), 8.95 (s, 1H), 7.86 (d, J = 7.8 Hz, 1H), 7.53 (d, J = 7.5 Hz, 1H), 7.43 (t, J = 7.7 Hz, 1H), 7.23 (t, J = 54.8 Hz, 1H), 6.69 (s, 1H), 6.10 (s, 1H), 5.24-5.07 (m, 1H), 3.66 (d, J = 2.5 Hz, 1H), 2.43-2.27 (m, 3H), 2.38 (d, J = 12.6 Hz, 2H), 2.27 (s, 3H), 2.18-2.10 (m, 3H), 2.16 (s, 3H), 1.67-1.52 (m, 4H). |

Compound 9 was subjected to chiral HPLC analysis under the following condition: CHIRALPAK®IC/SFC (4.6×250 mm, 5 mm); isocratic IPA/0.05% DEA; run time: 8 min; flow rate: 1.8 ml/min; and UV=220 nm. The compound shows two retention times, i.e., Rt=4.815 min (97.45% pure) and Rt=5.943 min (2.55% pure), and has [a]¹⁸_{D} of −153.4° (c 0.102, MeOH).

Racemic Compound 9 was also prepared in a manner identical to that used to prepare Compound 9 except that racemic benzylamine of moiety VIa was used. Racemic Compound 9 was subjected to chiral HPLC analysis under the same condition as used for Compound 9.

Different from Compound 9, this racemate exhibited the retention times of Rt=4.811 min (50.83% pure) and Rt=5.926 min (49.17% pure).

Compound 9 was also synthesized by coupling 6-(1-(difluoromethyl)cyclopropyl)-4-hydroxy-2-methylpyrido[4,3-d]pyrimidin-7 (6H)-one with (R)-1-(2-methyl-3-(trifluoromethyl)-phenyl)prop-2-yn-1-amine Via in the presence of HCCP and K₃PO₄ in CH₃CN in a similar manner described in the synthesis of compound 24 shown below.

### Synthesis of Compound 18: (R)-2-methyl-4-((1-(2-methyl-3-(trifluoromethyl)phenyl)prop-2-yn-1-yl)amino)-6-(1-methylcyclopropyl)pyrido[4,3-d]pyrimidin-7 (6H)-one

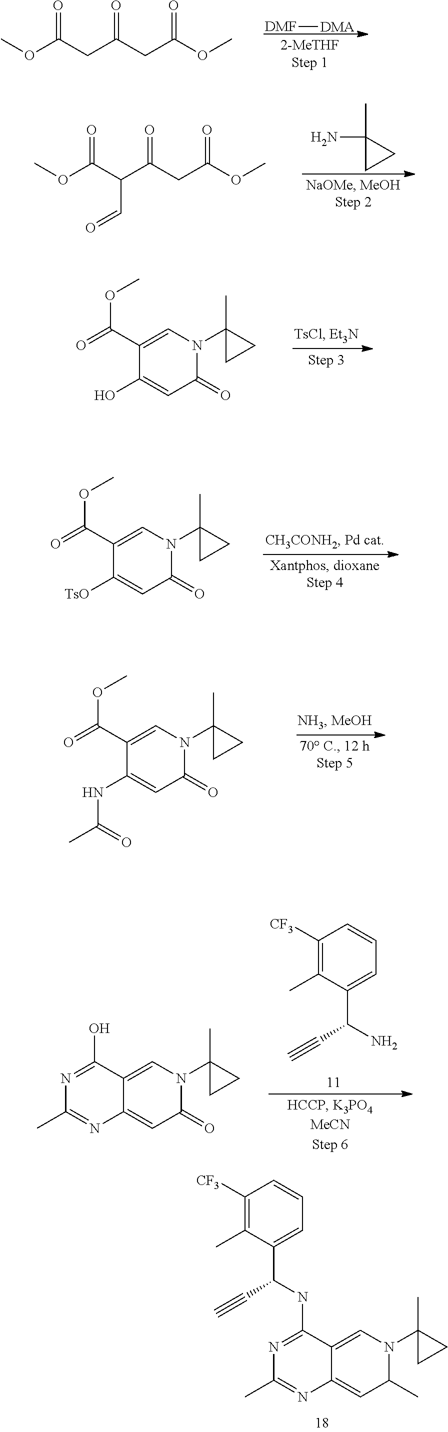

### Step 1. Synthesis of 1,5-dimethyl 2-formyl-3-oxopentanedioate

To a solution of 1,5-dimethyl 3-oxopentanedioate (20.3 mL, 137.8 mmol) in 2-methyltetrahydrofuran (200 mL), DMF-DMA (18.5 mL, 137.8 mmol) was added at 0° C. The resultant mixture was stirred at the temperature for 3 hours. 4 N aqueous HCl (75 mL) was then added. The mixture was stirred at room temperature for 1 hour, diluted with EtOAc (1 L), washed with brine, dried over anhydrous Na₂SO₄, and filtered. The filtrate was concentrated under reduced pressure and purified by chromatograph (gradient: PE to EtOAc:PE=1:10) to give the title compound (22.0 g, 108.8 mmol, 79% yield) as a yellow oil.

¹H NMR (400 MHz, CDCl₃) δ 8.99 (d, J=7.9 Hz, 1H), 3.95 (d, J=2.5 Hz, 2H), 3.79 (s, 3H), 3.74 (d, J=6.5 Hz, 4H) LC-MS m/e: 203 (MH⁺).

### Step 2. Synthesis of methyl 4-hydroxy-1-(1-methylcyclopropyl)-6-oxo-1,6-dihydropyridine-3-carboxylate

A mixture of 1,5-dimethyl 2-formyl-3-oxopentanedioate (12.0 g, 59.36 mmol) and 1-methylcyclopropan-1-amine (8.44 g, 118.7 mmol) in MeOH (40 mL) was stirred at 25° C. for 4 hours. Sodium methanolate in MeOH (5.4 M, 12.6 mL, 68.3 mmol) was then added and the resultant mixture was stirred for additional 2 hours. A pH value of the mixture was adjusted to ˜6 with 4N HCl aqueous solution. The resultant solution was then concentrated under reduced pressure to give a residue, which was purified by chromatography (gradient: PE to EtOAc:PE=1:10) to afford the title compound (7.80 g, 34.9 mmol, 59% yield) as a yellow solid.

¹H NMR (400 MHz, DMSO-d₆) δ 10.71 (s, 1H), 8.25 (s, 1H), 5.63 (s, 1H), 3.81 (s, 3H), 1.40 (s, 3H), 1.03-0.85 (m, 4H) LC-MS m/e: 224 (MH⁺).

### Step 3. Synthesis of methyl 4-[(4-methylbenzenesulfonyl)oxy]-1-(1-methylcyclopropyl)-6-oxo-1,6-dihydropyridine-3-carboxylate

To a mixture of methyl 4-hydroxy-1-(1-methylcyclopropyl)-6-oxo-1,6-dihydropyridine-3-carboxylate (500 mg, 2.24 mmol) and Et₃N (0.94 mL, 6.72 mmol) in DCM (5 mL) at 0° C., tosyl chloride (0.51 mL, 2.69 mmol) was added. The resultant mixture was stirred at 25° C. for 2 hours, quenched with H₂O (50 mL), and extracted with DCM (20 mL×3). The combined organic layers were dried over anhydrous Na₂SO₄ and filtered. The filtrate was concentrated under reduced pressure and purified by chromatography (gradient: PE to EtOAc:PE=1:5) to give the title compound (600 mg, 1.59 mmol, 71% yield) as a yellow solid.

¹H NMR (400 MHz, DMSO-d₆) δ 8.31 (s, 1H), 7.83 (d, J=8.0 Hz, 2H), 7.52 (d, J=8.0 Hz, 2H), 5.95 (s, 1H), 3.67 (s, 3H), 2.45 (s, 3H), 1.41 (s, 3H), 1.01-0.92 (m, 4H).

LC-MS m/e: 378 (MH⁺).

### Step 4. Synthesis of methyl 4-acetamido-1-(1-methylcyclopropyl)-6-oxo-1,6-dihydropyridine-3-carboxylate

To a mixture of methyl 4-[(4-methylbenzenesulfonyl)oxy]-1-(1-methylcyclopropyl)-6-oxo-1,6-dihydropyridine-3-carboxylate (9.20 g, 24.38 mmol), acetamide (1.49 mL, 29.25 mmol) and K₃PO₄ (15.52 g, 73.13 mmol) in DMF (100 mL), Pd₂(dba)₃ (2.23 g, 2.44 mmol) and X-PHOS (1.16 g, 2.438 mmol) were added under N₂. The resultant mixture was stirred at 120° C. under N₂ for 4 hours. The mixture was then cooled down to room temperature and filtered through a pad of Celite. The filtrate was diluted with water (100 mL) and extracted with EtOAc (100 mL×3). The combined organic layers were washed with brine, dried over anhydrous Na₂SO₄, and filtered. The filtrate was concentrated under reduced pressure and purified by chromatography (gradient: PE to EtOAc:PE=0 to 1:1) to give the title compound (4.2 g, 15.9 mmol, 65% yield) as a yellow solid.

¹H NMR (400 MHz, DMSO-d₆) δ 10.58 (s, 1H), 8.38 (s, 1H), 7.30 (s, 1H), 3.84 (s, 3H), 2.15 (s, 3H), 1.42 (s, 3H), 1.00-0.91 (m, 4H).

LC-MS m/e: 265 (MH⁺).

### Step 5. Synthesis of 4-hydroxy-2-methyl-6-(1-methylcyclopropyl)-6H,7H-pyrido[4,3-d]pyrimidin-7-one

A mixture of methyl 4-acetamido-1-(1-methylcyclopropyl)-6-oxo-1,6-dihydropyridine-3-carboxylate (3.00 g, 11.35 mmol) in 7 N NH₃ methanol solution (30 mL, 210 mmol) was stirred in a sealed tube at 70° C. for 16 hours. The mixture was then concentrated under reduced pressure and purified by chromatography (gradient: DCM to MeOH:DCM=1:10) to give the title compound (1.50 g, 6.49 mmol, 57% yield) as a yellow solid.

¹H NMR (400 MHz, DMSO-d₆) δ 11.61 (s, 1H), 8.52 (s, 1H), 6.12 (s, 1H), 2.23 (s, 3H), 1.46 (s, 3H), 1.06-0.98 (m, 4H).

LC-MS m/e: 232 (MH⁺).

### Step 6. Synthesis of (R)-2-methyl-4-((1-(2-methyl-3-(trifluoromethyl)phenyl)prop-2-yn-1-yl)amino)-6-(1-methylcyclopropyl)pyrido[4,3-d]pyrimidin-7 (6H)-one (Compound 18)

A mixture of 4-hydroxy-2-methyl-6-(1-methylcyclopropyl)-6H,7H-pyrido[4,3-d]pyrimidin-7-one (300 mg, 1.30 mmol), phosphonitrilic chloride trimer (541 mg, 1.56 mmol), and K₃PO₄ (688 mg, 3.24 mmol) in MeCN (6 mL) was stirred at 30° C. for 2 hours. (1R)-1-[2-methyl-3-(trifluoromethyl)phenyl]propan-1-amine (338 mg, 1.56 mmol) was then added and the resultant mixture was stirred at 30° C. for 2 hours. To the mixture, aqueous ammonium hydroxide (37%, 0.5 mL) was added and the solution was stirred at 30° C. for 0.5 hours. To the mixture, saturated potassium carbonate aqueous solution (6 mL) was added and the solution was stirred at 30° C. for additional 12 hours. The reaction mixture was then diluted with water and extracted with DCM (50 mL). The combined organic layers were dried over anhydrous Na₂SO₄ and filtered. The filtrate was concentrated under reduced pressure and the resultant residue was purified by reverse phase chromatography (SilaSep™ C18 silica flash cartridge, gradient, 5% to 55% MeCN in H₂O with 0.1% formic acid) to give the title compound (46 mg, 0.11 mmol, 8% yield) as a yellow solid.

¹H NMR (400 MHz, DMSO-d₆) δ 10.96 (s, 1H), 9.53 (s, 1H), 8.07 (d, J=7.7 Hz, 1H), 7.76 (d, J=7.8 Hz, 1H), 7.56 (t, J=7.8 Hz, 1H), 6.81 (s, 1H), 6.17 (s, 1H), 3.89 (d, J=2.3 Hz, 2H), 2.49 (s, 6H), 1.50 (s, 3H), 1.14 (br s, 2H), 1.04 (br s, 2H).

LC-MS m/e: 427 (MH⁺).

### Syntheses of Compounds 19-23

Compounds 19-23 shown in Table 4 were prepared in a manner similar to that used to prepare Compound 18.

TABLE 4

| | | LC-MS | |
|---|---|---|---|
| Compound | | m/e | |
| No. | Structure | (MH⁺) | ¹H NMR |
| 19 | 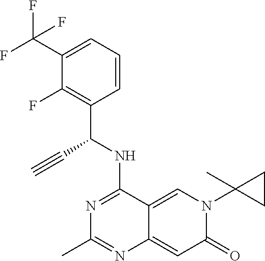 [SMILES: C#C[C@@H](Nc1nc(C)nc2cc(=O)n(C3(C)CC3)cc12)c1cccc(C(F)(F)F)c1F] | 427.1 | ¹H NMR (400 MHz, DMSO-d₆) δ 9.32 (br s, 1H), 9.22 (s, 1H), 7.86 (t, J = 7.8 Hz, 1H), 7.57 (t, J = 7.8 Hz, 1H), 7.39 (t, J = 7.8 Hz, 1H), 6.68 (s, 1H), 6.07 (s, 1H), 3.69 (s, 1H), 2.49 (s, 3H), 2.25 (s, 3H), 2.02 (t, J = 19.2 Hz, 3H), 1.49 (s, 3H), 1.14-1.08 (m, 2H), 1.02-0.98 (m, 2H). |
| 20 | 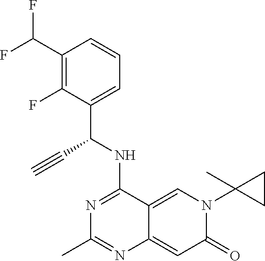 [SMILES: C#C[C@@H](Nc1nc(C)nc2cc(=O)n(C3(C)CC3)cc12)c1cccc(C(F)F)c1F] | 413.2 | ¹H NMR (400 MHz, DMSO-d₆) δ 11.00 (s, 1H), 9.50 (s, 1H), 7.98 (t, J = 7.2 Hz, 1H), 7.71 (t, J = 7.0 Hz, 1H), 7.49 (t, J = 7.8 Hz, 1H), 7.26 (t, J = 54.1 Hz, 1H), 6.86 (d, J = 4.4 Hz, 1H), 6.15 (s, 1H), 3.94 (d, J = 2.5 Hz, 1H), 2.50 (s, 3H), 1.50 (s, 3H), 1.13 (t, J = 8.2 Hz, 2H), 1.05 (br s, 2H). |
| 21 | 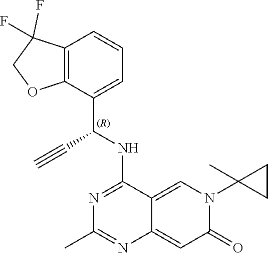 | 423.0 | ¹H NMR (400 MHz, DMSO-d₆) δ 9.20 (s, 1H), 8.14 (s, 1H), 7.85 (d, J = 7.5 Hz, 1H), 7.66 (d, J = 7.0 Hz, 1H), 7.23 (t, J = 7.6 Hz, 1H), 6.59 (s, 1H), 6.08 (s, 1H), 4.87 (td, J = 16.5, 3.0 Hz, 2H), 3.60 (d, J = 2.2 Hz, 1H), 2.28 (s, 3H), 1.49 (s, 3H), 1.10 (br s, 2H), 1.00 (br s, 2H). |
| 22 | 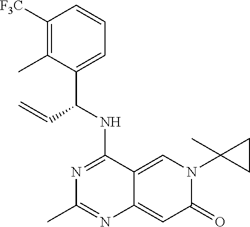 [SMILES: C=C[C@@H](Nc1nc(C)nc2cc(=O)n(C3(C)CC3)cc12)c1cccc(C(F)(F)F)c1C] | 429.2. | ¹H NMR (400 MHz, CDCl₃) δ 9.18 (br s, 1H), 7.76 (s, 1H), 7.53 (d, J = 8.0 Hz, 1H), 7.22-7.24 (m, 2H), 6.45 (br s, 2H), 6.33-6.16 (m, 1H), 5.28 (d, J = 8.0 Hz, 1H), 5.19 (d, J = 17.0 Hz, 1H), 2.56 (s, 3H), 2.43 (s, 3H), 1.54 (s, 3H), 1.22-1.10 (m, 2H), 1.08-0.98 (m, 2H). |
| 23 | 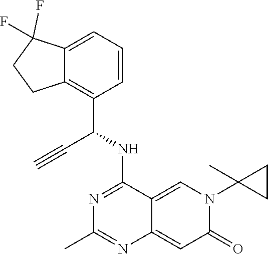 [SMILES: C#C[C@@H](Nc1nc(C)nc2cc(=O)n(C3(C)CC3)cc12)c1cccc2c1CCC2(F)F] | 421.2 | ¹H NMR (400 MHz, DMSO-d₆) δ 9.24 (d, J = 7.7 Hz, 1H), 9.20 (s, 1H), 7.86 (d, J = 7.3 Hz, 1H), 7.63-7.45 (m, 2H), 6.57 (d, J = 7.7 Hz, 1H), 6.08 (s, 1H), 3.68 (d, J = 2.4 Hz, 1H), 3.10 (m, 2H), 2.64-2.57 (m, 2H), 2.29 (s, 3H), 1.49 (s, 3H), 1.10 (br s, 2H), 0.99 (br s, 2H). |

### Synthesis of Compound 24: (R)-4-((1-(3-(difluoromethyl)-2 fluorophenyl)prop-2 yn-1-yl)amino)-6-(1-(difluoromethyl)cyclopropyl)-2-methylpyrido[4,3-d]pyrimidin-7 (6H)-one

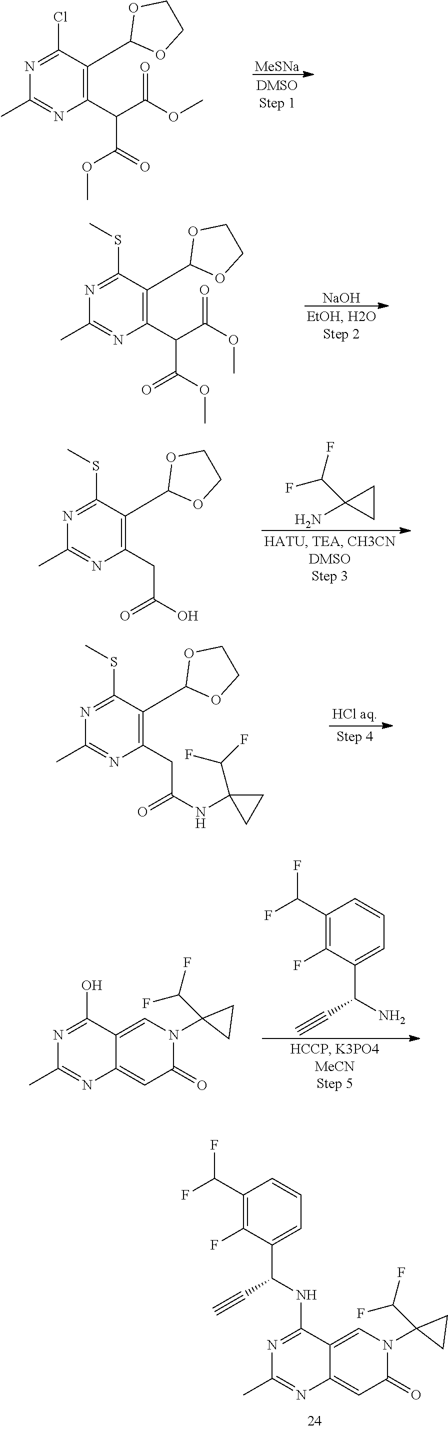

### Step 1. Synthesis of dimethyl 2-(5-(1,3-dioxolan-2-yl)-2-methyl-6-methylthio)pyrimidin-4-yl) malonate

A mixture of methyl 2-[6-chloro-5-(1,3-dioxolan-2-yl)-2-methylpyrimidin-4-yl]acetate (1.00 g, 0.37 mmol) and sodium methanethiolate (0.64 g, 9.1 mmol) in DMSO (5 mL) was stirred at 100° C. under N₂ atmosphere for 1 hour. The reaction mixture was then cooled to room temperature, diluted with water (30 mL), and extracted with EtOAc (50 mL). The organic layer was concentrated under reduced pressure to give the title compound (10.20 g, 3.51 mmol, 116% yield) as a yellow oil, which was used in the next step without further purification.

LC-MS m/e: 343.1 (MH⁺).

### Step 2. Synthesis of 2-(5-(1,3-dioxolan-2-yl)-2-methyl-6-(methylthio)pyrimidin-4-yl)acetic acid, sodium salt

A mixture of 1,3-dimethyl 2-[5-(1,3-dioxolan-2-yl)-2-methyl-6-(methylsulfanyl)pyrimidin-4-yl]propanedioate (1.20 g, 3.51 mmol), NaOH (0.56 g, 14.02 mmol) and EtOH (6 mL) in H₂O (2 mL) was stirred at 80° C. for 2 hours. The reaction mixture was then concentrated under reduced pressure. The resultant residue was dried in vacuum to give the title compound (1.00 g, 3.70 mmol, 105.5% yield) as a yellow solid, which was used in the next step without further purification.

LC-MS m/e: 271.1 (MH⁺).

### Step 3. Synthesis of 2-(5-(1,3-dioxolan-2-yl)-2-methyl-6-(methylthio)pyrimidin-4-yl)-N-(1-(difluoromethyl)cyclopropyl)acetamide

A mixture of 2-[5-(1,3-dioxolan-2-yl)-2-methyl-6-(methylsulfanyl)pyrimidin-4-yl]acetic acid (3.50 g, 12.95 mmol), 1-(difluoromethyl)cyclopropan-1-amine (2.08 g, 9.42 mmol), HATU (7.39 g, 19.42 mmol), TEA (6.4 ml, 38.84 mmol) in MeCN (20 mL) and DMSO (10 mL) was stirred at rt for 12 hours. The reaction mixture was then diluted with water and extracted with DCM. The combined organic layers were concentrated under reduced pressure and purified by flash (gradient: PE to EtOAc:PE=6:4) to give the title compound (2.60 g, 7.23 mmol, 56% yield) as a yellow solid.

LC-MS m/e: 360.1 (MH⁺).

### Step 4. Synthesis of 6-(1-(difluoromethyl)cyclopropyl)-4-hydroxy-2-methylpyrido[4,3-d]pyrimidin-7 (6H)-one

A mixture of N-[1-(difluoromethyl)cyclopropyl]-2-[5-(1,3-dioxolan-2-yl)-2-methyl-6-(methylsulfanyl)pyrimidin-4-yl]acetamide (600 mg, 1.67 mmol) and aqueous HCl (2.0 M, 4.2 mL, 8.4 mmol) in propan-2-ol (10 mL) was stirred at 80° C. for 2 hours. The reaction mixture was then cooled to room temperature, diluted with water (30 mL), and extracted with EtOAc (50 mL). The organic layer was dried over anhydrous Na₂SO₄ and then filtered. The filtrate was concentrated under reduced pressure. The resultant residue was dried in vacuum to the title compound (350 mg, 1.31 mmol, 78% yield) as a yellow solid, which was used in the next step without further purification.

LC-MS m/e: 268.2 (MH⁺). The material is used in the next step without further purification.

### Step 5. Synthesis of (R)-4-((1-(3-(difluoromethyl)-2-fluorophenyl)prop-2-yn-1-yl)amino)-6-(1-(difluoromethyl)cyclopropyl)-2-methylpyrido[4,3-d]pyrimidin-7 (6H)-one (Compound 24)

A mixture of 6-(1-(difluoromethyl)cyclopropyl)-4-hydroxy-2-methylpyrido[4,3-d]pyrimidin-7 (6H)-one (200 mg, 0.75 mmol), phosphonitrilic chloride trimer (312 mg, 0.90 mmol), K₃PO₄ (0.477 g, 2.25 mmol), and DIEA (0.4 mL, 2.25 mmol) in MeCN (10 mL) was stirred at 35° C. for 2 hours. (R)-1-(3-(difluoromethyl)-2-fluorophenyl)prop-2-yn-1-amine (224 mg, 1.12 mmol) was then added to the mixture. The resultant mixture was stirred at 90° C. for 2 hours. The mixture was diluted with water (50 mL) and extracted with DCM (3×50 mL). The combined organic layers were dried over anhydrous sodium sulfate, filtered, and concentrated under reduced pressure to give a residue, which was purified by flash chromatography (gradient: 0%-5% MeOH in DCM) and further purified by Prep HPLC (SilaSep™ C18 silica flash cartridge, 5%-55% MeCN in H₂O with 0.1% formic acid) to give the title compound (45 mg, 0.09 mmol, 12% yield) as a yellow solid.

¹H NMR (400 MHz, DMSO-d₆) δ 10.84 (s, 1H), 9.41 (s, 1H), 7.95 (t, J=7.4 Hz, 1H), 7.70 (t, J=6.9 Hz, 1H), 7.48 (t, J=7.7 Hz, 1H), 7.25 (t, J=54.1 Hz, 1H), 6.83 (s, 1H), 6.54-6.03 (m, 2H), 3.92 (s, 1H), 2.48 (s, 3H), 1.50 (s, 2H), 1.41 (s, 2H). ¹⁹FNMR (377 MHz, DMSO-d₆) δ −112.95 (d, J=2.7 Hz, 2F), −113.25 (d, J=3.9 Hz, 2F), −122.32 (s, 1F).

LC-MS m/e: 449.2 (MH⁺).

### Syntheses of Compounds 25 and 26

Compounds 25 and 26 shown in Table 5 were prepared in a manner similar to that used to prepare Compound 24.

TABLE 5

| | | LC-MS | |
|---|---|---|---|
| Compound | | m/e | |
| No. | Structure | (MH⁺) | ¹H NMR |
| 25 | 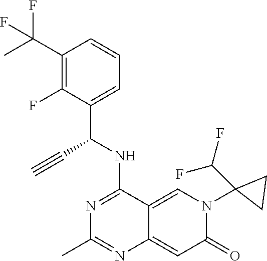 [SMILES: C#C[C@@H](Nc1nc(C)nc2cc(=O)n(C3(C(F)F)CC3)cc12)c1cccc(C(C)(F)F)c1F] | 463.2 | ¹H NMR (400 MHz, CD₃OD) δ 9.17 (s, 1H), 8.21 (s, 1H), 7.89 (t, J = 6.7 Hz, 1H), 7.57 (t, J = 6.8 Hz, 1H), 7.32 (t, J = 7.8 Hz, 1H), 6.82 (d, J = 2.4 Hz, 1H), 6.36 (s, 1H), 6.22 (t, J = 54.1 Hz, 1H), 3.09 (d, J = 2.5 Hz, 1H), 2.42 (d, J = 5.5 Hz, 3H), 1.97 (t, J = 18.6 Hz, 3H), 1.55 (br s, 2H), 1.39 (br s, 2H). |
| 26 | 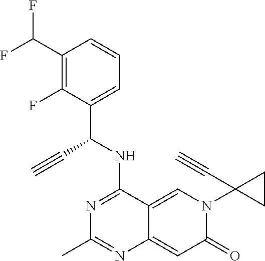 [SMILES: C#C[C@@H](Nc1nc(C)nc2cc(=O)n(C3(C#C)CC3)cc12)c1cccc(C(F)F)c1F] | 423.2 | ¹H NMR (400 MHz, DMSO-d₆) δ 9.37 (d, J = 7.5 Hz, 1H), 9.26 (s, 1H), 7.91 (t, J = 7.6 Hz, 1H), 7.65 (t, J = 7.4 Hz, 1H), 7.44 (t, J = 7.7 Hz, 1H), 7.26 (t, J = 54.3 Hz, 1H), 6.71 (dd, J = 7.3, 2.5 Hz, 1H), 6.12 (s, 1H), 3.72 (d, J = 2.5 Hz, 1H), 3.30 (s, 1H), 2.27 (s, 3H), 1.54 (s, 2H), 1.24 (s, 2H). |

### Synthesis of Compound 27: (R)-4-((1-(3-(difluoromethyl)-2-fluorophenyl)prop-2-yn-1-yl)amino)-6-(1-(difluoromethyl)cyclopropyl)-8-fluoro-2-methylpyrido[4,3-d]pyrimidin-7 (6H)-one

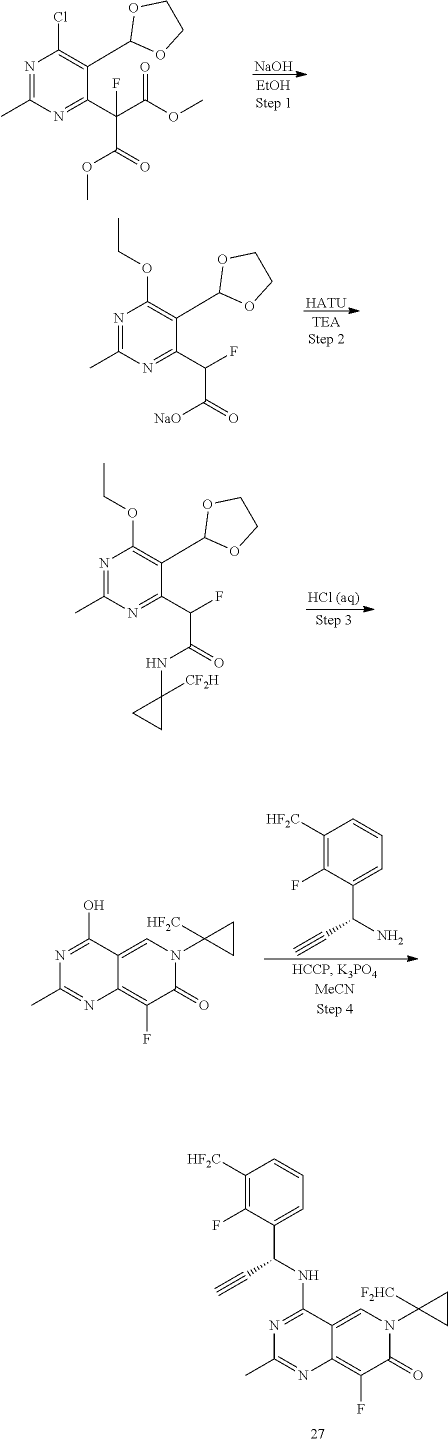

### Step 1. Synthesis of 2-(5-(1,3-dioxolan-2-yl)-6-ethoxy-2-methylpyrimidin-4-yl)-2-fluoroacetic acid, sodium salt

To a solution of dimethyl 2-(6-chloro-5-(1,3-dioxolan-2-yl)-2-methylpyrimidin-4-yl)-2-fluoromalonate (2.00 g, 5.74 mmol) (prepared according to the procedure described in WO 2019/122129) in EtOH (50 mL), NaOH (690 mg, 17.2 mmol) was added. The reaction mixture was stirred at 60° C. for 15 minutes and then concentrated under reduced pressure. The resultant residue was dried in vacuum to afford the title compound (1.50 g, 5.24 mmol, 91% yield) as a yellow oil.

LC-MS m/e: 286.9 (MH⁺).

### Step 2. Synthesis of 2-(5-(1,3-dioxolan-2-yl)-6-ethoxy-2-methylpyrimidin-4-yl)-N-(1-(difluoromethyl)cyclopropyl)-2-fluoroacetamide

To a mixture of 2-(5-(1,3-dioxolan-2-yl)-6-ethoxy-2-methylpyrimidin-4-yl)-2-fluoroacetic acid, sodium salt (1.0 g, 3.49 mmol), HATU (3.33 g, 10.48 mmol) and TEA (1.06 mg, 10.48 mmol) in DMF (25 mL) was added 1-(difluoromethyl)cyclopropan-1-amine (0.49 g, 4.54 mmol). The resultant mixture was stirred at room temperature for 1 hour and then diluted with EtOAc (50 mL) and water (50 mL). The organic layer was washed with brine, dried over Na₂SO₄, and filtered. The filtrate was concentrated under reduced pressure to give the title compound (800 mg, 2.13 mmol, 61% yield) as a yellow oil.

LC-MS m/e: 376.4 (MH⁺).

### Step 3. Synthesis of 6-(1-(difluoromethyl)cyclopropyl)-8-fluoro-4-hydroxy-2-methylpyrido[4,3-d]pyrimidin-7 (6H)-one

A solution of 2-(5-(1,3-dioxolan-2-yl)-6-ethoxy-2-methylpyrimidin-4-yl)-N-(1-(difluoromethyl)cyclopropyl)-2-fluoroacetamide (400 mg, 1.07 mmol) in 5 N HCl (1 mL, 5 mmol) and isopropyl alcohol (20 mL) was stirred at 50° C. for 6 hours. The solution was then cooled down to room temperature and diluted with EtOAc (20 mL). The organic layer was washed with saturated aqueous NaHCO₃ solution (50 mL), brine (50 mL), dried over Na₂SO₄, and filtered. The filtrate was concentrated under reduced pressure to give the title compound (200.0 mg, 0.71 mmol, 66% yield) as a green solid.

LC-MS m/e: 286.3 (MH⁺).

### Step 4. Synthesis of (R)-4-((1-(3-(difluoromethyl)-2-fluorophenyl)prop-2-yn-1-yl)amino)-6-(1-(difluoromethyl)cyclopropyl)-8-fluoro-2-methylpyrido[4,3-d]pyrimidin-7 (6H)-one (Compound 27)

A solution of 6-(1-(difluoromethyl)cyclopropyl)-8-fluoro-4-hydroxy-2-methylpyrido[4,3-d]pyrimidin-7 (6H)-one (100 mg, 0.35 mmol), K₃PO₄ (149 mg, 0.70 mmol) and HCCP (122 mg, 0.35 mmol) in CH₃CN (25 mL) was stirred at 50° C. for 2 hours. To the solution was added (R)-1-(3-(difluoromethyl)-2-fluorophenyl)prop-2-yn-1-amine (479.41 mg, 2.249 mmol). The resultant mixture was stirred at 50° C. for 2 hours. NH₄OH (0.2 mL) was then added and the resultant solution was stirred at the temperature for additional 2 hours. Potassium carbonate aqueous solution (2.0 mL) was added and the resultant mixture was stirred at 50° C. overnight. The mixture was then cooled down to room temperature and diluted with EtOAc (60 mL) and H₂O (60 mL). The organic layer was washed with brine, dried over Na₂SO₄, and filtered. The filtrate was concentrated under reduced pressure and the resultant residue was purified by prep-HPLC (SilaSep™ C18 silica flash cartridge, 0% to 55% MeCN in H₂O with 0.1% formic acid) to give the title compound (21.0 mg, 0.05 mmol, 13% yield) as a white solid.

¹H NMR (400 MHz, DMSO-d₆) δ 9.64-9.52 (br s, 1H), 9.04 (s, 1H), 7.95 (t, J=7.7 Hz, 1H), 7.70 (t, J=7.7 Hz, 1H), 7.44 (t, J=7.7 Hz, 1H), 7.26 (t, J=54.2 Hz, 1H), 6.83 (s, 1H), 6.27 (d, J=56.4 Hz, 1H), 6.19 (s, 1H), 3.75 (d, J=2.4 Hz, 1H), 2.48 (s, 3H), 2.33 (s, 3H), 1.53-1.46 (m, 2H), 1.42 (br s, 72H).

LC-MS m/e: 427.1 (MH⁺).

### Syntheses of Compounds 28-32

Compounds 28-32 shown in Table 6 were prepared in a manner similar to that used to prepare Compound 27.

TABLE 6

| | | LC-MS | |
|---|---|---|---|
| Compound | | m/e | |
| No. | Structure | (MH⁺) | ¹H NMR |
| 28 | 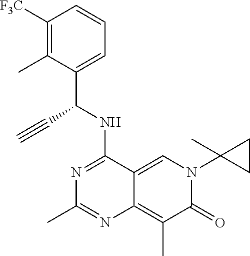 [SMILES: C#C[C@@H](Nc1nc(C)nc2c(C)c(=O)n(C3(C)CC3)cc12)c1cccc(C(F)(F)F)c1C] | 441.2 | ¹H NMR (400 MHz, DMSO-d₆) δ 9.20 (d, J = 7.3 Hz, 1H), 9.09 (s, 1H), 8.01 (d, J = 7.8 Hz, 1H), 7.69 (d, J = 7.8 Hz, 1H), 7.50 (t, J = 7.8 Hz, 1H), 6.65 (dd, J = 7.0, 2.1 Hz, 1H), 3.66 (d, J = 2.4 Hz, 1H), 2.50 (s, 3H), 2.30 (s, 3H), 2.13 (d, J = 7.1 Hz, 3H), 1.49 (s, 3H), 1.11-1.07 (m, 2H), 1.01-0.98 (m, 2H). |
| 29 | 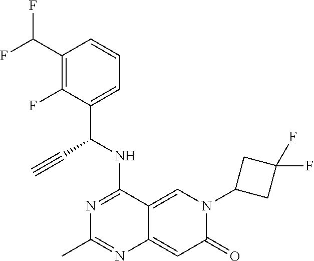 [SMILES: C#C[C@@H](Nc1nc(C)nc2cc(=O)n(C3CC(F)(F)C3)cc12)c1cccc(C(F)F)c1F] | 449.1 | ¹H NMR (400 MHz, DMSO-d₆) δ 9.38 (br s, 1H), 8.98 (s, 1H), 8.14 (t, J = 7.3 Hz, 1H), 7.90 (t, J = 7.3 Hz, 1H), 7.64 (t, J = 6.9 Hz, 1H), 7.43 (t, J = 7.7 Hz, 1H), 7.25 (t, J = 54.2 Hz, 1H), 6.77 (s, 1H), 6.14 (s, 1H), 4.83 (t, J = 7.0 Hz, 1H), 3.73 (s, 1H), 3.22-3.06 (m, 4H), 2.28 (s, 3H). |
| 30 | 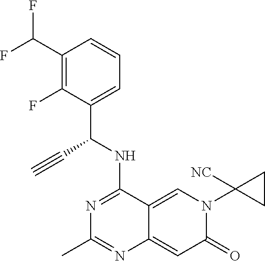 [SMILES: C#C[C@@H](Nc1nc(C)nc2cc(=O)n(C3(C#N)CC3)cc12)c1cccc(C(F)F)c1F] | 424.1 | ¹H NMR (400 MHz, CD₃OD δ 9.13 (s, 1H), 7.94 (t, J = 7.3 Hz, 1H), 7.62 (t, J = 7.0 Hz, 1H), 7.37 (t, J = 7.8 Hz, 1H), 7.00 (t, J = 54.7 Hz, 1H), 6.79 (d, J = 2.4 Hz, 1H), 6.39 (s, 1H), 3.10 (d, J = 2.3 Hz, 1H), 2.41 (s, 3H), 1.98-1.92 (m, 2H), 1.81-1.74 (m, 2H). |
| 31 | 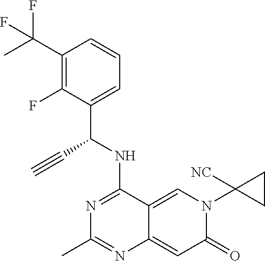 [SMILES: C#C[C@@H](Nc1nc(C)nc2cc(=O)n(C3(C#N)CC3)cc12)c1cccc(C(C)(F)F)c1F] | 438.2 | ¹H NMR (400 MHz, CD₃OD δ 9.14 (s, 1H), 7.89 (t, J = 7.0 Hz, 1H), 7.57 (t, J = 6.8 Hz, 1H), 7.32 (t, J = 7.8 Hz, 1H), 6.79 (d, J = 2.5 Hz, 1H), 6.40 (s, 1H), 3.08 (s, 1H), 2.41 (s, 3H), 2.05-1.92 (m, 2H), 1.98 (t, J = 18.4 Hz, 3H), 1.82-1.73 (m, 2H) |
| 32 | 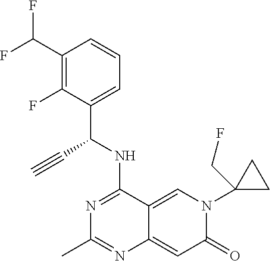 [SMILES: C#C[C@@H](Nc1nc(C)nc2cc(=O)n(C3(CF)CC3)cc12)c1cccc(C(F)F)c1F] | 431.1 | ¹H NMR (400 MHz, DMSO-d₆) δ 11.20 (d, J = 7.1 Hz, 1H), 9.52 (s, 1H), 7.98 (t, J = 7.2 Hz, 1H), 7.71 (t, J = 6.9 Hz, 1H), 7.48 (t, J = 7.8 Hz, 1H), 7.25 (t, J = 54.1 Hz, 1H), 6.87 (dd, J = 7.1, 2.4 Hz, 1H), 6.25 (s, 1H), 4.61 (d, J = 40.4 Hz, 2H), 3.94 (d, J = 2.5 Hz, 1H), 2.52 (s, 3H), 1.34 (br, 4H). |

### Synthesis of Compound 33: (R)-6-(I-acetyl-4-methylpiperidin-4-yl)-2-methyl-4-((1-(2-methyl-3-(trifluoromethyl) phenyl) prop-2-yn-1-yl)amino) pyrido[4,3-d]pyrimidin-7 (6H)-one

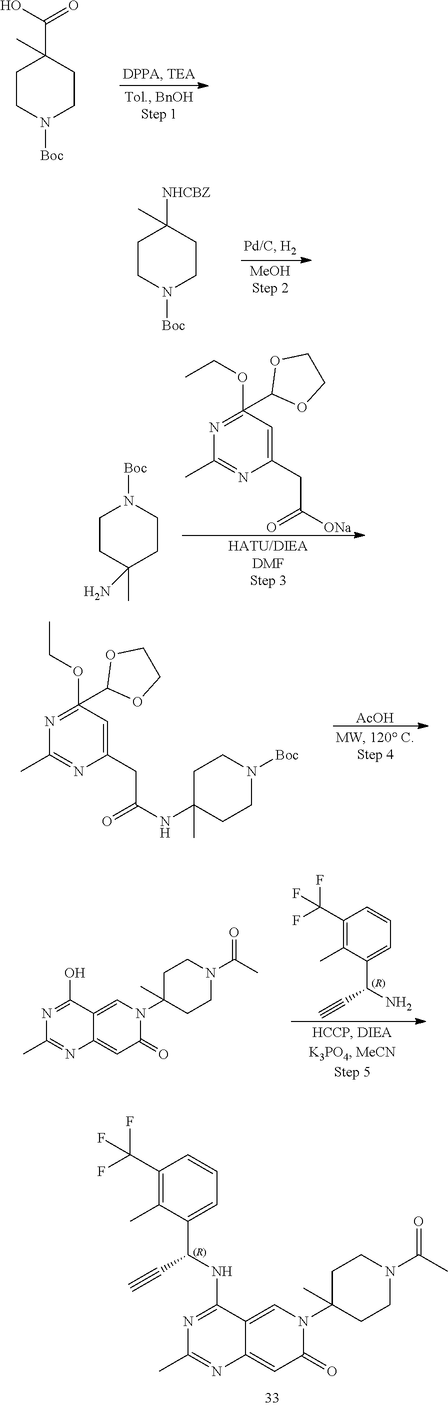

### Step 1. Synthesis of tert-butyl 4-(((benzyloxy)carbonyl)amino)-4-methylpiperidine-1-carboxylate

To a solution of 1-(tert-butoxycarbonyl)-4-methylpiperidine-4-carboxylic acid (6 g, 24.7 mmol) in toluene (60 mL) were added triethylamine (6.9 mL, 49.3 mmol), DPPA (6.4 mL, 29.6 mmol) and phenylmethanol (5.1 mL, 49.3 mmol. The mixture was stirred at 80° C. for 6 hours, then quenched with water (100 mL), and extracted with EtOAc (150 mL×3). The combined organic phase was dried over anhydrous Na₂SO₄, filtered, and concentrated in vacuum to dryness. The residue was purified by flash column chromatography on silica gel (gradient, PE/EtOAc=10/1 to 7/1) to give the title compound (6 g, 17.2 mmol, 69.8% yield) as a colorless oil.

LC-MS m/e: 349 (MH⁺).

### Step 2. Synthesis of tert-butyl 4-amino-4-methylpiperidine-1-carboxylate

To a solution of tert-butyl 4-(((benzyloxy)carbonyl)amino)-4-methylpiperidine-1-carboxylate (6 g, 17.2 mmol) in MeOH (60 mL) was added Pd/C (1.83 g, 10% and 55% wet).

The mixture was stirred at 20° C. for 5 hours under hydrogen, then filtered through a pad of filter cel, and concentrated in vacuum to dryness. The residue was purified by flash column chromatography on silica gel (gradient, PE/EtOAc=1/10 to 1/3) to give the title compound (3 g, 14.0 mmol, 81.3% yield) as a colorless oil.

LC-MS m/e: 215 (MH⁺).

### Step 3. Synthesis of tert-butyl 4-(2-(5-(1,3-dioxolan-2-yl)-6-ethoxy-2-methylpyrimidin-4-yl)acetamido)-4-methylpiperidine-1-carboxylate

To a solution of sodium 2-(5-(1,3-dioxolan-2-yl)-6-ethoxy-2-methylpyrimidin-4-yl)acetate (880 mg, 3.03 mmol) and tert-butyl 4-amino-4-methylpiperidine-1-carboxylate (500 mg, 2.33 mmol) in DMF (10 mL) were added triethylamine (1.0 mL, 6.99 mmol) and HATU (1.15 g, 3.03 mmol). The mixture was stirred at 20° C. for 3 hours, then quenched with water (80 mL), and extracted with EtOAc (90 mL×3). The combined organic phase was dried over anhydrous Na₂SO₄, filtered, and concentrated to dryness. The residue was purified by flash column chromatography on silica gel (gradient, PE/EtOAc=10/1 to 1/2) to give the title compound (900 mg, 1.94 mmol, 83.0% yield) as a colorless oil.

LC-MS m/e: 465 (MH⁺).

### Step 4. Synthesis of 6-(1-acetyl-4-methylpiperidin-4-yl)-4-hydroxy-2-methylpyrido[4,3-d]pyrimidin-7 (6H)-one

A solution of tert-butyl 4-(2-(5-(1,3-dioxolan-2-yl)-6-ethoxy-2-methylpyrimidin-4-yl)acetamido)-4-methylpiperidine-1-carboxylate (700 mg, 1.51 mmol) in AcOH (8 mL) was microwaved at 110° C. for 2 hours. The resultant mixture was cooled to room temperature and concentrated in vacuum to dryness. The residue was purified by prep-HPLC (SilaSep™ C18 silica flash cartridge, 0%-45% MeCN in H₂O with 0.1% HCOOH) to give the title compound (30 mg, 0.095 mmol, 6.3% yield) as a white solid.

LC-MS m/e: 317 (MH⁺).

### Step 5. Synthesis of (R)-6-(1-acetyl-4-methylpiperidin-4-yl)-2-methyl-4-((1-(2-methyl-3-(trifluoromethyl)phenyl)prop-2-yn-1-yl)amino)pyrido[4,3-d]pyrimidin-7 (6H)-one (Compound 33)

To a solution of 6-(1-acetyl-4-methylpiperidin-4-yl)-4-hydroxy-2-methylpyrido[4,3-d]pyrimidin-7 (6H)-one (30 mg, 0.095 mmol) in MeCN (6 mL) were added tripotassium phosphate (81 mg, 0.380 mmol), DIEA (37 mg, 0.287 mmol), and phosphonitrilic chloride trimer (50 mg, 0.144 mmol). The mixture was stirred at 50° C. for 3 hours, followed by adding (R)-1-(2-methyl-3-(trifluoromethyl)phenyl)prop-2-yn-1-amine (24 mg, 0.113 mmol) at 50° C. and stirring at 90° C. for 3 hours. The mixture was then cooled to room temperature, quenched with water (50 mL), and extracted with EtOAc (60 mL×3). The combined organic phase was dried over anhydrous Na₂SO₄, filtered, and concentrated in vacuum to dryness. The residue was purified by prep-HPLC (SilaSep™ C18 silica flash cartridge, 0%-45% MeCN in H₂O with 0.1% HCOOH) to give the title compound (5 mg, 0.01 mmol, 10.3% yield) as a yellow solid.

¹H NMR (400 MHz, DMSO-d6) δ 9.48 (s, 1H), 8.80 (s, 1H), 8.17 (s, 1H), 7.99 (t, J=6.6 Hz, 1H), 7.68 (d, J=7.1 Hz, 1H), 7.49 (t, J=7.7 Hz, 1H), 6.69 (s, 1H), 6.09 (s, 1H), 4.00 (t, J=13.1 Hz, 1H), 3.68 (s, 1H), 3.61 (dd, J=11.2, 7.2 Hz, 1H), 3.19-3.10 (m, 2H), 2.57 (s, 1H), 2.51 (s, 3H), 2.29-2.16 (m, 6H), 2.00 (s, 3H), 1.73 (s, 3H).

LC-MS m/e: 512 (MH⁺).

### Synthesis of Compound 34: 2-methyl-4-(((R)-1-(2-methyl-3-(trifluoromethyl) phenyl) prop-2-yn-1-yl) amino)-6-(1-(tetrahydrofuran-3-yl) cyclopropyl) pyrido[4,3-d]pyrimidin-7 (6H)-one

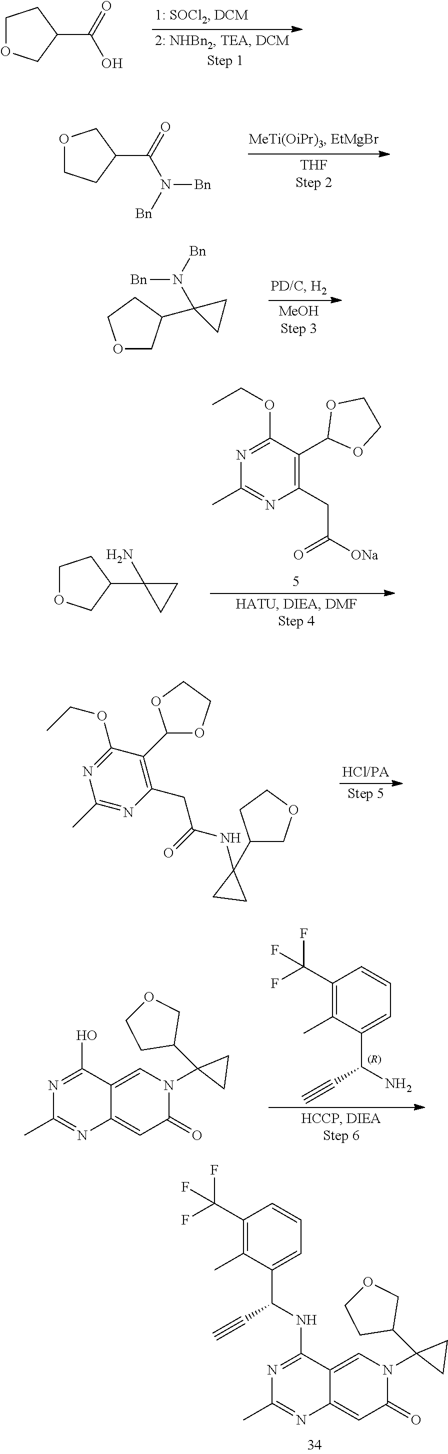

### Step 1. Synthesis of N, N-dibenzyltetrahydrofuran-3-carboxamide

To a solution of oxolane-3-carboxylic acid (5.00 g, 43.059 mmol) in DCM (210 mL) were added SOCl₂ (6.15 g, 51.7 mmol) and DMF (0.2 mL). The reaction mixture was stirred at room temperature for 1 hour, then concentrated to give crude oxolane-3-carbonyl chloride (5 g), which was added to a solution of TEA (10.9 g, 108 mmol) and dibenzylamine (12.7 g, 64.4 mmol) in DCM (210 mL). The resultant mixture was stirred at room temperature for 2 hours, then diluted with water (100 mL), and extracted with DCM (200 mL×2). The organic layer was washed with brine (150 mL), dried over anhydrous Na₂SO₄, filtered, concentrated, and purified by column chromatography (gradient, DCM/MeOH=20/1 to 10/1) to give the title compound (7.5 g, 25.4 mmol, 59.0% yield) as a yellow oil.

LC-MS m/e: 296.3 (MH⁺).

### Step 2. Synthesis of N,N-dibenzyl-1-(tetrahydrofuran-3-yl)cyclopropan-1-amine

To a solution of N, N-dibenzyloxolane-3-carboxamide (6 g, 20.3 mmol) and methyltitanium (IV) triisopropoxide (32.2 ml, 6.60 mmol, 0.5 M in THF) in THF (80 mL) was added ethylmagnesium bromide (7.4 mL, 22.2 mmol, 3 M in THF) at 0° C. The mixture was stirred at room temperature overnight, followed by adding H₂O (20 mL). The resulting mixture was stirred until a colorless precipitate was formed and then filtrated. The filtrate was concentrated and purified by column chromatography (gradient, PE/DCM=1/0 to 2/3) to give the title compound (3 g, 9.76 mmol, 48.0% yield) as a yellow oil.

LC-MS m/e: 308.2 (MH⁺).

### Step 3. Synthesis of 1-(tetrahydrofuran-3-yl)cyclopropan-1-amine

A solution of N, N N-dibenzyl-1-(tetrahydrofuran-3-yl)cyclopropan-1-amine (500 mg, 1.63 mmol) and Pd/C (173 mg, 10% and 55% wt) in MeOH (40 mL) was stirred at room temperature under H₂ overnight. The resulting mixture was filtered through a pad of filter cel and the filtrate was concentrated to give the title compound (150 mg, 1.18 mmol, 72.5% yield) as a yellow oil, which was used directly in the next step without further purification.

LC-MS m/e: 128.2 (MH⁺).

### Step 4. Synthesis of 2-(5-(1,3-dioxolan-2-yl)-6-ethoxy-2-methylpyrimidin-4-yl)-N-(1-(tetrahydrofuran-3-yl)cyclopropyl)acetamide

A solution of sodium 2-(5-(1,3-dioxolan-2-yl)-6-ethoxy-2-methylpyrimidin-4-yl)acetate (376 mg, 1.30 mmol), 1-(tetrahydrofuran-3-yl)cyclopropan-1-amine (150 mg, 1.18 mmol), DIEA (457 mg, 3.54 mmol) and HATU (494 mg, 1.30 mmol) in DMF (5 mL) was stirred at room temperature for 2 hours. Then the reaction mixture was diluted with water (50 mL) and extracted with EtOAc (100 mL×2). The organic layer was washed with brine (100 mL), dried over anhydrous Na₂SO₄, filtered, concentrated, and purified by column chromatography (gradient, PE/EtOAc=10/1 to 1/1) to give the title compound (200 mg, 0.530 mmol, 44.9% yield) as a yellow solid.

LC-MS m/e: 378.3 (MH⁺).

### Step 5. Synthesis of 4-hydroxy-2-methyl-6-(1-(tetrahydrofuran-3-yl)cyclopropyl) pyrido[4,3-d]pyrimidin-7 (6H)-one

To a solution of 2-(5-(1,3-dioxolan-2-yl)-6-ethoxy-2-methylpyrimidin-4-yl)-N-(1-(tetrahydrofuran-3-yl)cyclopropyl)acetamide (200 mg, 0.530 mmol) in IPA (5 mL) was added HCl (0.5 mL, 2.5 mmol, 5 N). The mixture was stirred at 50° C. overnight, then cooled down to room temperature, and concentrated to give the title compound (200 mg, 0.696 mmol, 131% yield) as a yellow solid, which is used directly in the next step without further purification.

LC-MS m/e: 288.1 (MH⁺).

### Step 6. Synthesis of 2-methyl-4-(((R)-1-(2-methyl-3-(trifluoromethyl) phenyl) prop-2-yn-1-yl) amino)-6-(1-(tetrahydrofuran-3-yl) cyclopropyl) pyrido[4,3-d] pyrimidin-7 (6H)-one (Compound 34)

A solution of 4-hydroxy-2-methyl-6-(1-(tetrahydrofuran-3-yl)cyclopropyl)pyrido[4,3-d]pyrimidin-7 (6H)-one (200 mg, 0.696 mmol), phosphonitrilicchloridetrimer (291 mg, 0.835 mmol) and DIEA (0.35 mL, 2.09 mmol) in acetonitrile (10 mL) was stirred at 50° C. for 1.5 hours, following by adding (R)-1-(2-methyl-3-(trifluoromethyl)phenyl)prop-2-yn-1-amine (193 mg, 0.905 mmol). The reaction mixture was stirred at 50° C. for 2 hours, then cooled down to room temperature, and filtered through a pad of filter cel. The filtrated was concentrated to dryness. The residue was purified by pre-HPLC (SilaSep™ C18 silica flash cartridge, 0%-45% MeCN in H₂O with 0.1% HCOOH) to give the title compound (5 mg, 0.010 mmol, 1.49% yield) as a yellow solid.

¹H NMR (400 MHz, DMSO-d6) δ 9.37 (s, 1H), 9.20 (s, 1H), 7.99 (d, J=7.7 Hz, 1H), 7.70 (d, J=7.9 Hz, 1H), 7.50 (t, J=7.7 Hz, 1H), 6.62 (s, 1H), 6.09 (s, 1H), 3.70 (s, 1H), 3.63-3.52 (m, 4H), 2.51 (s, 3H), 2.25 (s, 3H), 1.84-1.48 (m, 3H), 1.08-0.86 (m, 4H).

LC-MS m/e: 483.3 (MH⁺).

### Synthesis of Compound 35: (R)-4-((1-(3-amino-5-(trifluoromethyl)phenyl)prop-2-yn-1-yl)amino)-6-(1-(difluoromethyl)cyclopropyl)-2-methylpyrido[4,3-d]pyrimidin-7 (6H)-one

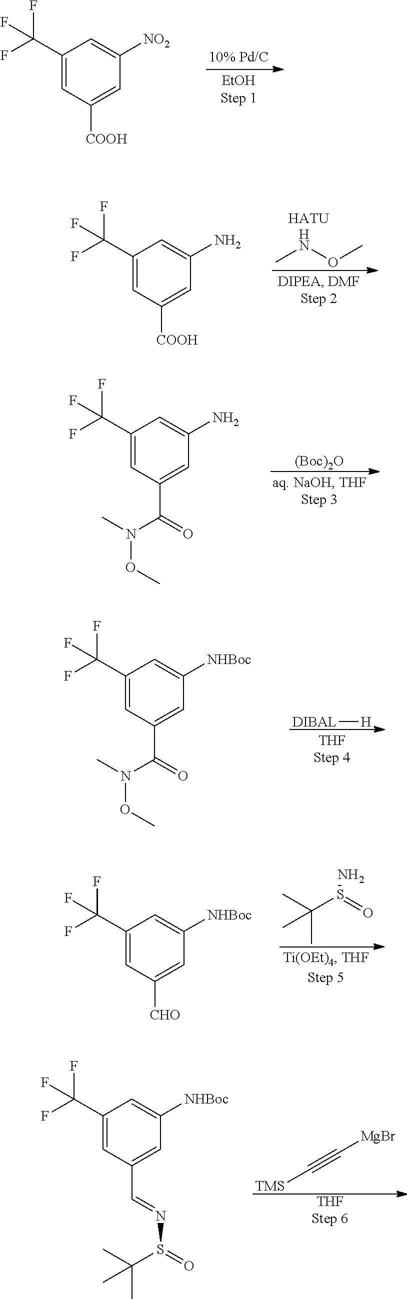

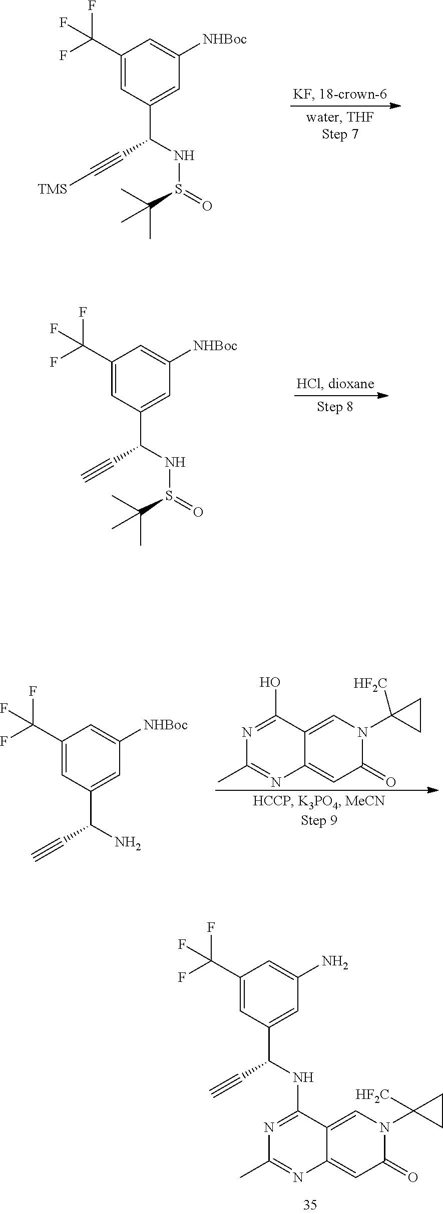

### Step 1. Synthesis of 3-amino-5-(trifluoromethyl)benzoic acid

To a solution of 3-nitro-5-(trifluoromethyl)benzoic acid (7.0 g, 29.7 mmol) in EtOH (100 mL) was added Pd/C (1.0 g, 10% and 55% wet.). The reaction mixture was stirred at 20° C. under H₂ for 18 hours and then filtrated through a pad of filter cel. The filtrate was concentrated under reduced pressure to give the title compound (6.0 g, 29.2 mmol, 98.2% yield) as a yellowish oil.

LC-MS m/e: 206 (MH⁺).

### Step 2. Synthesis of 3-amino-N-methoxy-N-methyl-5-(trifluoromethyl)benzamide

To a solution of 3-amino-5-(trifluoromethyl)benzoic acid (4.5 g, 21.9 mmol) and DIPEA (8.49 g, 65.8 mmol) in DMF (40 mL) were added methoxy(methyl)amine (2.5 mL, 32.9 mmol) and HATU (9.17 g, 24.1 mmol). The reaction mixture was stirred at 20° C. for 1 hour, then diluted with water (100 mL), and extracted with EtOAc (100 mL×2). The combined organic layers were washed with brine (200 mL), dried over anhydrous Na₂SO₄, filtered, and concentrated to dryness. The residue was purified by silica gel flash chromatography (gradient, PE to PE/EtOAc=10/1 to 1/1) to give the title compound (5.0 g, 20.1 mmol, 91.9% yield) as a yellow oil.

LC-MS m/e: 249 (MH⁺).

### Step 3. Synthesis of tert-butyl (3-(methoxy(methyl)carbamoyl)-5-(trifluoromethyl)phenyl)carbamate

To a solution of 3-amino-N-methoxy-N-methyl-5-(trifluoromethyl)benzamide (5.0 g, 20.1 mmol) in THF (70 mL) and water (70 mL) were added (Boc)₂O (13.1 g, 60.4 mmol) and NaOH (3.22 g, 80.5 mmol). The reaction mixture was stirred at 50° C. for 5 hours, then cooled down to room temperature, diluted with water (50 mL), and extracted with EtOAc (100 mL×2). The combined organic layers were washed with brine, dried over anhydrous Na₂SO₄, and filtered. The filtrate was concentrated under reduced pressure and purified by silica gel flash chromatography (gradient, PE/EtOAc=10/1 to 3/1) to give the title compound (2.65 g, 7.60 mmol, 37.7% yield) as a yellow oil.

LC-MS m/e: 349 (MH⁺).

### Step 4. Synthesis of tert-butyl (3-formyl-5-(trifluoromethyl)phenyl)carbamate

To a solution of tert-butyl (3-(methoxy(methyl)carbamoyl)-5-(trifluoromethyl)phenyl) carbamate (2.6 g, 7.46 mmol) in THF (30 mL) was added DIBAL-H (5.47 mL, 8.21 mmol) dropwise at −50° C. The resulting mixture was stirred at −50° C. for 1 hour, then quenched with sat. NH₄Cl solution (20 mL) and extracted with EtOAc (100 mL×2). The combined organic layers were washed with brine (50 mL), dried over anhydrous Na₂SO₄, filtered, and concentrated to dryness. The residue was purified by silica gel flash chromatography (gradient, PE/EtOAc=1/0 to 10/1) to give the title compound (1.8 g, 6.22 mmol, 83.3% yield) as a yellow solid.

LC-MS m/e: 288 (MH⁺).

### Step 5. Synthesis of tert-butyl (S,E)-(3-(((tert-butylsulfinyl)imino)methyl)-5-(trifluoromethyl)phenyl)carbamate

To a solution of tert-butyl (3-formyl-5-(trifluoromethyl)phenyl)carbamate (1.8 g, 6.22 mmol) and (S)-2-methylpropane-2-sulfinamide (1.13 g, 9.33 mmol) in THF (30 mL) was added Ti(OEt)₄ (4.26 g, 18.6 mmol). The resulting mixture was stirred at 70° C. for 3 hours, then cooled down to 10° C., diluted with water (100 mL), and extracted with EtOAc (100 mL×2). The combined organic layers were washed with brine, dried over anhydrous Na₂SO₄, filtered, and concentrated to dryness. The residue was purified by silica gel flash chromatography (gradient, PE/EtOAc=1/0 to 10/1) to give the title compound (2.0 g, 5.09 mmol, 81.9% yield) as a yellow solid.

LC-MS m/e: 393 (MH⁺).

### Step 6. Synthesis of tert-butyl (3-((S)-1-(((S)-tert-butylsulfinyl)amino)-3-(trimethylsilyl)prop-2-yn-1-yl)-5-(trifluoromethyl)phenyl)carbamate

To a solution of tert-butyl (S,E)-(3-(((tert-butylsulfinyl)imino)methyl)-5-(trifluoromethyl)phenyl)carbamate (2.0 g, 5.09 mmol) was added dropwise ((trimethylsilyl)ethynyl)magnesium bromide (25.4 mL, 25.4 mmol, 1 M in THF). The resulting mixture was stirred at 20° C. for 1 hour, then cooled to 0° C., diluted with water (30 mL), and extracted with EtOAc (100 mL×2). The combined organic layers were washed with brine, dried over anhydrous Na₂SO₄, filtered, and concentrated to dryness. The residue was purified by silica gel flash chromatography (gradient, PE/EtOAc=10/1 to 4/1) to give the title compound (1.75 g, 3.56 mmol, 69.9% yield) as a yellow solid.

LC-MS m/e: 491 (MH⁺).

### Step 7. Synthesis of tert-butyl (3-((R)-1-(((S)-tert-butylsulfinyl)amino)prop-2-yn-1-yl)-5-(trifluoromethyl)phenyl)carbamate

To a solution of (3-((S)-1-(((S)-tert-butylsulfinyl)amino)-3-(trimethylsilyl)prop-2-yn-1-yl)-5-(trifluoromethyl)phenyl)carbamate (1.75 g, 3.56 mmol) in THF (20 mL) and water (2 mL) was added KF (0.31 g, 5.35 mmol) and 18-crown-6 (0.19 g, 0.713 mmol). The resulting mixture was stirred at 20° C. for 1 hour, then diluted with water (20 mL), and extracted with EtOAc (30 mL×2). The combined organic layers were washed with brine, dried over anhydrous Na₂SO₄, filtered, and concentrated to dryness. The residue was purified by silica gel flash chromatography (gradient, PE/EtOAc=10/1 to 2/1) to give the title compound (1.0 g, 2.39 mmol, 67.1% yield) as a yellow solid.

LC-MS m/e: 419 (MH⁺).

### Step 8. Synthesis of (R)-3-(1-aminoprop-2-yn-1-yl)-5-(trifluoromethyl)aniline

To a solution of tert-butyl (3-((R)-1-(((S)-tert-butylsulfinyl)amino)prop-2-yn-1-yl)-5-(trifluoromethyl)phenyl)carbamate (1.00 g, 2.39 mmol) in dioxane (20 mL) was added HCl (2.0 mL, 8 mmol, 4 M in dioxane). The resulting mixture was stirred at 20° C. for 5 hours and concentrated under reduced pressure. The residue was triturated with MTBE (20 mL) and filtrated to give a precipitate, which was collected to give the title compound (500 mg, 2.33 mmol, 97.6% yield) as a yellow solid.

LC-MS m/e: 198 (M+H—NH₃)⁺.

### Step 9. Synthesis of (R)-4-((1-(3-amino-5-(trifluoromethyl)phenyl)prop-2-yn-1-yl)amino)-6-(1-(difluoromethyl)cyclopropyl)-2-methylpyrido[4,3-d]pyrimidin-7 (6H)-one (Compound 35)

To a solution of 6-(1-(difluoromethyl)cyclopropyl)-4-hydroxy-2-methylpyrido[4,3-d]pyrimidin-7 (6H)-one (155 mg, 0.580 mmol) in MeCN (30 mL) were added K₃PO₄ (615 mg, 2.90 mmol) and HCCP (302 mg, 0.869 mmol). The resulting mixture was stirred at 80° C. for 2 hours and then added (R)-3-(1-aminoprop-2-yn-1-yl)-5-(trifluoromethyl)aniline (161 mg, 0.752 mmol). The mixture was stirred at 80° C. for 3 hours, then cooled down to 10° C., diluted with water (10 mL), and extracted with EtOAc (20 mL×2). The combined organic layers were washed with brine, dried over anhydrous Na₂SO₄, filtered, and concentrated to dryness. The residue was purified by reverse-phase chromatography (SilaSep™ C18 silica flash cartridge, 0%-45% MeCN in H₂O with 0.1% NH₄HCO₃) to give the title compound (2.8 mg, 0.006 mmol, 1.04% yield) as a yellow solid.

¹H NMR (400 MHz, CD₃OD) δ 9.11 (s, 1H), 7.15 (s, 1H), 7.08 (s, 1H), 6.88 (s, 1H), 6.67 (d, J=2.0 Hz, 1H), 6.38 (s, 1H), 6.22 (t, J=57.5 Hz, 1H), 3.06 (d, J=2.4 Hz, 1H), 2.45 (s, 3H), 1.54 (m, 2H), 1.38 (m, 2H).

LC-MS m/e: 464.2 (MH⁺).

### Synthesis of Compound 36: (R)-4-((1-(3-(difluoromethyl)-2-methylphenyl)prop-2-yn-1-yl)amino)-6-(1-isopropyl-1H-pyrazol-4-yl)-2-methylpyrido[4,3-d]pyrimidin-7 (6H)-one

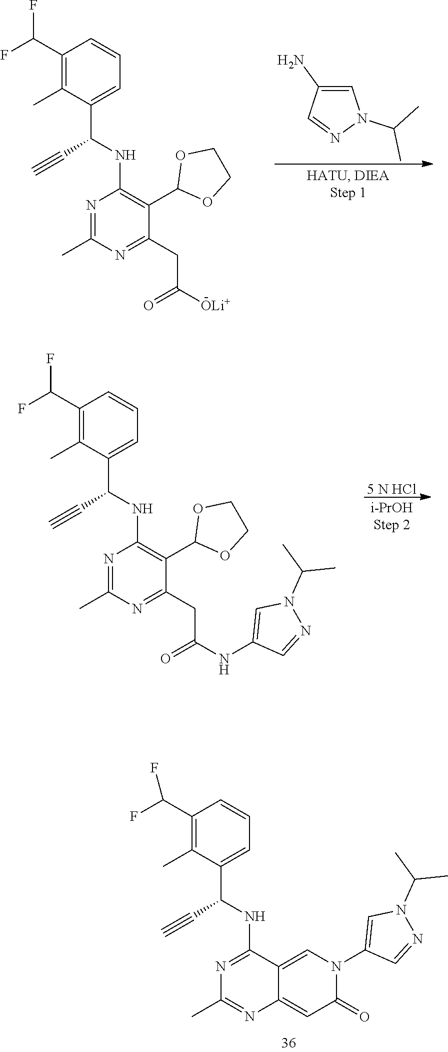

### Step 1. Synthesis of (R)-2-(6-((1-(3-(difluoromethyl)-2-methylphenyl)prop-2-yn-1-yl)amino)-5-(1,3-dioxolan-2-yl)-2-methylpyrimidin-4-yl)-N-(1-isopropyl-1H-pyrazol-4-yl)acetamide

To a solution of lithium (R)-2-(6-((1-(3-(difluoromethyl)-2-methylphenyl)prop-2-yn-1-yl)amino)-5-(1,3-dioxolan-2-yl)-2-methylpyrimidin-4-yl)acetate (150 mg, 0.375 mmol), 1-(propan-2-yl)-1H-pyrazol-4-amine (47 mg, 0.375 mmol) and DIPEA (145 mg, 1.12 mmol) in DMF (2 mL) was added HATU (214 mg, 0.563 mmol). The reaction mixture was stirred at 15° C. for 0.5 hour, then diluted with water (10 mL), and extracted with EtOAc (10 mL×3). The combined organic layers were washed with brine (10 mL), dried over anhydrous Na₂SO₄, filtered, and concentrated to give the title compound (180 mg, 0.343 mmol, 91.6% yield) as a yellow solid, which is used in the next step without further purification.

LC-MS m/e: 525.6 (MH⁺).

### Step 2. Synthesis of (R)-4-((1-(3-(difluoromethyl)-2-methylphenyl)prop-2-yn-1-yl)amino)-6-(1-isopropyl-1H-pyrazol-4-yl)-2-methylpyrido[4,3-d]pyrimidin-7 (6H)-one (Compound 36)

To a solution of (R)-2-(6-((1-(3-(difluoromethyl)-2-methylphenyl)prop-2-yn-1-yl)amino)-5-(1,3-dioxolan-2-yl)-2-methylpyrimidin-4-yl)-N-(1-isopropyl-1H-pyrazol-4-yl)acetamide (180 mg, 0.343 mmol) in propan-2-ol (20 mL) was added HCl (0.4 mL, 2.0 mmol, 5 N). The reaction mixture was stirred at 50° C. for 5 hours, then concentrated to dryness. The residue was purified by prep-HPLC (SilaSep™ C18 silica flash cartridge, 0%-45% MeCN in H₂O with 0.1% NH₄HCO₃) to give the title compound (16.2 mg, 0.035 mmol, 10.2% yield) as a yellow solid.

¹H NMR (400 MHz, DMSO-d6) δ 10.65 (s, 1H), 9.51 (s, 1H), 8.34 (s, 1H), 7.90 (d, J=10.4 Hz, 1H), 7.58 (d, J=7.6 Hz, 1H), 7.46 (t, J=7.8 Hz, 1H), 7.24 (t, J=54.7 Hz, 1H), 6.78 (d, J=4.3 Hz, 1H), 6.24 (s, 1H), 4.65-4.53 (m, 1H), 3.85 (d, J=2.4 Hz, 1H), 2.53 (s, 3H), 2.42 (s, 3H), 1.44 (d, J=6.7 Hz, 6H).

LC-MS m/e: 463.2 (MH⁺).

### Syntheses of Compounds 37 and 38: (R)-4-((1-(3-(difluoromethyl)-2-methylphenyl)prop-2-yn-1-yl)amino)-2-methyl-6-(2-azaspiro[3.3]heptan-6-yl)pyrido[4,3-d]pyrimidin-7 (6H)-one and (R)-4-((1-(3-(difluoromethyl)-2-methylphenyl)prop-2-yn-1-yl)amino)-2-methyl-6-(2-methyl-2-azaspiro[3.3]heptan-6-yl)pyrido[4,3-d]pyrimidin-7 (6H)-one

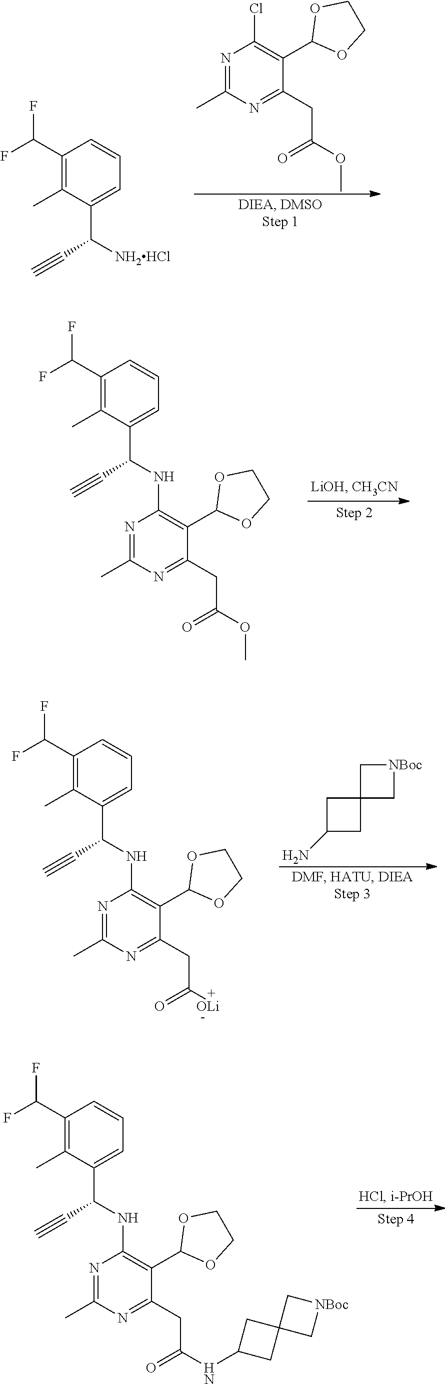

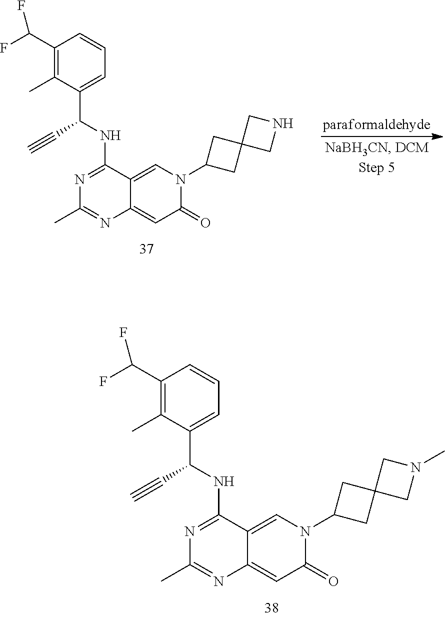 [SMILES: C#C[C@@H](Nc1nc(C)nc2cc(=O)n(C3CC4(CNC4)C3)cc12)c1cccc(C(F)F)c1C ; C#C[C@@H](Nc1nc(C)nc2cc(=O)n(C3CC4(C3)CN(C)C4)cc12)c1cccc(C(F)F)c1C]

### Step 1. Synthesis of methyl (R)-2-(6-((1-(3-(difluoromethyl)-2-methylphenyl)prop-2-yn-1-yl)amino)-5-(1,3-dioxolan-2-yl)-2-methylpyrimidin-4-yl)acetate

To a solution of (R)-1-(3-(difluoromethyl)-2-methylphenyl)prop-2-yn-1-amine hydrochloride (2.20 g, 11.2 mmol) and methyl 2-(6-chloro-5-(1,3-dioxolan-2-yl)-2-methylpyrimidin-4-yl)acetate (3.10 g, 11.2 mmol) in DMSO (10 mL) was added DIPEA (5.6 mL, 33.8 mmol). The resulting mixture was stirred at 100° C. for 18 hours, then cooled to room temperature, diluted with water (100 mL), and extracted with EtOAc (100 mL×2). The combined organic layers were washed with brine, dried over anhydrous Na₂SO₄, filtered, and concentrated to dryness. The residue was purified by silica gel flash chromatography (gradient, PE/EtOAc=10/1 to 2/1) to give the title compound (1.98 g, 4.60 mmol, 41.1% yield) as a brown oil.

LC-MS m/e: 432.3 (MH⁺).

### Step 2. Synthesis of lithium (R)-2-(6-((1-(3-(difluoromethyl)-2-methylphenyl)prop-2-yn-1-yl)amino)-5-(1,3-dioxolan-2-yl)-2-methylpyrimidin-4-yl)acetate

To a solution of (R)-2-(6-((1-(3-(difluoromethyl)-2-methylphenyl)prop-2-yn-1-yl)amino)-5-(1,3-dioxolan-2-yl)-2-methylpyrimidin-4-yl)acetate (1.00 g, 2.32 mmol) in acetonitrile (10 mL) was added LiOH monohydrate (126 mg, 3.0 mmol) in water (1 mL). The reaction mixture was stirred at 25° C. for 18 hours and then concentrated to dryness. The residue was triturated with EtOAc/PE (1:1 in v/v) to give the title compound (600 mg, 1.44 mmol, 62.0% yield) as a white solid.

LC-MS m/e: 418.3 (MH⁺).

### Step 3. Synthesis of tert-butyl (R)-6-(2-(6-((1-(3-(difluoromethyl)-2-methylphenyl)prop-2-yn-1-yl)amino)-5-(1,3-dioxolan-2-yl)-2-methylpyrimidin-4-yl)acetamido)-2-azaspiro[3.3]heptane-2-carboxylate

To a solution of (R)-2-(6-((1-(3-(difluoromethyl)-2-methylphenyl)prop-2-yn-1-yl)amino)-5-(1,3-dioxolan-2-yl)-2-methylpyrimidin-4-yl)acetate (400 mg, 0.96 mmol), tert-butyl 6-amino-2-azaspiro[3.3]heptane-2-carboxylate (306 mg, 1.44 mmol) and DIPEA (371 mg, 2.87 mmol) in DMF (20 mL) was added HATU (547 mg, 1.44 mmol). The reaction mixture was stirred at 25° C. for 2 hours, then diluted with water (20 mL), and extracted with EtOAc (30 mL×2). The organic layer was washed with brine (100 mL), dried over anhydrous Na₂SO₄, filtered, concentrated, and purified by column chromatography (gradient, DCM/MeOH=20/1 to 10/1) to give the title compound (380 mg, 0.621 mmol, 64.7% yield) as a green oil.

LC-MS m/e: 612.3 (MH⁺).

### Step 4. Synthesis of (R)-4-((1-(3-(difluoromethyl)-2-methylphenyl)prop-2-yn-1-yl)amino)-2-methyl-6-(2-azaspiro[3.3]heptan-6-yl)pyrido[4,3-d]pyrimidin-7 (6H)-one (Compound 37)

To a solution of tert-butyl (R)-6-(2-(6-((1-(3-(difluoromethyl)-2-methylphenyl)prop-2-yn-1-yl)amino)-5-(1,3-dioxolan-2-yl)-2-methylpyrimidin-4-yl)acetamido)-2-azaspiro[3.3]heptane-2-carboxylate (380 mg, 0.621 mmol) in i-PrOH (10 mL) is added HCl (0.8 mL, 4 mmol, 5 N). The reaction mixture was stirred at 50° C. for 18 hours, then cooled down to room temperature, and concentrated to give the title compound (250 mg, 89.5% yield) as a light-yellow solid.

¹H NMR (400 MHz, DMSO-d6) δ 9.42 (s, 1H), 8.99 (s, 1H), 7.89 (d, J=7.7 Hz, 1H), 7.53 (d, J=7.6 Hz, 1H), 7.43 (d, J=7.7 Hz, 1H), 7.23 (t, J=54.8 Hz, 1H), 6.69 (s, 1H), 6.09 (s, 1H), 4.88-4.74 (m, 1H), 3.80 (s, 1H), 3.64-3.60 (m, 4H), 2.72-2.63 (m, 2H), 2.54 (s, 2H), 2.43 (s, 3H), 2.27 (s, 3H).

LC-MS m/e: 450 (MH⁺).

### Step 5. Synthesis of (R)-4-((1-(3-(difluoromethyl)-2-methylphenyl)prop-2-yn-1-yl)amino)-2-methyl-6-(2-methyl-2-azaspiro[3.3]heptan-6-yl)pyrido[4,3-d]pyrimidin-7 (6H)-one (Compound 38)

To a solution of (R)-4-((1-(3-(difluoromethyl)-2-methylphenyl)prop-2-yn-1-yl)amino)-2-methyl-6-(2-azaspiro[3.3]heptan-6-yl)pyrido[4,3-d]pyrimidin-7 (6H)-one (200 mg, 0.45 mmol) and paraformaldehyde (134 mg, 4.45 mmol) in DCM (20 mL) was added sodium cyanoborohydride (84 mg, 1.34 mmol). The resulting mixture was stirred at 25° C. for 3 hours, quenched with water (10 mL), and extracted with DCM (50 mL×2). The combined organic layers were washed with brine, dried over anhydrous Na₂SO₄, filtered, and concentrated to dryness. The residue was purified by pre-HPLC (SilaSep™ C18 silica flash cartridge, 0%-45% MeCN in H₂O with 0.1% NH₄HCO₃) to give the title compound (46.3 mg, 0.100 mmol, 22.5% yield) as a yellow solid.

¹H NMR (400 MHz, DMSO-d6) δ 9.33 (d, J=7.5 Hz, 1H), 8.98 (s, 1H), 7.89 (d, J=7.7 Hz, 1H), 7.54 (d, J=7.6 Hz, 1H), 7.43 (t, J=7.7 Hz, 1H), 7.24 (t, J=54.8 Hz, 1H), 6.70 (m, 1H), 6.10 (s, 1H), 4.83 (m, 1H), 3.66 (d, J=2.5 Hz, 1H), 3.27 (s, 2H), 3.13 (s, 2H), 2.61-2.55 (m, 2H), 2.47 (s, 2H), 2.43 (s, 3H), 2.28 (d, J=5.5 Hz, 3H), 2.20 (s, 3H).

LC-MS m/e: 464 (MH⁺).

### Synthesis of Compound 39: (R)-8-bromo-4-((1-(3-(difluoromethyl)-2-methylphenyl)prop-2-yn-1-yl)amino)-6-(1-(difluoromethyl)cyclopropyl)-2-methylpyrido[4,3-d]pyrimidin-7 (6H)-one

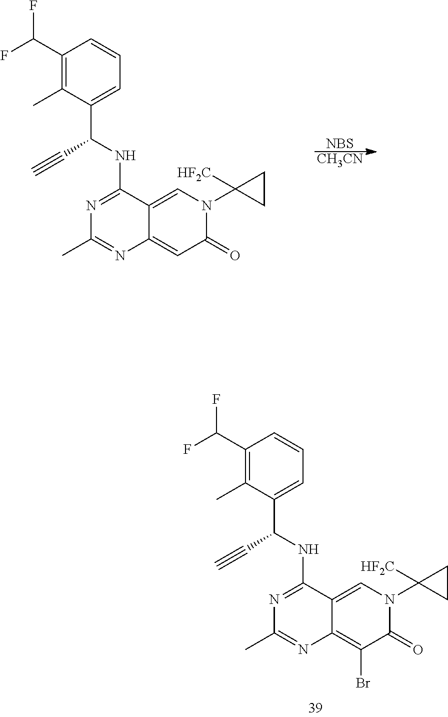

To a solution of (R)-4-((1-(3-(difluoromethyl)-2-methylphenyl)prop-2-yn-1-yl)amino)-6-(1-(difluoromethyl)cyclopropyl)-2-methylpyrido[4,3-d]pyrimidin-7 (6H)-one (50 mg, 0.113 mmol) in CH₃CN (15 mL) was added NBS (6.4 mg, 0.113 mmol) and stirred at room temperature for 1 hour. The reaction mixture was then diluted with H₂O (10 mL) and extracted with EtOAc (20 mL×3). The organic layer was washed with brine (50 mL), dried over anhydrous Na₂SO₄, filtered, and concentrated under reduced pressure to dryness. The residue was purified by prep-HPLC (SilaSep™ C18 silica flash cartridge, 0%-45% MeCN in H₂O with 0.1% HCOOH) to give the title compound (1.0 g, 3.13 mmol, 91.2% yield) as a light-yellow solid.

¹H NMR (400 MHz, DMSO-d6) δ 9.55 (d, J=7.3 Hz, 1H), 9.24 (s, 1H), 7.88 (d, J=7.7 Hz, 1H), 7.55 (d, J=7.5 Hz, 1H), 7.45 (t, J=7.8 Hz, 1H), 7.24 (t, J=54.8 Hz, 1H), 6.67 (dd, J=7.4, 2.4 Hz, 1H), 6.33 (t, J=56.7 Hz, 1H), 3.69 (d, J=2.4 Hz, 1H), 2.43 (s, 3H), 2.37 (s, 3H), 1.52-1.45 (m, 2H), 1.46-1.32 (m, 2H).

LC-MS m/e: 523.1 (MH⁺).

### Synthesis of Compound 40: (R)-4-((1-(3-(difluoromethyl)-2-methylphenyl)prop-2-yn-1-yl)amino)-6-(1-(difluoromethyl)cyclopropyl)pyrido[4,3-d]pyrimidin-7 (6H)-one

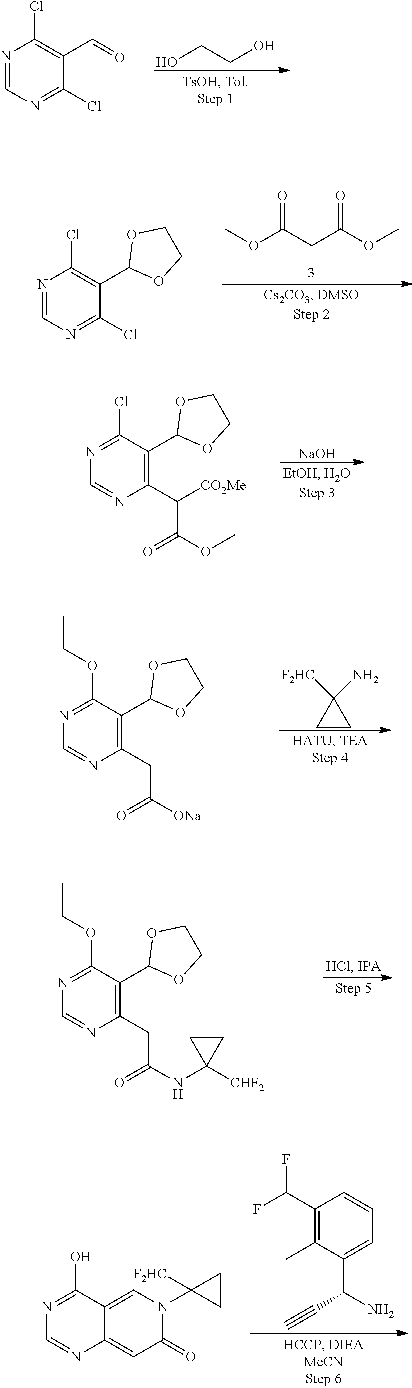

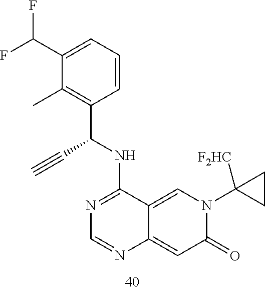 [SMILES: C#C[C@@H](Nc1ncnc2cc(=O)n(C3(C(F)F)CC3)cc12)c1cccc(C(F)F)c1C]

### Step 1. Synthesis of 4,6-dichloro-5-(1,3-dioxolan-2-yl)pyrimidine

A solution of 4,6-dichloropyrimidine-5-carbaldehyde (4.50 g, 25.4 mmol), ethane-1,2-diol (1.58 g, 25.4 mmol) and p-toluene sulphonic acid (0.38 g, 2.54 mmol) in toluene (50 mL) was stirred at 120° C. for 2 hours. The reaction mixture was then cooled down to room temperature and concentrated to dryness. The residue was purified by silica gel column chromatography (gradient, PE/EtOAc=50/1 to 10/1) to give the title compound (3.2 g, 14.5 mmol, 56.9% yield) as a white solid.

LC-MS m/e: 221,223 (MH⁺).

### Step 2. Synthesis of dimethyl 2-(6-chloro-5-(1,3-dioxolan-2-yl) pyrimidin-4-yl) malonate

To a solution of 4,6-dichloro-5-(1,3-dioxolan-2-yl) pyrimidine (3.2 g, 14.5 mmol) and 1,3-dimethyl propanedioate (2.49 g, 18.8 mmol) in DMSO (30 mL) was added Cs₂CO₃ (9.43 g, 29.0 mmol). The reaction mixture was stirred at 80° C. for 2 hours, then cooled down to room temperature, diluted with H₂O (50 mL), extracted with ethyl acetate (50 mL×3), washed with brine (50 mL), dried over anhydrous Na₂SO₄, filtered, and concentrated to dryness. The residue was purified by silica gel column chromatography (gradient, PE/EtOAc=10/1 to 2/1) to give the title compound (4.4 g, 13.9 mmol, 96.0% yield) as a yellow oil.

LC-MS m/e: 317.5 (MH⁺).

### Step 3. Synthesis of sodium 2-(5-(1,3-dioxolan-2-yl)-6-ethoxypyrimidin-4-yl) acetate

To a solution of dimethyl 2-(6-chloro-5-(1,3-dioxolan-2-yl)pyrimidin-4-yl)malonate (4.4 g, 13.9 mmol) in EtOH (90 mL) and water (10 mL) was added NaOH (1.71 g, 42.6 mmol). The reaction mixture was stirred at 60° C. for 6 hours, then cooled down to room temperature, and concentrated to dryness. The residue was suspended in EtOAc (30 mL), stirred at 10° C. for 0.5 hour, and filtered to give a precipitate. The precipitate was washed with EtOAc (20 mL) and dried in vacuum to give the title compound (4.5 g, 9.78 mmol, 68.8% yield, 60% purity) as a white solid.

LC-MS m/e: 300.2 (MH⁺).

### Step 4. Synthesis of 2-(5-(1,3-dioxolan-2-yl)-6-ethoxypyrimidin-4-yl)-N-(1-(difluoromethyl)cyclopropyl)acetamide

To a solution of sodium 2-(5-(1,3-dioxolan-2-yl)-6-ethoxypyrimidin-4-yl)acetate (4.00 g, 9.413 mmol), 1-(difluoro methyl)cyclopropan-1-amine (1.65 g, 10.4 mmol) and TEA (2.86 g, 28.2 mmol) in DMF (40 mL) was added HATU (5.37 g, 14.1 mmol) at 0˜5° C. The reaction mixture was stirred at 15° C. for 0.5 hour, then poured into water (200 mL), and extracted with ethyl acetate (100 mL×3). The combined organic layers were washed with brine (200 mL), dried over anhydrous Na₂SO₄, filtered, and concentrated to give the title compound (3.0 g, 8.74 mmol, 92.8% yield) as a yellow oil, which is used in the next step without further purification.

LC-MS m/e: 345.3 (MH⁺).

### Step 5. Synthesis of 6-(1-(difluoromethyl)cyclopropyl)-4-hydroxypyrido[4,3-d]pyrimidin-7 (6H)-one

To a solution of 2-(5-(1,3-dioxolan-2-yl)-6-ethoxypyrimidin-4-yl)-N-(1-(difluoromethyl)cyclopropyl)acetamide (3.0 g, 8.74 mmol) in i-PrOH (60 mL) was added HCl (2.6 mL, 13.1 mmol, 5 N). The reaction mixture was stirred at 50° C. for 3 hours, then cooled down to room temperature, and basified to a pH value of 10 by adding saturated Na₂CO₃. The resulting mixture was extracted with CHCl₃/i-PrOH (3:1 in v/v, 100 mL×3). The combined organic layers were concentrated to dryness. The residue was purified by flash column chromatography on silica gel (gradient, DCM/MeOH=20/1 to 5/1) to give the title compound (500 mg, 1.98 mmol, 22.6% yield) as a yellow solid.

LC-MS m/e: 253.6 (MH⁺).

### Step 6. Synthesis of (R)-4-((1-(3-(difluoromethyl)-2-methylphenyl)prop-2-yn-1-yl)amino)-6-(1-(difluoromethyl)cyclopropyl)pyrido[4,3-d]pyrimidin-7 (6H)-one (Compound 40)

To a solution of 6-(1-(difluoromethyl)cyclopropyl)-4-hydroxypyrido[4,3-d]pyrimidin-7 (6H)-one (200 mg, 0.790 mmol), and K₃PO₄ (503 mg, 2.37 mmol) in MeCN (15 mL) were added DIEA (306 mg, 2.37 mmol) and phosphonitrilic chloride trimer (347 mg, 1.58 mmol). The reaction mixture was stirred at 20° C. for 1.5 hours and then (R)-1-(3-(difluoromethyl)-2-methylphenyl)prop-2-yn-1-amine (365 mg, 1.58 mmol) was added. The resulting mixture was stirred at 20° C. for 2 hours, then diluted with H₂O (50 mL), extracted with ethyl acetate (50 mL×3), washed with brine (50 mL), dried over anhydrous Na₂SO₄, filtered, and concentrated to dryness. The residue was purified by prep-HPLC (SilaSep™ C18 silica flash cartridge, 0%-45% MeCN in H₂O with 0.1% NH₄HCO₃) to give the title compound (15 mg, 0.035 mmol, 4.41% yield) as a yellow solid.

¹H NMR (400 MHz, DMSO-d6) δ 9.54 (d, J=6.7 Hz, 1H), 9.26 (s, 1H), 8.22 (s, 1H), 7.87 (d, J=7.7 Hz, 1H), 7.55 (d, J=7.7 Hz, 1H), 7.44 (t, J=7.8 Hz, 1H), 7.23 (t, J=54.8 Hz, 1H), 6.61 (d, J=4.8 Hz, 1H), 6.48-6.14 (m, 2H), 3.67 (d, J=2.2 Hz, 1H), 2.38 (s, 3H), 1.46-1.36 (m, 4H).

LC-MS m/e: 431.2 (MH⁺).

### Synthesis of Compound 41: (R)-4-((1-(3-bromo-2-fluorophenyl)prop-2-yn-1-yl)amino)-6-(1-(difluoromethyl)cyclopropyl)-2-methylpyrido[4,3-d]pyrimidin-7 (6H)-one

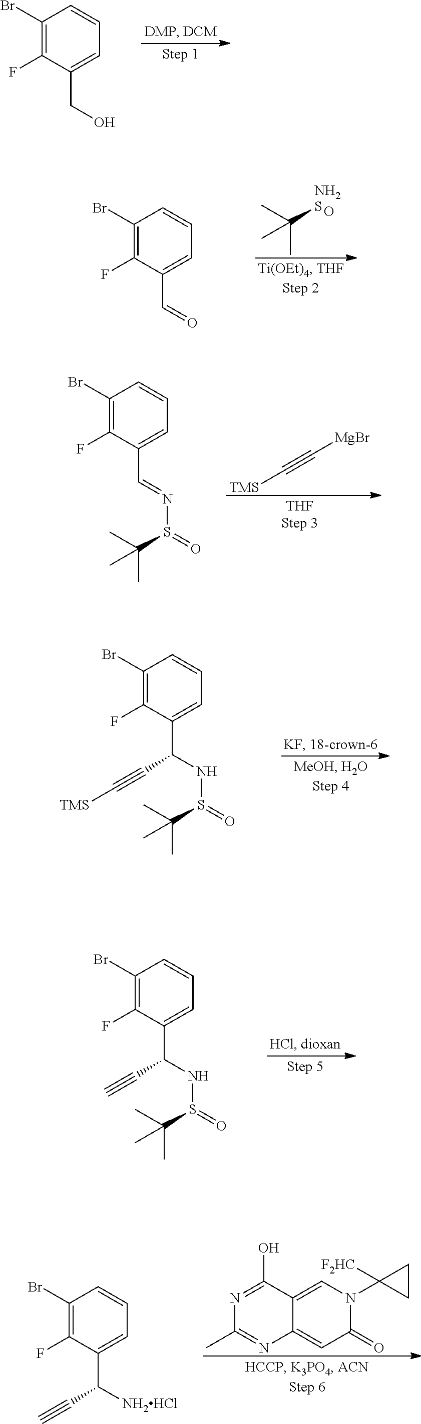

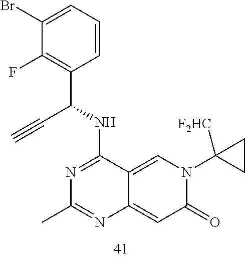 [SMILES: C#C[C@@H](Nc1nc(C)nc2cc(=O)n(C3(C(F)F)CC3)cc12)c1cccc(Br)c1F]

### Step 1. Synthesis of 3-bromo-2-fluorobenzaldehyde

To a solution of (3-bromo-2-fluorophenyl) methanol (5 g, 24.4 mmol) in DCM (70 mL) was added Dess-Martin periodinane (14.5 g, 34.1 mmol). The mixture was stirred at 20° C. for 2 hours and then quenched with water (100 mL). The aqueous layer was extracted with DCM (100 mL×3). The combined organic layers were washes with saturated Na₂S₂O₃ (100 mL), brine (100 mL), dried over anhydrous Na₂SO₄, filtered, and concentrated in vacuum to dryness. The residue was purified by flash column chromatography on silica gel (gradient, PE/EtOAc=1/0 to 25/1) to give the title compound (4.8 g, 23.6 mmol, 96.9% yield) as a white solid.

¹H NMR (400 MHz, DMSO-d6) δ 10.19 (s, 1H), 8.05 (ddd, J=8.2, 6.9, 1.7 Hz, 1H), 7.85 (ddd, J=7.9, 6.4, 1.7 Hz, 1H), 7.37 (t, J=7.8 Hz, 1H).

### Step 2. Synthesis of (S,E)-N-(3-bromo-2-fluorobenzylidene)-2-methylpropane-2-sulfinamide

To a solution of 3-bromo-2-fluorobenzaldehyde (4.8 g, 23.6 mmol) in THF (70 mL) were added (S)-2-methylpropane-2-sulfinamide (4.3 g, 35.5 mmol) and titanium ethoxide (16.2 g, 70.9 mmol). The mixture was stirred at 70° C. for 8 hours, then cooled down to room temperature, quenched with water (100 mL), and filtered. The filtrate was extracted with EtOAc (100 mL×3). The combined organic layers were dried over anhydrous Na₂SO₄, filtered, and concentrated in vacuum to dryness. The residue was purified by flash column chromatography on silica gel (gradient, PE/EtOAc=20/1 to 6/1) to give the title compound (5.5 g, 17.9 mmol, 75.9% yield) as a colorless oil.

LC-MS m/e: 306,308 (MH⁺).

### Step 3. Synthesis of (S)—N—((S)-1-(3-bromo-2-fluorophenyl)-3-(trimethylsilyl)prop-2-yn-1-yl)-2-methylpropane-2-sulfinamide

To a solution of (S,E)-N-(3-bromo-2-fluorobenzylidene)-2-methylpropane-2-sulfinamide (5.5 g, 17.9 mmol) in THF (80 mL) was added ((trimethylsilyl)ethynyl)magnesium bromide (35.8 mL, 35.8 mmol, 1 M in THF) at −78° C. The mixture was stirred at −78° C. for 2 hours and at −20° C. for 1 hour, then quenched with water (100 mL), and extracted with EtOAc (100 mL×3). The combined organic layers were dried over anhydrous Na₂SO₄, filtered, and concentrated in vacuum to dryness. The residue was purified by flash column chromatography on silica gel (gradient, PE/EtOAc=20/1 to 6/1) to give the title compound (6.2 g, 15.3 mmol, 85.4% yield) as a colorless oil.

LC-MS m/e: 404, 406 (MH⁺).

### Step 4. (S)—N—((R)-1-(3-bromo-2-fluorophenyl)prop-2-yn-1-yl)-2-methylpropane-2-sulfinamide

To a solution of (S)—N—((S)-1-(3-bromo-2-fluorophenyl)-3-(trimethylsilyl)prop-2-yn-1-yl)-2-methylpropane-2-sulfinamide (3 g, 7.42 mmol) in THF (32 mL) and water (2 mL) was added 18-crown-6 (2.16 g, 8.16 mmol) and potassium fluoride (0.47 g, 8.16 mmol). The mixture was stirred at 20° C. for 3 hours, then quenched with water (100 mL), and extracted with EtOAc (100 mL×3). The combined organic layers were dried over anhydrous Na₂SO₄, filtered, and concentrated in vacuum to dryness. The residue was purified by flash column chromatography on silica gel (gradient, PE/EtOAc=10/1 to 2/1) to give the title compound (2 g, 6.02 mmol, 81.2% yield) as a colorless oil.

LC-MS m/e: 332, 334 (MH⁺).

### Step 5. Synthesis of (R)-1-(3-bromo-2-fluorophenyl)prop-2-yn-1-amine hydrochloride

To a solution of (S)—N—((R)-1-(3-bromo-2-fluorophenyl)prop-2-yn-1-yl)-2-methylpropane-2-sulfinamide (2 g, 6.02 mmol) in dioxane (30 mL) was added HCl (4.5 mL, 18.0 mmol, 4 M in dioxane). The mixture was stirred at 20° C. for 2 hours, then concentrated and tritiated with MTBE (30 mL) to give the title compound (1.3 g, 4.93 mmol, 82.0% yield) as a white solid.

LC-MS m/e: 228, 230 (MH⁺).

### Step 6. Synthesis of (R)-4-((1-(3-bromo-2-fluorophenyl)prop-2-yn-1-yl)amino)-6-(1-(difluoromethyl)cyclopropyl)-2-methylpyrido[4,3-d]pyrimidin-7 (6H)-one (Compound 41)

To a solution of 6-(1-(difluoromethyl)cyclopropyl)-4-hydroxy-2-methylpyrido[4,3-d]pyrimidin-7 (6H)-one (100 mg, 0.37 mmol) in acetonitrile (6 mL) were added tripotassium phosphate (238 mg, 1.12 mmol) and phosphonitrilic chloride trimer (195 mg, 0.56 mmol). The mixture was stirred at 50° C. for 2 hours, followed by adding (R)-1-(3-bromo-2-fluorophenyl)prop-2-yn-1-amine hydrochloride (119 mg, 0.45 mmol) at 50° C. and stirring at 90° C. for 5 hours. The mixture was then quenched with water (50 mL) and extracted with EtOAc (50 mL×3). The combined organic layers were dried over anhydrous Na₂SO₄, filtered, and concentrated to dryness. The residue was purified by prep-HPLC (SilaSep™ C18 silica flash cartridge, 0%-45% MeCN in H₂O with 0.1% HCOOH) to give the title compound (13.9 mg, 0.029 mmol, 7.8%) as a yellow solid.

¹H NMR (400 MHz, DMSO-d6) δ 11.05 (s, 1H), 9.44 (s, 1H), 7.79 (t, J=7.3 Hz, 2H), 7.32 (t, J=7.8 Hz, 1H), 6.85 (d, J=4.7 Hz, 1H), 6.40-6.12 (m, 2H), 3.95 (d, J=2.4 Hz, 1H), 3.31 (s, 3H), 1.51-1.42 (m, 4H).

LC-MS m/e: 477.1, 479.1 (MH⁺).

### Synthesis of Compound 42: (R)-3-(1-((6-(1-(difluoromethyl)cyclopropyl)-2-methyl-7-oxo-6,7-dihydropyrido[4,3-d]pyrimidin-4-yl)amino)prop-2-yn-1-yl)-2-fluorobenzonitrile

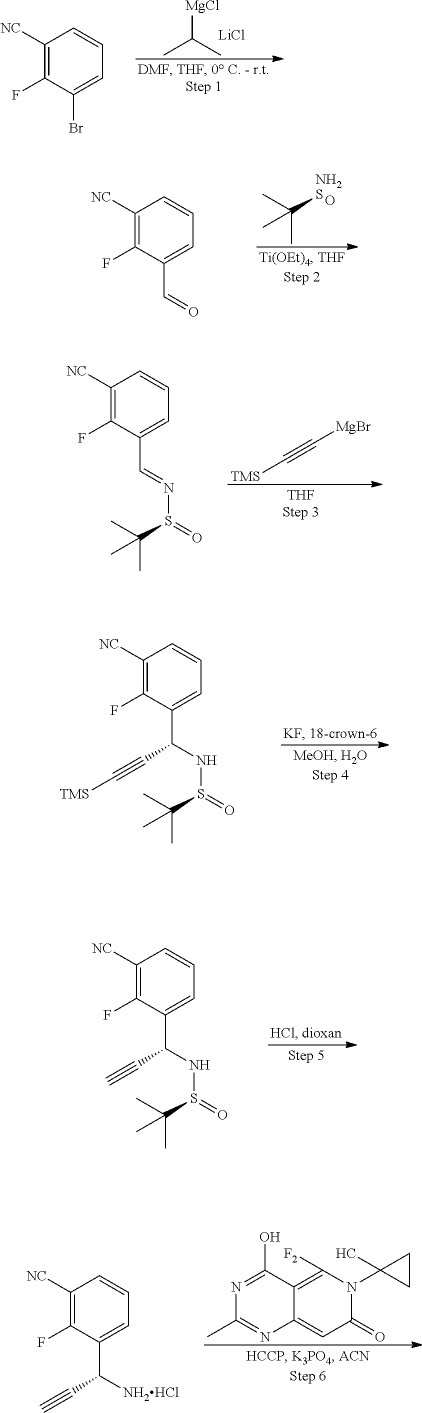

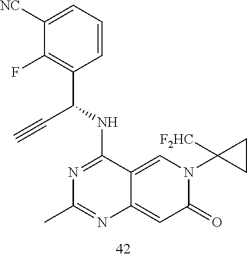 [SMILES: C#C[C@@H](Nc1nc(C)nc2cc(=O)n(C3(C(F)F)CC3)cc12)c1cccc(C#N)c1F]

### Step 1. Synthesis of 2-fluoro-3-formylbenzonitrile

To a solution of 3-bromo-2-fluorobenzonitrile (1 g, 5.00 mmol) in THF (15 mL) was added isopropylmagnesium chloride lithium chloride complex solution (4.6 mL, 6.00 mmol, 1.3 M in THF) at 0° C. The mixture was stirred at 20° C. for 1 hour, followed by adding DMF (1.16 mL, 15.0 mmol) at 0° C. and stirring at 20° C. for 8 hours. The mixture was then quenched with saturated NH₄Cl (50 mL) and extracted with EtOAc (80 mL×3). The combined organic layers were dried over anhydrous Na₂SO₄, filtered, and concentrated to dryness. The residue was purified by flash column chromatography on silica gel (gradient, PE/EtOAc=1/0 to 10/1) to give the title compound (400 mg, 2.68 mmol, 53.6% yield) as a colorless oil.

¹H NMR (400 MHz, DMSO-d6) δ 10.21 (s, 1H), 8.26 (ddd, J=7.9, 6.3, 1.8 Hz, 1H), 8.18 (ddd, J=7.8, 7.1, 1.8 Hz, 1H), 7.60 (t, J=7.8 Hz, 1H).

### Step 2. Synthesis of (S,E)-N-(3-cyano-2-fluorobenzylidene)-2-methylpropane-2-sulfinamide

To a solution of 2-fluoro-3-formylbenzonitrile (400 mg, 2.68 mmol) in THF (10 mL) was added (S)-2-methylpropane-2-sulfinamide (488 mg, 4.02 mmol) and titanium ethoxide (1.84 g, 8.05 mmol). The mixture was stirred at 70° C. for 5 hours, then cooled down to room temperature, quenched with water (20 mL), filtered, and extracted with EtOAc (60 mL×3). The combined organic layers were dried over anhydrous Na₂SO₄, filtered, and concentrated in vacuum to dryness. The residue was purified by flash column chromatography on silica gel (gradient, PE/EtOAc=10/1 to 4/1) to give the title compound (600 mg, 2.38 mmol, 88.7% yield) as a colorless oil.

LC-MS m/e: 253 (MH⁺).

### Step 3. Synthesis of (S)—N—((S)-1-(3-cyano-2-fluorophenyl)-3-(trimethylsilyl)prop-2-yn-1-yl)-2-methylpropane-2-sulfinamide

To a solution of (S,E)-N-(3-cyano-2-fluorobenzylidene)-2-methylpropane-2-sulfinamide (600 mg, 2.38 mmol) in THF (12 mL) was added ((trimethylsilyl)ethynyl)magnesium bromide (7.1 mL, 7.1 mmol, 1 M in THF) at −78° C. Then the mixture was stirred at −10° C. for 2 hours, then quenched with water (20 mL), and extracted with EtOAc (50 mL×3). The combined organic layers were dried over anhydrous Na₂SO₄, filtered, and concentrated to dryness. The residue was purified by flash column chromatography on silica gel (gradient, PE/EtOAc=10/1 to 3/1) to give the title compound (300 mg, 0.86 mmol, 36.0% yield) as a colorless oil.

LC-MS m/e: 351 (MH⁺).

### Step 4. Synthesis of (S)—N—((R)-1-(3-cyano-2-fluorophenyl)prop-2-yn-1-yl)-2-methylpropane-2-sulfinamide

To a solution of (S)—N—((S)-1-(3-cyano-2-fluorophenyl)-3-(trimethylsilyl)prop-2-yn-1-yl)-2-methylpropane-2-sulfinamide (300 mg, 0.86 mmol) in THF (6 mL) and water (0.5 mL) was added 18-crown-6 (249 mg, 0.94 mmol) and potassium fluoride (55 mg, 0.94 mmol). The mixture was stirred at 20° C. for 2 hours, then quenched with water (30 mL), and extracted with EtOAc (60 mL×3). The combined organic layers were dried over anhydrous Na₂SO₄, filtered, and concentrated to dryness. The residue was purified by flash column chromatography on silica gel (gradient, DCM/MeOH=20/1 to 8/1) to give the title compound (150 mg, 0.54 mmol, 63.0% yield) as a colorless oil.

LC-MS m/e: 279 (MH⁺).

### Step 5. Synthesis of (R)-3-(1-aminoprop-2-yn-1-yl)-2-fluorobenzonitrile hydrochloride

To a solution of (S)—N—((R)-1-(3-cyano-2-fluorophenyl)prop-2-yn-1-yl)-2-methylpropane-2-sulfinamide (150 mg, 0.54 mmol) in dioxane (6 mL) was added HCl (0.4 mL, 1.6 mmol, 4 M in dioxane). The mixture was stirred at 20° C. for 2 hours, then concentrated and tritiated with MTBE (40 mL). The solid was collected to give the title compound (70 mg, 0.332 mmol, 61.5%) as a colorless oil.

¹H NMR (400 MHz, DMSO-d6) δ 9.22 (s, 2H), 8.21-8.01 (m, 2H), 7.58 (t, J=7.9 Hz, 1H), 5.69 (d, J=2.2 Hz, 1H), 4.05 (d, J=2.4 Hz, 1H).

### Step 6. Synthesis of (R)-3-(1-((6-(1-(difluoromethyl)cyclopropyl)-2-methyl-7-oxo-6,7-dihydropyrido[4,3-d]pyrimidin-4-yl)amino)prop-2-yn-1-yl)-2-fluorobenzonitrile (Compound 42)

To a solution of 6-(1-(difluoromethyl)cyclopropyl)-4-hydroxy-2-methylpyrido[4,3-d]pyrimidin-7 (6H)-one (80 mg, 0.299 mmol) in MeCN (5 mL) was added phosphonitrilic chloride trimer (156 mg, 0.449 mmol) and tripotassium phosphate (191 mg, 0.898 mmol). The mixture was stirred at 50° C. for 1 hour, followed by adding (R)-3-(1-aminoprop-2-yn-1-yl)-2-fluorobenzonitrile hydrochloride (76 mg, 0.359 mmol) in MeCN (5 mL) and stirring at 90° C. for 2 hours. The mixture was then cooled to room temperature, quenched with water (30 mL), and extracted with EtOAc (60 mL×3). The combined organic layers were dried over anhydrous Na₂SO₄, filtered, and concentrated to dryness. The residue was purified by prep-HPLC (SilaSep™ C18 silica flash cartridge, 0%-45% MeCN in H₂O with 0.1% NH₄HCO₃) to give the title compound (24 mg, 0.057 mmol, 18.9% yield) as a white solid.

¹H NMR (400 MHz, DMSO-d6) δ 9.48 (s, 1H), 9.15 (s, 1H), 8.05 (t, J=7.4 Hz, 1H), 7.95 (t, J=6.4 Hz, 1H), 7.51 (t, J=7.8 Hz, 1H), 6.67 (s, 1H), 6.28 (dd, J=91.8, 34.8 Hz, 2H), 3.79 (d, J=2.3 Hz, 1H), 2.28 (s, 3H), 1.42 (d, J=34.0 Hz, 4H).

LC-MS m/e: 424 (MH⁺).

### Syntheses of Compounds 43-45

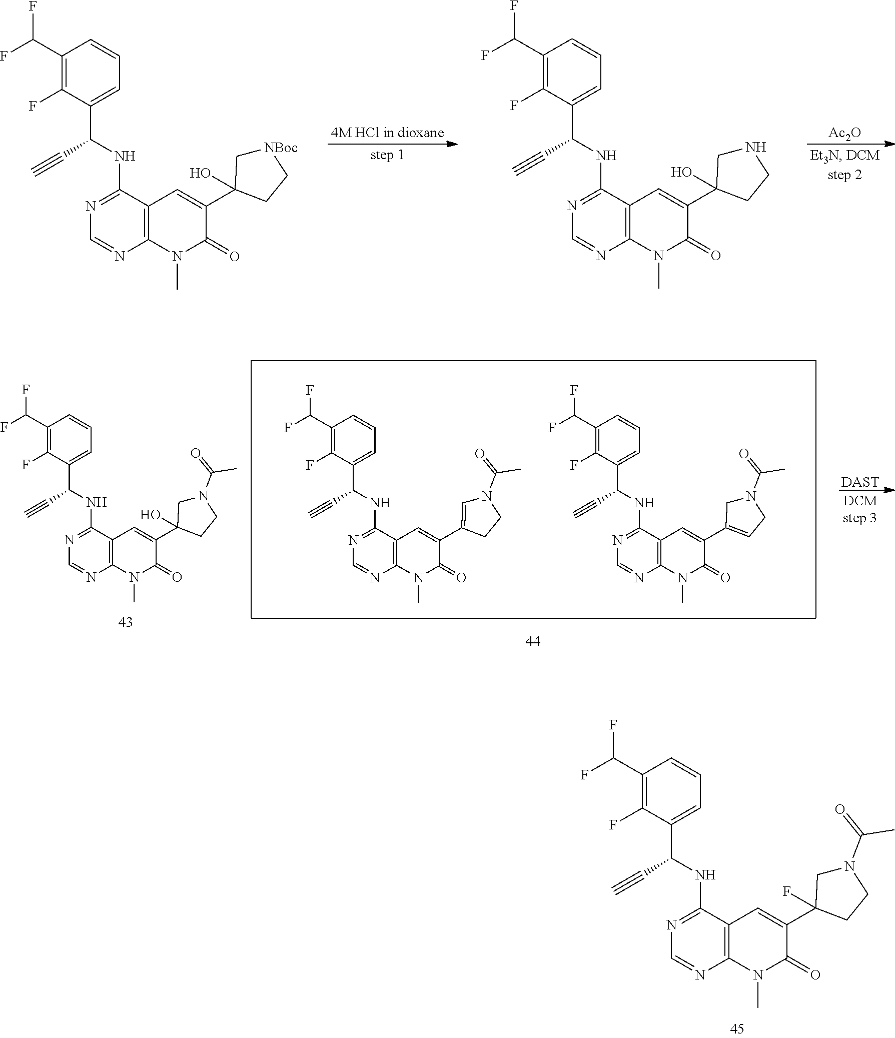

### Step 1. Synthesis of 4-{[(1R)-1-[3-(difluoromethyl)-2-fluorophenyl]prop-2-yn-1-yl]amino}-6-(3-hydroxypyrrolidin-3-yl)-8-methyl-7H,8H-pyrido[2,3-d]pyrimidin-7-one

To a solution of tert-butyl 3-(4-{[(1R)-1-[3-(difluoromethyl)-2-fluorophenyl]prop-2-yn-1-yl]amino}-8-methyl-7-oxo-7H,8H-pyrido[2,3-d]pyrimidin-6-yl)-3-hydroxypyrrolidine-1-carboxylate (330 mg, 0.607 mmol) in dioxane (10 mL) was added HCl (1.5 mL, 4 M in dioxane) dropwise. The resulting mixture was stirred at 20° C. for 4 hours and then concentrated under reduced pressure to give the title compound (250 mg, 0.564 mmol, 92.8% yield) as a brown solid, which was used in next step directly.

LC-MS m/e: 444 (MH⁺).

### Step 2. Synthesis of 6-(1-acetyl-3-hydroxypyrrolidin-3-yl)-4-{[(1R)-1-[3-(difluoromethyl)-2-fluorophenyl]prop-2-yn-1-yl]amino}-8-methyl-7H,8H-pyrido[2,3-d]pyrimidin-7-one (Compound 43) and a mixture of 6-(1-acetyl-2,5-dihydro-1H-pyrrol-3-yl)-4-{[(1R)-1-[3-(difluoromethyl)-2-fluorophenyl]prop-2-yn-1-yl]amino}-8-methyl-7H,8H-pyrido[2,3-d]pyrimidin-7-one and 6-(1-acetyl-4,5-dihydro-1H-pyrrol-3-yl)-4-{[(1R)-1-[3-(difluoromethyl)-2-fluorophenyl]prop-2-yn-1-yl]amino}-8-methyl-7H,8H-pyrido[2,3-d]pyrimidin-7-one (Compound 44)

To a solution of 4-{[(1R)-1-[3-(difluoromethyl)-2-fluorophenyl]prop-2-yn-1-yl] amino}-6-(3-hydroxypyrrolidin-3-yl)-8-methyl-7H,8H-pyrido[2,3-d]pyrimidin-7-one (100 mg, 0.226 mmol) and Et₃N (114 mg, 1.13 mmol) in DCM (10 mL) was added Ac₂O (69.0 mg, 0.677 mmol) dropwise at 0° C. The mixture was stirred at 20° C. for 2 hours, then diluted with water (10 mL), and extracted with DCM (20 mL×2). The combined organic layers were washed with brine, dried over anhydrous Na₂SO₄, filtered, and concentrated under reduced pressure to dryness. The residue was purified by reverse-phase column chromatography (SilaSep™ C18 silica flash cartridge, 0%-40% MeCN in H₂O with 0.1% formic acid) to give Compound 43 (3.7 mg, 0.0076 mmol, 3.4% yield) as a yellow solid and Compound 44 (16.0 mg, 0.034 mmol, 15.2% yield) as a yellow solid.

Compound 43: ¹H NMR (400 MHz, CD₃OD) δ 8.47 (s, 1H), 8.42 (d, J=2.6 Hz, 1H), 7.95 (t, J=7.4 Hz, 1H), 7.60 (t, J=6.8 Hz, 1H), 7.35 (t, J=7.7 Hz, 1H), 6.99 (t, J=54.7 Hz, 1H), 6.80 (d, J=4.1 Hz, 1H), 4.07-3.84 (m, 1H), 3.79 (m, 2H), 3.72 (d, J=3.1 Hz, 3H), 3.61 (m, 1H), 3.05-2.99 (m, 1H), 2.73 (m, 1H), 2.08 (m, 4H); LC-MS m/e: 486 (MH⁺).

Compound 44: ¹H NMR (400 MHz, MeOD) δ 8.45 (s, 1H), 8.06 (s, 1H), 8.00-7.91 (m, 1H), 7.61 (t, J=7.2 Hz, 1H), 7.36 (t, J=7.7 Hz, 1H), 7.16 (m, 1H), 7.00 (t, J=54.7 Hz, 1H), 6.81 (m, 1H), 4.71 (s, 1H), 4.53 (s, 2H), 4.37 (s, 1H), 3.73 (s, 3H), 3.05 (m, 1H), 2.14 (m, 3H); LC-MS m/e: 468 (MH⁺).

### Step 3. Synthesis of 6-(1-acetyl-3-fluoropyrrolidin-3-yl)-4-{[(1R)-1-[3-(difluoromethyl)-2-fluorophenyl]prop-2-yn-1-yl]amino}-8-methyl-7H,8H-pyrido[2,3-d]pyrimidin-7-one (Compound 45)

To a solution of 6-(1-acetyl-3-hydroxypyrrolidin-3-yl)-4-{[(1R)-1-[3-(difluoromethyl)-2-fluorophenyl]prop-2-yn-1-yl]amino}-8-methyl-7H,8H-pyrido[2,3-d]pyrimidin-7-one (60 mg, 0.124 mmol) in DCM (3 mL) was added DAST (0.04 mL, 0.31 mmol) dropwise at 0° C. under N₂. The mixture was stirred at 20° C. for 1.5 hours, quenched with ice water (5 mL), and extracted with DCM (10 mL×2). The combined organic layers were washed with brine and purified by reverse-phase column chromatography (SilaSep™ C18 silica flash cartridge, 0%-40% MeCN in H₂O with 0.1% formic acid) to give the title compound (11.5 mg, 0.024 mmol, 19.1% yield) as a yellow solid.

¹H NMR (400 MHz, MeOD) δ 8.51 (m, 1H), 8.50-8.46 (m, 1H), 7.93 (m, 1H), 7.59 (m, 1H), 7.34 (m, 1H), 6.99 (t, J=54.7 Hz, 1H), 6.79 (m, 1H), 4.20 (m, 1H), 3.86 (m, 3H), 3.72 (d, J=3.7 Hz, 3H), 3.67-3.45 (m, 1H), 3.02 (m, 1H), 2.29 (m, 1H), 2.15-2.03 (m, 3H).

LC-MS m/e: 488 (MH⁺).

### Syntheses of Compounds 46 and 47

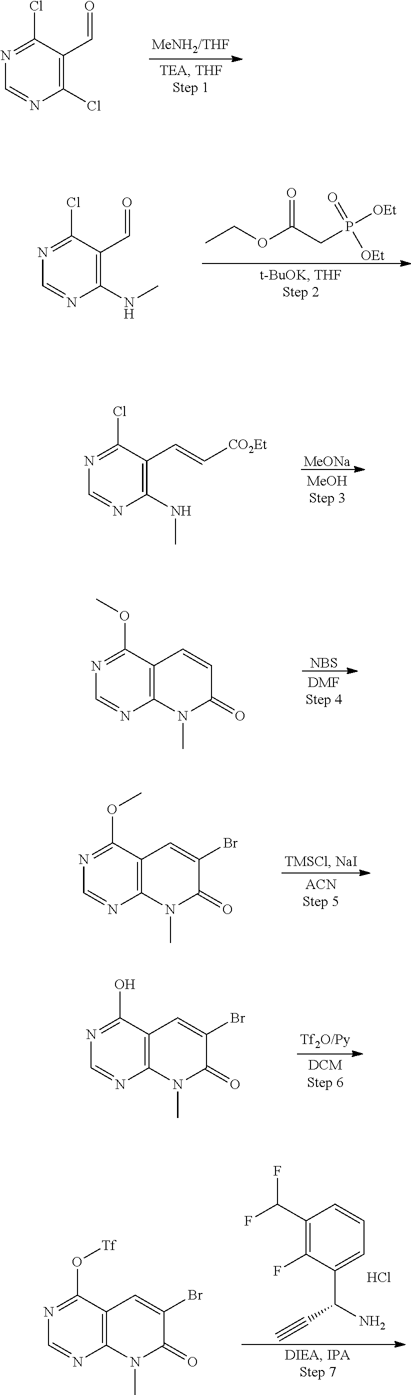

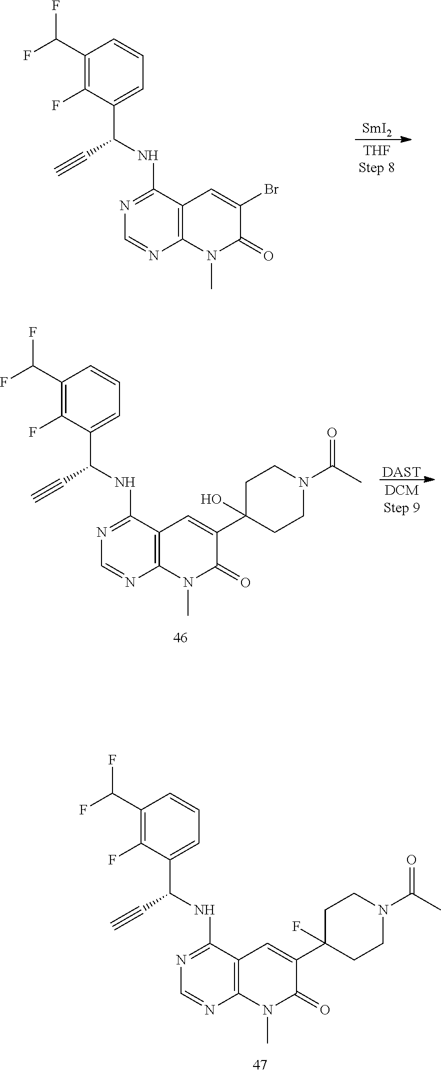

### Step 1. Synthesis of 4-chloro-6-(methylamino)pyrimidine-5-carbaldehyde

To a solution of 4,6-dichloropyrimidine-5-carbaldehyde (25.0 g, 141 mmol) and TEA (28.6 g, 282 mmol) in THF (200 mL) was added methylamine solution (74.2 mL, 2.0 N in THF) at 0° C. The mixture was stirred at room temperature for 1.5 hours to give a precipitate, which was filtered. The filtrate was concentrated, triturated with MTBE (70 mL), and stirred for 4 hours to give another precipitate, which was collected and dried in vacuum to give the title compound (12.0 g, 69.5 mmol, 49.2% yield) as a light yellow solid.

LC-MS m/e: 172.1 (MH⁺).

### Step 2. Synthesis of ethyl (2E)-3-[4-chloro-6-(methylamino) pyrimidin-5-yl] prop-2-enoate

A solution of ethyl 2-(diethoxyphosphoryl) acetate (18.8 g, 83.9 mmol) and potassium-butoxide (10.2 g, 90.9 mmol) in THF (200 mL) was stirred at room temperature for 1.5 hours. Then 4-chloro-6-(methylamino) pyrimidine-5-carbaldehyde (12.0 g, 69.9 mmol) was added to the solution. The mixture was stirred for another 2 hours at room temperature and then diluted with saturate NH₄Cl (100 mL). The organic layer was washed with brine, dried over Na₂SO₄, and filtered. The filtrate was concentrated under reduced pressure and purified by flash chromatography (gradient, PE to PE:EtOAc=4:1) to give the title compound (8.50 g, 35.2 mmol, 50.3% yield) as a light yellow solid.

LC-MS m/e: 242.1 (MH⁺).

### Step 3. Synthesis of 4-methoxy-8-methyl-7H,8H-pyrido[2,3-d] pyrimidin-7-one

A solution of ethyl (2E)-3-[4-chloro-6-(methylamino) pyrimidin-5-yl] prop-2-enoate (10.0 g, 41.4 mmol) and sodium methanolate (4.47 g, 82.8 mmol) in methanol (100 mL) was stirred at 50° C. for 4 hours, then cooled down to room temperature, diluted with saturated NH₄Cl (50 mL), and concentrated. The residue was washed with water (20 mL×2) to give a precipitate, which was collected and dried under vacuum at 50° C. to give the title compound (5.50 g, 28.8 mmol, 69.5% yield) as a light yellow solid.

LC-MS m/e: 192.1 (MH⁺).

### Step 4. Synthesis of 6-bromo-4-methoxy-8-methyl-7H,8H-pyrido[2,3-d] pyrimidin-7-one

To a solution of 4-methoxy-8-methyl-7H,8H-pyrido[2,3-d] pyrimidin-7-one (5.10 g, 26.7 mmol) in DMF (50 mL) was added NBS (9.50 g, 53.4 mmol). The mixture was stirred at 50° C. for 2 hours, then cooled down to room temperature, diluted with water (100 mL), and extracted with EtOAc (100 mL×2). The organic layer was washed with brine (80 mL×2), dried over anhydrous Na₂SO₄, and filtered. The filtrate was concentrated under reduced pressure and purified by flash chromatography (gradient, PE to PE:EtOAc=1:1) to give the title compound (5.50 g, 20.4 mmol, 76.3% yield) as a yellow solid.

LC-MS m/e: 270.1 (MH⁺).

### Step 5. Synthesis of 6-bromo-4-hydroxy-8-methyl-7H,8H-pyrido[2,3-d] pyrimidin-7-one

A solution of 6-bromo-4-methoxy-8-methyl-7H,8H-pyrido[2,3-d] pyrimidin-7-one (5.00 g, 18.5 mmol), NaI (8.32 g, 55.5 mmol), and TMSCl (6.03 g, 55.5 mmol) in acetonitrile (50 mL) was stirred at room temperature for 2 hours. Then the resulting mixture was diluted with EtOAc (50 mL) to give a precipitate, which was collected, rinsed with water (40 mL×2), and dried in vacuum at 70° C. to give the title compound (4.20 g, 16.4 mmol, 88.6% yield) as a light yellow solid.

LC-MS m/e: 256.1 (MH⁺).

### Step 6. Synthesis of 6-bromo-8-methyl-7-oxo-7H,8H-pyrido[2,3-d] pyrimidin-4-yl trifluoromethanesulfonate

To a solution of 6-bromo-4-hydroxy-8-methyl-7H,8H-pyrido[2,3-d] pyrimidin-7-one (1.00 g, 3.91 mmol) and pyridine (0.93 g, 11.7 mmol) in DCM (20 mL) was added trifluoromethanesulfonic anhydride (2.75 g, 9.76 mmol) at 0° C. The mixture was stirred at room temperature for 2 hours, then diluted with water (20 mL), and extracted with EtOAc (40 mL×2). The organic layer was washed with brine (40 mL), dried over anhydrous Na₂SO₄, and filtered. The filtrate was concentrated to give the title compound (1.44 g, 3.7 mmol, 95.0% yield) as a yellow solid, which was used directly in next step without further purification.

LC-MS m/e: 387.9 (MH⁺).

### Step 7. Synthesis of 6-bromo-4-{[(1R)-1-[3-(difluoromethyl)-2-fluorophenyl] prop-2-yn-1-yl] amino}-8-methyl-7H,8H-pyrido[2,3-d] pyrimidin-7-one

A solution of 6-bromo-8-methyl-7-oxo-7H,8H-pyrido[2,3-d] pyrimidin-4-yl trifluoromethanesulfonate (1.40 g, 3.61 mmol), DIEA (1.79 mL, 10.8 mmol) and (1R)-1-[3-(difluoromethyl)-2-fluorophenyl] prop-2-yn-1-amine (0.79 g, 3.97 mmol) in dioxane (15 mL) was stirred at 100° C. for 1 hour, then diluted with water (20 mL), and extracted with EtOAc (50 mL×2). The organic layer was washed with brine, dried over anhydrous Na₂SO₄, and filtered. The filtrate was concentrated under reduced pressure and purified by flash chromatography (gradient, PE:EtOAc=1:1 to 100% EtOAc) to give the title compound (1.10 g, 2.52 mmol, 69.8% yield).

LC-MS m/e: 437.0 (MH⁺).

### Step 8. Synthesis of 6-(1-acetyl-4-hydroxypiperidin-4-yl)-4-{[(1R)-1-[3-(difluoromethyl)-2-fluorophenyl] prop-2-yn-1-yl] amino}-8-methyl-7H,8H-pyrido[2,3-d]pyrimidin-7-one (Compound 46)

A solution of 6-bromo-4-{[(1R)-1-[3-(difluoromethyl)-2-fluorophenyl] prop-2-yn-1-yl]amino}-8-methyl-7H,8H-pyrido[2,3-d] pyrimidin-7-one (1.10 g, 2.52 mmol) and 1-acetylpiperidin-4-one (1.07 g, 7.55 mmol) in SmI₂ (126 mL, 12.6 mmol, 0.10 M in THF) was stirred at −78° C. for 30 minutes, then warmed to room temperature, and stirred at room temperature for 4 hours. The mixture was then quenched with saturated NaHCO₃ (60 mL) and extracted with EtOAc (100 mL×2). The organic layer was washed with brine (100 mL), dried over anhydrous Na₂SO₄, and filtered. The filtrate was concentrated and purified by pre-HPLC to the title compound (600 mg, 1.20 mmol, 47.7% yield) as a light yellow solid.

¹H NMR (400 MHz, DMSO-d6) δ 9.18 (d, J=7.4 Hz, 1H), 8.48 (s, 2H), 7.94 (s, 1H), 7.64 (s, 1H), 7.43 (t, J=7.7 Hz, 1H), 7.24 (t, J=54.1 Hz, 1H), 6.78 (d, J=7.4 Hz, 1H), 5.43 (s, 1H), 4.33 (d, J=11.5 Hz, 1H), 3.70 (d, J=12.0 Hz, 1H), 3.65 (s, 1H), 3.60 (s, 3H), 3.44 (s, 1H), 2.91 (t, J=12.5 Hz, 1H), 2.39-2.25 (m, 2H), 2.02 (s, 3H), 1.59 (d, J=12.4 Hz, 1H), 1.51 (d, J=13.2 Hz, 1H).

LC-MS m/e: 500.2 (MH⁺).

### Step 9. Synthesis of 6-(1-acetyl-4-fluoropiperidin-4-yl)-4-{[(1R)-1-[3-(difluoromethyl)-2-fluorophenyl] prop-2-yn-1-yl] amino}-8-methyl-7H,8H-pyrido[2,3-d] pyrimidin-7-one (Compound 47)

To a solution of 6-(1-acetyl-4-hydroxypiperidin-4-yl)-4-{[(1R)-1-[3-(difluoromethyl)-2-fluorophenyl] prop-2-yn-1-yl] amino}-8-methyl-7H,8H-pyrido[2,3-d] pyrimidin-7-one (100 mg, 0.200 mmol) in DCM (2.0 mL) was added DAST (48.0 mg, 0.300 mmol) at 0° C. The mixture was stirred at 0° C. for 2 hours, then quenched with saturated NaHCO₃ (20 mL) at 0° C., and extracted with DCM (40 mL×2). The combined organic layer was washed with brine (20 mL×2), dried over anhydrous Na₂SO₄, and filtered. The filtrate was concentrated and purified by pre-HPLC to give the title compound (19 mg, 0.038 mmol, 19% yield) as a light yellow solid.

¹H NMR (400 MHz, DMSO-d6) δ 9.20 (d, J=7.2 Hz, 1H), 8.50 (d, J=2.0 Hz, 2H), 7.92 (t, J=7.6 Hz, 1H), 7.64 (t, J=6.2 Hz, 1H), 7.43 (t, J=7.8 Hz, 1H), 7.24 (t, J=54.2 Hz, 1H), 6.75 (d, J=8.0 Hz, 1H), 4.43 (d, J=13.2 Hz, 1H), 3.84 (d, J=12.8 Hz, 1H), 3.66 (d, J=2.4 Hz, 1H), 3.59 (s, 3H), 3.37 (s, 1H), 2.82 (t, J=13.0 Hz, 1H), 2.67-2.55 (s, 2H), 2.06 (s, 3H), 1.84-1.64 (m, 2H).

LC-MS m/e: 502.2 (MH⁺).

### Synthesis of Compound 48: (R)-6-(1-acetyl-4-methoxypiperidin-4-yl)-4-((1-(3-(difluoromethyl)-2-fluorophenyl) prop-2-yn-1-yl) amino)-8-methylpyrido[2,3-d]pyrimidin-7 (8H)-one

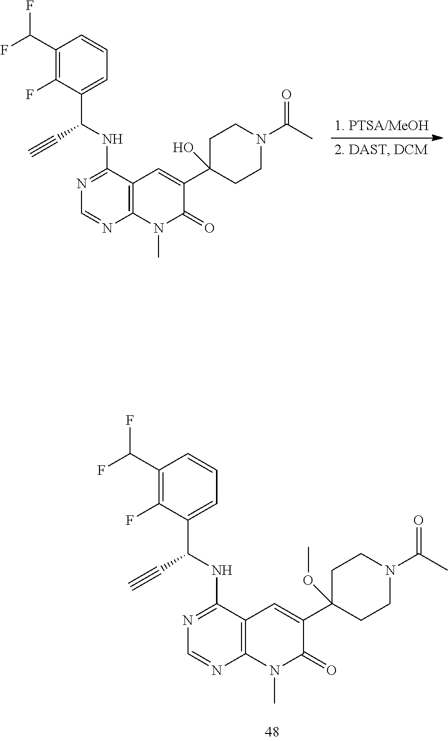 [SMILES: C#C[C@@H](Nc1ncnc2c1cc(C1(O)CCN(C(C)=O)CC1)c(=O)n2C)c1cccc(C(F)F)c1F ; C#C[C@@H](Nc1ncnc2c1cc(C1(OC)CCN(C(C)=O)CC1)c(=O)n2C)c1cccc(C(F)F)c1F]

A solution of 6-(1-acetyl-4-hydroxypiperidin-4-yl)-4-{[(1R)-1-[3-(difluoromethyl)-2-fluorophenyl] prop-2-yn-1-yl] amino}-8-methyl-7H,8H-pyrido[2,3-d] pyrimidin-7-one (100 mg, 0.200 mmol) and p-toluenesulfonic acid (52.0 mg, 0.300 mmol) in MeOH (3.0 mL) was stirred at 60° C. for 16 hours. The mixture was concentrated to dryness. The residue was diluted with water (10 mL) and extracted with EtOAc (20 mL×2). The organic layer was washed with brine (15 mL×2), dried over anhydrous Na₂SO₄, and filtered. The filtrate was concentrated and dissolved in DCM (3.0 mL), to which DAST (0.02 mL, 0.16 mmol) was added at 0° C. The mixture was stirred at 0° C. for 1 hour, then quenched with saturated NaHCO₃ (5.0 mL) and DCM (20 mL) was added. The organic layer was washed with brine (15 mL×2), dried over anhydrous Na₂SO₄, and filtered. The filtrate was concentrated and purified by pre-HPLC to the title compound (8.0 mg, 0.016 mmol, 7.8% yield) as a light yellow solid.

¹H NMR (400 MHz, DMSO-d6) δ 9.17 (d, J=4.0 Hz, 1H), 8.46 (s, 1H), 8.25 (s, 1H), 7.90 (s, 1H), 7.63 (d, J=7.2 Hz, 1H), 7.42 (t, J=7.8 Hz, 1H), 7.25 (t, J=54.2 Hz, 1H), 6.77 (d, J=7.2 Hz, 1H), 4.29 (d, J=12.8 Hz, 1H), 3.69 (d, J=11.6 Hz, 2H), 3.57 (s, 3H), 3.39 (s, 1H), 3.06 (s, 3H), 2.81 (t, J=10.8 Hz, 1H), 2.16 (m, 2H), 2.08 (m, 2H), 2.02 (s, 3H).

LC-MS m/e: 514.2 (MH⁺).

### Synthesis of Compound 49: 6-(1-acetyl-3-methoxypyrrolidin-3-yl)-4-{[(1R)-1-[3-(difluoromethyl)-2-fluorophenyl]prop-2-yn-1-yl]amino}-8-methyl-7H,8H-pyrido[2,3-d]pyrimidin-7-one

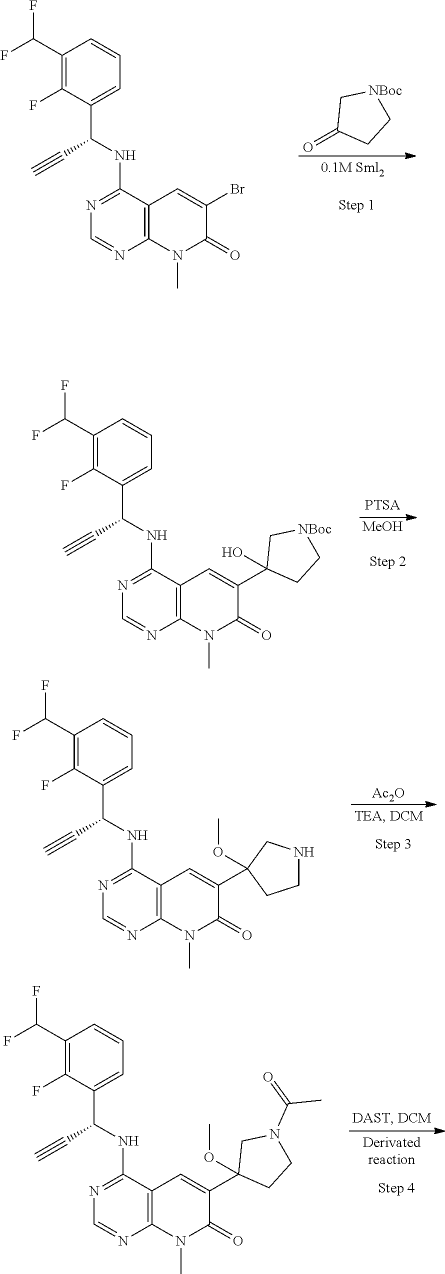

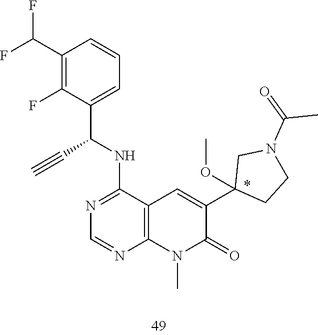 [SMILES: C#C[C@@H](Nc1ncnc2c1cc(C1(OC)CCN(C(C)=O)C1)c(=O)n2C)c1cccc(C(F)F)c1F]

### Step 1. Synthesis of tert-butyl 3-(4-{[(1R)-1-[3-(difluoromethyl)-2-fluorophenyl]prop-2-yn-1-yl]amino}-8-methyl-7-oxo-7H,8H-pyrido[2,3-d]pyrimidin-6-yl)-3-hydroxypyrrolidine-1-carboxylate

To a solution of 6-bromo-4-{[(1R)-1-[3-(difluoromethyl)-2-fluorophenyl]prop-2-yn-1-yl]amino}-8-methyl-7H,8H-pyrido[2,3-d]pyrimidin-7-one (1.60 g, 3.66 mmol) and tert-butyl 3-oxopyrrolidine-1-carboxylate (1.36 g, 7.3 mmol) in THF (6.0 mL) was added dropwise with SmI₂ (3.27 mL, 0.1 M in THF, 0.327 mmol) at 0° C. The resulting mixture was stirred at 20° C. for 0.5 hour and quenched with saturated NH₄Cl (10 mL) and extracted with EtOAc (40 mL×2). The combined organic layers were washed with brine and dried over anhydrous Na₂SO₄, filtered, and concentrated under reduced pressure to dryness. The residue was purified by flash chromatography (eluent: 100% PE to PE:EtOAc=2:1) to give the title compound (430 mg, 0.791 mmol, 21.6% yield) as a yellow solid.

LC-MS m/e: 544 (MH⁺).

### Step 2. Synthesis of 4-{[(1R)-1-[3-(difluoromethyl)-2-fluorophenyl]prop-2-yn-1-yl]amino}-6-(3-methoxypyrrolidin-3-yl)-8-methyl-7H,8H-pyrido[2,3-d]pyrimidin-7-one

To a solution of tert-butyl 3-(4-{[(1R)-1-[3-(difluoromethyl)-2-fluorophenyl] prop-2-yn-1-yl]amino}-8-methyl-7-oxo-7H,8H-pyrido[2,3-d]pyrimidin-6-yl)-3-hydroxypyrrolidine-1-carboxylate (350 mg, 0.644 mmol) in MeOH (20 mL) was added TsOH (553 mg, 3.22 mmol) in portions at 0° C. The mixture was stirred at 60° C. for 3 hours, then cooled down to room temperature, diluted with water, and extracted with DCM (30 mL×2). The combined organic layers were washed with brine, dried over anhydrous Na₂SO₄, filtered, and concentrated under reduced pressure to give the title compound (300 mg, 0.656 mmol, 101% yield) as a brown solid, which is used directly without further purification.

LC-MS m/e: 458 (MH⁺).

### Step 3. Synthesis of 6-(1-acetyl-3-hydroxypyrrolidin-3-yl)-4-{[(1R)-1-[3-(difluoromethyl)-2-fluorophenyl]prop-2-yn-1-yl]amino}-8-methyl-7H,8H-pyrido[2,3-d]pyrimidin-7-one

To a solution of 4-{[(1R)-1-[3-(difluoromethyl)-2-fluorophenyl]prop-2-yn-1-yl]amino}-6-(3-methoxypyrrolidin-3-yl)-8-methyl-7H,8H-pyrido[2,3-d]pyrimidin-7-one (300 mg, 0.656 mmol) and Et₃N (265 mg, 2.62 mmol) in DCM (5.0 mL) was added dropwise Ac₂O (133 mg, 1.31 mmol) at 0° C. The mixture was stirred for 4 hours at 30° C., diluted with water (10 mL), and extracted with DCM (30 mL×2). The combined organic layers were washed with brine, dried over anhydrous Na₂SO₄, filtered, and concentrated under reduced pressure to give the title compound (320 mg, 0.641 mmol, 97.6% yield) as a brown oil.

LC-MS m/e: 500 (MH⁺).

### Step 4. Synthesis of 6-(1-acetyl-3-fluoropyrrolidin-3-yl)-4-{[(1R)-1-[3-(difluoromethyl)-2-fluorophenyl]prop-2-yn-1-yl]amino}-8-methyl-7H,8H-pyrido[2,3-d]pyrimidin-7-one (Compound 49)

To a solution of crude 6-(1-acetyl-3-methoxypyrrolidin-3-yl)-4-{[(1R)-1-[3-(difluoromethyl)-2-fluorophenyl]prop-2-yn-1-yl]amino}-8-methyl-7H,8H-pyrido[2,3-d]pyrimidin-7-one (280 mg, 0.561 mmol) in DCM (5 mL) was added dropwise DAST (225 mg, 1.40 mmol) at 0° C. under N₂. The mixture was stirred at 10° C. for 2 hours, quenched with ice water (5 mL), and extracted with DCM (20 mL×2). The combined organic layers were washed with brine, dried over anhydrous Na₂SO₄, filtered, and concentrated under reduced pressure to dryness. The residue was purified by reverse-phase chromatography (SilaSep™ C18 silica flash cartridge, 0%-40% MeCN in H₂O with 0.1% FA) and preparative thin-layer chromatography (10% MeOH in DCM) to give the title compound (4.1 mg, 0.008 mmol, 1.5% yield) as a yellow solid.

¹H NMR (400 MHz, CDCl₃) δ 8.63-8.47 (m, 1H), 8.00-7.79 (m, 2H), 7.61 (t, J=6.3 Hz, 1H), 7.31 (m, 1H), 6.86 (m, 3H), 4.08 (m, 1H), 3.74 (m, 4H), 3.70-3.39 (m, 2H), 3.17 (m, 3H), 2.79-2.62 (m, 1H), 2.61-2.54 (m, 1H), 2.45 (m, 1H), 2.07-1.96 (m, 3H).

LC-MS m/e: 500 (MH⁺).

### Syntheses of Compounds 50-53

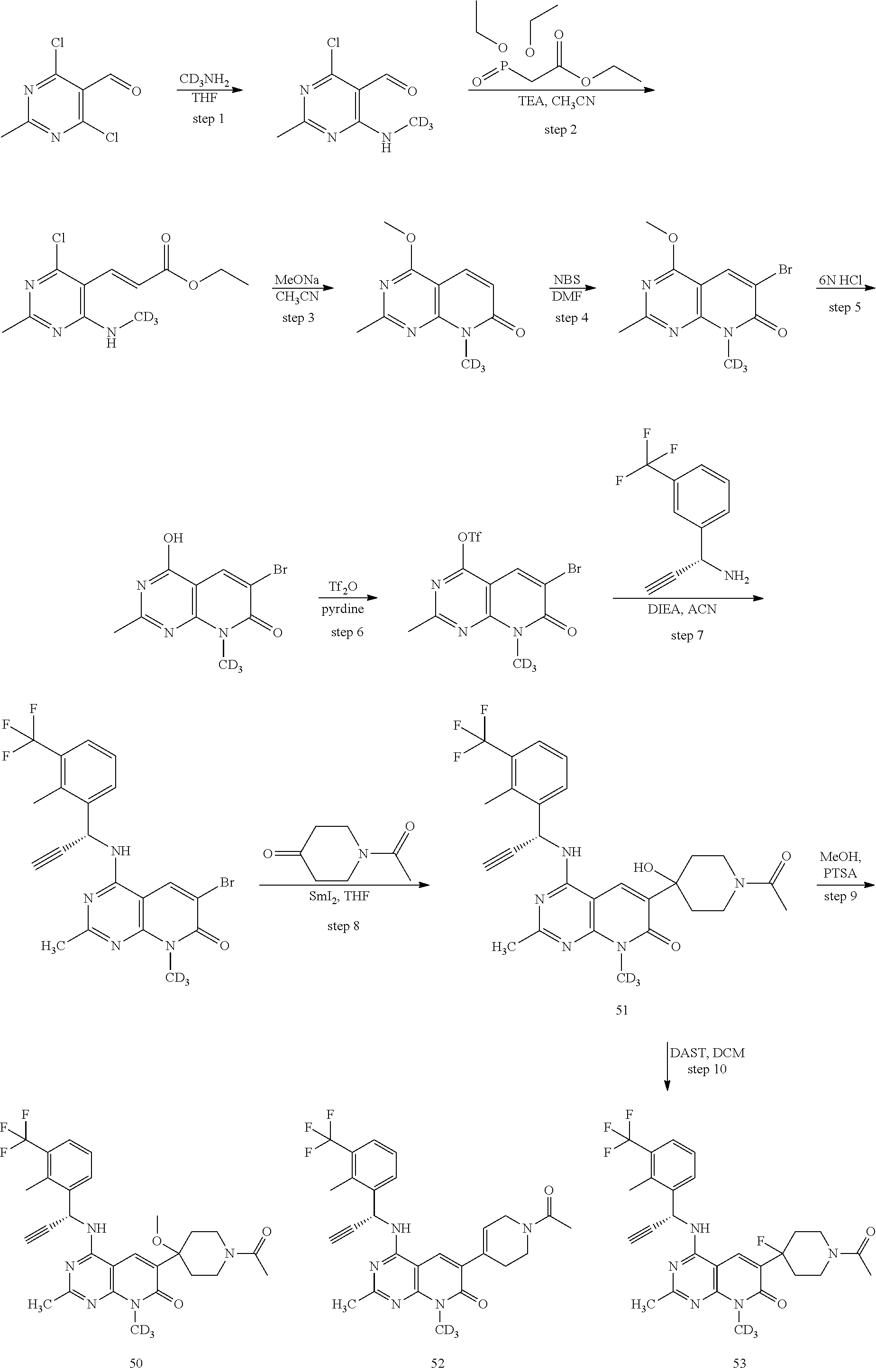

### Step 1. Synthesis of 4-chloro-2-methyl-6-[(\hat{ }2H₃)methylamino]pyrimidine-5-carbaldehyde

A solution of trideuteriomethanamine, hydrochloride (6.69 g, 94.2 mmol), and 4,6-dichloro-2-methylpyrimidine-5-carbaldehyde (15.0 g, 78.5 mmol) in THF (150 mL) was stirred at 0° C. till solid completely dissolved, then to the solution, TEA (31.7 g, 314 mmol) was slowly added. The reaction was monitored by TLC. After completion, the reaction mixture was quenched with water (100 mL) at 0° C. and extracted with EtOAc (100 mL×3). The combined organic layers were dried over Na₂SO₄, filtered, and concentrate under reduced pressure to give the title compound (11.0 g, 58.3 mmol, 74.3% yield) as a brown solid.

LC-MS m/e: 189 (MH⁺).

### Step 2. Synthesis of ethyl (2E)-3-{4-chloro-2-methyl-6-[(\hat{ }2H₃)methylamino]pyrimidin-5-yl}prop-2-enoate

To a solution of 4-chloro-2-methyl-6-[(\hat{ }2H₃)methylamino]pyrimidine-5-carbaldehyde (11.0 g, 58.3 mmol), TEA (24.7 mL, 178 mmol), LiCl (3.67 g, 87.4 mmol) in acetonitrile (110 mL) was added ethyl 2-(diethoxyphosphoryl)acetate (23.2 g, 87.4 mmol). The resulting mixture was stirred at 80° C. overnight, then cooled to room temperature, quenched with water (100 mL), and extracted with EtOAc (100 mL×3). The combined organic layers were dried over anhydrous Na₂SO₄ and filtered. The filtrate was concentrated under reduced pressure and purified by flash chromatography (gradient, PE/EtOAc=10/1 to 1/1) to give the title compound (2.60 g, 10.0 mmol, 17.2% yield) as a yellow solid.

LC-MS m/e: 259.0 (MH⁺).

### Step 3. Synthesis of 4-methoxy-8-(\hat{ }2H₃)methyl-2-methyl-7H,8H-pyrido[2,3-d]pyrimidin-7-one

To a solution of ethyl (2E)-3-{4-chloro-2-methyl-6-[(\hat{ }2H₃)methylamino]pyrimidin-5-yl}prop-2-enoate (1 g, 3.87 mmol) in MeOH (10 mL) was added sodium methoxide (0.52 mL, 9.3 mmol, 30% in MeOH). The mixture was stirred at 60° C. for 3 hours, then cooled down to room temperature, and concentrated to dryness. The residue was diluted with water (30 mL). The resulting solution was adjusted to have a pH value of 4 by adding 1 M HCl. A precipitate was formed, which was collected and dried under vacuum to give the title compound (600 mg, 2.88 mmol, 74.5% yield) as a brown solid.

LC-MS m/e: 209 (MH⁺).

### Step 4. Synthesis of 6-bromo-4-methoxy-8-(\hat{ }2H₃)methyl-2-methyl-7H,8H-pyrido[2,3-d]pyrimidin-7-one

To a solution of 4-methoxy-8-(\hat{ }2H₃)methyl-2-methyl-7H,8H-pyrido[2,3-d]pyrimidin-7-one (400 mg, 1.92 mmol) in DMF (5.0 mL) was added N-bromosuccinimide (690 mg, 3.88 mmol) at 0° C. The reaction mixture was stirred at room temperature for 4 hours, then diluted with EtOAc (30 mL), and washed with brine (50 mL). The organic layer was concentrated under reduced pressure to give the title compound (500 mg, 1.74 mmol, 90.7% yield) as a light yellow solid.

LC-MS m/e: 287,289 (MH⁺).

### Step 5. Synthesis of 6-bromo-4-hydroxy-8-(\hat{ }2H₃)methyl-2-methyl-7H,8H-pyrido[2,3-d]pyrimidin-7-one

6-Bromo-4-methoxy-8-(\hat{ }2H₃)methyl-2-methyl-7H,8-pyrido[2,3-d]pyrimidin-7-one (500 mg, 1.74 mmol) was charged in 6 N HCl (5.0 mL, 30 mmol) and stirred at 100° C. for 30 minutes. The reaction mixture was cooled down to room temperature and basified with saturated Na₂CO₃ aqueous solution (60 mL). The aqueous layer was extracted by EtOAc (50 mL×3). The combined organic layers were dried over Na₂SO₄ and filtered. The filtrate was concentrated under reduced pressure to give the title compound (400 mg, 1.47 mmol, 84.1% yield) as a brown oil.

LC-MS m/e: 273,275 (MH⁺).

### Step 6. Synthesis of 6-bromo-8-(\hat{ }2H₃)methyl-2-methyl-7-oxo-7H,8H-pyrido[2,3-d]pyrimidin-4-yl trifluoromethanesulfonate

To a solution of 6-bromo-4-hydroxy-8-(\hat{ }2H₃)methyl-2-methyl-7H,8H-pyrido[2,3-d]pyrimidin-7-one (400 mg, 1.47 mmol) and pyridine (0.36 mL, 4.4 mmol) in DCM (5 mL) was added trifluoromethanesulfonic anhydride (0.59 mL, 3.5 mmol) at 0° C. and stirred at room temperature for 3 hours. The reaction mixture was then concentrated under reduced pressure and diluted with EtOAc (50 mL). The organic layer was washed by brine (50 mL), dried over Na₂SO₄, and filtered. The filtrate was concentrated under reduced pressure to give the title compound (630 mg, 1.24 mmol, 85.0% yield) as a brown oil.

LC-MS m/e: 404,406 (MH⁺).

### Step 7. Synthesis of 6-bromo-8-(\hat{ }2H₃)methyl-2-methyl-4-{[(1R)-1-[2-methyl-3-(trifluoromethyl)phenyl]prop-2-yn-1-yl]amino}-7H,8H-pyrido[2,3-d]pyrimidin-7-one

To a solution of 6-bromo-8-(\hat{ }2H₃)methyl-2-methyl-7-oxo-7H,8H-pyrido[2,3-d]pyrimidin-4-yl trifluoromethanesulfonate (630 mg, 1.24 mmol) and (1R)-1-[2-methyl-3-(trifluoromethyl)phenyl]prop-2-yn-1-amine (265 mg, 1.24 mmol) in dioxane (5 mL) was added DIEA (0.21 mL, 1.25 mmol). The reaction mixture was stirred at 100° C. for 30 minutes, then cooled to room temperature, quenched with water (20 mL), and extracted with EtOAc (50 mL×3). The combined organic layers were dried over Na₂SO₄ and filtered. The filtrate was concentrated under reduced pressure and purified by flash chromatography (gradient, PE to PE/EtOAc=3/1) to give the title compound (500 mg, 1.07 mmol, 85.8% yield) as a brown oil.

LC-MS m/e: 469 (MH⁺).

### Step 8. Synthesis of 6-(1-acetyl-4-hydroxypiperidin-4-yl)-8-(\hat{ }2H₃)methyl-2-methyl-4-{[(1R)-1-[2-methyl-3-(trifluoromethyl)phenyl]prop-2-yn-1-yl]amino}-7H,8H-pyrido[2,3-d]pyrimidin-7-one (Compound 51)

To SmI₂ (50 mL, 5.0 mmol, 0.1 M in THF) at 0° C. under N₂ were added 1-acetylpiperidin-4-one (0.158 mL, 1.28 mmol) and 6-bromo-8-(\hat{ }2H₃)methyl-2-methyl-4-{[(1R)-1-[2-methyl-3-(trifluoromethyl)phenyl]prop-2-yn-1-yl]amino}-7H,8H-pyrido[2,3-d]pyrimidin-7-one (500 mg, 1.07 mmol). The resulting mixture was stirred at room temperature for 3 hours, then quenched by air, and filtrated. The filtrate was concentrated under reduced pressure to dryness. The residue was purified by prep-HPLC to give the title compound (200 mg, 0.380 mmol, 35.3% yield) as a white solid.

¹H NMR (400 MHz, DMSO-d6) δ 9.04 (d, J=5.6 Hz, 1H), 8.44 (d, J=2.6 Hz, 1H), 8.06 (d, J=7.6 Hz, 1H), 7.68 (d, J=7.8 Hz, 1H), 7.48 (t, J=7.8 Hz, 1H), 6.69 (d, J=7.8 Hz, 1H), 5.42 (s, 1H), 4.32 (d, J=10.8 Hz, 1H), 3.68 (d, J=13.6 Hz, 1H), 3.60 (d, J=2.4 Hz, 1H), 2.90 (t, J=12.4 Hz, 1H), 2.48 (s, 3H), 2.45 (s, 3H), 2.34-2.23 (m, 2H), 2.01 (s, 3H), 1.61 (d, J=13.7 Hz, 1H), 1.54 (s, 1H).

LC-MS m/e: 531 (MH⁺).

### Step 9. Synthesis of 6-(1-acetyl-4-methoxypiperidin-4-yl)-8-(\hat{ }2H₃)methyl-2-methyl-4-{[(1R)-1-[2-methyl-3-(trifluoromethyl)phenyl]prop-2-yn-1-yl]amino}-7H,8H-pyrido[2,3-d]pyrimidin-7-one (Compound 50) and 6-(1-acetyl-1,2,3,6-tetrahydropyridin-4-yl)-8-(\hat{ }2H₃)methyl-2-methyl-4-{[(1R)-1-[2-methyl-3-(trifluoromethyl)phenyl]prop-2-yn-1-yl]amino}-7H,8H-pyrido[2,3-d]pyrimidin-7-one (Compound 52)

To a solution of 6-(1-acetyl-4-hydroxypiperidin-4-yl)-8-(\hat{ }2H₃)methyl-2-methyl-4-{[(1R)-1-[2-methyl-3-(trifluoromethyl)phenyl]prop-2-yn-1-yl]amino}-7H,8H-pyrido[2,3-d]pyrimidin-7-one (30 mg, 0.057 mmol) in MeOH (4.0 mL) was added p-toluenesulfonic acid (19 mg, 0.11 mmol). The reaction mixture was stirred at 60° C. overnight, then cooled down to room temperature, concentrated, and purified by prep-HPLC to give Compound 50 (10 mg, 0.018 mmol, 33% yield) as a white solid and Compound 52 (5 mg, 0.010 mmol, 17.3% yield) as a white solid.

Compound 50: ¹H NMR (400 MHz, DMSO-d6) δ 9.06 (s, 1H), 8.42 (s, 1H), 8.24 (s, 1H), 8.03 (s, 1H), 7.68 (d, J=8.0 Hz, 1H), 7.49 (t, J=7.6 Hz, 1H), 6.68 (d, J=7.6 Hz, 1H), 4.30 (s, 1H), 3.60-3.63 (m, 2H), 3.36 (s, 3H), 3.04 (s, 3H), 2.80 (s, 1H), 2.43 (s, 3H), 2.15-2.07 (m, 4H), 2.02 (s, 3H); LC-MS m/e: 545 (MH⁺).

Compound 52: ¹H NMR (400 MHz, DMSO-d6) δ 8.89 (s, 1H), 8.24 (s, 1H), 8.03 (d, J=7.6 Hz, 1H), 7.69 (d, J=7.2 Hz, 1H), 7.50 (d, J=8.0 Hz, 1H), 6.67 (d, J=6.0 Hz, 1H), 6.51 (d, J=48.8 Hz, 1H), 4.12 (d, J=23.6 Hz, 2H), 3.63 (m, 3H), 3.36 (s, 3H), 2.44 (s, 4H), 2.05 (d, J=12.8 Hz, 4H), 1.24 (s, 1H); LC-MS m/e: 513 (MH⁺).

### Step 10. Synthesis of 6-(1-acetyl-4-fluoropiperidin-4-yl)-8-(\hat{ }2H₃)methyl-2-methyl-4-{[(1R)-1-[2-methyl-3-(trifluoromethyl)phenyl]prop-2-yn-1-yl]amino}-7,8-dihydroquinazolin-7-one (Compound 53)

To a solution of 6-(1-acetyl-4-hydroxypiperidin-4-yl)-8-(\hat{ }2H₃)methyl-2-methyl-4-{[(1R)-1-[2-methyl-3-(trifluoromethyl)phenyl]prop-2-yn-1-yl]amino}-7H,8H-pyrido[2,3-d]pyrimidin-7-one (30 mg, 0.057 mmol) in DCM (4 mL) was added DAST (0.007 mL, 0.057 mmol) at 0° C. The reaction mixture was stirred at room temperature for 30 minutes, then quenched with cooled saturated NaHCO₃ (10 mL), and extracted with DCM (10 mL×3). The organic layer was concentrated and purified by prep-HPLC to give title compound (8 mg, 0.015 mmol, 27% yield) as a white solid.

¹H NMR (400 MHz, DMSO-d6) δ 9.07 (d, J=8.0 Hz, 1H), 8.49 (s, 1H), 8.05 (d, J=8.0 Hz, 1H), 7.69 (d, J=7.6 Hz, 1H), 7.49 (t, J=7.6 Hz, 1H), 6.67 (d, J=7.6 Hz, 1H), 4.41 (s, 1H), 3.83 (d, J=13.6 Hz, 1H), 3.61 (d, J=2.4 Hz, 1H), 2.83 (t, J=12.4 Hz, 1H), 2.68-2.51 (m, 3H), 2.49 (s, 3H), 2.46 (s, 3H), 2.06 (s, 3H), 1.82-1.65 (m, 2H).

LC-MS m/e: 532 (MH⁺).

### Synthesis of Compound 54: 6-(1-acetyl-4-hydroxypiperidin-4-yl)-2,8-dimethyl-4-{[(1R)-1-[2-methyl-3-(trifluoromethyl)phenyl]prop-2-yn-1-yl]amino}-7H,8H-pyrido[2,3-d]pyrimidin-7-one

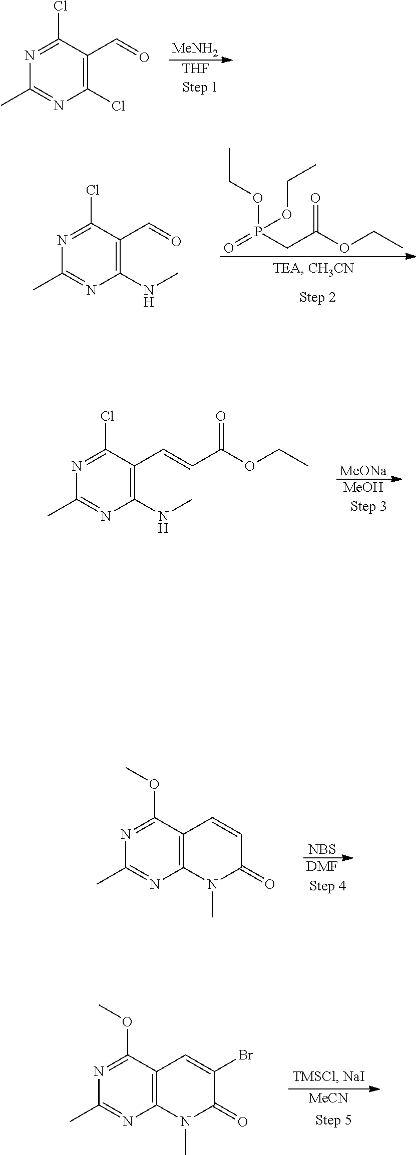

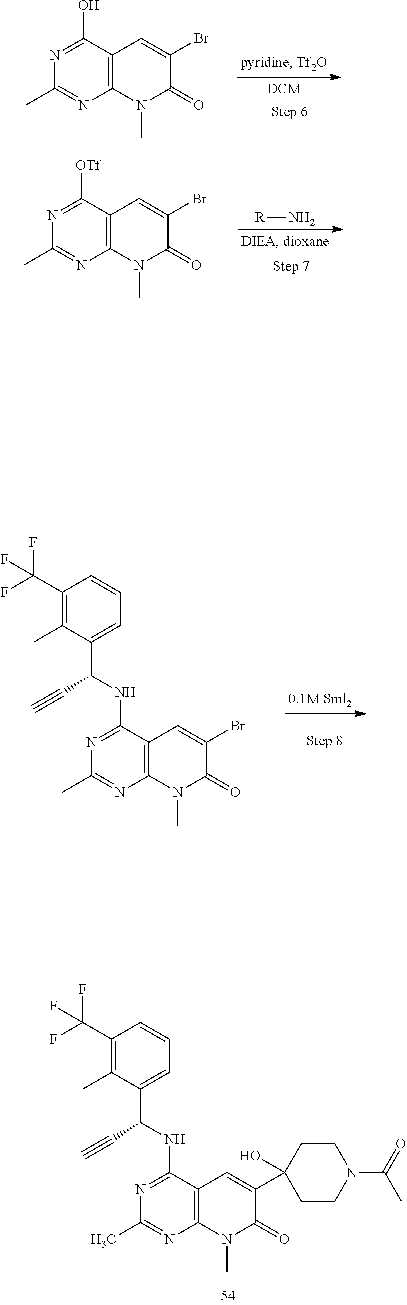

### Step 1. Synthesis of 4-chloro-2-methyl-6-(methylamino)pyrimidine-5-carbaldehyde

To a solution of 4,6-dichloro-2-methylpyrimidine-5-carbaldehyde (10.0 g, 52.3 mmol) and Et₃N (10.5 g, 104 mmol) in THF (50 mL) was added dropwise MeNH₂ (28.7 mL, 2M in THF) at 0° C. The mixture was stirred at 20° C. for 2 hours and concentrated under reduced pressure to dryness. The residue was triturated with PE (100 mL) and filtered to give the title compound (9.00 g, 48.4 mmol, 92.6% yield) as a yellow solid.

LC-MS m/e: 186 (MH⁺).

### Step 2. Synthesis of ethyl (2E)-3-[4-chloro-2-methyl-6-(methylamino)pyrimidin-5-yl]prop-2-enoate

To a solution of 4-chloro-2-methyl-6-(methylamino)pyrimidine-5-carbaldehyde (8.00 g, 43.1 mmol) and ethyl 2-(diethoxyphosphoryl)acetate (12.8 mL, 64.6 mmol) in MeCN (40 mL) was added Et₃N (8.72 g, 86.2 mmol) and LiCl (2.74 g, 64.6 mmol). The mixture was stirred at 80° C. under N₂ for 4 hours, then concentrated, diluted with water (50 mL), and extracted with EtOAc (100 mL×2). The combined organic layers were washed with brine and dried over anhydrous Na₂SO₄, filtered, and concentrated under reduced pressure to dryness. The residue was purified by flash chromatography (eluent: 100% PE to PE:EtOAc=3:1) to give the title compound (5.70 g, 22.2 mmol, 51.7% yield) as a yellow solid.

LC-MS m/e: 256 (MH⁺).

### Step 3. Synthesis of 4-methoxy-2,8-dimethyl-7H,8H-pyrido[2,3-d]pyrimidin-7-one

To a solution of ethyl (2E)-3-[4-chloro-2-methyl-6-(methylamino)pyrimidin-5-yl]prop-2-enoate (7.00 g, 27.3 mmol) in MeOH (50 mL) was added sodium methoxide (9.85 g, 54.7 mmol, 30% in MeOH). The mixture was stirred at 60° C. under N₂ for 5 hours, then cooled down to room temperature, and concentrated to dryness. The residue was diluted with water (30 mL) and then added 1.0 M HCl to give a solution having a pH value of 4. A precipitate was formed, which was collected and dried under vacuum to give the title compound (4.40 g, 21.4 mmol, 78.3% yield) as a yellow solid.

LC-MS m/e: 206 (MH⁺).

### Step 4. Synthesis of 6-bromo-4-methoxy-2,8-dimethyl-7H,8H-pyrido[2,3-d]pyrimidin-7-one

To a solution of 4-methoxy-2,8-dimethyl-7H,8H-pyrido[2,3-d]pyrimidin-7-one (4.40 g, 21.4 mmol) in DMF (25 mL) was added NBS (4.77 g, 26.8 mmol) in portions at 0° C. under N₂. The mixture was stirred at 60° C. for 5 hours, then cooled down to room temperature, and quenched with ice water (100 mL). A precipitate was formed, which was collected and dried under vacuum to give the title compound (5.10 g, 17.9 mmol, 83.7% yield) as a yellow oil.

LC-MS m/e: 284 (MH⁺).

### Step 5. Synthesis of 6-bromo-4-hydroxy-2,8-dimethyl-7H,8H-pyrido[2,3-d]pyrimidin-7-one

To a solution of 6-bromo-4-methoxy-2,8-dimethyl-7H,8H-pyrido[2,3-d]pyrimidin-7-one (2.50 g, 8.79 mmol) in MeCN (40 mL) was added TMSCl (4.40 mL, 35.1 mmol) and NaI (1.44 mL, 35.1 mmol). The mixture was stirred at 30° C. under N₂ for 2 hours and was filtrated. The filter was collected, washed with water (30 mL), and dried under vacuum to give the title compound (2.40 g, 8.88 mmol, 100% yield) as a yellow solid.

LC-MS m/e: 270 (MH⁺).

### Step 6. Synthesis of 6-bromo-2,8-dimethyl-7-oxo-7H,8H-pyrido[2,3-d]pyrimidin-4-yl trifluoromethanesulfonate

To a solution of 6-bromo-4-hydroxy-2,8-dimethyl-7H,8H-pyrido[2,3-d]pyrimidin-7-one (1.50 g, 5.55 mmol) in DCM (30 mL) was added pyridine (2.2 mL, 28 mmol). The mixture was cooled down to 0° C., and then Tf₂O (4.70 g, 16.6 mmol) was added at 0° C. The resulting mixture was stirred at 30° C. under N₂ for 3 hours, then diluted with water (20 mL), and extracted with EtOAc (40 mL×2). The combined organic layers were washed with brine, dried over anhydrous Na₂SO₄, and concentrated under reduced pressure to give the title compound (1.50 g, 3.73 mmol, 67.1% yield) as a yellow solid.

LC-MS m/e: 402 (MH⁺).

### Step 7. Synthesis of 6-bromo-2,8-dimethyl-4-{[(1R)-1-[2-methyl-3-(trifluoromethyl)phenyl]prop-2-yn-1-yl]amino}-7H,8H-pyrido[2,3-d]pyrimidin-7-one

To a solution of 6-bromo-2,8-dimethyl-7-oxo-7H,8H-pyrido[2,3-d]pyrimidin-4-yl trifluoromethanesulfonate (1.50 g, 3.73 mmol) and (1R)-1-[2-methyl-3-(trifluoromethyl)phenyl]prop-2-yn-1-amine hydrochloride (1.02 g, 4.10 mmol) in dioxane (20 mL) was added DIPEA (1.45 g, 11.1 mmol). The mixture was stirred at 100° C. under N₂ for one hour, then diluted with ice water (20 mL), and extracted with EtOAc (30 mL×2). The combined organic layers were washed with brine and dried over anhydrous Na₂SO₄, filtered, and concentrated under reduced pressure to dryness. The residue was purified by flash chromatography (eluent: 100% PE to PE:EtOAc=2:1) to give the title compound (470 mg, 1.01 mmol, 27.0% yield) as a yellow solid.

LC-MS m/e: 465 (MH⁺).

### Step 8. Synthesis of 6-(1-acetyl-4-hydroxypiperidin-4-yl)-2,8-dimethyl-4-{[(1R)-1-[2-methyl-3-(trifluoromethyl)phenyl]prop-2-yn-1-yl]amino}-7H,8H-pyrido[2,3-d]pyrimidin-7-one (Compound 54)

SmI₂ (19.3 mL, 0.10 M in THF) was added to a stirred solution of 6-bromo-2,8-dimethyl-4-{[(1R)-1-[2-methyl-3-(trifluoromethyl)phenyl]prop-2-yn-1-yl]amino}-7H,8H-pyrido[2,3-d]pyrimidin-7-one (300 mg, 0.645 mol) and 1-acetylpiperidin-4-one (109 mg, 0.774 mmol) in THF (5.0 mL) at 0° C. under N₂. The mixture was stirred at 30° C. for one hour, then quenched with saturated NH₄Cl (5 mL), and extracted with EtOAc (20 mL×2). The combined organic layers were washed with brine and dried over anhydrous Na₂SO₄, filtered, and concentrated under reduced pressure to dryness. The residue was purified by flash chromatography (eluent: 100% PE to PE:EtOAc=2:1) and further purified with prep-HPLC (SilaSep™ C18 silica flash cartridge, 0%-40% MeCN in H₂O with 0.1% formic acid) to give the title compound (30 mg, 0.057 mmol, 8.8% yield) as a yellow solid.

¹H NMR (400 MHz, MeOD) δ 8.25 (d, J=1.6 Hz, 1H), 8.05 (d, J=8.0 Hz, 1H), 7.64 (d, J=7.6 Hz, 1H), 7.39 (t, J=8.0 Hz, 1H), 6.74 (s, 1H), 4.45 (d, J=10.8 Hz, 1H), 3.81 (d, J=14.4 Hz, 1H), 3.71 (s, 3H), 3.61 (m, 1H), 3.11 (m, 1H), 2.98 (d, J=2.4 Hz, 1H), 2.51 (s, 6H), 2.28-2.15 (m, 2H), 2.13 (s, 3H), 1.86 (dd, J=34.4, 13.2 Hz, 2H).

LC-MS m/e: 528 (MH⁺).

### Syntheses of Compounds 55 and 56: (R)-6-(1-acetyl-4-methoxypiperidin-4-yl)-2,8-dimethyl-4-((1-(2-methyl-3-(trifluoromethyl)phenyl)prop-2-yn-1-yl)amino)pyrido[2,3-d]pyrimidin-7 (8H)-one (Compound 55) and (R)-6-(1-acetyl-4-fluoropiperidin-4-yl)-2,8-dimethyl-4-((1-(2-methyl-3-(trifluoromethyl)phenyl)prop-2-yn-1-yl)amino)pyrido[2,3-d]pyrimidin-7 (8H)-one (Compound 56)

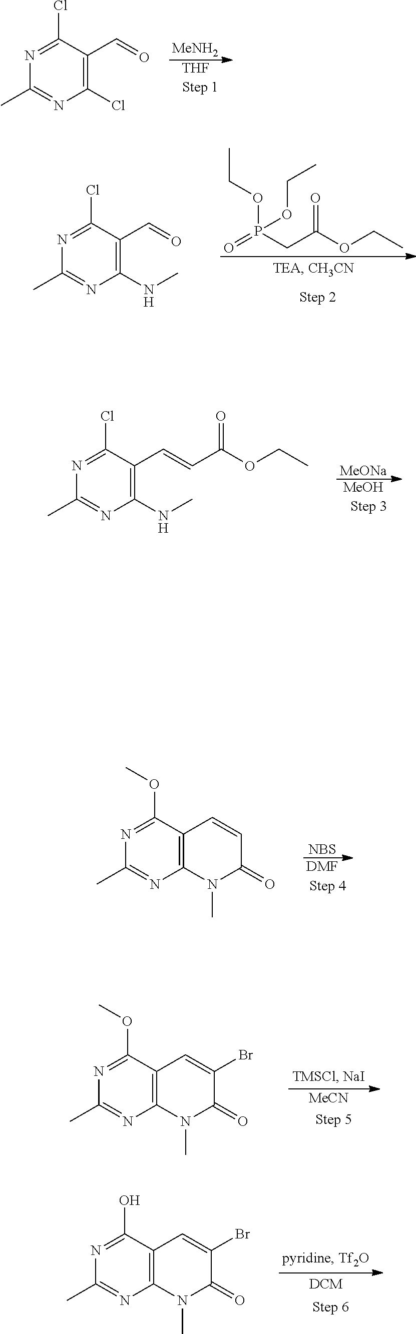

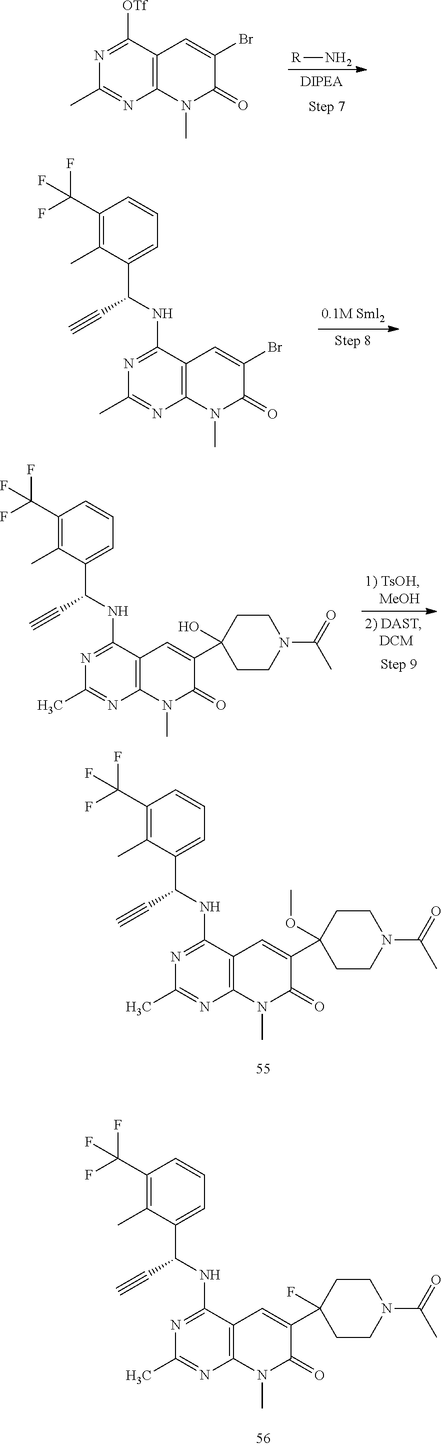

### Step 1. Synthesis of 4-chloro-2-methyl-6-(methylamino)pyrimidine-5-carbaldehyde

To a solution of 4,6-dichloro-2-methylpyrimidine-5-carbaldehyde (10 g, 52.3 mmol) and Et₃N (10.5 g, 104 mmol) in THF (50 mL) was added dropwise MeNH₂ (28.7 mL, 57.4 mmol, 2M in THF) at 0° C. The mixture was stirred at 20° C. for 2 hours and concentrated under reduced pressure to dryness. The residue was triturated with PE (100 mL) and filtered. The cake was dried under vacuum to give the title compound (9 g, 48.4 mmol, 92.6% yield) as a yellow solid.

LC-MS m/e: 186 (MH⁺).

### Step 2. Synthesis of ethyl (E)-3-(4-chloro-2-methyl-6-(methylamino)pyrimidin-5-yl)acrylate

To a solution of 4-chloro-2-methyl-6-(methylamino)pyrimidine-5-carbaldehyde (8 g, 43.1 mmol) and ethyl 2-(diethoxyphosphoryl)acetate (12.8 mL, 64.6 mmol) in MeCN (40 mL) were added Et₃N (8.72 g, 86.2 mmol) and LiCl (2.74 g, 64.6 mmol). The mixture was stirred at 80° C. for 4 hours under N₂. The resulting mixture was cooled to room temperature and concentrated to dryness. The residue was partitioned between water (50 mL) and EtOAc (200 mL). The organic layer was washed with brine (100 mL), dried over anhydrous Na₂SO₄, filtered, concentrated under reduced pressure, and purified by flash chromatography on silica gel (gradient, PE to PE:EtOAc=3:1) to give the title compound (5.7 g, 22.2 mmol, 51.7% yield) as a yellow solid.

LC-MS m/e: 256 (MH⁺).

### Step 3. Synthesis of 4-methoxy-2,8-dimethylpyrido[2,3-d]pyrimidin-7 (8H)-one

To a solution of ethyl (E)-3-(4-chloro-2-methyl-6-(methylamino)pyrimidin-5-yl)acrylate (7.0 g, 27.3 mmol) in MeOH (50 mL) was added MeONa (9.85 g, 54.7 mmol, 30% in MeOH). The mixture was stirred at 60° C. for 5 hrs under N₂, cooled to room temperature, evaporated, diluted with water (30 mL), and then added 1 M HCl to adjust to pH=4. The resulting suspension was filtered. The cake was dried under vacuum to give the title compound (4.4 g, 21.4 mmol, 78.3% yield) as a yellow solid.

LC-MS m/e: 206 (MH⁺).

### Step 4. Synthesis of 6-bromo-4-methoxy-2,8-dimethylpyrido[2,3-d]pyrimidin-7 (8H)-one

To a solution of 4-methoxy-2,8-dimethylpyrido[2,3-d]pyrimidin-7 (8H)-one (4.4 g, 21.4 mmol) in DMF (25 mL) was added NBS (4.77 g, 26.8 mmol) portionwise at 0° C. under N₂. The mixture was stirred at 60° C. for 5 hours, then diluted with water (100 mL) at 10° C., and filtrated. The cake was dried under vacuum to give the title compound (5.1 g, 17.9 mmol, 83.7% yield) as a yellow oil.

LC-MS m/e: 284,286 (MH⁺).

### Step 5. Synthesis of 6-bromo-4-hydroxy-2,8-dimethylpyrido[2,3-d]pyrimidin-7 (8H)-one

To a solution of 6-bromo-4-methoxy-2,8-dimethylpyrido[2,3-d]pyrimidin-7 (8H)-one (2.5 g, 8.79 mmol) in MeCN (40 mL) were added TMSCl (4.4 mL, 35.1 mmol) and NaI (5.27 g, 35.1 mmol). The mixture was stirred at 30° C. for 2 hours under N₂ and then filtrated. The cake was washed with water (30 mL) and dried under vacuum to give the title compound (2.4 g, 8.88 mmol, 100% yield) as a yellow solid.

LC-MS m/e: 270, 272 (MH⁺).

### Step 6. Synthesis of 6-bromo-2,8-dimethyl-7-oxo-7,8-dihydropyrido[2,3-d]pyrimidin-4-yl trifluoromethanesulfonate

To a solution of 6-bromo-4-hydroxy-2,8-dimethylpyrido[2,3-d]pyrimidin-7 (8H)-one (1.5 g, 5.55 mmol) in DCM (30 mL) were added pyridine (2.2 mL, 27.7 mmol) and Tf₂O (4.70 g, 16.6 mmol). The mixture was stirred at 30° C. for 3 hours under N₂, then diluted with water (20 mL), and extracted with EtOAc (40 mL×2). The combined organic layers were washed with brine (40 mL), dried over anhydrous Na₂SO₄, filtered, and concentrated under reduced pressure to give the title compound (1.5 g, 3.73 mmol, 67.1% yield) as a yellow solid.

LC-MS m/e: 402,404 (MH⁺).

### Step 7. Synthesis of (R)-6-bromo-2,8-dimethyl-4-((1-(2-methyl-3-(trifluoromethyl)phenyl)prop-2-yn-1-yl)amino)pyrido[2,3-d]pyrimidin-7 (8H)-one

To a solution of 6-bromo-2,8-dimethyl-7-oxo-7,8-dihydropyrido[2,3-d]pyrimidin-4-yl trifluoromethanesulfonate (1.5 g, 3.73 mmol) and (R)-1-(2-methyl-3-(trifluoromethyl)phenyl)prop-2-yn-1-amine hydrochloride (1.02 g, 4.1 mmol) in dioxane (20 mL) was added DIPEA (1.45 g, 11.1 mmol). The mixture was stirred at 100° C. for 1 hour under N₂, then diluted with water (20 mL) at 10° C., and extracted with EtOAc (50 mL×2). The combined organic layers were washed with brine (30 mL), dried over anhydrous Na₂SO₄, filtered, and concentrated to dryness. The residue was purified by flash chromatography on silica gel (gradient, PE to PE:EtOAc=2:1) to give the title compound (470 mg, 1.01 mmol, 27% yield) as a yellow solid.

LC-MS m/e: 465, 467 (MH⁺).

### Step 8. Synthesis of (R)-6-(1-acetyl-4-hydroxypiperidin-4-yl)-2,8-dimethyl-4-((1-(2-methyl-3-(trifluoromethyl)phenyl)prop-2-yn-1-yl)amino)pyrido[2,3-d]pyrimidin-7 (8H)-one

SmI₂ (19.3 mL, 1.93 mmol, 0.1 M in THF) was added dropwise to a solution of (R)-6-bromo-2,8-dimethyl-4-((1-(2-methyl-3-(trifluoromethyl)phenyl)prop-2-yn-1-yl)amino)pyrido[2,3-d]pyrimidin-7 (8H)-one (300 mg, 0.645 mmol) and 1-acetylpiperidin-4-one (109 mg, 0.774 mmol) in THF (1.0 mL) at 0° C. under N₂. The mixture was stirred at 0° C. for 1.5 hours, then quenched with saturated NH₄Cl solution (20 mL), and extracted with EtOAc (20 mL×2). The combined organic layers were washed with brine (20 mL), dried over anhydrous Na₂SO₄, filtered, and concentrated under reduced pressure to dryness. The residue was purified by flash chromatography on silica gel (gradient, PE to PE:EtOAc=2:1) to give the title compound (100 mg, 0.190 mmol, 29.4% yield) as a yellow solid.

LC-MS m/e: 528 (MH⁺).

### Step 9. Syntheses of Compounds 55 and 56

To a solution of (R)-6-(1-acetyl-4-hydroxypiperidin-4-yl)-2,8-dimethyl-4-((1-(2-methyl-3-(trifluoromethyl)phenyl)prop-2-yn-1-yl)amino)pyrido[2,3-d]pyrimidin-7 (8H)-one (210 mg, 0.398 mmol) in MeOH (2.0 mL) was added TsOH (171 mg, 0.990 mmol). The mixture was stirred at 50° C. for 18 hours, then cooled down to room temperature, and partitioned between saturated NaHCO₃ solution (10 mL) and DCM (20 mL). The organic layer was dried over anhydrous Na₂SO₄ and filtered. The filtrate was cooled to 0° C. and added DAST (0.11 mL, 0.796 mmol) dropwise at 0° C. The mixture was stirred at 0° C. for 1 hour, quenched with water (3 mL), and extracted with DCM (10 mL×2). The combined organic layers were washed with brine (10 mL), dried over anhydrous Na₂SO₄, filtered, and concentrated to dryness. The residue was purified by prep-HPLC (SilaSep™ C18 silica flash cartridge, 0%-40% MeCN in H₂O with 0.1% formic acid) to give Compound 55 (70.2 mg, 0.130 mmol, 32.6% yield) as a yellow solid and Compound 56 (6.3 mg, 0.012 mmol, 2.99% yield) as a yellow solid.

Compound 55: ¹H NMR (400 MHz, CD₃OD) δ 8.18 (s, 1H), 8.05 (d, J=7.7 Hz, 1H), 7.64 (d, J=7.8 Hz, 1H), 7.39 (t, J=7.8 Hz, 1H), 6.75 (d, J=1.7 Hz, 1H), 4.39 (d, J=13.1 Hz, 1H), 3.77 (d, J=13.4 Hz, 1H), 3.68 (s, 3H), 3.51 (t, J=12.0 Hz, 1H), 3.12 (s, 3H), 3.02 (d, J=12.9 Hz, 1H), 2.98 (d, J=2.2 Hz, 1H), 2.53 (s, 3H), 2.51 (s, 3H), 2.39 (t, J=10.9 Hz, 1H), 2.33-2.21 (m, 1H), 2.22-2.00 (m, 5H).

LC-MS m/e: 542 (MH⁺).

Compound 56: ¹H NMR (400 MHz, CD₃OD) δ 8.42 (s, 1H), 8.04 (d, J=7.4 Hz, 1H), 7.63 (d, J=8.0 Hz, 1H), 7.38 (t, J=8.0 Hz, 1H), 6.73 (s, 1H), 4.54 (m, 1H), 3.92 (d, J=12.0 Hz, 1H), 3.68 (s, 3H), 3.51 (t, J=14.0 Hz, 1H), 3.03-2.96 (m, 2H), 2.72 (m, 2H), 2.53 (s, 3H), 2.50 (s, 3H), 2.16 (s, 3H), 1.78 (m, 2H).

LC-MS m/e: 530 (MH⁺).

### Synthesis of Compound 57. (R)—N-(1-acetyl-4-(8-methyl-4-((1-(2-methyl-3-(trifluoromethyl)phenyl)prop-2-yn-1-yl)amino)-7-oxo-7,8-dihydropyrido[2,3-d]pyrimidin-6-yl)piperidin-4-yl)acetamide

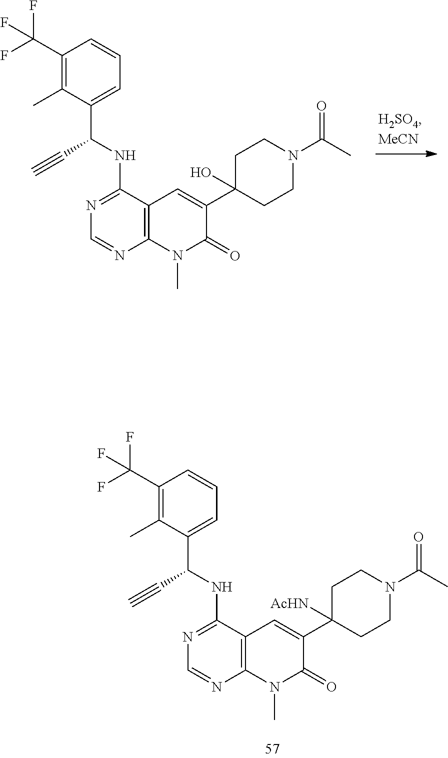 [SMILES: C#C[C@@H](Nc1ncnc2c1cc(C1(O)CCN(C(C)=O)CC1)c(=O)n2C)c1cccc(C(F)(F)F)c1C ; C#C[C@@H](Nc1ncnc2c1cc(C1(NC(C)=O)CCN(C(C)=O)CC1)c(=O)n2C)c1cccc(C(F)(F)F)c1C]

To a solution of (R)-6-(1-acetyl-4-hydroxypiperidin-4-yl)-8-methyl-4-((1-(2-methyl-3-(trifluoromethyl)phenyl)prop-2-yn-1-yl)amino)pyrido[2,3-d]pyrimidin-7 (8H)-one (40 mg, 0.078 mmol) in CH₃CN (2 mL) was added H₂SO₄ (0.2 mL) slowly at −20° C. The mixture was stirred at 25° C. for 3 hours, then quenched with saturated NaHCO₃ solution (10 mL), and extracted with EtOAc (20 mL×3). The combined organic layers were washed with brine (20 mL), dried over anhydrous Na₂SO₄, filtered, and concentrated to dryness. The residue was purified by prep-HPLC (SilaSep™ C18 silica flash cartridge, 0%-40% MeCN in H₂O with 0.1% formic acid) to give the title compound (16.1 mg, 0.029 mmol, 37.3% yield) as a green solid.

¹H NMR (400 MHz, DMSO-d6) δ 9.02 (t, J=6.0 Hz, 1H), 8.47 (d, J=4.0 Hz, 1H), 8.14 (d, J=4.0 Hz, 1H), 8.03-8.00 (m, 2H), 7.70 (dd, J=8.0, 4.0 Hz, 1H), 7.50 (t, J=6.0 Hz, 1H), 6.69 (dd, J=8.0, 4.0 Hz, 1H), 4.28 (t, J=14.0 Hz, 1H), 3.75-3.66 (m, 1H), 3.65-3.61 (m, 1H), 3.58 (s, 3H), 3.45-3.39 (m, 1H), 2.92-2.71 (m, 3H), 2.45 (s, 3H), 2.00 (d, J=4.0 Hz, 3H), 1.79 (s, 3H), 1.76-1.57 (m, 2H).

LC-MS m/e: 555 (MH⁺).

### Synthesis of Compound 58: (R)—N-(4-(8-methyl-4-((1-(2-methyl-3-(trifluoromethyl)phenyl)prop-2-yn-1-yl)amino)-7-oxo-7,8-dihydropyrido[2,3-d]pyrimidin-6-yl)tetrahydro-2H-pyran-4-yl)acetamide

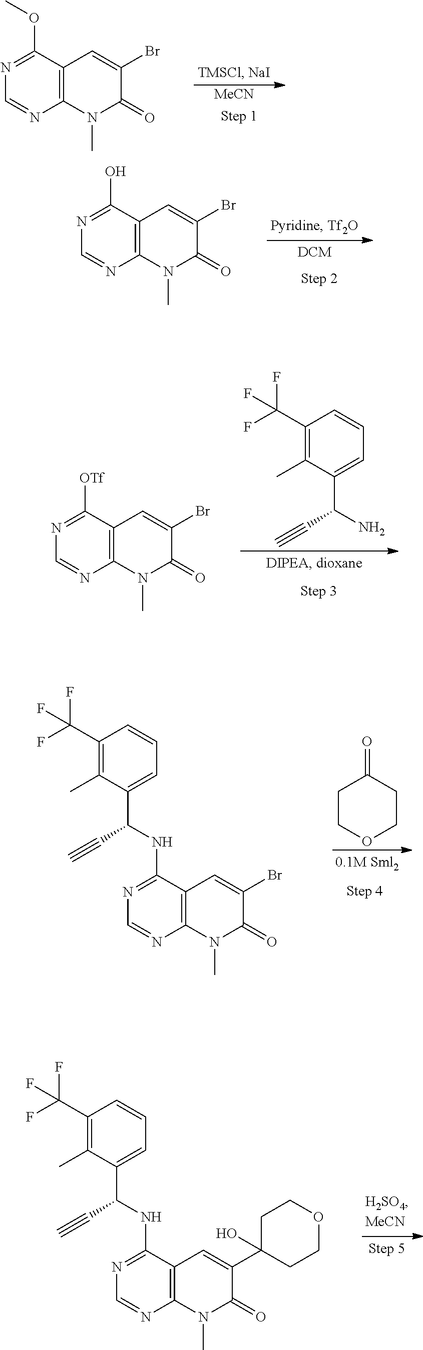

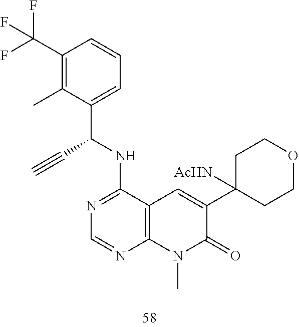 [SMILES: C#C[C@@H](Nc1ncnc2c1cc(C1(NC(C)=O)CCOCC1)c(=O)n2C)c1cccc(C(F)(F)F)c1C]

### Step 1. Synthesis of 6-bromo-4-hydroxy-8-methylpyrido[2,3-d]pyrimidin-7 (8H)-one

To a solution of 6-bromo-4-methoxy-8-methylpyrido[2,3-d]pyrimidin-7 (8H)-one (4 g, 14.8 mmol) in MeCN (40 mL) were added NaI (8.88 g, 59.2 mmol) and then TMSCl (7.6 mL, 59.5 mmol) dropwise at 0° C. The mixture was stirred at 30° C. for 3 hours and then filtrated. The cake was washed with water (30 mL) and MeCN (30 mL), and dried under reduced pressure to give the title compound (2.53 g, 9.90 mmol, 66.8% yield) as a pale yellow solid.

LC-MS m/e: 256 (MH⁺).

### Step 2. Synthesis of 6-bromo-8-methyl-7-oxo-7,8-dihydropyrido[2,3-d]pyrimidin-4-yl trifluoromethanesulfonate

To a solution of 6-bromo-4-hydroxy-8-methylpyrido[2,3-d]pyrimidin-7 (8H)-one (850 mg, 3.32 mmol) in DCM (17 mL) and pyridine (1.4 mL, 17.3 mmol) was added Tf₂O (2.81 g, 9.95 mmol) dropwise at 0° C. The mixture was stirred at 30° C. for 3 hours, diluted with water (10 mL), and extracted with DCM (30 mL×2). The combined organic layers were washed with brine (20 mL), dried over anhydrous Na₂SO₄, filtered, and concentrated under reduced pressure to give the title compound (850 mg, 2.19 mmol, 66.0% yield) as a pale yellow solid.

LC-MS m/e: 388 (MH⁺).

### Step 3. Synthesis of (R)-6-bromo-8-methyl-4-((1-(2-methyl-3-(trifluoromethyl)phenyl)prop-2-yn-1-yl)amino)pyrido[2,3-d]pyrimidin-7 (8H)-one

To a solution of 6-bromo-8-methyl-7-oxo-7,8-dihydropyrido[2,3-d]pyrimidin-4-yl trifluoromethanesulfonate. (800 mg, 2.06 mmol) and (R)-1-(2-methyl-3-(trifluoromethyl)phenyl)prop-2-yn-1-amine (617 mg, 2.47 mmol) in dioxane (10 mL) was added DIPEA (1.0 mL, 6.18 mmol) dropwise at 0° C. The mixture was stirred at 100° C. for 3 hours, cooled to room temperature, diluted with water (10 mL), and extracted with EtOAc (50 mL×3). The combined organic layers were washed with brine (20 mL), dried over anhydrous Na₂SO₄, filtered, and concentrated under reduced pressure to dryness. The residue was purified by flash chromatography on silica gel (gradient, PE to PE:EtOAc=2:1) to give the title compound (405 mg, 0.898 mmol, 43.5% yield) as a pale yellow solid.

LC-MS m/e: 451 (MH⁺).

### Step 4. Synthesis of (R)-6-(4-hydroxytetrahydro-2H-pyran-4-yl)-8-methyl-4-((1-(2-methyl-3-(trifluoromethyl)phenyl)prop-2-yn-1-yl)amino)pyrido[2,3-d]pyrimidin-7 (8H)-one

To a solution of (R)-6-bromo-8-methyl-4-((1-(2-methyl-3-(trifluoromethyl)phenyl)prop-2-yn-1-yl)amino)pyrido[2,3-d]pyrimidin-7 (8H)-one (400 mg, 0.886 mmol) and oxan-4-one (0.164 mL, 1.77 mmol) in THF (12 mL) was added SmI₂ (42 mL, 4.20 mmol, 0.1 M in THF) dropwise at −60° C. The mixture was stirred at −60° C. for 1.5 hours, quenched with saturated NH₄Cl solution (20 mL), and extracted with EtOAc (20 mL×2). The combined organic layers were washed with brine (20 mL), dried over anhydrous Na₂SO₄, filtered, and concentrated under reduced pressure to dryness. The residue was purified by flash chromatography on silica gel (gradient, PE to PE:EtOAc=3:1) to give the title compound (310 mg, 0.656 mmol, 74.0% yield) as a pale yellow solid.

LC-MS m/e: 473 (MH⁺).

### Step 5. Synthesis of (R)—N-(4-(8-methyl-4-((1-(2-methyl-3-(trifluoromethyl)phenyl)prop-2-yn-1-yl)amino)-7-oxo-7,8-dihydropyrido[2,3-d]pyrimidin-6-yl)tetrahydro-2H-pyran-4-yl)acetamide (Compound 58)

To a solution of (R)-6-(4-hydroxytetrahydro-2H-pyran-4-yl)-8-methyl-4-((1-(2-methyl-3-(trifluoromethyl)phenyl)prop-2-yn-1-yl)amino)pyrido[2,3-d]pyrimidin-7 (8H)-one (300 mg, 0.635 mmol) in CH₃CN (15 mL) was added H₂SO₄ (3 mL) dropwise in CH₃CN (10 mL) at −60° C. under N₂. The mixture was stirred at −50° C. for 1.5 hours and diluted with water (10 mL) at 0° C. The mixture was adjusted to pH=7-8 with saturated NaHCO₃ solution and extracted with DCM (30 mL×2). The combined organic layers were washed with brine (20 mL), dried over anhydrous Na₂SO₄, filtered, and concentrated under reduced pressure to dryness. The residue was purified by flash chromatography on silica gel (gradient, PE to PE:EtOAc=1:2) to give the title compound (186 mg, 0.362 mmol, 57.0% yield) as an off-white solid.

¹H NMR (400 MHz, CD₃OD) δ 8.44 (s, 1H), 8.19 (s, 1H), 8.07 (d, J=8.0 Hz, 1H), 7.65 (d, J=8.0 Hz, 1H), 7.40 (t, J=7.8 Hz, 1H), 6.72 (d, J=2.4 Hz, 1H), 3.84-3.78 (m, 4H), 3.68 (s, 3H), 2.99 (d, J=2.8 Hz, 1H), 2.80-2.71 (m, 2H), 2.49 (s, 3H), 2.03 (m, 2H), 1.91 (s, 3H).

LC-MS m/e: 514 (MH⁺).

### Synthesis of Compound 59: (R)-6-(1-acetyl-4-aminopiperidin-4-yl)-8-methyl-4-((1-(2-methyl-3-(trifluoromethyl)phenyl)prop-2-yn-1-yl)amino)pyrido[2,3-d]pyrimidin-7 (8H)-one

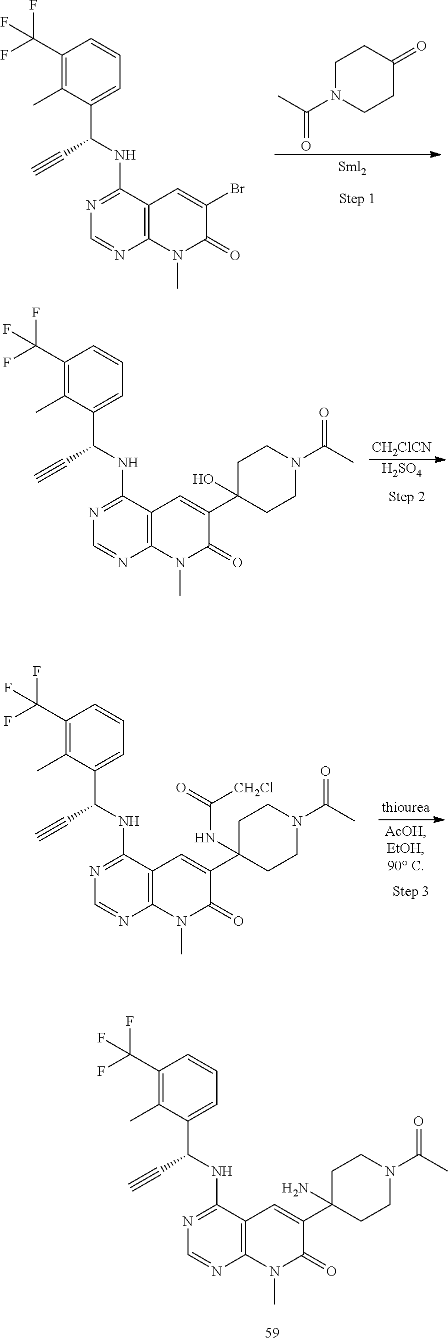

### Step 1. Synthesis of (R)-6-(1-acetyl-4-hydroxypiperidin-4-yl)-8-methyl-4-((1-(2-methyl-3-(trifluoromethyl)phenyl)prop-2-yn-1-yl)amino)pyrido[2,3-d]pyrimidin-7 (8H)-one

To a solution of (R)-6-bromo-8-methyl-4-((1-(2-methyl-3-(trifluoromethyl) phenyl) prop-2-yn-1-yl)amino)pyrido[2,3-d]pyrimidin-7 (8H)-one (800 mg, 1.77 mmol) and 1-acetylpiperidin-4-one (501 mg, 3.55 mmol) in THF (10 mL) was added SmI₂ (89 mL, 8.9 mmol, 0.1 M in THF) at −50° C. The mixture was stirred at −50° C. for 2 hours, quenched with saturated NH₄Cl solution (20 mL), and extracted with EtOAc (50 mL×2). The combined organic layers were washed with brine (50 mL), dried over anhydrous Na₂SO₄, filtered, and concentrated to dryness. The residue was purified by flash chromatography on silica gel (gradient, PE to PE:EtOAc=3:1) to give the title compound (700 mg, 1.37 mmol, 77.0% yield) as a white solid.

LC-MS m/e: 514 (MH⁺).

### Step 2. Synthesis of (R)—N-(1-acetyl-4-(8-methyl-4-((1-(2-methyl-3-(trifluoromethyl) phenyl) prop-2-yn-1-yl)amino)-7-oxo-7,8-dihydropyrido[2,3-d]pyrimidin-6-yl) piperidin-4-yl)-2-chloroacetamide

To a solution of (R)-6-(1-acetyl-4-hydroxypiperidin-4-yl)-8-methyl-4-((1-(2-methyl-3-(trifluoromethyl)phenyl)prop-2-yn-1-yl)amino)pyrido[2,3-d]pyrimidin-7 (8H)-one (300 mg, 0.584 mmol) in 2-chloroacetonitrile (5 mL) was added H₂SO₄ (1.5 mL) dropwise at −60° C. The mixture was stirred at −60° C. for 2 hours, quenched with saturated NaHCO₃ solution (20 mL), and extracted with EtOAc (50 mL×3). The combined organic layers were washed with brine (40 mL), dried over anhydrous Na₂SO₄, filtered, and concentrated to dryness. The residue was purified by flash chromatography on silica gel (gradient, 0-7% MeOH in DCM) to give the title compound (300 mg, 0.509 mmol, 87.2%) as a yellow solid.

LC-MS m/e: 589 (MH⁺).

### Step 3. Synthesis of (R)-6-(1-acetyl-4-aminopiperidin-4-yl)-8-methyl-4-((1-(2-methyl-3-(trifluoromethyl)phenyl)prop-2-yn-1-yl)amino)pyrido[2,3-d]pyrimidin-7 (8H)-one (Compound 59)

To a solution of (R)—N-(1-acetyl-4-(8-methyl-4-((1-(2-methyl-3-(trifluoromethyl) phenyl) prop-2-yn-1-yl)amino)-7-oxo-7,8-dihydropyrido[2,3-d]pyrimidin-6-yl) piperidin-4-yl)-2-chloroacetamide (300 mg, 0.509 mmol) in EtOH (5 mL) and acetic acid (0.1 mL) was added thiourea (58 mg, 0.764 mmol) at 25° C. The mixture was stirred at 80° C. for 3 hours. The reaction mixture was cooled to room temperature, quenched with saturated NaHCO₃ solution (10 mL) and extracted with EtOAc (20 mL×3). The combined organic layers were washed with brine (20 mL), dried over Na₂SO₄, filtered, and concentrated to dryness. The residue was purified by prep-HPLC (SilaSep™ C18 silica flash cartridge, 0%-40% MeCN in H₂O with 0.1% formic acid) to give the title compound (25.5 mg, 0.050 mmol, 9.8% yield) as a white solid.

¹H NMR (400 MHz, DMSO-d6) δ 9.03-8.91 (m, 1H), 8.47 (d, J=4.0 Hz, 1H), 8.17 (s, 1H), 8.01 (d, J=8.0 Hz, 1H), 7.70 (d, J=8.0 Hz, 1H), 7.50 (t, J=8.0 Hz, 1H), 6.67 (d, J=8.0 Hz, 1H), 4.25 (d, J=12.0 Hz, 1H), 3.70-3.59 (m, 7H), 3.10 (t, J=12.0 Hz, 1H), 2.45 (s, 3H), 2.00 (s, 3H), 1.94-1.80 (m, 4H).

LC-MS m/e: 513 (MH⁺).

### Synthesis of Compound 60: (R)-6-(4-aminotetrahydro-2H-pyran-4-yl)-8-methyl-4-((1-(2-methyl-3-(trifluoromethyl)phenyl)prop-2-yn-1-yl)amino)pyrido[2,3-d]pyrimidin-7 (8H)-one

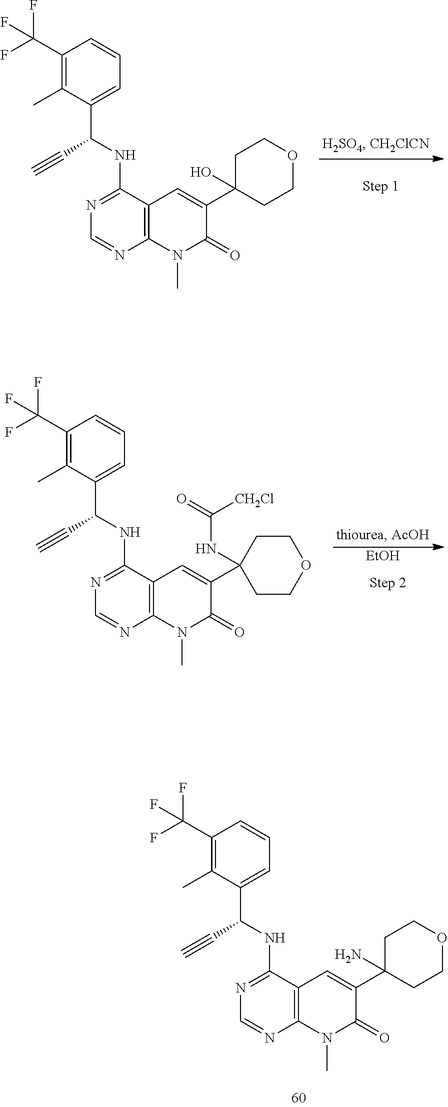

### Step 1. Synthesis of (R)-2-chloro-N-(4-(8-methyl-4-((1-(2-methyl-3-(trifluoromethyl)phenyl)prop-2-yn-1-yl)amino)-7-oxo-7,8-dihydropyrido[2,3-d]pyrimidin-6-yl)tetrahydro-2H-pyran-4-yl)acetamide

To a solution of (R)-6-(4-hydroxytetrahydro-2H-pyran-4-yl)-8-methyl-4-((1-(2-methyl-3-(trifluoromethyl)phenyl)prop-2-yn-1-yl)amino)pyrido[2,3-d]pyrimidin-7 (8H)-one (350 mg, 0.741 mmol) in chloroacetonitrile (4 mL) was added H₂SO₄ (1 mL) dropwise in chloroacetonitrile (3 mL) at −60° C. The mixture was stirred at −60° C. for 2 hours, quenched with saturated NaHCO₃ solution (15 mL), and extracted with EtOAc (30 mL×2). The combined organic layers were washed with brine (20 mL), dried over anhydrous Na₂SO₄, filtered, and concentrated under reduced pressure to dryness. The residue was purified by flash chromatography on silica gel (gradient, PE to PE:EtOAc=2:1) to give the title compound (364 mg, 0.847 mmol, 89.4% yield) as a pale yellow solid.

LC-MS m/e: 548 (MH⁺).

### Step 2. Synthesis of (R)-6-(4-aminotetrahydro-2H-pyran-4-yl)-8-methyl-4-((1-(2-methyl-3-(trifluoromethyl)phenyl)prop-2-yn-1-yl)amino)pyrido[2,3-d]pyrimidin-7 (8H)-one (Compound 60)

To a solution of (R)-2-chloro-N-(4-(8-methyl-4-((1-(2-methyl-3-(trifluoromethyl)phenyl)prop-2-yn-1-yl)amino)-7-oxo-7,8-dihydropyrido[2,3-d]pyrimidin-6-yl)tetrahydro-2H-pyran-4-yl)acetamide (100 mg, 0.182 mmol) in EtOH (3 mL) and AcOH (0.3 mL) was added thiourea (17 mg, 0.219 mmol). The mixture was stirred at 90° C. for 3 hours, cooled to room temperature, basified with saturated NaHCO₃ solution (15 mL), and extracted with EtOAc (30 mL×2). The combined organic layers were washed with brine (20 mL), dried over anhydrous Na₂SO₄, filtered, and concentrated under reduced pressure to dryness. The residue was purified by prep-HPLC (SilaSep™ C18 silica flash cartridge, 0%-40% MeCN in H₂O with 0.1% formic acid) to give the title compound (8 mg, 0.170 mmol, 9.30% yield) as a white solid.

¹H NMR (400 MHz, DMSO-d6) δ 8.97 (d, J=7.6 Hz, 1H), 8.47 (s, 1H), 8.15 (s, 1H), 8.02 (d, J=7.6 Hz, 1H), 7.70 (d, J=8.0 Hz, 1H), 7.50 (t, J=8.0 Hz, 1H), 6.68 (dd, J=8.0, 4.0 Hz, 1H), 3.92 (t, J=12.0 Hz, 2H), 3.64 (m, 3H), 3.61 (s, 3H), 2.45 (s, 3H), 2.16 (s, 2H), 2.02 (m, 2H), 1.82-1.72 (m, 2H).

LC-MS m/e: 472 (MH⁺).

### Syntheses of Compounds 61 and 62: N-(3-(5-(((R)-1-(3-(difluoromethyl)-2-fluorophenyl)prop-2-yn-1-yl)amino)-1-methyl-2-oxo-1,2-dihydro-1,8-naphthyridin-3-yl)-8-oxabicyclo[3.2.1]octan-3-yl)acetamide (Compound 61) and 6-(8-oxabicyclo[3.2.1]oct-2-en-3-yl)-4-(((R)-1-(3-(difluoromethyl)-2-fluorophenyl)prop-2-yn-1-yl)amino)-8-methylpyrido[2,3-d]pyrimidin-7 (8H)-one (Compound 62)

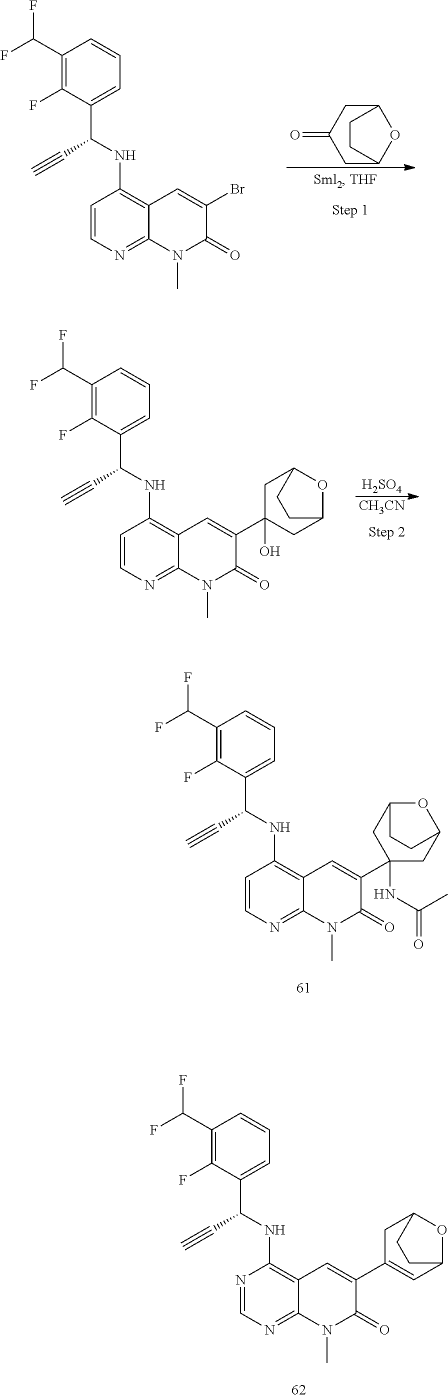

### Step 1. Synthesis of 5-(((R)-1-(3-(difluoromethyl)-2-fluorophenyl)prop-2-yn-1-yl)amino)-3-(3-hydroxy-8-oxabicyclo[3.2.1]octan-3-yl)-1-methyl-1,8-naphthyridin-2 (1H)-one

To a solution of (R)-3-bromo-5-((1-(3-(difluoromethyl)-2-fluorophenyl)prop-2-yn-1-yl)amino)-1-methyl-1,8-naphthyridin-2 (1H)-one (1.0 g, 2.29 mmol) and 8-oxabicyclo[3.2.1]octan-3-one (578 mg, 4.58 mmol) in THF (25 mL) was added SmI₂ (16 mmol, 160 mL, 0.1 M in THF) at −60° C. Then the reaction mixture was stirred at −10° C. for 2 hours, quenched with saturated NH₄Cl solution (80 mL), and extracted with EtOAc (50 mL×2). The combined organic layers were washed with brine (50 mL), dried over anhydrous Na₂SO₄, filtered, and concentrated under reduced pressure to dryness. The residue was purified by flash chromatography on silica gel (gradient, PE to PE:EtOAc=1:1) to give the title compound (720 mg, 1.49 mmol, 65.1% yield) as a yellow solid.

LC-MS m/e: 484.5 (MH⁺).

### Step 2. Syntheses of N-(3-(5-(((R)-1-(3-(difluoromethyl)-2-fluorophenyl)prop-2-yn-1-yl)amino)-1-methyl-2-oxo-1,2-dihydro-1,8-naphthyridin-3-yl)-8-oxabicyclo[3.2.1]octan-3-yl)acetamide (Compound 61) and 6-(8-oxabicyclo[3.2.1]oct-2-en-3-yl)-4-(((R)-1-(3-(difluoromethyl)-2-fluorophenyl)prop-2-yn-1-yl)amino)-8-methylpyrido[2,3-d]pyrimidin-7 (8H)-one (Compound 62)

To a solution of 5-(((R)-1-(3-(difluoromethyl)-2-fluorophenyl)prop-2-yn-1-yl)amino)-3-(3-hydroxy-8-oxabicyclo[3.2.1]octan-3-yl)-1-methyl-1,8-naphthyridin-2 (1H)-one (500 mg, 1.03 mmol) in CH₃CN (15 mL) was added H₂SO₄ (1.5 mL) at −60° C. The mixture was stirred at −60° C. for 2 hours, quenched with saturated NaHCO₃ solution (30 mL), and extracted with EtOAc (50 mL×2). The combined organic layers were washed with brine (50 mL), dried over anhydrous Na₂SO₄, filtered, and concentrated under reduced pressure to dryness. The residue was purified by prep-HPLC (SilaSep™ C18 silica flash cartridge, 0%-40% MeCN in H₂O with 0.1% formic acid) to give Compound 61 (40 mg, 0.076 mmol, 7.4% yield) as a yellow solid and Compound 62 (10.7 mg, 0.022 mmol, 2.1% yield) as a yellow solid.

Compound 61: ¹H NMR (400 MHz, CD₃OD) δ 8.41 (s, 1H), 8.14 (s, 2H), 7.94 (t, J=8.0 Hz, 1H), 7.60 (t, J=6.0 Hz, 1H), 7.35 (t, J=6.0 Hz, 1H), 7.00 (t, J=56.0 Hz, 1H), 6.79 (d, J=4.0 Hz, 1H), 4.45 (s, 2H), 3.66 (s, 3H), 3.12-2.95 (m, 3H), 2.27-2.13 (m, 4H), 2.01-1.90 (m, 2H), 1.87 (s, 3H).

LC-MS m/e: 526.3 (MH⁺).

Compound 62: ¹H NMR (400 MHz, CD₃OD) δ 8.43 (s, 1H), 8.10 (s, 1H), 7.93 (t, J=8.0 Hz, 1H), 7.60 (t, J=6.0 Hz, 1H), 7.34 (t, J=8.0 Hz, 1H), 7.00 (t, J=52.0 Hz, 1H), 6.84 (d, J=1.6 Hz, 1H), 6.78 (d, J=2.4 Hz, 1H), 4.65 (t, J=6.0 Hz, 1H), 4.57 (t, J=6.0 Hz, 1H), 3.70 (s, 3H), 3.02 (d, J=2.4 Hz, 1H), 3.02-2.91 (m, 1H), 2.30-2.21 (m, 1H), 2.20-2.08 (m, 2H), 2.03-1.90 (m, 1H), 1.90-1.79 (m, 1H).

LC-MS m/e: 467.2 (MH⁺).

### Synthesis of Compound 63: 6-(8-acetyl-3-hydroxy-8-azabicyclo[3.2.1]octan-3-yl)-4-(((R)-1-(3-(difluoromethyl)-2-fluorophenyl)prop-2-yn-1-yl)amino)-8-methylpyrido[2,3-d]pyrimidin-7 (8H)-one

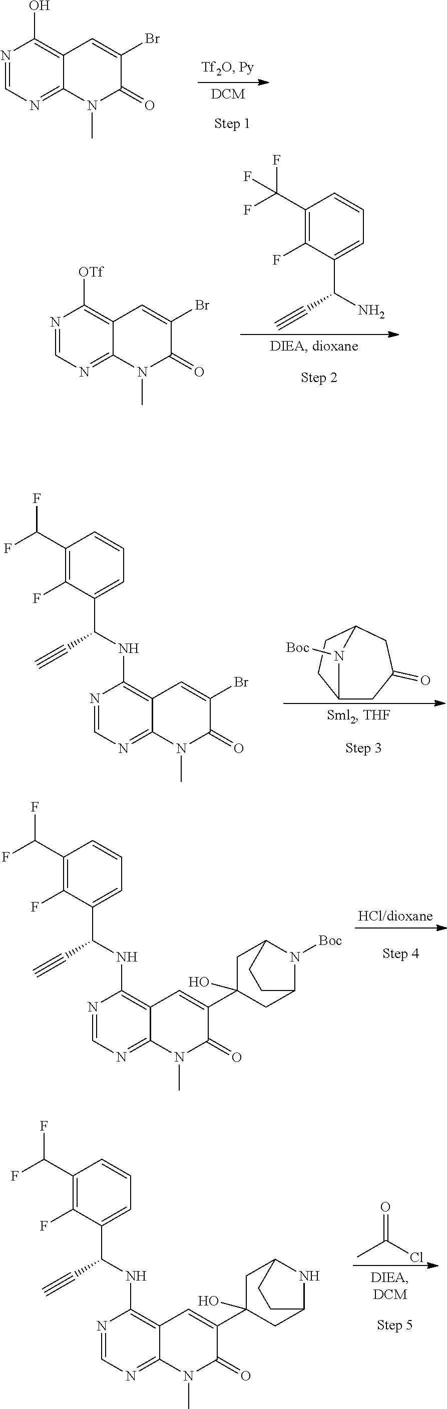

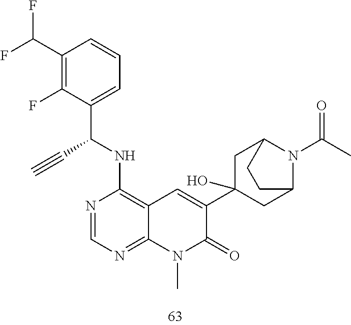 [SMILES: C#C[C@@H](Nc1ncnc2c1cc(C1(O)CC3CCC(C1)N3C(C)=O)c(=O)n2C)c1cccc(C(F)F)c1F]

### Step 1. Synthesis of 6-bromo-8-methyl-7-oxo-7,8-dihydropyrido[2,3-d]pyrimidin-4-yl trifluoromethanesulfonate

To a solution of 6-bromo-4-hydroxy-8-methyl-7H,8H-pyrido[2,3-d]pyrimidin-7-one (4 g, 15.6 mmol) in DCM (60 mL) was added Tf₂O (13.2 g, 46.9 mmol) dropwise at 0° C. under N₂ for 10 mins. Then pyridine (6.3 mL, 78.0 mmol) was slowly added into the mixture solution at 0° C. under N₂ and stirred at 0° C. for 2 hours. The resulting mixture was poured into ice water (100 mL) and DCM (100 mL). The organic layer was washed with brine (100 mL), dried over Na₂SO₄ and filtered. The filtrate was concentrated under reduced pressure to give the title compound (5.3 g, 13.7 mmol, 87.4% yield) as yellow solid.

LC-MS m/e: 388,390 (MH⁺).

### Step 2. Synthesis of (R)-6-bromo-4-((1-(3-(difluoromethyl)-2-fluorophenyl)prop-2-yn-1-yl)amino)-8-methylpyrido[2,3-d]pyrimidin-7 (8H)-one

A mixture of 6-bromo-8-methyl-7-oxo-7H,8H-pyrido[2,3-d]pyrimidin-4-yl trifluoromethanesulfonate (5.3 g, 13.7 mmol), (1R)-1-[3-(difluoromethyl)-2-fluorophenyl]ethan-1-amine hydrochloride salt (4.85 g, 20.6 mmol) and DIEA (6.8 mL, 41.0 mmol) in dioxane (100 mL) was stirred at 100° C. for 2 hours. The resulting mixture was cooled to room temperature, poured into water (100 mL), and extracted with EtOAc (100 mL×2). The combined organic layers were washed with brine (100 mL), dried over anhydrous Na₂SO₄ and concentrated under reduced pressure to dryness. The residue was purified by flash chromatography on silica gel (gradient, PE to PE:EtOAc=3:1) to give the title compound (4.41 g, 10.1 mmol, 72.7%) as yellow solid.

LC-MS m/e: 437, 439 (MH⁺).

### Step 3. Synthesis of tert-butyl 3-(4-(((R)-1-(3-(difluoromethyl)-2-fluorophenyl)prop-2-yn-1-yl)amino)-8-methyl-7-oxo-7,8-dihydropyrido[2,3-d]pyrimidin-6-yl)-3-hydroxy-8-azabicyclo[3.2.1]octane-8-carboxylate

To a solution of (R)-6-bromo-4-((1-(3-(difluoromethyl)-2-fluorophenyl)prop-2-yn-1-yl)amino)-8-methylpyrido[2,3-d]pyrimidin-7 (8H)-one (1.3 g, 2.97 mmol) and tert-butyl 3-oxo-8-azabicyclo[3.2.1]octane-8-carboxylate (1.34 g, 5.95 mmol) in THF (20 mL) was added SmI₂ (149 mL, 14.9 mmol, 0.1 M in THF) at −60° C. The mixture was stirred for 1.5 hours at −60° C., quenched with saturated NH₄Cl solution (100 mL), and extracted with EtOAc (100 mL×2). The combined organic layers were washed with brine (50 mL), dried over anhydrous Na₂SO₄ and concentrated under reduced pressure to dryness. The residue was purified by flash chromatography on silica gel (gradient, PE to PE:EtOAc=3:1) to give the title compound (1.3 g, 2.23 mmol, 74.9% yield) as yellow solid.

LC-MS m/e: 584 (MH⁺).

### Step 4. Synthesis of 4-(((R)-1-(3-(difluoromethyl)-2-fluorophenyl)prop-2-yn-1-yl)amino)-6-(3-hydroxy-8-azabicyclo[3.2.1]octan-3-yl)-8-methylpyrido[2,3-d]pyrimidin-7 (8H)-one

To a solution of tert-butyl 3-(4-(((R)-1-(3-(difluoromethyl)-2-fluorophenyl)prop-2-yn-1-yl)amino)-8-methyl-7-oxo-7,8-dihydropyrido[2,3-d]pyrimidin-6-yl)-3-hydroxy-8-azabicyclo[3.2.1]octane-8-carboxylate (1.3 g, 2.23 mmol) in dioxane (20 mL) was added HCl solution (10 mL, 40 mmol, 4 M in dioxane). The reaction mixture was stirred at room temperature for 2 hours. The resulting mixture was concentrated under vacuum to give the title compound as a HCl salt (1 g, 2.07 mmol, 92.9% yield).

LC-MS m/e: 484 (MH⁺).

### Step 5. Synthesis of 6-(8-acetyl-3-hydroxy-8-azabicyclo[3.2.1]octan-3-yl)-4-(((R)-1-(3-(difluoromethyl)-2-fluorophenyl)prop-2-yn-1-yl)amino)-8-methylpyrido[2,3-d]pyrimidin-7 (8H)-one

To a solution of 4-(((R)-1-(3-(difluoromethyl)-2-fluorophenyl)prop-2-yn-1-yl)amino)-6-(3-hydroxy-8-azabicyclo[3.2.1]octan-3-yl)-8-methylpyrido[2,3-d]pyrimidin-7 (8H)-one (1 g, 2.07 mmol) and DIEA (801 mg, 6.21 mmol) in DCM (20 mL) was added acetyl chloride (325 mg, 4.14 mmol) at 0° C. and stirred at 0° C. for 30 mins. The resulting mixture was quenched with water (100 mL) and extracted with DCM (50 mL×2). The combined organic layers were washed with brine (50 mL), dried over anhydrous Na₂SO₄ and concentrated under reduced pressure to dryness. The residue was purified by flash chromatography on silica gel (gradient, DCM to DCM: MeOH=30:1) to give the title compound (780 mg, 1.484 mmol, 71.8%) as yellow oil.

¹H NMR (400 MHz, DMSO-d6) δ 9.17 (dd, J=7.6, 4.4 Hz, 1H), 8.53 (s, 1H), 8.47 (s, 1H), 7.96 (t, J=7.4 Hz, 1H), 7.64 (t, J=7.2 Hz, 1H), 7.43 (t, J=7.8 Hz, 1H), 7.24 (t, J=54.2 Hz, 1H), 6.80-6.76 (m, 1H), 5.18 (s, 1H), 4.45 (s, 1H), 4.19 (s, 1H), 3.62 (t, J=2.6 Hz, 1H), 3.57 (s, 3H), 2.71-2.57 (m, 2H), 2.33-2.20 (m, 2H), 2.00 (s, 3H), 1.97-1.68 (m, 2H), 1.61-1.36 (m, 2H).

LC-MS m/e: 526 (MH⁺).

### Syntheses of Compounds 64 and 65: N-(8-acetyl-3-(4-(((R)-1-(3-(difluoromethyl)-2-fluorophenyl)prop-2-yn-1-yl)amino)-8-methyl-7-oxo-7,8-dihydropyrido[2,3-d]pyrimidin-6-yl)-8-azabicyclo[3.2.1]octan-3-yl)acetamide (Compound 64) and 6-(8-acetyl-8-azabicyclo[3.2.1]oct-2-en-3-yl)-4-(((R)-1-(3-(difluoromethyl)-2-fluorophenyl)prop-2-yn-1-yl)amino)-8-methylpyrido[2,3-d]pyrimidin-7 (8H)-one (Compound 65)

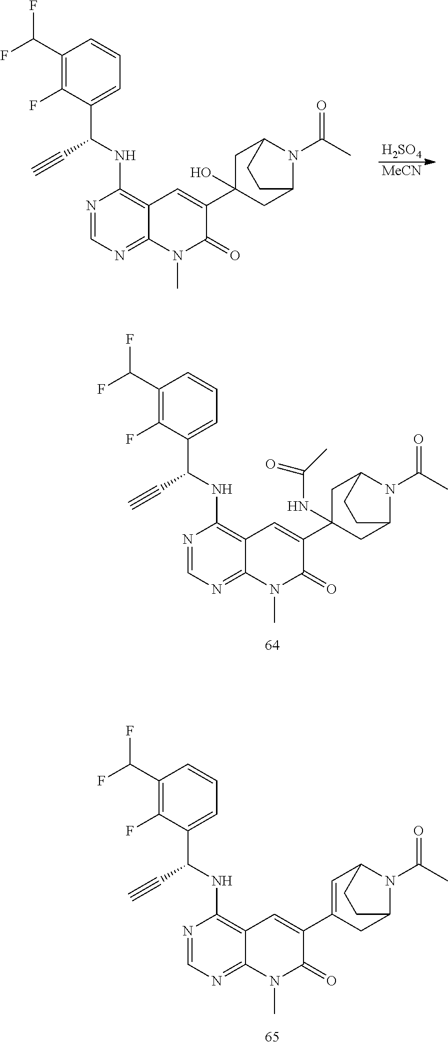

To a solution of 6-(8-acetyl-3-hydroxy-8-azabicyclo[3.2.1]octan-3-yl)-4-(((R)-1-(3-(difluoromethyl)-2-fluorophenyl)prop-2-yn-1-yl)amino)-8-methylpyrido[2,3-d]pyrimidin-7 (8H)-one (80 mg, 0.152 mmol) in CH₃CN (6 mL) was added H₂SO₄ (2 mL) at −60° C. The mixture was stirred at −60° C. for 2 hours, quenched with saturated NaHCO₃ solution (30 mL), and extracted with EtOAc (50 mL×2). The combined organic layers were washed with brine (50 mL), dried over anhydrous Na₂SO₄, filtered, and concentrated under reduced pressure to dryness. The residue was purified by prep-HPLC (SilaSep™ C18 silica flash cartridge, 0%-40% MeCN in H₂O with 0.1% formic acid) to give Compound 64 (29.1 mg, 0.051 mmol, 33.7% yield) as light-yellow solid and Compound 65 (17.3 mg, 0.034 mmol, 22.4% yield) as yellow solid.

Compound 64: ¹H NMR (400 MHz, DMSO-d6) δ 9.17 (t, J=7.2 Hz, 1H), 8.44 (d, J=4.4 Hz, 1H), 8.11 (d, J=4.0 Hz, 1H), 8.08 (d, J=2.4 Hz, 1H), 7.92 (q, J=6.0 Hz, 1H), 7.64 (s, 1H), 7.44 (t, J=7.6 Hz, 1H), 7.26 (t, J=54.2 Hz, 1H), 6.90-6.70 (m, 1H), 4.54 (s, 1H), 4.22 (s, 1H), 3.68 (t, J=2.2 Hz, 1H), 3.57 (s, 3H), 3.13-2.98 (m, 2H), 2.39-2.15 (m, 2H), 2.05-1.89 (m, 5H), 1.77 (d, J=2.8 Hz, 3H).

LC-MS m/e: 567 (MH⁺).

Compound 65: ¹H NMR (400 MHz, DMSO-d6) δ 9.12-9.01 (m, 1H), 8.45 (d, J=4.0 Hz, 1H), 8.17 (d, J=2.0 Hz, 1H), 7.92-7.87 (m, 1H), 7.64 (t, J=6.8 Hz, 1H), 7.43 (t, J=7.6 Hz, 1H), 7.39-6.87 (m, 2H), 6.77-6.72 (m, 1H), 4.82-4.62 (m, 1H), 4.59-4.41 (m, 1H), 3.69-3.68 (m, 1H), 3.59 (d, J=1.6 Hz, 3H), 3.11-2.82 (m, 1H), 2.62-2.53 (m, 1H), 2.37-2.19 (m, 1H), 2.01 (s, 3H), 1.99-1.66 (m, 3H).

LC-MS m/e: 508 (MH⁺).

Compounds 66-69 can be prepared following the procedure used for preparation of Compound 40.

As described below, compounds of formula (I) thus prepared were tested using a) HTRF Based Protein-Protein Interaction Assay to determine their potency in inhibiting the protein-protein interaction between SOS1 and KRAS-G12D, b) pERK Potency Assay to measure the ability of test compounds to inhibit SOS1 function in cells, and c) 3D Proliferation Assay to examine the potency of test compounds for inhibiting SOS1-mediated proliferation, growth, and apoptosis of cancer cell lines in vitro.

### HTRF Based Protein-Protein Interaction Assay

A KRAS-G12D and SOS1 binding/interaction assay was designed to measure the interaction between KRAS-G12D and SOS1 proteins. Utilizing HTRF (Homogeneous Time-Resolved Fluorescence) technology, the assay enabled simple and rapid characterization of compound and protein interaction in a high throughput format. This assay was used to examine potency of the compounds in inhibiting the protein-protein interaction between SOS1 and KRAS-G12D. The assay demonstrates the molecular mode of action of compounds. Low IC₅₀ values are indicative of high potency of the SOS1 inhibitor compound to disrupt SOS1 and Kras-G12D interaction.

Protein Expression and Purification. The corresponding gene sequence to human SOS1 (residues 564-1049, UniProt ID: Q07889; SEQ ID NO: 2) was synthesized and fused in frame with GST expression vector. After the desired sequence was confirmed, plasmid containing the sequence was transformed into the E. coli strain BL21 (DE3). Bacteria were growing at 37° C. in LB media containing 50 μg/ml Kanamycin to give OD₆₀₀ of 0.6-0.8. Then 0.2 mM IPTG was added to induce protein expression for 16 hours at 17° C. Bacteria were harvested by centrifugation and stored at −80° C. Bacteria pellets were resuspended in lysis buffer (25 mM Tris-HCl, 500 mM NaCl, 2 mM DTT, 2.3% sucrose, 0.3% dextran-10, 1 mM PMSF, pH 7.5) and lysed using high-pressure homogenizer. The lysate was cleared by centrifugation for 30 minutes (12000 rpm at 4° C.). The supernatant containing GST-SOS1 fragment was purified sequentially through Glutathione column and gel filtration (Hiload 16/600 Superdex 200 μg column, Cytiva). The purified GST-SOS1 fragment was confirmed by SDS-PAGE and stored in 25 mM Tris-HCl, 100 mM NaCl, 1 mM DTT, 2.3% sucrose, 0.3% dextran-10, pH 7.5 at −80° C.

Kras-G12D Protein Purification. The sequence corresponding to human KRAS-G12D (residues 1-169, UniProt P01116-2; SEQ ID NO: 1) was synthesized and fused in frame with His-AVI-TEV vector. The plasmid was transformed into the E. coli strain BL21 (containing a plasmid that can generate BirA enzyme). Bacteria were growing at 37° C. in TB media containing 100 μg/ml ampicillin and 50 μg/ml Kanamycin to give OD₆₀₀ of 0.6-0.8. The 0.5 mM IPTG and 50 mg/l biotin were added to induce protein expression for 16 hours at 25° C. Bacteria were harvested by centrifugation and stored at −80° C. Bacteria pellets were re-suspended in lysis buffer (20 mM Tris-HCl, 500 mM NaCl, 5 mM MgCl₂, 2 mM 3-ME, 5% glycerol, pH 8.0) and lysed using high-pressure homogenizer. The lysate was cleared by centrifugation for 30 minutes (12000 rpm at 4° C.). The supernatant containing HIS-AVI-TEV-KRAS-G12D fragment was purified sequentially through Ni-NTA column (SMART), Streptactin column (SMART), and gel filtration (Hiload 16/600 Superdex 75 pg, GE). The purified HIS-AVI-TEV-KRAS-G12D fragment was confirmed by SDS-PAGE and stored in 50 mM HEPES-NaOH, 100 mM NaCl, 1 mM DTT, 5 mM MgCl₂, pH 7.5 at −80° C.

Protein-Protein Interaction Assay. An assay buffer containing 50 mM HEPES, pH 7.5, 50 mM NaCl, 0.01% Brij-35, 1 mM TCEP, 0.1% BSA was prepared, and concentration series of test compounds were generated spanning from 0.5 nM to 10 μM over 10 3-fold serial dilutions in a 384-well assay plate at a volume of 20 pL. The purified GST-SOS1 catalytic domain (residues 564-1049) was first diluted in assay buffer and 5 μl of SOS1 (final concentration 2.5 nM in assay mixture) was directly dispensed into compound plates. After a spin down at 1000 RPM for 30 seconds, the SOS1/compound mixture was incubated at 25° C. for 15 minutes to allow the reaction between SOS1 and the compound. A KRAS-G12D mixture was prepared by incubation of avi-tagged Kras-G12D (residue 1-169) and GDP in assay buffer containing 10 mM MgCl₂ at room temperature for 10 minutes. A KRAS-G12D and GDP mixture (5 μL) was added to the assay plate (final KRAS-G12D was 100 nM and GDP was 10 uM). The plate was centrifuged at 1000 rpm for 30 sec and incubated at 25° C. for 60 minutes. A monoclonal antibody to GST-conjugated with Tb cryptate and Streptavidin-XL665 in 1× assay buffer was prepared and 10 μL of the detection mixture was added to each well. The plate was incubated at 25° C. for 5 hours. A reading in HTRF mode with PerkinElmer Envision plate reader was taken at the end of incubation. An assay buffer containing KRAS-G12D and DMSO and a mixture of SOS1, KRAS-G12D, and DMSO were used as negative controls (minimum signal, columns 1 and 2) and positive controls (maximum signal, columns 23 and 24), respectively. Percent (%) inhibition for each dilution of compound was calculated by the following: [percent (%) inhibition=(1−(sample signal-negative control)/(positive control-negative control))*100]. Two to three separate experiments were performed for each compound and the data was analyzed using a four-parameter logistic fit.

Compounds of formula (I) were evaluated in the HTRF Based Protein-Protein Interaction Assay described above. Unexpectedly, Compounds 1-15, 18-29, 32, 34, 37-40, 42, 46, 47, 49, 54-57, and 65 each exhibited an IC₅₀ value less than 100 nM; and Compounds 16, 41, 43, 45, 48, 50, 51, 53, and 58 each exhibited an IC₅₀ value between 100 nM and 200 nM.

The results demonstrate that the compounds of the present invention are potent inhibitors of the protein-protein interaction between SOS1 and KRAS-G12D.

pERK Potency Assay

The purpose of this assay is to measure the ability of test compounds to inhibit SOS1 function in cells. SOS1 activates RAS proteins by catalyzing the conversion of RAS-GDP to RAS-GTP in response to receptor tyrosine kinase activation. Activation of RAS induces a sequence of cellular signaling events that results in increased phosphorylation of ERK at Threonine 202 and Tyrosine 204 (pERK). The procedure described below measured the level of cellular pERK in response to test compounds in NCI-H1975 cells (EGFR/L858R-T790M).

NCI-H1975 cells were grown and maintained using media and procedures recommended by the ATCC. On the day prior to compound addition, cells were plated in 24-well cell culture plates (0.9 ml/well) and grown overnight in a 37° C., 5% CO₂ incubator. Test compounds were prepared with 3-fold serial dilutions in DMSO, with a top concentration of 10 mM. On the day of the assay, 100 uL of each test compound diluted at 1:100 in media was added to each well of cell culture plate with final concentrations of the compound spanning 0.5 nM to 10 uM. After the compound was added, the cells were incubated for 1 hour at 37° C., 5% CO₂. Following incubation, culture medium was removed, and the cells were washed once with phosphate buffered saline and lysed with lysis buffer (20 mM Tris-HCl, pH 7.5, 150 mM NaCl, 1 mM EDTA, 1 mM EGTA, 0.8% NP-40 plus 2× protease and phosphatase inhibitors). Cellular pERK level was determined using the Western blot analysis and quantified with Liqor software. Signal from Western blot was plotted and IC₅₀ values were determined by fitting a 4-parameter sigmoidal concentration response model.

Compounds of formula (I) were evaluated. Unexpectedly, Compounds 4, 33, 43, 45, 46, 48, 49-51, 53-60, and 65 each exhibited an IC₅₀ value less than 100 nM; and Compounds 1, 9, 24, 52, and 61-64 each exhibited an IC₅₀ value between 100 nM and 500 nM.

### Cell Proliferation Assay

Cell proliferation assays were used to examine the potency with which compounds inhibit SOS1-mediated proliferation, growth, and apoptosis of cancer cell lines in vitro. This assay supports the molecular mode of action of compounds. Low IC₅₀ values are indicative of high potency of the SOS1 inhibition. In particular, it is observed that SOS1 inhibitors demonstrate potent inhibitory effect on the proliferation of EGFR mutant and KRAS mutant human cancer cell lines as well as SOS1 oncogenic mutation cancer cell lines. This supports the molecular mode of action of the SOS1 inhibitors as selectively targeting cancer cells dependent on receptor tyrosine kinase-RAS/SOS1-family protein function. Cell proliferation assays were performed in three-dimensional (3D) anchorage-independent conditions in 96 well ultra-low attachment plate with the following human cell lines:

NCI-H1975: human non-small cell lung cancer (NSCLC) with wild type KRAS and EGFR L858R/T790M mutation;

PC-9: human non-small cell lung cancer (NSCLC) with wild-type KRAS and an EGFR del19 mutation;

A549: human non-small cell lung cancer (NSCLC) with a KRAS G12S mutation;

NCI-H520: human non-small cell lung cancer (NSCLC) with wild-type KRAS;

MIA PaCa-2: human pancreatic cancer cell (PAC) with a KRAS G12C mutation;

Panc-1 human pancreatic cancer cell with KRAS G12D mutation;

HOP-92 lung cancer cell with SOS1 N233Y oncogenic mutation; or

LXF-289 lung cancer cells with SOS1 N233Y oncogenic mutation.

Cell lines were purchased from the American Type Culture Collection (ATCC), NCI, or European Collection of Authenticated Cell Cultures (ECACC). All cell lines were maintained in RPMI-1640 or DMEM with 10% heat inactivated fetal bovine serum.

3D Cell Proliferation Assay

Cells growing in log phase were detached with Gibco™ TrypLE™ Express Enzyme and plated in 96 well ultra low attachment plate at 15000 to 20000 cells/well in 100 μl of media. After overnight incubation at 37° C. and 5% CO₂, the cells were treated with compounds (50 μL/well) at the final concentrations spanning 1 nM to 20 μM. Cells were incubated at 37° C., 5% CO₂ and 95% humidity incubator for 96 hours. At the end of incubation, a 3D CTG reagent from Promega was added to each well according to vendors recommendation and mixed for 10 minutes in dark. The luminescence signals were determined using Biotek plate reader. The data was fitted using a sigmoidal curve analysis program (GraphPAD Prism) with variable hill slope.

Compounds of formula (I) were evaluated in the 3D cell proliferation assay using NCI-H1975 cells. Unexpectedly, Compounds 9, 33, 46, 48, 49, and 54-58 each exhibited an IC₅₀ value less than 100 nM and Compounds 4 and 43 each exhibited an IC₅₀ value between 100 nM and 500 nM.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the compounds of this invention also can be made, screened for their efficacy in treating a condition that relates to SOS1. Thus, other embodiments are also within the claims.

What is claimed is:

1. A compound of formula (I): 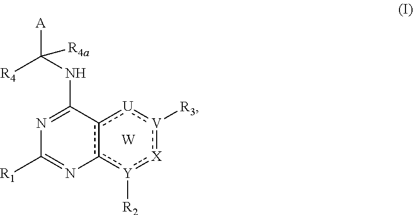
wherein
R₁ is H, D, —CD₃, halogen, —CN, —CONHR₁ₐ, —NHR₁ₐ, —OR₁ₐ, C₁₋₆ alkyl, C₃₋₆ cycloalkyl, C₁₋₆ heterocycloalkyl, C₂₋₄ alkenyl, or C₂₋₄ alkynyl, in which R₁ₐ is H, C₁₋₆ alkyl, C₃₋₆ cycloalkyl, or C₁₋₆ heterocycloalkyl;
R₂ is deleted, H, D, —CD₃, halogen, —CN, C₁₋₆ alkyl, C₁₋₆ alkoxy, C₃₋₆ cycloalkyl, C₁₋₆ heterocycloalkyl, C₂₋₆ alkenyl, or C₂₋₆ alkynyl;
R₃ is L₁-R₅;
R₄ is C₂₋₄ alkenyl or C₂₋₄ alkynyl;
R₄ₐ is H or D;
L₁ is a bond, —C(O)—, —C(O)O—, —C(O)NH(CH₂)_{q}—, —S—, —S(O)₂—, —S(O)—, —C(O)NR₆—, —SO₂NR₆—, —NR₆—, —NR₆C(O), —NR₆S(O)₂—, —NHC(O)NR₆—, —NHC(O)—, —NH(CH₂)_{q}NHC(O)—, —NH(CH₂)_{q}—, —O—, —O—(CH₂)_{q}—, C₁₋₄ alkylene, C₂₋₄ alkenylene, or C₂₋₄ alkynylene, in which q is 0, 1, or 2;
R₅ is H, D, C₁₋₁₀ alkyl, C₃₋₁₀ cycloalkyl, C₁₋₁₀ heterocycloalkyl, C₂₋₁₀ alkenyl, C₂₋₁₀ alkynyl, aryl, or heteroaryl;
R₆ is H, C₁₋₁₀ alkyl, C₃₋₁₀ cycloalkyl, C₁₋₁₀ heterocycloalkyl, aryl, or heteroaryl;
A is aryl heteroaryl, C₃₋₁₀ cycloalkyl, or C₁₋₁₀ heterocycloalkyl; [symbol]
is a single bond or a double bond, provided that the number of double bonds represented by [symbol] is 1, 2, or 3;
each of U and X, independently, is CH, C═O, or N;
each of V and Y, independently, is C, CH or N; and
W is 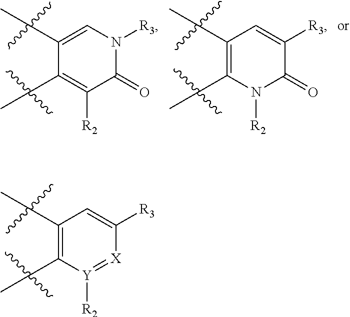
in which X is CH or N and Y is C or N.

2. The compound of claim 1, wherein the compound is of formula (IA) 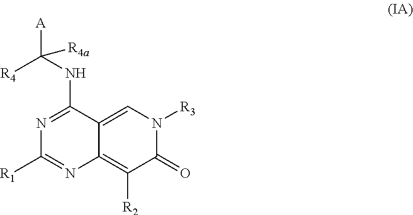
in which
R₁ is H, D, —CD₃, halogen, —CN, —NHR₁ₐ, —OR₁ₐ, C₁₋₃ alkyl, cyclopropyl, or C₂₋₃ alkenyl, R₁ₐ being H, C₁₋₃ alkyl, C₂₋₃ alkenyl, or cyclopropyl;
R₂ is H, D, halogen, —CD₃, —CN, —OR₁ₐ, C₁₋₄ alkyl, C₃₋₆ cycloalkyl, or C₁₋₆ heterocycloalkyl;
R₃ is L₁-R₅;
L₁ is a bond or —NR₆—;
R₅ is C₁₋₁₀ alkyl, C₃₋₁₀ cycloalkyl, C₁₋₁₀ heterocycloalkyl, C₂₋₁₀ alkenyl, C₂₋₁₀ alkynyl, aryl, or heteroaryl; and
R₆ is H, C₁₋₁₀ alkyl, C₃₋₁₀ cycloalkyl, C₁₋₁₀ heterocycloalkyl, aryl, or heteroaryl.

3. The compound of claim 2, wherein R₂ is H, halogen, —CN, —OR₁ₐ, C₁₋₄ alkyl, or C₃₋₆ cycloalkyl.

4. The compound of claim 3, wherein R₁ is H, D, —CD₃, cyclopropyl, or methyl and R₂ is H, halogen, or methyl.

5. The compound of claim 2, wherein R₃ is L₁-R₅, L₁ being a bond or —NH— and R₅ being C₁₋₁₀ alkyl, C₃₋₁₀ cycloalkyl, C₁₋₁₀ heterocycloalkyl, aryl, or heteroaryl.

6. The compound of claim 5, wherein R₃ is 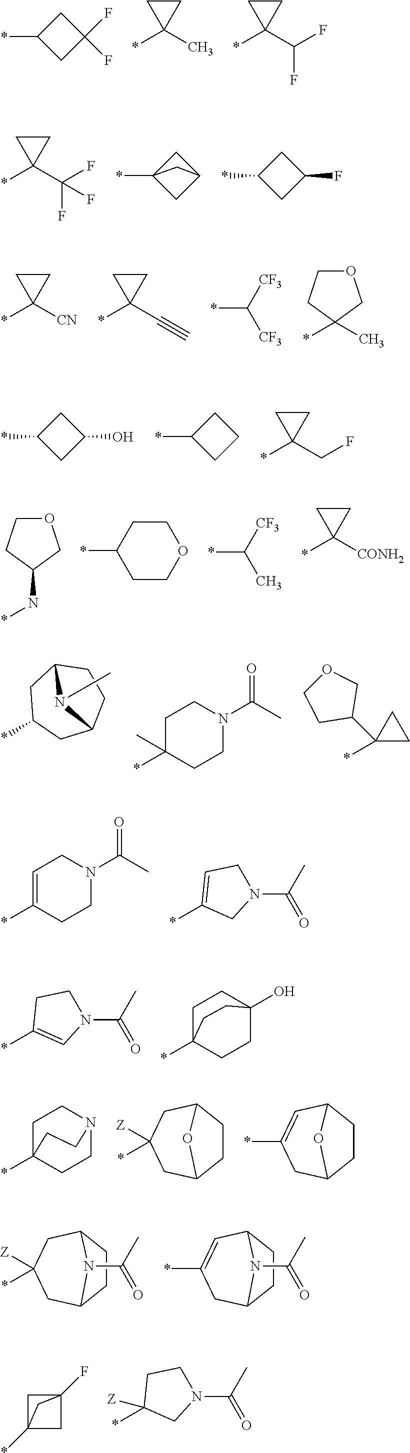 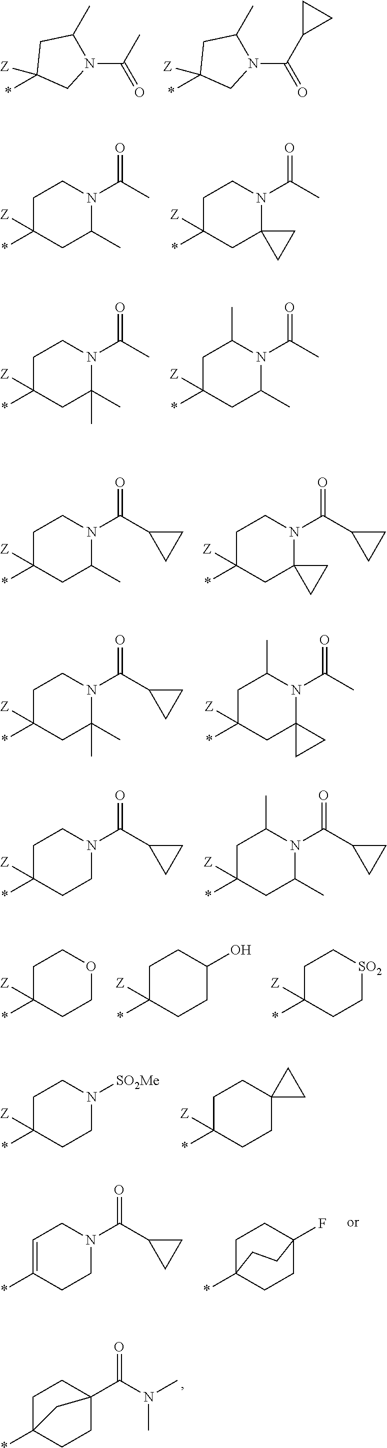
Z being H or methyl.

7. The compound of claim 6, wherein R₂ is H, halogen, —CN, —OR₁ₐ, C₁₋₄ or C₃₋₆ cycloalkyl; R₄ is 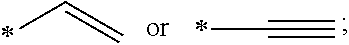
and A is phenyl or phenyl fused with a C₄₋₆ cycloalkyl, C₁₋₆ heterocycloalkyl, or C₁₋₆ heteroaryl, each of which is optionally substituted with one or more moieties selected from the group consisting of halogens, —OH, —NH₂, —CN, methyl, ethyl, C₁₋₃ alkoxy, C₁₋₃ haloalkoxy, fluoromethyl, difluoromethyl, trifluoromethyl, fluoroethyl, difluoroethyl, trifluoroethyl,

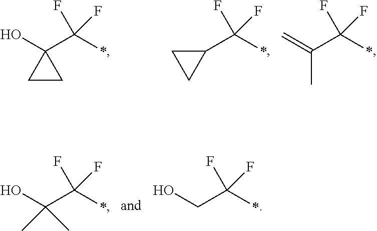

8. The compound of claim 7, wherein R₁ is H, D, —CD₃, cyclopropyl, or methyl; R₂ is H, halogen, methyl, or cyclopropyl; R₄ is 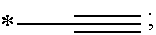 [SMILES: *C#C]
and A is

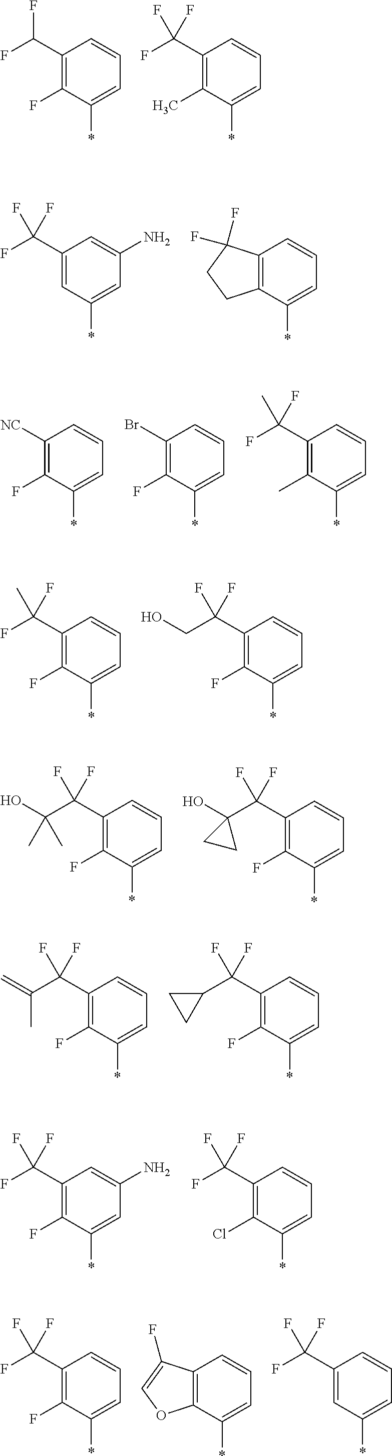

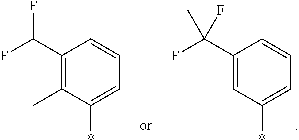 [SMILES: *c1cccc(C(F)F)c1C ; *c1cccc(C(C)(F)F)c1]

9. The compound of claim 5, wherein R₄ is 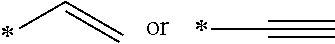
and A is phenyl or phenyl fused with a C₄₋₆ cycloalkyl, C₁₋₆ heterocycloalkyl, or C₁₋₆ heteroaryl, each of which is optionally substituted with one or more moieties selected from the group consisting of halogens, —OH, —NH₂, —CN, methyl, ethyl, C₁₋₃ alkoxy, C₁₋₃ haloalkoxy, fluoromethyl, difluoromethyl, trifluoromethyl, fluoroethyl, difluoroethyl, trifluoroethyl,

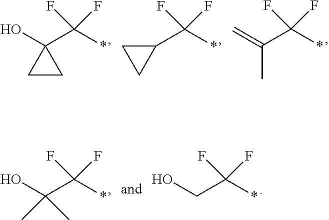

10. The compound of claim 9, wherein R₄ is 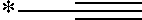 [SMILES: *C#C]
and A is

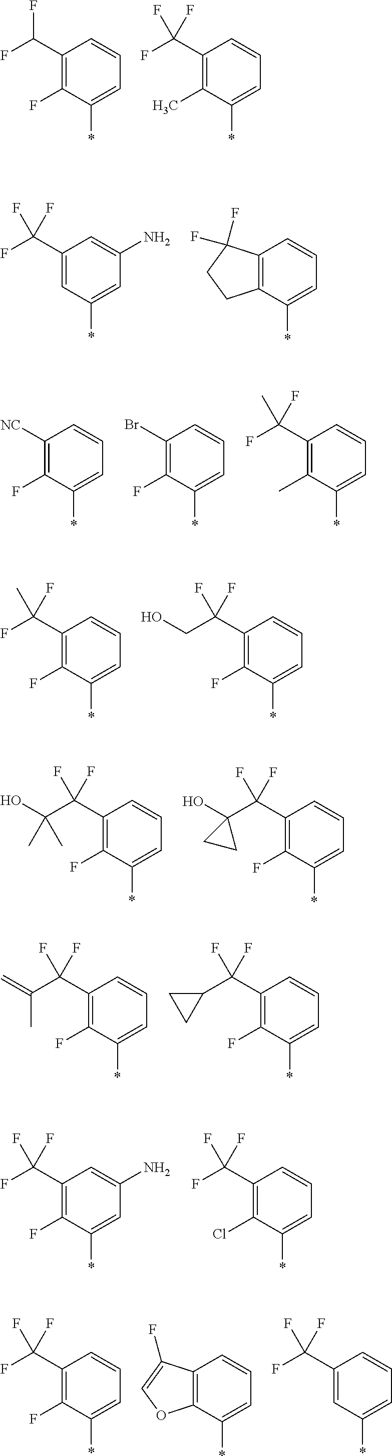

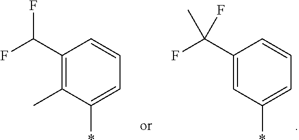 [SMILES: *c1cccc(C(F)F)c1C ; *c1cccc(C(C)(F)F)c1]

11. The compound of claim 2, wherein R₄ is

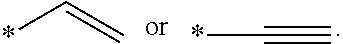

12. The compound of claim 11, wherein R₄ is

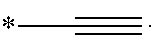 [SMILES: *C#C]

13. The compound of claim 12, wherein R₂ is H, halogen, —CN, —OR₁a, C₁₋₄ alkyl, or C₃₋₆ cycloalkyl; and A is phenyl or phenyl fused with a C₄₋₆ cycloalkyl, C₁₋₆ heterocycloalkyl, or C₁₋₆ heteroaryl, each of which is optionally substituted with one or more moieties selected from the group consisting of halogens, —OH, —NH₂, —CN, methyl, ethyl, C₁₋₃ alkoxy, C₁₋₃ haloalkoxy, fluoromethyl, difluoromethyl a C₄₋₆ cycloalkyl, C₁₋₆ heterocycloalkyl, C₁₋₆ heteroaryl, trifluoromethyl, fluoroethyl, difluoroethyl, trifluoroethyl,

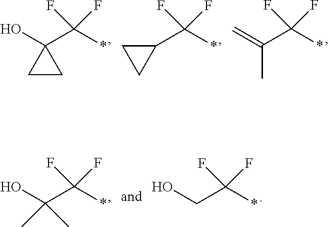

14. The compound of claim 13, wherein R₁ is H, D, —CD₃, cyclopropyl, or methyl; R₂ is H, halogen, or methyl; and A is

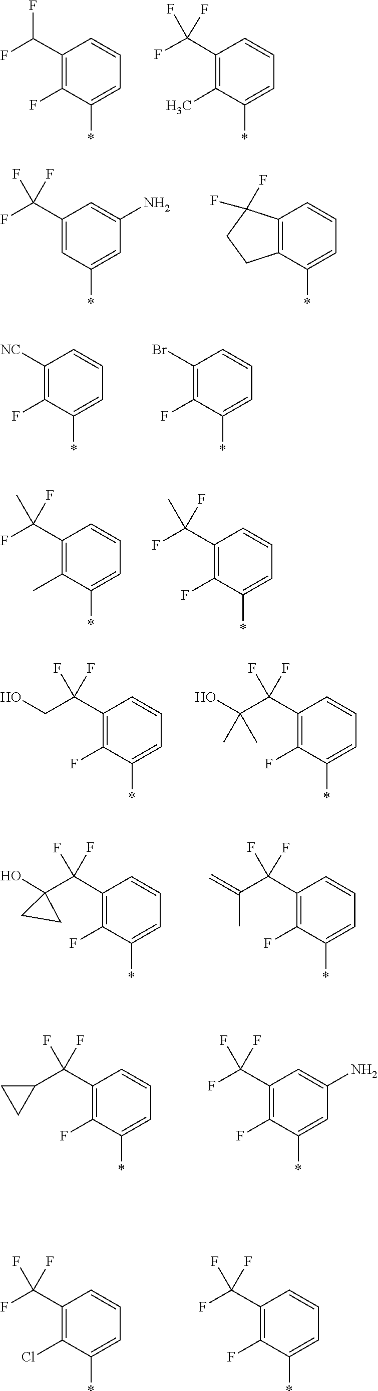

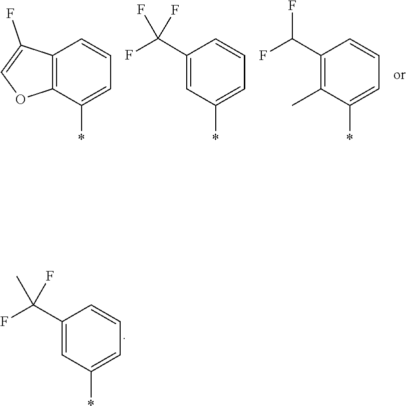

15. The compound of claim 11, wherein R₂ is H, halogen, —CN, —OR₁a, C₁₋₄ alkyl, or C₃₋₆ cycloalkyl; and A is phenyl or phenyl fused with a C₄₋₆ cycloalkyl, C₁₋₆ heterocycloalkyl, or C₁₋₆ heteroaryl, each of which is optionally substituted with one or more moieties selected from the group consisting of halogens, —OH, —NH₂, —CN, methyl, ethyl, C₁₋₃ alkoxy, C₁₋₃ haloalkoxy, fluoromethyl, difluoromethyl, trifluoromethyl, fluoroethyl, difluoroethyl, trifluoroethyl,

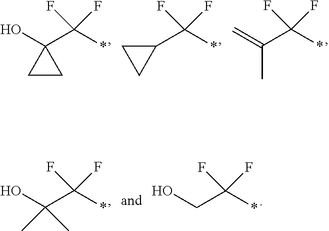

16. The compound of claim 15, wherein R₁ is H, D, —CD₃, cyclopropyl, or methyl; R₂ is H, halogen, or methyl; and A is

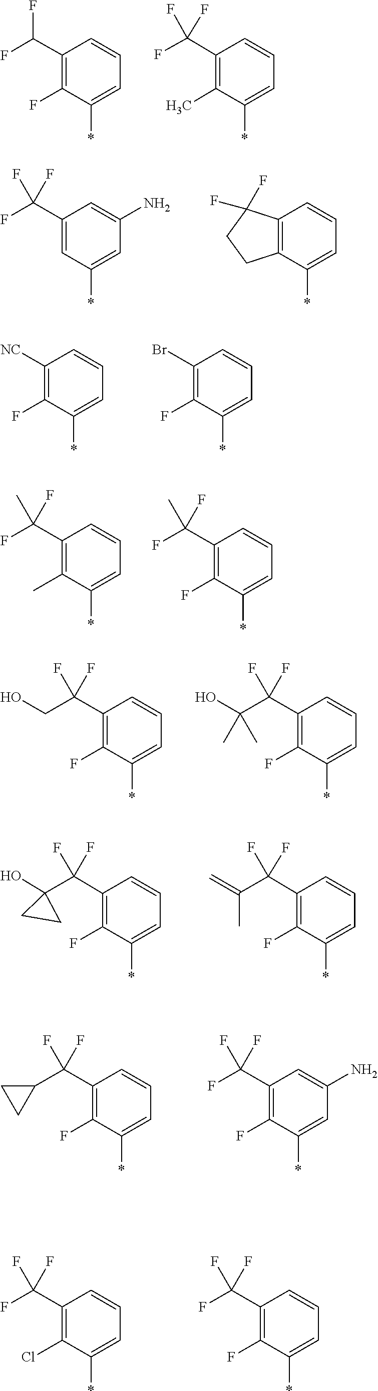

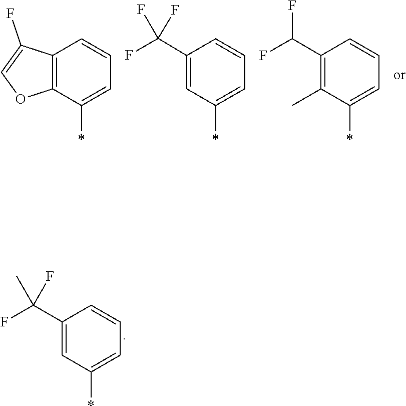

17. The compound of claim 2, wherein A is phenyl or phenyl fused with a C₄₋₆ cycloalkyl, C₁₋₆ heterocycloalkyl, or C₁₋₆ heteroaryl, each of which is optionally substituted with one or more moieties selected from the group consisting of halogens, —OH, —NH₂, —CN, methyl, ethyl, C₁₋₃ alkoxy, C₁₋₃ haloalkoxy, fluoromethyl, difluoromethyl, trifluoromethyl, fluoroethyl, difluoroethyl, trifluoroethyl,

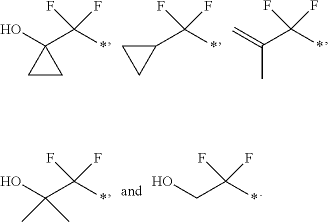

18. The compound of claim 17, wherein A is

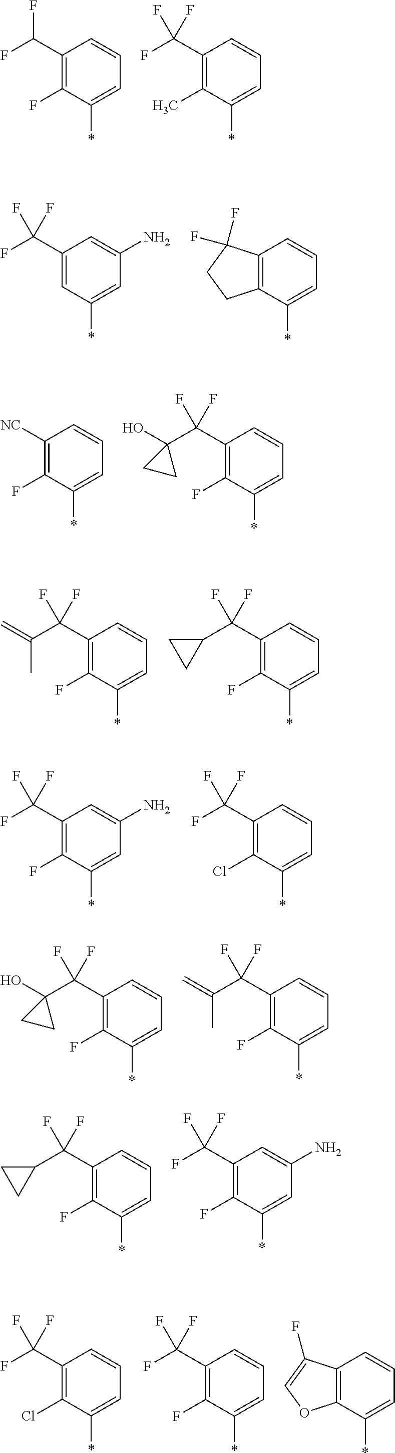

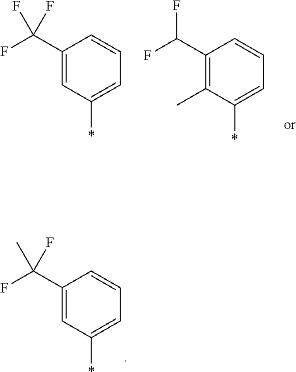

19. The compound of claim 16, wherein
A is 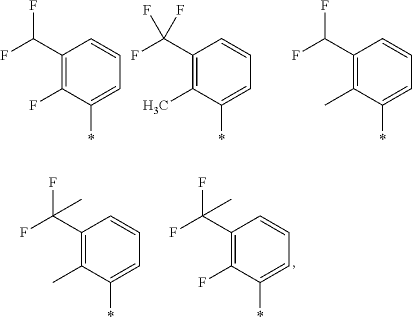
and
R₃ is

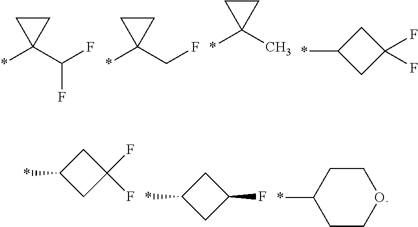

20. The compound of claim 1, wherein the compound is of formula (IB) 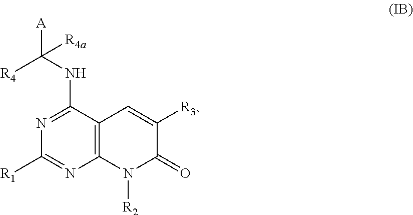
in which
R₁ is H, D, —CD₃, halogen, —CN, —NHR₁ₐ, —OR₁ₐ, C₁₋₃ alkyl, cyclopropyl, or C₂₋₃ alkenyl, R₁ₐ being H, C₁₋₃ alkyl, C₂₋₃ alkenyl, or cyclopropyl;
R₂ is H, —CD₃, C₁₋₄ alkyl, C₃₋₆ cycloalkyl, or C₁₋₆ heterocycloalkyl; and
R₅ is C₁₋₁₀ alkyl, C₃₋₁₀ cycloalkyl, C₁₋₁₀ heterocycloalkyl, C₂₋₁₀ alkenyl, C₂₋₁₀ alkynyl, aryl, or heteroaryl.

21. The compound of claim 20, wherein R₂ is H, —CD₃, C₁₋₄ alkyl, or C₃₋₆ cycloalkyl.

22. The compound of claim 21, wherein R₁ is H, —CD₃, or methyl and R₂ is H, methyl, or cyclopropyl.

23. The compound of claim 20, wherein L₁ is a bond, —NR₆—, —O—, —NR₆CO—, or —CO—NR₆—, in which R₆ is H, C₁₋₃ alkyl, or C₃₋₆ cycloalkyl, and R₅ is C₁₋₁₀ alkyl, C₃₋₁₀ cycloalkyl, C₁₋₁₀ heterocycloalkyl, aryl, or heteroaryl.

24. The compound of claim 23, wherein R₃ is 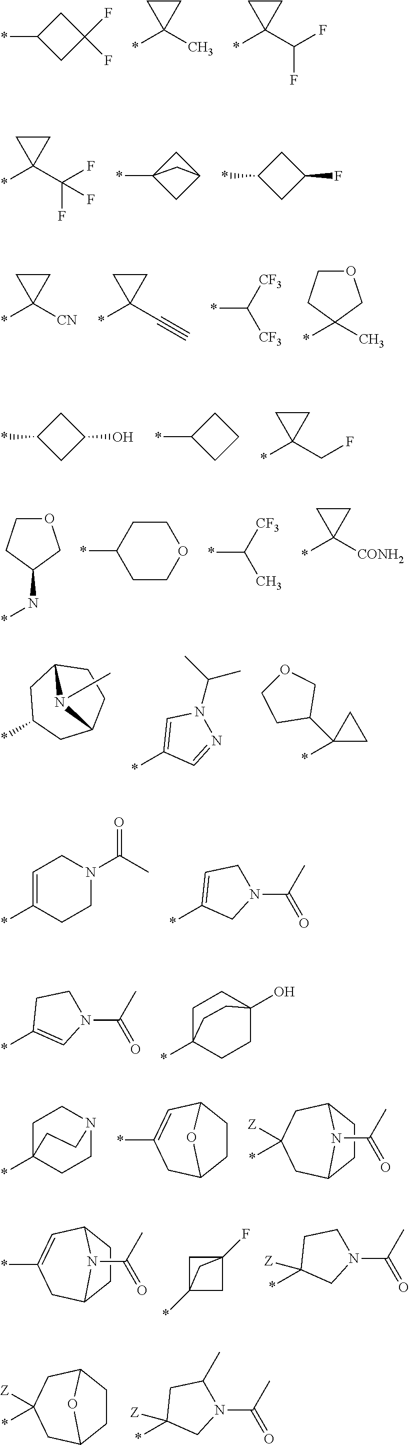 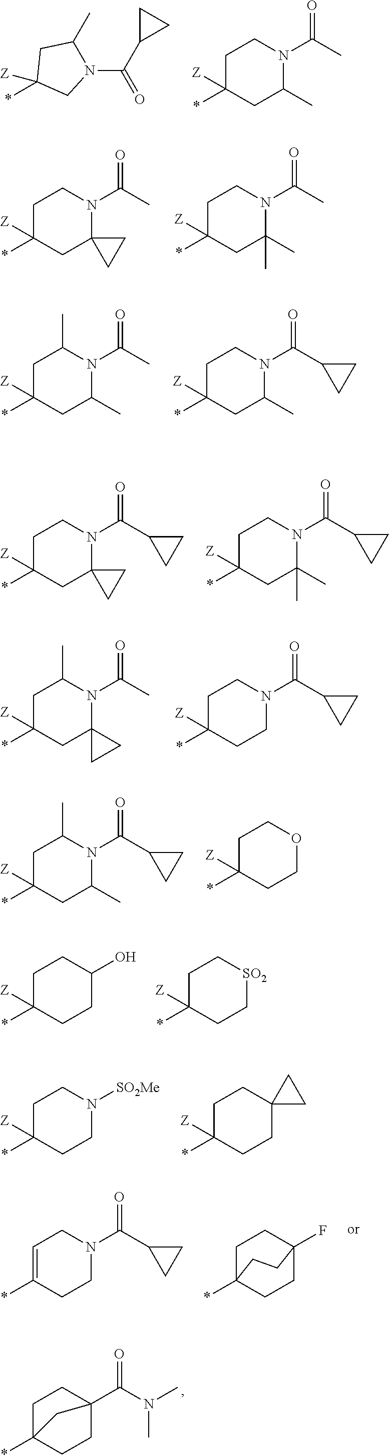
Z being H, methyl, methoxy, F, —OH, —CN, —NH₂, or —NH—COCH₃.

25. The compound of claim 24, wherein R₃ is 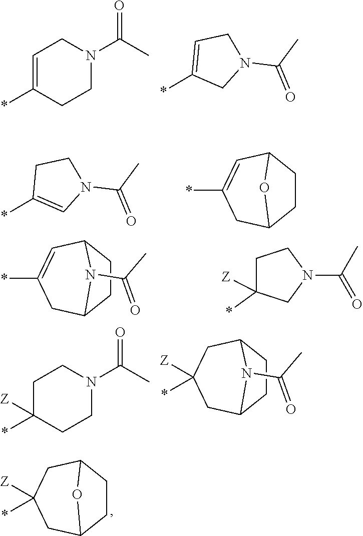
Z being OH, methoxy, F, —CN, NH₂, —NH—COCH₃.

26. The compound of claim 20, wherein R₄ is

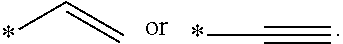

27. The compound of claim 26, wherein R₄ is

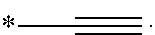 [SMILES: *C#C]

28. The compound of claim 24, wherein A is phenyl or phenyl fused with a C₄₋₆ cycloalkyl, C₁₋₆ heterocycloalkyl, or C₁₋₆ heteroaryl, each of which is optionally substituted with one or more moieties selected from the group consisting of halogens, —OH, —NH₂, —CN, methyl, ethyl, C₁₋₃ alkoxy, C₁₋₃ haloalkoxy, fluoromethyl, difluoromethyl, trifluoromethyl, fluoroethyl, difluoroethyl, trifluoroethyl,

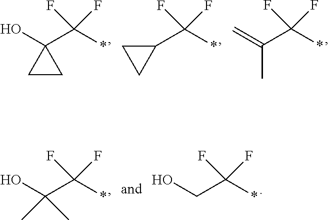

29. The compound of claim 28, wherein A is

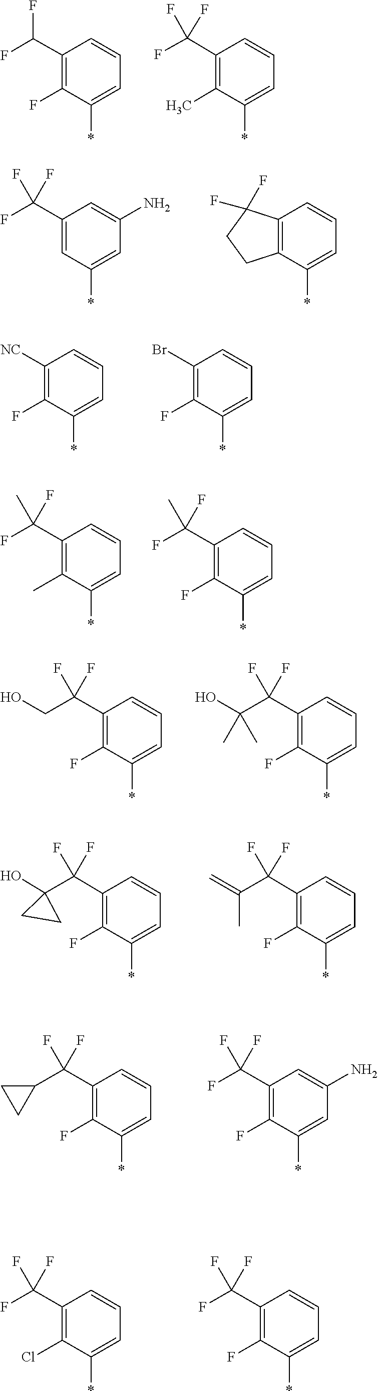

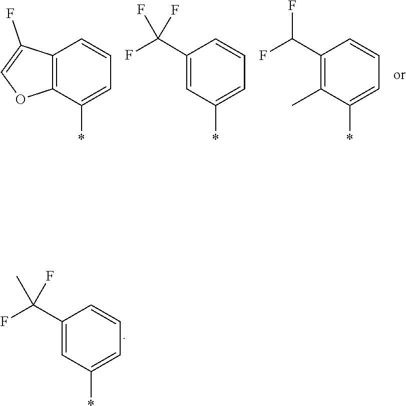

30. The compound of claim 29, wherein
A is 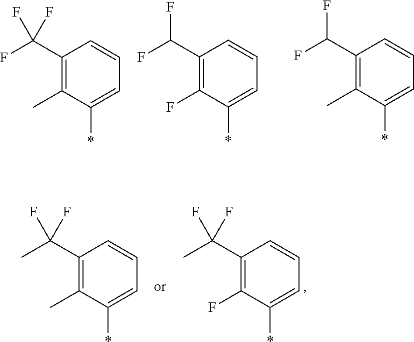
and
R₃ is 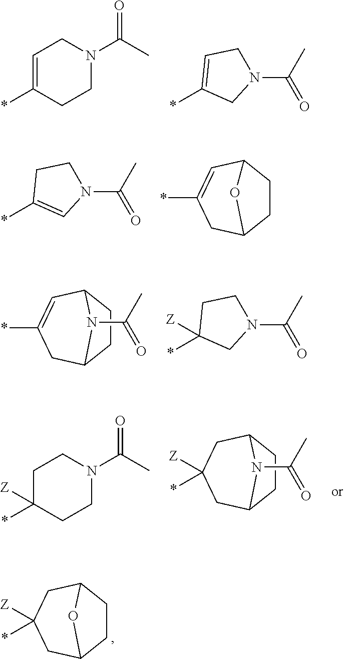
Z being OH, methoxy, F, —CN, NH₂, —NH—COCH₃.

31. The compound of claim 1, wherein the compound is one of Compounds 1-69.

32. The compound of claim 1, wherein the compound is one of Compounds 1, 4, 9, 24, and 25.

33. The compound of claim 1, wherein the compound is one of Compounds 40 and 66-69.

34. A method of treating cancer comprising administering to a subject in need thereof an effective amount of a compound of claim 1.

35. A method of inhibiting SOS1 comprising administering to a subject in need thereof an effective amount of a compound of claim 1.

36. A pharmaceutical composition comprising a compound of claim 1 and a pharmaceutically acceptable carrier thereof.
